SEQUENCE LISTING (115 sequences)

SEQ ID NO: 1 - protein, 10 aa, synthetic construct
  REGION 1..10: note = Synthetic Construct
YSISSGYYWG

SEQ ID NO: 2 - protein, 16 aa, synthetic construct
  REGION 1..16: note = Synthetic Construct
TIYHSGSTYYNPSLKS

SEQ ID NO: 3 - protein, 16 aa, synthetic construct
  REGION 1..16: note = Synthetic Construct
TIYHSGSTYYNPSLES

SEQ ID NO: 4 - protein, 16 aa, synthetic construct
  REGION 1..16: note = Synthetic Construct
  VARIANT 15: note = Xaa = K or E
TIYHSGSTYYNPSLXS

SEQ ID NO: 5 - protein, 15 aa, synthetic construct
  REGION 1..15: note = Synthetic Construct
ARQGSIQQGYYGMDV

SEQ ID NO: 6 - protein, 15 aa, synthetic construct
  REGION 1..15: note = Synthetic Construct
ARQGSIKQGYYGMDV

SEQ ID NO: 7 - protein, 15 aa, synthetic construct
  REGION 1..15: note = Synthetic Construct
  VARIANT 7: note = Xaa = Q or K
ARQGSIXQGYYGMDV

SEQ ID NO: 8 - protein, 16 aa, synthetic construct
  REGION 1..16: note = Synthetic Construct
RSSQSLLRSNGYNYLD

SEQ ID NO: 9 - protein, 16 aa, synthetic construct
  REGION 1..16: note = Synthetic Construct
RSSQSLLRSTGYNYLD

SEQ ID NO: 10 - protein, 16 aa, synthetic construct
  REGION 1..16: note = Synthetic Construct
RSSQSLLRSSGYNYLD

SEQ ID NO: 11 - protein, 16 aa, synthetic construct
  REGION 1..16: note = Synthetic Construct
RSSQSLLRSGGYNYLD

SEQ ID NO: 12 - protein, 16 aa, synthetic construct
  REGION 1..16: note = Synthetic Construct
RSSQSLLRSRGYNYLD

SEQ ID NO: 13 - protein, 16 aa, synthetic construct
  REGION 1..16: note = Synthetic Construct
RSSQSLLRSDGYNYLD

SEQ ID NO: 14 - protein, 16 aa, synthetic construct
  REGION 1..16: note = Synthetic Construct
RSSQSLLRSHGYNYLD

SEQ ID NO: 15 - protein, 16 aa, synthetic construct
  REGION 1..16: note = Synthetic Construct
RSSQSLLRSKGYNYLD

SEQ ID NO: 16 - protein, 16 aa, synthetic construct
  REGION 1..16: note = Synthetic Construct
RSSQSLLRSQGYNYLD

SEQ ID NO: 17 - protein, 16 aa, synthetic construct
  REGION 1..16: note = Synthetic Construct
RSSQSLLRSYGYNYLD

SEQ ID NO: 18 - protein, 16 aa, synthetic construct
  REGION 1..16: note = Synthetic Construct
RSSQSLLRSEGYNYLD

SEQ ID NO: 19 - protein, 16 aa, synthetic construct
  REGION 1..16: note = Synthetic Construct
RSSQSLLRSWGYNYLD

SEQ ID NO: 20 - protein, 16 aa, synthetic construct
  REGION 1..16: note = Synthetic Construct
RSSQSLLRSFGYNYLD

SEQ ID NO: 21 - protein, 16 aa, synthetic construct
  REGION 1..16: note = Synthetic Construct
RSSQSLLRSIGYNYLD

SEQ ID NO: 22 - protein, 16 aa, synthetic construct
  REGION 1..16: note = Synthetic Construct
RSSQSLLRSVGYNYLD

SEQ ID NO: 23 - protein, 16 aa, synthetic construct
  REGION 1..16: note = Synthetic Construct
RSSQSLLRSAGYNYLD

SEQ ID NO: 24 - protein, 16 aa, synthetic construct
  REGION 1..16: note = Synthetic Construct
RSSQSLLRSMGYNYLD

SEQ ID NO: 25 - protein, 16 aa, synthetic construct
  REGION 1..16: note = Synthetic Construct
RSSQSLLRSLGYNYLD

SEQ ID NO: 26 - protein, 16 aa, synthetic construct
  REGION 1..16: note = Synthetic Construct
RSSQSLLHSNGYNYLD

SEQ ID NO: 27 - protein, 16 aa, synthetic construct
  REGION 1..16: note = Synthetic Construct
RSSQGLLRSNGYNYLD

SEQ ID NO: 28 - protein, 16 aa, synthetic construct
  REGION 1..16: note = Synthetic Construct
  VARIANT 5: note = Xaa = S or G
  VARIANT 8: note = Xaa = R or H
  VARIANT 10: note = Xaa = N, T, S, G, R, D, H, K, Q, Y, E, W, F, I, V, A, M, or L
RSSQXLLXSXGYNYLD

SEQ ID NO: 29 - protein, 7 aa, synthetic construct
  REGION 1..7: note = Synthetic Construct
LGSNRAS

SEQ ID NO: 30 - protein, 7 aa, synthetic construct
  REGION 1..7: note = Synthetic Construct
LGSNRVS

SEQ ID NO: 31 - protein, 7 aa, synthetic construct
  REGION 1..7: note = Synthetic Construct
  VARIANT 6: note = Xaa = A or V
LGSNRXS

SEQ ID NO: 32 - protein, 9 aa, synthetic construct
  REGION 1..9: note = Synthetic Construct
MQQQEAPLT

SEQ ID NO: 33 - protein, 9 aa, synthetic construct
  REGION 1..9: note = Synthetic Construct
MQQQETPLT

SEQ ID NO: 34 - protein, 9 aa, synthetic construct
  REGION 1..9: note = Synthetic Construct
  VARIANT 6: note = Xaa = A or T
MQQQEXPLT

SEQ ID NO: 35 - protein, 26 aa, synthetic construct
  REGION 1..26: note = Synthetic Construct
QVQLQESGPGLVKPSETLSLTCAVSG

SEQ ID NO: 36 - protein, 14 aa, synthetic construct
  REGION 1..14: note = Synthetic Construct
WIRQPPGKGLEWIG

SEQ ID NO: 37 - protein, 30 aa, synthetic construct
  REGION 1..30: note = Synthetic Construct
QVTISVDTSKNQFSLELSSVTAADTAVYYC

SEQ ID NO: 38 - protein, 30 aa, synthetic construct
  REGION 1..30: note = Synthetic Construct
RVTISVDTSKNQFSLKLSSVTAADTAVYYC

SEQ ID NO: 39 - protein, 30 aa, synthetic construct
  REGION 1..30: note = Synthetic Construct
  VARIANT 1: note = Xaa = Q or R
  VARIANT 16: note = Xaa = E or K
XVTISVDTSKNQFSLXLSSVTAADTAVYYC

SEQ ID NO: 40 - protein, 11 aa, synthetic construct
  REGION 1..11: note = Synthetic Construct
WGQGTTVTVSS

SEQ ID NO: 41 - protein, 23 aa, synthetic construct
  REGION 1..23: note = Synthetic Construct
DIVMTQSPLSLPVTPGEPASISC

SEQ ID NO: 42 - protein, 23 aa, synthetic construct
  REGION 1..23: note = Synthetic Construct
DIVMTQSPLSLPVTPGGPASISC

SEQ ID NO: 43 - protein, 23 aa, synthetic construct
  REGION 1..23: note = Synthetic Construct
DIVMTQSPLSLPVTPGESASISC

SEQ ID NO: 44 - protein, 23 aa, synthetic construct
  REGION 1..23: note = Synthetic Construct
  VARIANT 17: note = Xaa = E or G
  VARIANT 18: note = Xaa = P or S
DIVMTQSPLSLPVTPGXXASISC

SEQ ID NO: 45 - protein, 15 aa, synthetic construct
  REGION 1..15: note = Synthetic Construct
WYLQKPGQSPQLLIY

SEQ ID NO: 46 - protein, 15 aa, synthetic construct
  REGION 1..15: note = Synthetic Construct
WYLQKPGQPPQLLIY

SEQ ID NO: 47 - protein, 15 aa, synthetic construct
  REGION 1..15: note = Synthetic Construct
  VARIANT 9: note = Xaa = S or P
WYLQKPGQXPQLLIY

SEQ ID NO: 48 - protein, 32 aa, synthetic construct
  REGION 1..32: note = Synthetic Construct
GVPDRFSGSGSGTDFTLKISRAEAEDVGVYYC

SEQ ID NO: 49 - protein, 32 aa, synthetic construct
  REGION 1..32: note = Synthetic Construct
GVPDRFSGSGSGTDFTLKISRVEAEDVGVYYC

SEQ ID NO: 50 - protein, 32 aa, synthetic construct
  REGION 1..32: note = Synthetic Construct
GVPDRFSGSGSGTDFTLKISRVEAEDVGAYYC

SEQ ID NO: 51 - protein, 32 aa, synthetic construct
  REGION 1..32: note = Synthetic Construct
GVPDRLSGSGSGTDFTLKISRVEAEDVGVYYC

SEQ ID NO: 52 - protein, 32 aa, synthetic construct
  REGION 1..32: note = Synthetic Construct
  VARIANT 6: note = Xaa = F or L
  VARIANT 22: note = Xaa = A or V
  VARIANT 29: note = Xaa = V or A
GVPDRXSGSGSGTDFTLKISRXEAEDVGXYYC

SEQ ID NO: 53 - protein, 10 aa, synthetic construct
  REGION 1..10: note = Synthetic Construct
FGGGTKVEIK

SEQ ID NO: 54 - protein, 122 aa, synthetic construct
  REGION 1..122: note = Synthetic Construct
QVQLQESGPGLVKPSETLSLTCAVSGYSISSGYYWGWIRQPPGKGLEWIGTIYHSGSTYYNPSLKSQVTISVDTSKNQFSLELSSVTAADTAVYYCARQGSIQQGYYGMDVWGQGTTVTVSS

SEQ ID NO: 55 - protein, 122 aa, synthetic construct
  REGION 1..122: note = Synthetic Construct
QVQLQESGPGLVKPSETLSLTCAVSGYSISSGYYWGWIRQPPGKGLEWIGTIYHSGSTYYNPSLESRVTISVDTSKNQFSLKLSSVTAADTAVYYCARQGSIQQGYYGMDVWGQGTTVTVSS

SEQ ID NO: 56 - protein, 122 aa, synthetic construct
  REGION 1..122: note = Synthetic Construct
QVQLQESGPGLVKPSETLSLTCAVSGYSISSGYYWGWIRQPPGKGLEWIGTIYHSGSTYYNPSLKSRVTISVDTSKNQFSLKLSSVTAADTAVYYCARQGSIKQGYYGMDVWGQGTTVTVSS

SEQ ID NO: 57 - protein, 112 aa, synthetic construct
  REGION 1..112: note = Synthetic Construct
DIVMTQSPLSLPVTPGEPASISCRSSQSLLRSNGYNYLDWYLQKPGQSPQLLIYLGSNRASGVPDRFSGSGSGTDFTLKISRAEAEDVGVYYCMQQQEAPLTFGGGTKVEIK

SEQ ID NO: 58 - protein, 112 aa, synthetic construct
  REGION 1..112: note = Synthetic Construct
DIVMTQSPLSLPVTPGEPASISCRSSQSLLRSNGYNYLDWYLQKPGQSPQLLIYLGSNRASGVPDRFSGSGSGTDFTLKISRVEAEDVGVYYCMQQQEAPLTFGGGTKVEIK

SEQ ID NO: 59 - protein, 112 aa, synthetic construct
  REGION 1..112: note = Synthetic Construct
DIVMTQSPLSLPVTPGEPASISCRSSQSLLRSNGYNYLDWYLQKPGQPPQLLIYLGSNRVSGVPDRFSGSGSGTDFTLKISRVEAEDVGVYYCMQQQETPLTFGGGTKVEIK

SEQ ID NO: 60 - protein, 112 aa, synthetic construct
  REGION 1..112: note = Synthetic Construct
DIVMTQSPLSLPVTPGEPASISCRSSQSLLRSTGYNYLDWYLQKPGQSPQLLIYLGSNRASGVPDRFSGSGSGTDFTLKISRAEAEDVGVYYCMQQQEAPLTFGGGTKVEIK

SEQ ID NO: 61 - protein, 112 aa, synthetic construct
  REGION 1..112: note = Synthetic Construct
DIVMTQSPLSLPVTPGEPASISCRSSQSLLRSSGYNYLDWYLQKPGQSPQLLIYLGSNRASGVPDRFSGSGSGTDFTLKISRAEAEDVGVYYCMQQQEAPLTFGGGTKVEIK

SEQ ID NO: 62 - protein, 112 aa, synthetic construct
  REGION 1..112: note = Synthetic Construct
DIVMTQSPLSLPVTPGEPASISCRSSQSLLRSGGYNYLDWYLQKPGQSPQLLIYLGSNRASGVPDRFSGSGSGTDFTLKISRAEAEDVGVYYCMQQQEAPLTFGGGTKVEIK

SEQ ID NO: 63 - protein, 112 aa, synthetic construct
  REGION 1..112: note = Synthetic Construct
DIVMTQSPLSLPVTPGEPASISCRSSQSLLRSRGYNYLDWYLQKPGQSPQLLIYLGSNRASGVPDRFSGSGSGTDFTLKISRAEAEDVGVYYCMQQQEAPLTFGGGTKVEIK

SEQ ID NO: 64 - protein, 112 aa, synthetic construct
  REGION 1..112: note = Synthetic Construct
DIVMTQSPLSLPVTPGEPASISCRSSQSLLRSDGYNYLDWYLQKPGQSPQLLIYLGSNRASGVPDRFSGSGSGTDFTLKISRAEAEDVGVYYCMQQQEAPLTFGGGTKVEIK

SEQ ID NO: 65 - protein, 112 aa, synthetic construct
  REGION 1..112: note = Synthetic Construct
DIVMTQSPLSLPVTPGEPASISCRSSQSLLRSHGYNYLDWYLQKPGQSPQLLIYLGSNRASGVPDRFSGSGSGTDFTLKISRAEAEDVGVYYCMQQQEAPLTFGGGTKVEIK

SEQ ID NO: 66 - protein, 112 aa, synthetic construct
  REGION 1..112: note = Synthetic Construct
DIVMTQSPLSLPVTPGEPASISCRSSQSLLRSKGYNYLDWYLQKPGQSPQLLIYLGSNRASGVPDRFSGSGSGTDFTLKISRAEAEDVGVYYCMQQQEAPLTFGGGTKVEIK

SEQ ID NO: 67 - protein, 112 aa, synthetic construct
  REGION 1..112: note = Synthetic Construct
DIVMTQSPLSLPVTPGEPASISCRSSQSLLRSQGYNYLDWYLQKPGQSPQLLIYLGSNRASGVPDRFSGSGSGTDFTLKISRAEAEDVGVYYCMQQQEAPLTFGGGTKVEIK

SEQ ID NO: 68 - protein, 112 aa, synthetic construct
  REGION 1..112: note = Synthetic Construct
DIVMTQSPLSLPVTPGEPASISCRSSQSLLRSYGYNYLDWYLQKPGQSPQLLIYLGSNRASGVPDRFSGSGSGTDFTLKISRAEAEDVGVYYCMQQQEAPLTFGGGTKVEIK

SEQ ID NO: 69 - protein, 112 aa, synthetic construct
  REGION 1..112: note = Synthetic Construct
DIVMTQSPLSLPVTPGEPASISCRSSQSLLRSEGYNYLDWYLQKPGQSPQLLIYLGSNRASGVPDRFSGSGSGTDFTLKISRAEAEDVGVYYCMQQQEAPLTFGGGTKVEIK

SEQ ID NO: 70 - protein, 112 aa, synthetic construct
  REGION 1..112: note = Synthetic Construct
DIVMTQSPLSLPVTPGEPASISCRSSQSLLRSWGYNYLDWYLQKPGQSPQLLIYLGSNRASGVPDRFSGSGSGTDFTLKISRAEAEDVGVYYCMQQQEAPLTFGGGTKVEIK

SEQ ID NO: 71 - protein, 112 aa, synthetic construct
  REGION 1..112: note = Synthetic Construct
DIVMTQSPLSLPVTPGEPASISCRSSQSLLRSFGYNYLDWYLQKPGQSPQLLIYLGSNRASGVPDRFSGSGSGTDFTLKISRAEAEDVGVYYCMQQQEAPLTFGGGTKVEIK

SEQ ID NO: 72 - protein, 112 aa, synthetic construct
  REGION 1..112: note = Synthetic Construct
DIVMTQSPLSLPVTPGEPASISCRSSQSLLRSIGYNYLDWYLQKPGQSPQLLIYLGSNRASGVPDRFSGSGSGTDFTLKISRAEAEDVGVYYCMQQQEAPLTFGGGTKVEIK

SEQ ID NO: 73 - protein, 112 aa, synthetic construct
  REGION 1..112: note = Synthetic Construct
DIVMTQSPLSLPVTPGEPASISCRSSQSLLRSVGYNYLDWYLQKPGQSPQLLIYLGSNRASGVPDRFSGSGSGTDFTLKISRAEAEDVGVYYCMQQQEAPLTFGGGTKVEIK

SEQ ID NO: 74 - protein, 112 aa, synthetic construct
  REGION 1..112: note = Synthetic Construct
DIVMTQSPLSLPVTPGEPASISCRSSQSLLRSAGYNYLDWYLQKPGQSPQLLIYLGSNRASGVPDRFSGSGSGTDFTLKISRAEAEDVGVYYCMQQQEAPLTFGGGTKVEIK

SEQ ID NO: 75 - protein, 112 aa, synthetic construct
  REGION 1..112: note = Synthetic Construct
DIVMTQSPLSLPVTPGEPASISCRSSQSLLRSMGYNYLDWYLQKPGQSPQLLIYLGSNRASGVPDRFSGSGSGTDFTLKISRAEAEDVGVYYCMQQQEAPLTFGGGTKVEIK

SEQ ID NO: 76 - protein, 112 aa, synthetic construct
  REGION 1..112: note = Synthetic Construct
DIVMTQSPLSLPVTPGEPASISCRSSQSLLRSLGYNYLDWYLQKPGQSPQLLIYLGSNRASGVPDRFSGSGSGTDFTLKISRAEAEDVGVYYCMQQQEAPLTFGGGTKVEIK

SEQ ID NO: 77 - protein, 112 aa, synthetic construct
  REGION 1..112: note = Synthetic Construct
DIVMTQSPLSLPVTPGEPASISCRSSQSLLRSNGYNYLDWYLQKPGQSPQLLIYLGSNRASGVPDRFSGSGSGTDFTLKISRVEAEDVGAYYCMQQQEAPLTFGGGTKVEIK

SEQ ID NO: 78 - protein, 112 aa, synthetic construct
  REGION 1..112: note = Synthetic Construct
DIVMTQSPLSLPVTPGGPASISCRSSQSLLRSNGYNYLDWYLQKPGQSPQLLIYLGSNRASGVPDRLSGSGSGTDFTLKISRVEAEDVGVYYCMQQQETPLTFGGGTKVEIK

SEQ ID NO: 79 - protein, 112 aa, synthetic construct
  REGION 1..112: note = Synthetic Construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQQQETPLTFGGGTKVEIK

SEQ ID NO: 80 - protein, 112 aa, synthetic construct
  REGION 1..112: note = Synthetic Construct
DIVMTQSPLSLPVTPGESASISCRSSQGLLRSNGYNYLDWYLQKPGQSPQLLIYLGSNRASGVPDRFSGSGSGTDFTLKISRVEAEDVGVYYCMQQQEAPLTFGGGTKVEIK

SEQ ID NO: 81 - protein, 831 aa, Homo sapiens
MERPWGAADGLSRWPHGLGLLLLLQLLPPSTLSQDRLDAPPPPAAPLPRWSGPIGVSWGLRAAAAGGAFPRGGRWRRSAPGEDEECGRVRDFVAKLANNTHQHVFDDLRGSVSLSWVGDSTGVILVLTTFHVPLVIMTFGQSKLYRSEDYGKNFKDITDLINNTFIRTEFGMAIGPENSGKVVLTAEVSGGSRGGRIFRSSDFAKNFVQTDLPFHPLTQMMYSPQNSDYLLALSTENGLWVSKNFGGKWEEIHKAVCLAKWGSDNTIFFTTYANGSCKADLGALELWRTSDLGKSFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSVLSEDNSIQTMITFDQGGRWTHLRKPENSECDATAKNKNECSLHIHASYSISQKLNVPMAPLSEPNAVGIVIAHGSVGDAISVMVPDVYISDDGGYSWTKMLEGPHYYTILDSGGIIVAIEHSSRPINVIKFSTDEGQCWQTYTFTRDPIYFTGLASEPGARSMNISIWGFTESFLTSQWVSYTIDFKDILERNCEEKDYTIWLAHSTDPEDYEDGCILGYKEQFLRLRKSSVCQNGRDYVVTKQPSICLCSLEDFLCDFGYYRPENDSKCVEQPELKGHDLEFCLYGREEHLTTNGYRKIPGDKCQGGVNPVREVKDLKKKCTSNFLSPEKQNSKSNSVPIILAIVGLMLVTVVAGVLIVKKYVCGGRFLVHRYSVLQQHAEANGVDGVDALDTASHTNKSGYHDDSDEDLLE

SEQ ID NO: 82 - protein, 825 aa, Mus musculus
MERPRGAADGLLRWPLGLLLLLQLLPPAAVGQDRLDAPPPPAPPLLRWAGPVGVSWGLRAAAPGGPVPRAGRWRRGAPAEDQDCGRLPDFIAKLTNNTHQHVFDDLSGSVSLSWVGDSTGVILVLTTFQVPLVIVSFGQSKLYRSEDYGKNFKDITNLINNTFIRTEFGMAIGPENSGKVILTAEVSGGSRGGRVFRSSDFAKNFVQTDLPFHPLTQMMYSPQNSDYLLALSTENGLWVSKNFGEKWEEIHKAVCLAKWGPNNIIFFTTHVNGSCKADLGALELWRTSDLGKTFKTIGVKIYSFGLGGRFLFASVMADKDTTRRIHVSTDQGDTWSMAQLPSVGQEQFYSILAANEDMVFMHVDEPGDTGFGTIFTSDDRGIVYSKSLDRHLYTTTGGETDFTNVTSLRGVYITSTLSEDNSIQSMITFDQGGRWEHLRKPENSKCDATAKNKNECSLHIHASYSISQKLNVPMAPLSEPNAVGIVIAHGSVGDAISVMVPDVYISDDGGYSWAKMLEGPHYYTILDSGGIIVAIEHSNRPINVIKFSTDEGQCWQSYVFTQEPIYFTGLASEPGARSMNISIWGFTESFITRQWVSYTVDFKDILERNCEEDDYTTWLAHSTDPGDYKDGCILGYKEQFLRLRKSSVCQNGRDYVVAKQPSVCPCSLEDFLCDFGYFRPENASECVEQPELKGHELEFCLYGKEEHLTTNGYRKIPGDKCQGGMNPAREVKDLKKKCTSNFLNPTKQNSKSNSVPIILAIVGLMLVTVVAGVLIVKKYVCGGRFLVHRYSVLQQHAEADGVEALDSTSHAKSGYHDDSDEDLLE

SEQ ID NO: 83 - protein, 825 aa, Rattus norvegicus
MERPRGAADGLLRWPLGLLLLLQLLPPAAVGQDRLDAPPPPAPPLLRWAGPVGVSWGLRAAAPGGPVPRAGRWRRGAPAEDQDCGRLPDFIAKLTNNTHQHVFDDLSGSVSLSWVGDSTGVILVLTTFQVPLVIVSFGQSKLYRSEDYGKNFKDITNLINNTFIRTEFGMAIGPENSGKVILTAEVSGGSRGGRVFRSSDFAKNFVQTDLPFHPLTQMMYSPQNSDYLLALSTENGLWVSKNFGEKWEEIHKAVCLAKWGPNNIIFFTTHVNGSCKADLGALELWRTSDLGKTFKTIGVKIYSFGLGGRFLFASVMADKDTTRRIHVSTDQGDTWSMAQLPSVGQEQFYSILAANDDMVFMHVDEPGDTGFGTIFTSDDRGIVYSKSLDRHLYTTTGGETDFTNVTSLRGVYITSTLSEDNSIQSMITFDQGGRWEHLQKPENSKCDATAKNKNECSLHIHASYSISQKLNVPMAPLSEPNAVGIVIAHGSVGDAISVMVPDVYISDDGGYSWAKMLEGPHYYTILDSGGIIVAIEHSNRPINVIKFSTDEGQCWQSYVFSQEPVYFTGLASEPGARSMNISIWGFTESFLTRQWVSYTIDFKDILERNCEENDYTTWLAHSTDPGDYKDGCILGYKEQFLRLRKSSVCQNGRDYVVAKQPSICPCSLEDFLCDFGYFRPENASECVEQPELKGHELEFCLYGKEEHLTTNGYRKIPGDRCQGGMNPAREVKDLKKKCTSNFLNPKKQNSKSSSVPIILAIVGLMLVTVVAGVLIVKKYVCGGRFLVHRYSVLQQHAEADGVEALDTASHAKSGYHDDSDEDLLE

SEQ ID NO: 84 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 86 - protein, 452 aa, synthetic construct
  REGION 1..452: note = Synthetic Construct
QVQLQESGPGLVKPSETLSLTCAVSGYSISSGYYWGWIRQPPGKGLEWIGTIYHSGSTYYNPSLKSQVTISVDTSKNQFSLELSSVTAADTAVYYCARQGSIQQGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 451 aa, synthetic construct
  REGION 1..451: note = Synthetic Construct
QVQLQESGPGLVKPSETLSLTCAVSGYSISSGYYWGWIRQPPGKGLEWIGTIYHSGSTYYNPSLKSQVTISVDTSKNQFSLELSSVTAADTAVYYCARQGSIQQGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 88 - protein, 452 aa, synthetic construct
  REGION 1..452: note = Synthetic Construct
QVQLQESGPGLVKPSETLSLTCAVSGYSISSGYYWGWIRQPPGKGLEWIGTIYHSGSTYYNPSLESRVTISVDTSKNQFSLKLSSVTAADTAVYYCARQGSIQQGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 89 - protein, 451 aa, synthetic construct
  REGION 1..451: note = Synthetic Construct
QVQLQESGPGLVKPSETLSLTCAVSGYSISSGYYWGWIRQPPGKGLEWIGTIYHSGSTYYNPSLESRVTISVDTSKNQFSLKLSSVTAADTAVYYCARQGSIQQGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 90 - protein, 452 aa, synthetic construct
  REGION 1..452: note = Synthetic Construct
QVQLQESGPGLVKPSETLSLTCAVSGYSISSGYYWGWIRQPPGKGLEWIGTIYHSGSTYYNPSLKSRVTISVDTSKNQFSLKLSSVTAADTAVYYCARQGSIKQGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 91 - protein, 451 aa, synthetic construct
  REGION 1..451: note = Synthetic Construct
QVQLQESGPGLVKPSETLSLTCAVSGYSISSGYYWGWIRQPPGKGLEWIGTIYHSGSTYYNPSLKSRVTISVDTSKNQFSLKLSSVTAADTAVYYCARQGSIKQGYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 92 - protein, 219 aa, synthetic construct
  REGION 1..219: note = Synthetic Construct
DIVMTQSPLSLPVTPGEPASISCRSSQSLLRSNGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 93 - protein, 219 aa, synthetic construct
  REGION 1..219: note = Synthetic Construct
DIVMTQSPLSLPVTPGEPASISCRSSQSLLRSNGYNYLDWYLQKPGQSPQLLIYLGSNRASGVPDRFSGSGSGTDFTLKISRVEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - protein, 219 aa, synthetic construct
  REGION 1..219: note = Synthetic Construct
DIVMTQSPLSLPVTPGEPASISCRSSQSLLRSNGYNYLDWYLQKPGQPPQLLIYLGSNRVSGVPDRFSGSGSGTDFTLKISRVEAEDVGVYYCMQQQETPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 95 - protein, 219 aa, synthetic construct
  REGION 1..219: note = Synthetic Construct
DIVMTQSPLSLPVTPGEPASISCRSSQSLLRSTGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 96 - protein, 219 aa, synthetic construct
  REGION 1..219: note = Synthetic Construct
DIVMTQSPLSLPVTPGEPASISCRSSQSLLRSSGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 97 - protein, 219 aa, synthetic construct
  REGION 1..219: note = Synthetic Construct
DIVMTQSPLSLPVTPGEPASISCRSSQSLLRSGGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 98 - protein, 219 aa, synthetic construct
  REGION 1..219: note = Synthetic Construct
DIVMTQSPLSLPVTPGEPASISCRSSQSLLRSRGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 219 aa, synthetic construct
  REGION 1..219: note = Synthetic Construct
DIVMTQSPLSLPVTPGEPASISCRSSQSLLRSDGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 100 - protein, 219 aa, synthetic construct
  REGION 1..219: note = Synthetic Construct
DIVMTQSPLSLPVTPGEPASISCRSSQSLLRSHGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 219 aa, synthetic construct
  REGION 1..219: note = Synthetic Construct
DIVMTQSPLSLPVTPGEPASISCRSSQSLLRSKGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 102 - protein, 219 aa, synthetic construct
  REGION 1..219: note = Synthetic Construct
DIVMTQSPLSLPVTPGEPASISCRSSQSLLRSQGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 219 aa, synthetic construct
  REGION 1..219: note = Synthetic Construct
DIVMTQSPLSLPVTPGEPASISCRSSQSLLRSYGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 104 - protein, 219 aa, synthetic construct
  REGION 1..219: note = Synthetic Construct
DIVMTQSPLSLPVTPGEPASISCRSSQSLLRSEGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 105 - protein, 219 aa, synthetic construct
  REGION 1..219: note = Synthetic Construct
DIVMTQSPLSLPVTPGEPASISCRSSQSLLRSWGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 106 - protein, 219 aa, synthetic construct
  REGION 1..219: note = Synthetic Construct
DIVMTQSPLSLPVTPGEPASISCRSSQSLLRSFGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 219 aa, synthetic construct
  REGION 1..219: note = Synthetic Construct
DIVMTQSPLSLPVTPGEPASISCRSSQSLLRSIGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 108 - protein, 219 aa, synthetic construct
  REGION 1..219: note = Synthetic Construct
DIVMTQSPLSLPVTPGEPASISCRSSQSLLRSVGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 219 aa, synthetic construct
  REGION 1..219: note = Synthetic Construct
DIVMTQSPLSLPVTPGEPASISCRSSQSLLRSAGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 110 - protein, 219 aa, synthetic construct
  REGION 1..219: note = Synthetic Construct
DIVMTQSPLSLPVTPGEPASISCRSSQSLLRSMGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 219 aa, synthetic construct
  REGION 1..219: note = Synthetic Construct
DIVMTQSPLSLPVTPGEPASISCRSSQSLLRSLGYNYLDWYLQKPGQSPQLLIYLGSNRASGVPDRFSGSGSGTDFTLKISRAEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 219 aa, synthetic construct
  REGION 1..219: note = Synthetic Construct
DIVMTQSPLSLPVTPGEPASISCRSSQSLLRSNGYNYLDWYLQKPGQSPQLLIYLGSNRASGVPDRFSGSGSGTDFTLKISRVEAEDVGAYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 113 - protein, 219 aa, synthetic construct
  REGION 1..219: note = Synthetic Construct
DIVMTQSPLSLPVTPGGPASISCRSSQSLLRSNGYNYLDWYLQKPGQSPQLLIYLGSNRASGVPDRLSGSGSGTDFTLKISRVEAEDVGVYYCMQQQETPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 114 - protein, 219 aa, synthetic construct
  REGION 1..219: note = Synthetic Construct
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQQQETPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 219 aa, synthetic construct
  REGION 1..219: note = Synthetic Construct
DIVMTQSPLSLPVTPGESASISCRSSQGLLRSNGYNYLDWYLQKPGQSPQLLIYLGSNRASGVPDRFSGSGSGTDFTLKISRVEAEDVGVYYCMQQQEAPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

(19) United States
(12) Patent Application Publication
(10) Pub. No.: US 2024/0132597 A1
(43) Pub. Date: Apr. 25, 2024

(54) METHODS OF USE OF ANTI-SORTILIN ANTIBODIES

(71) Applicant: Alector LLC, South San Francisco, CA (US)

(72) Inventors: Robert PAUL, San Francisco, CA (US); Sam JACKSON, Oakland, CA (US); Omer Rizwan SIDDIQUI, Redwood City, CA (US); Michael F. WARD, San Francisco, CA (US); Felix Leejia YEH, Belmont, CA (US); Julie Y. HUANG, San Bruno, CA (US); Whedy WANG, Palo Alto, CA (US); Yijie LIAO, Pleasanton, CA (US); Brian C. MANGAL, North Vancouver (CA)

(73) Assignee: Alector LLC, South San Francisco, CA (US)

(21) Appl. No.: 18/532,382

(22) Filed: Dec. 7, 2023

Related U.S. Application Data
(63) Continuation of application No. PCT/US2022/072827, filed on Jun. 8, 2022.
(60) Provisional application No. 63/311,379, filed on Feb. 17, 2022.
(60) Provisional application No. 63/304,522, filed on Jan. 28, 2022.
(60) Provisional application No. 63/277,069, filed on Nov. 8, 2021.
(60) Provisional application No. 63/225,916, filed on Jul. 26, 2021.
(60) Provisional application No. 63/209,360, filed on Jun. 10, 2021.
(60) Provisional application No. 63/208,238, filed on Jun. 8, 2021.

(51) Int. Cl. C07K 16/28; A61P 25/28; G01N 33/68

(52) U.S. Cl. CPC C07K 16/286; A61P 25/28; G01N 33/6896; A61K 2039/505; G01N 2800/2814; G01N 2800/52

(57) ABSTRACT

The present disclosure is generally directed to the use of compositions that include antibodies, e.g., monoclonal, chimeric, affinity-matured or humanized antibodies, antibody fragments, etc., that specifically bind one or more epitopes within a Sortilin protein, e.g., human Sortilin or mammalian Sortilin, and have improved and/or enhanced functional characteristics, in treating and/or delaying progression of a disease or injury in an individual in need thereof.

### CROSS REFERENCE TO RELATED APPLICATIONS

This application claims the priority benefit of U.S. Provisional Application No. 63/208,238, filed Jun. 8, 2021, U.S. Provisional Application No. 63/209,360, filed Jun. 10, 2021, U.S. Provisional Application No. 63/225,916, filed Jul. 26, 2021, U.S. Provisional Application No. 63/277,069, filed Nov. 8, 2021, U.S. Provisional Application No. 63/304,522, filed Jan. 28, 2022, and U.S. Provisional Application No. 63/311,379, filed Feb. 17, 2022, each of which is incorporated herein by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 735022003740SEQLIST.TXT, date recorded: May 27, 2022, size: 137,728 bytes).

### FIELD

The present disclosure relates to therapeutic uses of anti-Sortilin antibodies.

### BACKGROUND

Sortilin (SORT1) is a Type I transmembrane protein that acts both as a receptor of several ligands, and in the sorting of select cargo from the trans-Golgi network (TGN) to late endosomes and lysosomes for degradation. Sortilin binds the secreted protein Progranulin (PGRN) and targets it for lysosomal degradation, thus negatively regulating extracellular levels of PGRN (Hu, F et al. (2010) Neuron 68, 654-667). In line with this, deficiency of Sortilin significantly increases plasma PGRN levels both in mouse models in vivo and human cells in vitro (Carrasquillo, M. M et al., (2010) Am J Hum Genet 87, 890-897; Lee, W. C et al., (2014) Hum Mol Genet 23, 1467-1478). Moroever, a polymorphism in Sortilin was shown to be strongly associated with PGRN serum levels in humans (Carrasquillo M M e al., (2010), Am J Hum Genet. 10; 87(6):890-7).

Progranulin (PGRN) is a secreted, growth factor-like, trophic, and anti-inflammatory protein, which also plays a role as an adipokine involved in diet-induced obesity and insulin resistance (Nguyen D A et al., (2013). Trends in Endocrinology and Metabolism, 24, 597-606). Progranulin deficiency accounts for roughly 25% of all heritable forms of frontotemporal dementia (FTD), an early-onset neurodegenerative disease. Patients with heterozygous loss-of-function mutations in PGRN have ˜50% reduced extracellular levels of the protein and invariably develop FTD, making PGRN a causal gene for the disease (Baker, M et al., (2006) Nature 442, 916-919; Carecchio M et al., (2011) J Alzheimers Dis 27, 781-790; Cruts, M et al., (2008) Trends Genet 24, 186-194; Galimberti, D et al., (2010) J Alzheimers Dis 19, 171-177). In addition, PGRN mutant alleles have been identified in Alzheimer's disease patients (Seelaar, H et al., (2011). Journal of neurology, neurosurgery, and psychiatry 82, 476-486). Importantly, PGRN acts protectively in several disease models, with increased PGRN levels accelerating behavioral recovery from ischemia (Tao, J et al., (2012) Brain Res 1436, 130-136; Egashira, Y. et al., (2013) J Neuroinflammation 10, 105), reducing TDP-43 aggregation and prolonging survival in a mouse model of TDP-43 pathology (Beel et al. (2018) Molecular Neurodegener 13(1):55), suppressing locomotor deficits in a Parkinson's disease model (Van Kampen, J. M et al. (2014). PLoS One 9, e97032), attenuating pathology in a model of amyotrophic lateral sclerosis (Laird, A. S et al., (2010). PLoS One 5, e13368) and arthritis (Tang, W et al., (2011). Science 332, 478-484), and preventing memory deficits in an Alzheimer's disease model (Minami, S. S et al., (2014). Nat Med 20, 1157-1164).

Through its various interactions with proteins, such as Progranulin, Sortilin and its multiple ligands have been shown to be involved in various diseases, disorders, and conditions, such as frontotemporal dementia (FTD), amyotrophic lateral sclerosis (ALS), amyotrophic lateral sclerosis-frontotemporal dementia phenotypes, Alzheimer's disease, Parkinson's disease, depression, neuropsychiatric disorders, vascular dementia, seizures, retinal dystrophy, age related macular degeneration, glaucoma, traumatic brain injury, aging, seizures, wound healing, stroke, arthritis, and atherosclerotic vascular diseases.

Novel therapeutic antibodies targeting Sortilin are one solution to treating diseases associated with Sortilin activity. Systemically administered monoclonal antibodies normally exhibit a biphasic pharmacokinetic profile, being first distributed relatively quickly and then eliminated more slowly (Ovacik, M and Lin, L, (2018) Clin Transl Sci 11, 540-552). Circulation of systemically administered antibodies is typically confined to the vasculature and interstitial space (Ovacik, M and Lin, L, (2018) Clin Transl Sci 11, 540-552). This is because of their size, polarity, recycling and clearance kinetics, and typically relatively long half-lives, which are often 11-30 days in humans (Ovacik, M and Lin, L, (2018) Clin Transl Sci 11, 540-552).

Administration of monoclonal antibodies presents a challenge for therapeutic use. Monoclonal antibodies have limited oral bioavailability, so they are typically administered intravenously, subcutaneously, or intramuscularly (Ovacik, M and Lin, L, (2018) Clin Transl Sci 11, 540-552). Of those options, subcutaneous administration is the most convenient because it can be done at home and often by the patient himself, but intravenous administration delivers higher systemic exposures. Delivery to the cerebrospinal fluid (CSF) requires high systemic doses. Thus, when treatment requires impacting the CSF, intravenous administration is usually required because subcutaneous administration cannot deliver sufficiently high doses.

However, intravenous administration is particularly challenging for patients with neurodegenerative diseases, such as FTD. These diseases affect patients for long periods of time and thus require regular treatment over the course of many years. As intravenous administration cannot be done at home, patients must be transported to infusion centers on a regular basis, which is a burden on both the patient and caregiver. Finally, the memory loss, mood swings, aggression, and other behavioral symptoms of these diseases make patient compliance difficult.

Accordingly, there is a need for therapeutic antibodies that specifically bind Sortilin proteins and block the binding of Sortilin to its ligands, such as Progranulin, or otherwise modulate the effective concentration of the ligands, in order to treat one or more diseases, disorders, and conditions associated with Sortilin activity. Furthermore, due to the limitations on modes of administration and dosing, there are additional needs for identifying methods of treating patients with the correct dose of the antibodies, and of administering that dose in ways that ease patient compliance.

All references cited herein, including patents, patent applications and publications, are hereby incorporated by reference in their entirety.

### SUMMARY

The present disclosure is generally directed to methods of using compositions that include antibodies, e.g., monoclonal, chimeric, humanized antibodies, antibody fragments, etc., that specifically bind human Sortilin.

In some aspects, provided herein are methods of treating and/or delaying the progression of a disease or injury in an individual, comprising administering to the individual an anti-Sortilin antibody intravenously at a dose of about 60 mg/kg about once every four weeks, wherein the antibody comprises: (i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33); (iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32); (vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33); (vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or (viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32).

In other aspects, provided herein are methods of treating and/or delaying the progression of frontotemporal dementia in an individual at risk for developing symptomatic frontotemporal dementia, comprising administering to the individual an anti-Sortilin antibody intravenously at a dose of about 60 mg/kg about once every four weeks, wherein the individual has an elevated serum neurofilament light chain level, and wherein the antibody comprises: (i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33); (iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32); (vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33); (vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or (viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32).

In other aspects, provided herein are methods of treating and/or delaying the progression of frontotemporal dementia in an individual at risk for developing symptomatic frontotemporal dementia, wherein the individual has a serum neurofilament light chain level of at least about 13.6 pg/mL or at least about 19.8 pg/mL, and further wherein the method comprises administering to the individual an anti-Sortilin antibody intravenously at a dose of about 60 mg/kg about once every four weeks, wherein the antibody comprises: (i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33); (iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32); (vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33); (vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or (viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32). In some embodiments, the methods further comprise assessing the serum neurofilament light chain level in the individual prior to administration of the anti-Sortilin antibody.

In another aspect, provided herein is an anti-Sortilin antibody for use in a method of treating and/or delaying the progression of frontotemporal dementia in an individual classified as being at risk for developing symptomatic frontotemporal dementia, wherein the method comprises: measuring a serum neurofilament light chain level of the individual; determining that the individual is at risk for developing symptomatic frontotemporal dementia if the individual has a serum neurofilament light chain level of at least about 13.6 pg/mL or at least about 19.8 pg/mL; administering to the individual the anti-Sortilin antibody intravenously at a dose of about 60 mg/kg about once every four weeks, wherein the anti-Sortilin antibody comprises: (i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33); (iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32); (vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33); (vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or (viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32).

In other aspects, provided herein are methods of treating and/or delaying the progression of frontotemporal dementia in an individual, comprising administering to the individual an anti-Sortilin antibody intravenously at a dose of about 60 mg/kg about once every four weeks, wherein the antibody comprises: (i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33); (iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32); (vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33); (vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or (viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32); and wherein administration of the anti-Sortilin antibody to the individual results in a reduction or delay of frontotemporal dementia disease progression of at least about 40% as compared to disease progression in a corresponding individual not treated with the anti-Sortilin antibody. In another aspect, provided herein is an anti-Sortilin antibody for use in methods of treating and/or delaying the progression of frontotemporal dementia in an individual, wherein the anti-Sortilin antibody is administered to the individual intravenously at a dose of about 60 mg/kg about once every four weeks, wherein the anti-sortilin antibody comprises: (i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33); (iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32); (vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33); (vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or (viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32); and wherein administration of the anti-Sortilin antibody to the individual results in a reduction or delay of frontotemporal dementia disease progression of at least about 40% as compared to disease progression in a corresponding individual not treated with the anti-Sortilin antibody. In some embodiments, frontotemporal dementia disease progression is assessed using the Clinical Dementia Rating Dementia Staging Instrument PLUS National Alzheimer's Disease Coordinating Center frontotemporal lobar degeneration Behavior and Language Domains Sum of Boxes (CDR® plus NACC FTLD-SB) assessment.

In some embodiments of any of the methods provided herein, the heavy chain variable region comprises an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and the light chain variable region comprises an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32). In some embodiments of any of the methods provided herein, the heavy chain variable region comprises an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and the light chain variable region comprises an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32).

In some embodiments of any of the methods provided herein, the antibody comprises: a heavy chain variable region comprising the amino acid sequence of SEQ ID NO: 54, and a light chain variable region comprising the amino acid sequence of SEQ ID NO: 57; a heavy chain variable region comprising the amino acid sequence of SEQ ID NO: 54, and a light chain variable region comprising the amino acid sequence of SEQ ID NO: 58; a heavy chain variable region comprising the amino acid sequence of SEQ ID NO: 54, and a light chain variable region comprising the amino acid sequence of SEQ ID NO: 59; a heavy chain variable region comprising the amino acid sequence of SEQ ID NO: 55, and a light chain variable region comprising the amino acid sequence of SEQ ID NO: 57; a heavy chain variable region comprising the amino acid sequence of SEQ ID NO: 55, and a light chain variable region comprising the amino acid sequence of SEQ ID NO: 58; a heavy chain variable region comprising the amino acid sequence of SEQ ID NO: 56, and a light chain variable region comprising the amino acid sequence of SEQ ID NO: 57; a heavy chain variable region comprising the amino acid sequence of SEQ ID NO: 56, and a light chain variable region comprising the amino acid sequence of SEQ ID NO: 60; a heavy chain variable region comprising the amino acid sequence of SEQ ID NO: 56, and a light chain variable region comprising the amino acid sequence of SEQ ID NO: 77; a heavy chain variable region comprising the amino acid sequence of SEQ ID NO: 56, and a light chain variable region comprising the amino acid sequence of SEQ ID NO: 78; a heavy chain variable region comprising the amino acid sequence of SEQ ID NO: 54, and a light chain variable region comprising the amino acid sequence of SEQ ID NO: 79; or a heavy chain variable region comprising the amino acid sequence of SEQ ID NO: 56, and a light chain variable region comprising the amino acid sequence of SEQ ID NO: 80.

In some embodiments of any of the methods provided herein, the antibody comprises: (i) a heavy chain variable region comprising the amino acid sequence of SEQ ID NO: 56, and a light chain variable region comprising the amino acid sequence of SEQ ID NO: 57; or (ii) a heavy chain variable region comprising the amino acid sequence of SEQ ID NO: 56, and a light chain variable region comprising the amino acid sequence of SEQ ID NO: 60.

In some embodiments of any of the methods provided herein, the antibody comprises a heavy chain comprising the amino acid sequence of SEQ ID NO: 90 or SEQ ID NO: 91, and a light chain comprising the amino acid sequence of SEQ ID NO: 95.

In some embodiments of any of the methods provided herein, the antibody has an IgG1 isotype and the Fc region comprises amino acid substitutions at positions L234A, L235A, and P331S, wherein the numbering of the residue position is according to EU numbering.

In some embodiments of any of the methods provided herein, the disease or injury is selected from frontotemporal dementia, progressive supranuclear palsy, Alzheimer's disease, vascular dementia, seizures, retinal dystrophy, amyotrophic lateral sclerosis, traumatic brain injury, a spinal cord injury, dementia, stroke, Parkinson's disease, acute disseminated encephalomyelitis, retinal degeneration, age related macular degeneration, glaucoma, multiple sclerosis, septic shock, bacterial infection, arthritis, or osteoarthritis.

In some embodiments of any of the methods provided herein, the disease or injury is frontotemporal dementia. In some embodiments, the individual is heterozygous for a mutation in the Progranulin gene (GRN). In some embodiments, the GRN mutation is a loss-of-function mutation. In some embodiments, the GRN mutation is causative of frontotemporal dementia.

In some embodiments of any of the methods provided herein, the individual does not show symptoms of frontotemporal dementia prior to administration of the anti-Sortilin antibody. In some embodiments of any of the methods provided herein, the individual is at risk for developing symptomatic frontotemporal dementia prior to administration of the anti-Sortilin antibody. In some embodiments, the individual has an elevated serum neurofilament light chain level prior to administration of the anti-Sortilin antibody. In some embodiments, the elevated serum neurofilament light chain level comprises a serum neurofilament light chain level of at least about 13.6 pg/mL. In some embodiments, the elevated serum neurofilament light chain level comprises a serum neurofilament light chain level of at least about 19.8 pg/mL. In some embodiments, the individual has a Clinical Dementia Rating Dementia Staging Instrument PLUS National Alzheimer's Disease Coordinating Center Frontotemporal Lobar Degeneration Behavior and Language Domains Sum of Boxes (CDR plus NACC FTLD-SB) score of 0.5 or less prior to administration of the anti-Sortilin antibody.

In some embodiments of any of the methods provided herein, the individual has symptomatic frontotemporal dementia prior to administration of the anti-Sortilin antibody. In some embodiments, the individual has a CDR plus NACC FTLD-SB score greater than 0.5 prior to administration of the anti-Sortilin antibody.

In some embodiments, the individual is heterozygous for a hexanucleotide repeat expansion C9orf72 mutation. In some embodiments, the hexanucleotide repeat expansion C9orf72 mutation is causative of FTD. In some embodiments, the individual has symptomatic frontotemporal dementia prior to administration of the anti-Sortilin antibody.

In some embodiments, the individual has one or more symptoms required for a diagnosis of possible behavioral variant frontotemporal dementia (bvFTD) prior to administration of the anti-Sortilin antibody. In some embodiments, the one or more symptoms are selected from disinhibition, apathy or inertia, loss of sympathy or empathy, perseverative or compulsive behaviors, hyperorality, or dysexecutive neuropsychological profile. In some embodiments, the individual has a diagnosis of primary progressive aphasia (PPA) prior to administration of the anti-Sortilin antibody.

In some embodiments of any of the methods provided herein, the individual has a Clinical Dementia Rating Dementia Staging Instrument PLUS National Alzheimer's Disease Coordinating Center frontotemporal lobar degeneration Behavior and Language Domains (CDR plus NACC FTLD) score of between 0 and 2 prior to administration of the anti-Sortilin antibody. In some embodiments, the individual has a CDR plus NACC FTLD score of 0.5, 1, or 2.

In some embodiments of any of the methods provided herein, the individual is treated for a treatment period of 96 weeks. In some embodiments, administration of the anti-Sortilin antibody occurs on the first day of the treatment period and every four weeks thereafter. In some embodiments, a total of 25 doses of the anti-Sortilin antibody are administered to the individual during the treatment period. In some embodiments, the methods provided herein further comprise continuing administration of the anti-Sortilin antibody to the individual once every four weeks after the end of the 96-week treatment period. In some embodiments, administration of the anti-Sortilin antibody to the individual continues once every four weeks for up to 96 weeks. In some embodiments, administration of the anti-Sortilin antibody to the individual continues once every four weeks for up to 25 doses.

In some embodiments of any of the methods provided herein, the individual is a human adult.

In some embodiments of any of the methods provided herein, the methods further comprise assessing the individual for the presence of one or more GRN mutations prior to administration of the anti-Sortilin antibody. In some embodiments of any of the methods provided herein, the methods further comprise assessing the individual for the presence of a hexanucleotide repeat expansion C9orf72 mutation prior to administration of the anti-Sortilin antibody.

In some embodiments of any of the methods provided herein, the methods further comprise assessing the individual for the presence of an elevated level of neurofilament light chain prior to administration of the anti-Sortilin antibody to the individual, wherein the level of neurofilament light chain is assessed in a sample of serum obtained from the individual.

In some embodiments of any of the methods provided herein, the methods further comprise performing one or more clinical outcome assessments on the individual before and after the individual has received one or more doses of the anti-Sortilin antibody, wherein the one or more clinical outcome assessments are selected from CDR plus NACC FTLD, CDR plus NACC FTLD-SB, Clinical Global Impression-Severity (CGI-S), Clinical Global Impression Improvement (CGI I), Repeatable Battery for the Assessment of Neuropsychological Status (RBANS), European Quality of Life-5 Dimensions (EQ 5D), Zarit Burden Interview (ZBI), Resource Utilization in Dementia-Lite Version (RUD Lite), Frontotemporal Dementia Rating Scale (FRS), or Winterlight Labs Speech Assessment (WLA).

In some embodiments of any of the methods provided herein, the methods further comprise measuring the level of Progranulin protein (PGRN) in a sample of blood plasma obtained from the individual before and after the individual has received one or more doses of the anti-Sortilin antibody.

In some embodiments of any of the methods provided herein, the methods further comprise measuring the level of neurofilament light chain in a sample of serum obtained from the individual before and after the individual has received one or more doses of the anti-Sortilin antibody.

In some embodiments of any of the methods provided herein, the methods further comprise measuring the level of Progranulin protein (PGRN) in a sample of cerebrospinal fluid obtained from the individual before and after the individual has received one or more doses of the anti-Sortilin antibody.

In some embodiments of any of the methods provided herein, the methods further comprise measuring the level of neurofilament light chain in a sample of cerebrospinal fluid obtained from the individual before and after the individual has received one or more doses of the anti-Sortilin antibody.

In some embodiments of any of the methods provided herein, the methods further comprise measuring the level of one or more biomarkers of neurodegeneration in a sample of whole blood, plasma, or cerebrospinal fluid obtained from the individual before and after the individual has received one or more doses of the anti-Sortilin antibody. In some embodiments, the one or more biomarkers of neurodegeneration comprise tau and phosphorylated tau.

In some embodiments of any of the methods provided herein, the methods further comprise measuring the level of one or more biomarkers of lysosomal function in a sample of whole blood, plasma, or cerebrospinal fluid obtained from the individual before and after the individual has received one or more doses of the anti-Sortilin antibody. In some embodiments, the one or more biomarkers of lysosomal function comprise one or more cathepsins.

In some embodiments of any of the methods provided herein, the methods further comprise measuring the level of one or more biomarkers of glial activity in a sample of whole blood, plasma, or cerebrospinal fluid obtained from the individual before and after the individual has received one or more doses of the anti-Sortilin antibody. In some embodiments, the one or more biomarkers of glial activity comprise YKL40 and IL-6.

In some embodiments of any of the methods provided herein, the methods further comprise assessing global and regional brain volumes in the individual before and after the individual has received one or more doses of the anti-Sortilin antibody.

In some embodiments of any of the methods provided herein, the methods further comprise assessing volume of white matter hyperintensities in the individual before and after the individual has received one or more doses of the anti-Sortilin antibody.

In some embodiments of any of the methods provided herein, the methods further comprise assessing brain perfusion in the individual before and after the individual has received one or more doses of the anti-Sortilin antibody.

In some embodiments of any of the methods provided herein, the methods further comprise assessing fractional anisotropy, mean diffusivity, axial diffusivity, and/or radial diffusivity in the individual before and after the individual has received one or more doses of the anti-Sortilin antibody.

In some embodiments of any of the methods provided herein, the methods further comprise measuring the level of one or more biomarkers of astrogliosis in a sample of whole blood, plasma, or cerebrospinal fluid obtained from the individual before and after the individual has received one or more doses of the anti-Sortilin antibody. In some embodiments, the one or more biomarkers of astrogliosis comprise glial fibrillary acidic protein (GFAP).

In some embodiments of any of the methods provided herein, the methods further comprise measuring the level of one or more biomarkers of neuroinflammation in a sample of cerebrospinal fluid obtained from the individual before and after the individual has received one or more doses of the anti-Sortilin antibody. In some embodiments, the one or more biomarkers of neuroinflammation comprise macrophage migration inhibitory factor (MIF).

In some embodiments of any of the methods provided herein, the methods further comprise measuring the level of the anti-Sortilin antibody in a sample of blood or cerebrospinal fluid obtained from the individual before and after the individual has received one or more doses of the anti-Sortilin antibody.

In other aspects, provided herein are methods of monitoring treatment of an individual being administered an anti-Sortilin antibody, comprising performing one or more clinical outcome assessments on the individual before and after the individual has received one or more doses of an anti-Sortilin antibody, wherein the one or more clinical outcome assessments are selected from CDR plus NACC FTLD, CDR plus NACC FTLD-SB, CGI-S, CGI I, RBANS, EQ 5D, ZBI, RUD Lite, FRS, or WLA. In some embodiments, the methods further comprise assessing the activity of the anti-Sortilin antibody in the individual based on a result of the one or more clinical outcome assessments. In some embodiments, the anti-Sortilin antibody is determined to be active in the individual if a result of the one or more clinical outcome assessments improves after the individual has received one or more doses of the anti-Sortilin antibody compared to a corresponding result before the individual received one or more doses of the anti-Sortilin antibody.

In other aspects, provided herein are methods of monitoring treatment of an individual being administered an anti-Sortilin antibody, comprising measuring the level of Progranulin protein (PGRN) in a sample obtained from the individual before and after the individual has received one or more doses of an anti-Sortilin antibody. In some embodiments, the methods further comprise assessing the activity of the anti-Sortilin antibody in the individual based on the level of PGRN in a sample obtained from the individual. In some embodiments, the anti-Sortilin antibody is determined to be active in the individual if the level of PGRN in a sample obtained after the individual has received one or more doses of the anti-Sortilin antibody is increased compared to the level of PGRN in a sample obtained before the individual received one or more doses of the anti-Sortilin antibody. In some embodiments, the sample is a blood plasma sample or a cerebrospinal fluid sample.

In other aspects, provided herein are methods of monitoring treatment of an individual being administered an anti-Sortilin antibody, comprising measuring the level of neurofilament light chain in a sample obtained from the individual before and after the individual has received one or more doses of an anti-Sortilin antibody. In some embodiments, the methods further comprise assessing the activity of the anti-Sortilin antibody in the individual based on the level of neurofilament light chain in a sample obtained from the individual. In some embodiments, the anti-Sortilin antibody is determined to be active in the individual if the level of neurofilament light chain in a sample obtained after the individual has received one or more doses of the anti-Sortilin antibody is decreased compared to the level of neurofilament light chain in a sample obtained before the individual received one or more doses of the anti-Sortilin antibody. In some embodiments, the sample is a serum sample or a cerebrospinal fluid sample.

In other aspects, provided herein are methods of monitoring treatment of an individual being administered an anti-Sortilin antibody, comprising measuring the level of one or more biomarkers of neurodegeneration, lysosomal function, astrogliosis, neuroinflammation, or glial activity in a sample obtained from the individual before and after the individual has received one or more doses of an anti-Sortilin antibody. In some embodiments, the methods further comprise assessing the activity of the anti-Sortilin antibody in the individual based on the level of the one or more biomarkers of neurodegeneration, lysosomal function, astrogliosis, neuroinflammation, or glial activity in a sample obtained from the individual. In some embodiments, the sample is a whole blood, plasma, or cerebrospinal fluid sample. In some embodiments, the one or more biomarkers of neurodegeneration comprise tau and phosphorylated tau. In some embodiments, the one or more biomarkers of lysosomal function comprise one or more cathepsins. In some embodiments, the one or more biomarkers of glial activity comprise YKL40 and IL-6. In some embodiments, the one or more biomarkers of astrogliosis comprise GFAP. In some embodiments, the one or more biomarkers of neuroinflammation comprise macrophage migration inhibitory factor (MIF).

In other aspects, provided herein are methods of monitoring treatment of an individual being administered an anti-Sortilin antibody, comprising assessing global and regional brain volumes, volume of white matter hyperintensities, brain perfusion, fractional anisotropy, mean diffusivity, axial diffusivity, and/or radial diffusivity in the individual before and after the individual has received one or more doses of an anti-Sortilin antibody. In some embodiments, the methods further comprise assessing the activity of the anti-Sortilin antibody in the individual based on global and regional brain volumes, volume of white matter hyperintensities, brain perfusion, fractional anisotropy, mean diffusivity, axial diffusivity, and/or radial diffusivity.

In another aspect, provided herein is an anti-sortilin antibody at a dose of about 60 mg/kg intravenously about once every four weeks for use in a method of treating and/or delaying the progression of a disease or injury in an individual, wherein the antibody comprises (i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33); (iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32); (vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33); (vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or (viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32).

In another aspect, provided herein is an anti-sortilin antibody at a dose of about 60 mg/kg intravenously about once every four weeks for use in a method of treating and/or delaying the progression of frontotemporal dementia in an individual at risk for developing symptomatic frontotemporal dementia, wherein the individual has an elevated serum neurofilament light chain level, and wherein the antibody comprises (i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33); (iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32); (vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33); (vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or (viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32).

In another aspect, provided herein is a use of an anti-sortilin antibody at a dose of about 60 mg/kg intravenously about once every four weeks in the manufacture of a medicament for treating and/or delaying the progression of a disease or injury in an individual, wherein the antibody comprises (i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33); (iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32); (vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33); (vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or (viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32).

In another aspect, provided herein is a use of an anti-sortilin antibody at a dose of about 60 mg/kg intravenously about once every four weeks in the manufacture of a medicament for treating and/or delaying the progression of frontotemporal dementia in an individual at risk for developing symptomatic frontotemporal dementia, wherein the individual has an elevated serum neurofilament light chain level, and wherein the antibody comprises (i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33); (iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32); (v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32); (vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33); (vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or (viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32).

### BRIEF DESCRIPTION OF THE DRAWINGS

FIG. 1 provides an overview of the study described in Example 1. bvFTD, behavioral variant frontotemporal dementia; CDR® plus NACC FTLD-SB, Clinical Dementia Rating Dementia Staging Instrument PLUS National Alzheimer's Disease Coordinating Center Frontotemporal Lobar Degeneration Behavior and Language Domains Sum of Boxes; COA, clinical outcome assessment; CSF, cerebrospinal fluid; GRN, Progranulin gene; IV, intravenous(ly); MRI, magnetic resonance imaging; NfL, neurofilament light chain; PD, pharmacodynamic; PK, pharmacokinetic; PPA, primary progressive aphasia; q4w, every 4 weeks; EOS, end of study; OLE, open-label extension.

FIG. 2 provides an overview of the study described in Example 2. COA, clinical outcome assessment; CSF, cerebrospinal fluid; GRN, Progranulin gene; IV, intravenous; MRI, magnetic resonance imaging; q4w, every 4 weeks. EOS, end of study; OLE, open-label extension.

FIGS. 3A-3B show the levels of PGRN in plasma and cerebrospinal fluid (CSF) of symptomatic carriers of loss-of-function Granulin mutations causative of FTD (FTD-GRN participants; N=12) in the study described in Example 2. The levels of PGRN in plasma (FIG. 3A) and CSF (FIG. 3B) are shown as the mean (ng/mL; mean±standard error of the mean [SEM]) at the times during the study indicated on the x-axis (weeks). The x-axes in FIGS. 3A-3B also indicate the number of participants included in the analysis at each time point (“n”). The shaded regions in the graphs show the normal range of plasma PGRN levels (FIG. 3A) or CSF PGRN levels (FIG. 3B) in age-matched controls.

FIGS. 4A-4C show the levels of lysosome and complement biomarkers in the CSF of FTD-GRN participants in the study described in Example 2 at the indicated times. FIG. 4A shows the levels of Cathepsin D; FIG. 4B shows the levels of LAMP1; and FIG. 4C shows the levels of C1QB. The levels of each biomarker in FIGS. 4A-4C are shown as the mean (fmol/μL)±SEM. Only FTD-GRN participants with baseline and post-treatment data available were included in the results. “Procured Ctrl” refers to age-matched control individuals; “FTD baseline” refers to the mean levels of the indicated biomarker in FTD-GRN participants at baseline (prior to administration of anti-Sortilin antibody S-60-15.1 [N33T] LALAPS); “FTD+6 months” refers to the mean levels of the indicated biomarker in FTD-GRN participants at 6 months after the start of treatment with anti-Sortilin antibody S-60-15.1 [N33T] LALAPS; and “FTD+12 months” refers to the mean levels of the indicated biomarker in FTD-GRN participants at 12 months after the start of treatment with anti-Sortilin antibody S-60-15.1 [N33T] LALAPS. ¹n=44; ²n=9; ³n=8; and ⁴n=8.

FIGS. 5A-5B show the levels of neurofilament light chain (NfL) in the plasma and CSF of FTD-GRN participants in the study described in Example 2. The levels of NfL in plasma (FIG. 5A) and in CSF (FIG. 5B) are shown as the mean (pg/mL)±SEM at the times during the study indicated on the x-axis (weeks). The x-axes in FIGS. 5A-5B also indicate the number of participants included in the analysis at each time point (“n”).

FIG. 6 shows an analysis of brain atrophy in FTD-GRN participants in the study described in Example 2, and in a synthetic matched control group generated as described in Example 2. The ventricles, whole brain, and frontotemporal cortex were analyzed by volumetric magnetic resonance imaging (vMRI) over one year. *For the synthetic control only, there was *n=8 for whole brain, and n=7 for Tensor-based Morphometry (TBM) measures (TBM could not be produced from one subject in the synthetic matched control); one subject in the synthetic matched control was excluded from the analysis as they displayed cortical volume increases (2.58% annual volume increase in the frontotemporal cortex), indicating possible imaging artifact.

FIG. 7 shows an analysis of clinical disease progression assessed using the CDR® plus NACC FTLD-SB assessment in FTD-GRN participants in the study described in Example 2, and in a synthetic matched control group generated as described in Example 2. The CDR® plus NACC FTLD-SB assessment is the Clinical Dementia Rating Dementia Staging Instrument PLUS National Alzheimer's Disease Coordinating Center Frontotemporal Lobar Degeneration Behavior and Language Domains Sum of Boxes. The y-axis shows the change from baseline in CDR® plus NACC FTLD-SB scores at the times indicated on the x-axis, from baseline (prior to treatment with anti-Sortilin antibody S-60-15.1 [N33T] LALAPS) to 12 months after the start of treatment with anti-Sortilin antibody S-60-15.1 [N33T] LALAPS.

FIGS. 8A-8B show the levels of GFAP in the plasma and CSF of FTD-GRN participants in the study described in Example 2. The levels of GFAP in plasma (FIG. 8A) and in CSF (FIG. 8B) are shown as the mean (pg/mL or ng/mL)±SEM at the times during the study indicated on the x-axis (weeks). The x-axes in FIGS. 8A-8B also indicate the number of participants included in the analysis at each time point (“n”). ¹Range is of baseline GFAP levels in asymptomatic FTD-GRN patients enrolled in this study.

FIG. 9 depicts a colorimetric sandwich enzyme-linked immunosorbent assay (ELISA) for the quantitative determination of macrophage migration inhibitory factor (MIF) levels in human cerebrospinal fluid (CSF). As described in Example 2 herein, in the ELISA method, an anti-MIF monoclonal antibody (#1, “α-MIF mAb”) is coated onto 96-well microtiter plates (e.g., 96-well NUNC High Binding ELISA Plates). Standards and samples containing MIF protein (#2) are added to the wells for binding to the coated antibody. After washing, MIF is detected by the addition of a biotinylated (“B”) anti-MIF polyclonal antibody (#3, “Biotinylated (α-MIF pAb”). Following a wash to remove excess biotinylated antibody, horseradish peroxidase (“HRP”)-labeled streptavidin (“SA”) is added (#4, “Streptavidin-HRP”). A final wash is performed, and 3,3′,5,5′-tetramethylbenzidine substrate solution is added to the wells to allow color development (#5, “TMB”). Color development is stopped with stop solution (#6, “Stop Solution”), and the intensity of the color development is measured using a plate reader (#7, e.g., using a SpectraMax M5 Plate Reader).

FIG. 10 shows the levels of MIF protein in the CSF of FTD-GRN participants from the Phase 1 and Phase 2 studies of anti-Sortilin antibody S-60-15.1 [N33T] LALAPS as described in Example 2 herein. The levels of MIF in CSF are provided as pg/mL (mean±SEM) at the indicated times after the start of treatment with anti-Sortilin antibody S-60-15.1 [N33T] LALAPS (D0=Day 0; D57=Day 57; 6 mos=6 months; and 12 mos=12 months). “HV” refers to healthy volunteers from the Phase 1 study of anti-Sortilin antibody S-60-15.1 [N33T] LALAPS as described in Example 2 herein. “Sym-FTD” and “Sym FTD” refer to FTD-GRN participants. “Procured HV” refers to age-matched procured controls that were used to compare with data from the Phase 2 study of anti-Sortilin antibody S-60-15.1 [N33T] LALAPS as described in Example 2 herein.

FIG. 11 shows the levels of MIF protein in the CSF of symptomatic carriers of C9orf72 hexanucleotide repeat expansion mutations causative of FTD (FTD-C9orf72) from the Phase 2 study in Example 2 herein. The levels of MIF in CSF are provided as pg/mL (mean±SEM) at the indicated times after the start of treatment with anti-Sortilin antibody S-60-15.1 [N33T] LALAPS. “Ph 1 HV” refers to healthy volunteers from the Phase 1 study of anti-Sortilin antibody S-60-15.1 [N33T] LALAPS as described in Example 2 herein. “Procured Ctrl” refers to age-matched procured controls that were used to compare with data from the Phase 2 study of anti-Sortilin antibody S-60-15.1 [N33T] LALAPS as described in Example 2 herein.

FIGS. 12A-12B show the levels of PGRN in plasma and CSF of symptomatic carriers of a hexanucleotide repeat expansion C9orf72 mutation causative of FTD (FTD-C9orf72 participants) in the study described in Example 2. The levels of PGRN in plasma (FIG. 12A) and CSF (FIG. 12B) are shown as the mean (ng/mL)±standard error of the mean (SEM) at the times during the study indicated on the x-axis (weeks). The x-axes in FIGS. 12A-12B also indicate the number of participants included in the analysis at each time point (“n”).

FIGS. 13A-13B show the levels of neurofilament light chain (NfL) in the plasma and CSF of FTD-C9orf72 participants in the study described in Example 2. The levels of NfL in plasma (FIG. 13A) and in CSF (FIG. 13B) are shown as the mean (pg/mL)±SEM at the times during the study indicated on the x-axis (weeks). The x-axes in FIGS. 13A-13B also indicate the number of participants included in the analysis at each time point (“n”).

FIGS. 14A-14B show the levels of GFAP in the plasma and CSF of FTD-C9orf72 participants in the study described in Example 2. The levels of GFAP in plasma (FIG. 14A) and in CSF (FIG. 14B) are shown as the mean (pg/mL or ng/mL)±SEM at the times during the study indicated on the x-axis (weeks). The x-axes in FIGS. 14A-14B also indicate the number of participants included in the analysis at each time point (“n”).

FIG. 15 shows an analysis of clinical disease progression in FTD-C9orf72 participants in the study described in Example 2, and in a historical control cohort generated as described in Example 2. Clinical disease progression was evaluated using the CDR® plus NACC FTLD-SB assessment. The y-axis shows the change from baseline in CDR® plus NACC FTLD-SB scores at the times indicated on the x-axis, up to 12 months after the start of treatment with anti-Sortilin antibody S-60-15.1 [N33T] LALAPS. “Control” refers to the historical control cohort, and “FTD-C9orf72” refers to FTD-C9orf72 participants in the study.

### DETAILED DESCRIPTION

### Definitions

As used herein, the term “preventing” includes providing prophylaxis with respect to occurrence or recurrence of a particular disease, disorder, or condition in an individual. An individual may be predisposed to, susceptible to a particular disease, disorder, or condition, or at risk of developing such a disease, disorder, or condition, but has not yet been diagnosed with the disease, disorder, or condition.

As used herein, an individual “at risk” of developing a particular disease, disorder, or condition may or may not have detectable disease or symptoms of disease, and may or may not have displayed detectable disease or symptoms of disease prior to the treatment methods described herein. “At risk” denotes that an individual has one or more risk factors, which are measurable parameters that correlate with development of a particular disease, disorder, or condition, as known in the art. An individual having one or more of these risk factors has a higher probability of developing a particular disease, disorder, or condition than an individual without one or more of these risk factors.

As used herein, the term “treatment” refers to clinical intervention designed to alter the natural course of the individual being treated during the course of clinical pathology. Desirable effects of treatment include decreasing the rate of progression, ameliorating or palliating the pathological state, and remission or improved prognosis of a particular disease, disorder, or condition. An individual is successfully “treated”, for example, if one or more symptoms associated with a particular disease, disorder, or condition are mitigated or eliminated.

An “effective amount” refers to at least an amount effective, at dosages and for periods of time necessary, to achieve the desired therapeutic or prophylactic result. An effective amount can be provided in one or more administrations. An effective amount herein may vary according to factors such as the disease state, age, sex, and weight of the individual, and the ability of the treatment to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. An effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved

As used herein, administration “in conjunction” with another compound or composition includes simultaneous administration and/or administration at different times. Administration in conjunction also encompasses administration as a co-formulation or administration as separate compositions, including at different dosing frequencies or intervals, and using the same route of administration or different routes of administration.

An “individual” for purposes of treatment, prevention, or reduction of risk refers to any animal classified as a mammal, including humans, domestic and farm animals, and zoo, sport, or pet animals, such as dogs, horses, rabbits, cattle, pigs, hamsters, gerbils, mice, ferrets, rats, cats, and the like. Preferably, the individual is human.

The terms “Sortilin” or “Sortilin polypeptide” are used interchangeably herein to refer to any native Sortilin from any mammalian source, including primates (e.g., humans and cynos) and rodents (e.g., mice and rats), unless otherwise indicated. In some embodiments, the term encompasses both wild-type sequences and naturally occurring variant sequences, e.g., splice variants or allelic variants. In some embodiments, the term encompasses “full-length,” unprocessed Sortilin as well as any form of Sortilin that results from processing in the cell. In some embodiments, the Sortilin is human Sortilin. In some embodiments, the amino acid sequence of an exemplary human Sortilin is SEQ ID NO: 81.

The terms “anti-Sortilin antibody,” an “antibody that binds to Sortilin,” and “antibody that specifically binds Sortilin” refer to an antibody that is capable of binding Sortilin with sufficient affinity such that the antibody is useful as a diagnostic and/or therapeutic agent in targeting Sortilin. In one embodiment, the extent of binding of an anti-Sortilin antibody to an unrelated, non-Sortilin polypeptide is less than about 10% of the binding of the antibody to Sortilin as measured, e.g., by a radioimmunoassay (RIA). In certain embodiments, an antibody that binds to Sortilin has a dissociation constant (KD) of <1 μM, <100 nM, <10 nM, <1 nM, <0.1 nM, <0.01 nM, or <0.001 nM (e.g., 10⁻⁸ M or less, e.g. from 10⁻⁸ M to 10⁻¹³ M, e.g., from 10⁻⁹ M to 10⁻¹³ M). In certain embodiments, an anti-Sortilin antibody binds to an epitope of Sortilin that is conserved among Sortilin from different species.

The term “immunoglobulin” (Ig) is used interchangeably with “antibody” herein. The term “antibody” herein is used in the broadest sense and specifically covers monoclonal antibodies, polyclonal antibodies, multispecific antibodies (e.g., bispecific antibodies) including those formed from at least two intact antibodies, and antibody fragments so long as they exhibit the desired biological activity.

“Native antibodies” are usually heterotetrameric glycoproteins of about 150,000 Daltons, composed of two identical Light (“L”) chains and two identical heavy (“H”) chains. Each light chain is linked to a heavy chain by one covalent disulfide bond, while the number of disulfide linkages varies among the heavy chains of different immunoglobulin isotypes. Each heavy and light chain also has regularly spaced intra-chain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain, and the light chain variable domain is aligned with the variable domain of the heavy chain. Particular amino acid residues are believed to form an interface between the light chain and heavy chain variable domains.

For the structure and properties of the different classes of antibodies, see, e.g., Basic and Clinical Immunology, 8th Ed., Daniel P. Stites, Abba I. Terr and Tristram G. Parslow (eds.), Appleton & Lange, Norwalk, C T, 1994, page 71 and Chapter 6.

The light chain from any vertebrate species can be assigned to one of two clearly distinct types, called kappa (“κ”) and lambda (“λ”), based on the amino acid sequences of their constant domains. Depending on the amino acid sequence of the constant domain of their heavy chains (CH), immunoglobulins can be assigned to different classes or isotypes. There are five classes of immunoglobulins: IgA, IgD, IgE, IgG, and IgM, having heavy chains designated alpha (“a”), delta (“δ”), epsilon (“ε”), gamma (“γ”), and mu (“μ”), respectively. The γ and α classes are further divided into subclasses (isotypes) on the basis of relatively minor differences in the CH sequence and function, e.g., humans express the following subclasses: IgG1, IgG2, IgG3, IgG4, IgA1, and IgA2. The subunit structures and three-dimensional configurations of different classes of immunoglobulins are well known and described generally in, for example, Abbas et al., Cellular and Molecular Immunology, 4^{th} ed. (W.B. Saunders Co., 2000).

The “variable region” or “variable domain” of an antibody, such as an anti-Sortilin antibody of the present disclosure, refers to the amino-terminal domains of the heavy or light chain of the antibody. The variable domains of the heavy chain and light chain may be referred to as “V_{H}” and “V_{L}”, respectively. These domains are generally the most variable parts of the antibody (relative to other antibodies of the same class) and contain the antigen binding sites.

The term “variable” refers to the fact that certain segments of the variable domains differ extensively in sequence among antibodies, such as anti-Sortilin antibodies of the present disclosure. The variable domain mediates antigen binding and defines the specificity of a particular antibody for its particular antigen. However, the variability is not evenly distributed across the entire span of the variable domains. Instead, it is concentrated in three segments called hypervariable regions (HVRs), both in the light chain and the heavy chain variable domains. The more highly conserved portions of variable domains are called the framework regions (FR). The variable domains of native heavy and light chains each comprise four FR regions, largely adopting a beta-sheet configuration, connected by three HVRs, which form loops connecting, and in some cases forming part of, the beta-sheet structure. The HVRs in each chain are held together in close proximity by the FR regions and, with the HVRs from the other chain, contribute to the formation of the antigen-binding site of antibodies (see Kabat et al., Sequences of Immunological Interest, Fifth Edition, National Institute of Health, Bethesda, MD (1991)). The constant domains are not involved directly in the binding of antibody to an antigen, but exhibit various effector functions, such as participation of the antibody in antibody-dependent-cellular toxicity.

An “isolated” antibody, such as an anti-Sortilin antibody of the present disclosure, is one that has been identified, separated and/or recovered from a component of its production environment (e.g., naturally or recombinantly). Preferably, the isolated antibody is free of association with all other contaminant components from its production environment. Contaminant components from its production environment, such as those resulting from recombinant transfected cells, are materials that would typically interfere with research, diagnostic or therapeutic uses for the antibody, and may include enzymes, hormones, and other proteinaceous or non-proteinaceous solutes. In preferred embodiments, the antibody will be purified: (1) to greater than 95% by weight of antibody as determined by, for example, the Lowry method, and in some embodiments, to greater than 99% by weight; (2) to a degree sufficient to obtain at least 15 residues of N-terminal or internal amino acid sequence by use of a spinning cup sequenator, or (3) to homogeneity by SDS-PAGE under non-reducing or reducing conditions using Coomassie blue or, preferably, silver stain. Isolated antibody includes the antibody in situ within recombinant T cells since at least one component of the antibody's natural environment will not be present. Ordinarily, however, an isolated polypeptide or antibody will be prepared by at least one purification step.

The term “monoclonal antibody” as used herein refers to an antibody, such as a monoclonal anti-Sortilin antibody of the present disclosure, obtained from a population of substantially homogeneous antibodies, i.e., the individual antibodies comprising the population are identical except for possible naturally occurring mutations and/or post-translation modifications (e.g., isomerizations, amidations, etc.) that may be present in minor amounts. Monoclonal antibodies are highly specific, being directed against a single antigenic site. In contrast to polyclonal antibody preparations which typically include different antibodies directed against different determinants (epitopes), each monoclonal antibody is directed against a single determinant on the antigen. In addition to their specificity, monoclonal antibodies are advantageous in that they may be synthesized by hybridoma culture, uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, but not limited to one or more of the following methods, immunization methods of animals including, but not limited to rats, mice, rabbits, guinea pigs, hamsters and/or chickens with one or more of DNA(s), virus-like particles, polypetide(s), and/or cell(s), the hybridoma methods, B-cell cloning methods, recombinant DNA methods, and technologies for producing human or human-like antibodies in animals that have parts or all of the human immunoglobulin loci or genes encoding human immunoglobulin sequences.

The terms “full-length antibody,” “intact antibody” or “whole antibody” are used interchangeably to refer to an antibody, such as an anti-Sortilin antibody of the present disclosure, in its substantially intact form, as opposed to an antibody fragment. Specifically, whole antibodies include those with heavy and light chains including an Fc region. The constant domains may be native sequence constant domains (e.g., human native sequence constant domains) or amino acid sequence variants thereof. In some cases, the intact antibody may have one or more effector functions.

An “antibody fragment” comprises a portion of an intact antibody, preferably the antigen binding and/or the variable region of the intact antibody. Examples of antibody fragments include Fab, Fab′, F(ab′)₂ and Fv fragments; diabodies; linear antibodies (see U.S. Pat. No. 5,641,870, Example 2; Zapata et al., Protein Eng. 8(10):1057-1062 (1995)); single-chain antibody molecules and multispecific antibodies formed from antibody fragments.

Papain digestion of antibodies, such as anti-Sortilin antibodies of the present disclosure, produces two identical antigen-binding fragments, called “Fab” fragments, and a residual “Fc” fragment, a designation reflecting the ability to crystallize readily. The Fab fragment consists of an entire L chain along with the variable region domain of the H chain (V_{H}), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having different antigen-binding activity and is still capable of cross-linking antigen. Fab′ fragments differ from Fab fragments by having a few additional residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, the region which is also recognized by Fc receptors (FcR) found on certain types of cells.

“Fv” is the minimum antibody fragment which contains a complete antigen-recognition and antigen-binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three HVRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of the sFv, see Pluckthun in The Pharmacology of Monoclonal Antibodies, vol. 113, Rosenburg and Moore eds., Springer-Verlag, New York, pp. 269-315 (1994).

“Functional fragments” of antibodies, such as anti-Sortilin antibodies of the present disclosure, comprise a portion of an intact antibody, generally including the antigen binding or variable region of the intact antibody or the Fc region of an antibody which retains or has modified FcR binding capability. Examples of antibody fragments include linear antibody, single-chain antibody molecules and multispecific antibodies formed from antibody fragments.

The term “diabodies” refers to small antibody fragments prepared by constructing sFv fragments (see above) with short linkers (about 5-10 residues) between the V_{H} and V_{L} domains such that inter-chain but not intra-chain pairing of the variable domains is achieved, thereby resulting in a bivalent fragment, i.e., a fragment having two antigen-binding sites. Bispecific diabodies are heterodimers of two “crossover” sFv fragments in which the V_{H} and V_{L} domains of the two antibodies are present on different polypeptide chains.

As used herein, a “chimeric antibody” refers to an antibody (immunoglobulin), such as a chimeric anti-Sortilin antibody of the present disclosure, in which a portion of the heavy and/or light chain is identical with or homologous to corresponding sequences in antibodies derived from a particular species or belonging to a particular antibody class or subclass, while the remainder of the chain(s) is(are) identical with or homologous to corresponding sequences in antibodies derived from another species or belonging to another antibody class or subclass, as well as fragments of such antibodies, so long as they exhibit the desired biological activity. Chimeric antibodies of interest herein include PRIMATIZED© antibodies wherein the antigen-binding region of the antibody is derived from an antibody produced by, e.g., immunizing macaque monkeys with an antigen of interest. As used herein, “humanized antibody” is used a subset of “chimeric antibodies.”

“Humanized” forms of non-human (e.g., murine) antibodies, such as humanized forms of anti-Sortilin antibodies of the present disclosure, are chimeric antibodies comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

A “human antibody” is one that possesses an amino acid sequence corresponding to that of an antibody, such as an anti-Sortilin antibody of the present disclosure, produced by a human and/or has been made using any of the techniques for making human antibodies as disclosed herein or known in the art. This definition of a human antibody specifically excludes a humanized antibody comprising non-human antigen-binding residues. Human antibodies can be produced using various techniques known in the art, including phage-display libraries and yeast-based platform technologies. Human antibodies can be prepared by administering the antigen to a transgenic animal that has been modified to produce such antibodies in response to antigenic challenge, but whose endogenous loci have been disabled, e.g., immunized xenomice as well as generated via a human B-cell hybridoma technology.

The term “hypervariable region,” “HVR,” or “HV,” when used herein refers to the regions of an antibody variable domain, such as that of an anti-Sortilin antibody of the present disclosure, that are hypervariable in sequence and/or form structurally defined loops. Generally, antibodies comprise six HVRs; three in the V_{H} (H1, H2, H3), and three in the V_{L} (L1, L2, L3). In native antibodies, H3 and L3 display the most diversity of the six HVRs, and H3 in particular is believed to play a unique role in conferring fine specificity to antibodies. Naturally occurring camelid antibodies consisting of a heavy chain only are functional and stable in the absence of light chain.

A number of HVR delineations are in use and are encompassed herein. In some embodiments, the HVRs may be Kabat complementarity-determining regions (CDRs) based on sequence variability and are the most commonly used (Kabat et al., supra). In some embodiments, the HVRs may be Chothia CDRs. Chothia refers instead to the location of the structural loops (Chothia and Lesk J. Mol. Biol. 196:901-917 (1987)). In some embodiments, the HVRs may be AbM HVRs. The AbM HVRs represent a compromise between the Kabat CDRs and Chothia structural loops, and are used by Oxford Molecular's AbM antibody-modeling software. In some embodiments, the HVRs may be “contact” HVRs. The “contact” HVRs are based on an analysis of the available complex crystal structures. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B (Kabat |
| | | | | numbering) |
| H1 | H31-H35 | H26-H35 | H26-H32 | H30-H35 (Chothia |
| | | | | numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the V_{L}, and 26-35 (H1), 50-65 or 49-65 (a preferred embodiment) (H2), and 93-102, 94-102, or 95-102 (H3) in the V_{H}. The variable-domain residues are numbered according to Kabat et al., supra, for each of these extended-HVR definitions.

“Framework” or “FR” residues are those variable domain residues other than the HVR residues as herein defined.

An “acceptor human framework” as used herein is a framework comprising the amino acid sequence of a V_{L} or V_{H} framework derived from a human immunoglobulin framework or a human consensus framework. An acceptor human framework “derived from” a human immunoglobulin framework or a human consensus framework may comprise the same amino acid sequence thereof, or it may comprise pre-existing amino acid sequence changes. In some embodiments, the number of pre-existing amino acid changes are 10 or less, 9 or less, 8 or less, 7 or less, 6 or less, 5 or less, 4 or less, 3 or less, or 2 or less. Where pre-existing amino acid changes are present in a V_{H}, preferably those changes occur at only three, two, or one of positions 71H, 73H and 78H; for instance, the amino acid residues at those positions may be 71A, 73T and/or 78A. In one embodiment, the V_{L} acceptor human framework is identical in sequence to the V_{L} human immunoglobulin framework sequence or human consensus framework sequence.

A “human consensus framework” is a framework that represents the most commonly occurring amino acid residues in a selection of human immunoglobulin V_{L} or V_{H} framework sequences. Generally, the selection of human immunoglobulin V_{L} or V_{H} sequences is from a subgroup of variable domain sequences. Generally, the subgroup of sequences is a subgroup as in Kabat et al., Sequences of Proteins of Immunological Interest, 5th Ed. Public Health Service, National Institutes of Health, Bethesda, MD (1991). Examples include, for the V_{L}, the subgroup may be subgroup kappa I, kappa II, kappa III or kappa IV as in Kabat et al., supra. Additionally, for the V_{H}, the subgroup may be subgroup I, subgroup II, or subgroup III as in Kabat et al., supra.

An “amino acid modification” at a specified position, e.g., of an anti-Sortilin antibody of the present disclosure, refers to the substitution or deletion of the specified residue, or the insertion of at least one amino acid residue adjacent the specified residue. Insertion “adjacent” to a specified residue means insertion within one to two residues thereof. The insertion may be N-terminal or C-terminal to the specified residue. The preferred amino acid modification herein is a substitution.

An “affinity-matured” antibody, such as an anti-Sortilin antibody of the present disclosure, is one with one or more alterations in one or more HVRs thereof that result in an improvement in the affinity of the antibody for antigen, compared to a parent antibody that does not possess those alteration(s). In one embodiment, an affinity-matured antibody has nanomolar or even picomolar affinities for the target antigen. Affinity-matured antibodies are produced by procedures known in the art. For example, Marks et al., Bio/Technology 10:779-783 (1992) describes affinity maturation by VH- and VL-domain shuffling. Random mutagenesis of HVR and/or framework residues is described by, for example: Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154(7):3310-9 (1995); and Hawkins et al, J. Mol. Biol. 226:889-896 (1992).

As used herein, the term “specifically recognizes” or “specifically binds” refers to measurable and reproducible interactions such as attraction or binding between a target and an antibody, such as an anti-Sortilin antibody of the present disclosure, that is determinative of the presence of the target in the presence of a heterogeneous population of molecules including biological molecules. For example, an antibody, such as an anti-Sortilin antibody of the present disclosure, that specifically or preferentially binds to a target or an epitope is an antibody that binds this target or epitope with greater affinity, avidity, more readily, and/or with greater duration than it binds to other targets or other epitopes of the target. It is also understood by reading this definition that, for example, an antibody (or a moiety) that specifically or preferentially binds to a first target may or may not specifically or preferentially bind to a second target. As such, “specific binding” or “preferential binding” does not necessarily require (although it can include) exclusive binding. An antibody that specifically binds to a target may have an association constant of at least about 10³M⁻¹ or 10⁴M⁻¹, sometimes about 10⁵M⁻¹ or 10⁶M⁻¹, in other instances about 10⁶M⁻¹ or 10⁷M⁻¹, about 10⁸ M⁻¹ to 10⁹M⁻¹, or about 10¹⁰ M⁻¹ to 10¹¹ M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

As used herein, an “interaction” between a Sortilin protein and a second protein encompasses, without limitation, protein-protein interaction, a physical interaction, a chemical interaction, binding, covalent binding, and ionic binding. As used herein, an antibody “inhibits interaction” between two proteins when the antibody disrupts, reduces, or completely eliminates an interaction between the two proteins. An antibody of the present disclosure, or fragment thereof, “inhibits interaction” between two proteins when the antibody or fragment thereof binds to one of the two proteins.

An “agonist” antibody or an “activating” antibody is an antibody, such as an agonist anti-Sortilin antibody of the present disclosure, that induces (e.g., increases) one or more activities or functions of the antigen after the antibody binds the antigen.

A “blocking” antibody, an “antagonist” antibody, or an “inhibitory” antibody is an antibody, such as an anti-Sortilin antibody of the present disclosure, that inhibits or reduces (e.g., decreases) antigen binding to one or more ligands after the antibody binds the antigen, and/or that inhibits or reduces (e.g., decreases) one or more activities or functions of the antigen after the antibody binds the antigen. In some embodiments, blocking antibodies, antagonist antibodies, or inhibitory antibodies substantially or completely inhibit antigen binding to one or more ligands and/or one or more activities or functions of the antigen.

Antibody “effector functions” refer to those biological activities attributable to the Fc region (a native sequence Fc region or amino acid sequence variant Fc region) of an antibody, and vary with the antibody isotype.

The term “Fc region” herein is used to define a C-terminal region of an immunoglobulin heavy chain, including native-sequence Fc regions and variant Fc regions. Although the boundaries of the Fc region of an immunoglobulin heavy chain might vary, the human IgG heavy-chain Fc region is usually defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. Suitable native-sequence Fc regions for use in the antibodies of the present disclosure include human IgG1, IgG2, IgG3 and IgG4.

A “native sequence Fc region” comprises an amino acid sequence identical to the amino acid sequence of an Fc region found in nature. Native sequence human Fc regions include a native sequence human IgG1 Fc region (non-A and A allotypes); native sequence human IgG2 Fc region; native sequence human IgG3 Fc region; and native sequence human IgG4 Fc region, as well as naturally occurring variants thereof.

A “variant Fc region” comprises an amino acid sequence which differs from that of a native sequence Fc region by virtue of at least one amino acid modification, preferably one or more amino acid substitution(s). Preferably, the variant Fc region has at least one amino acid substitution compared to a native sequence Fc region or to the Fc region of a parent polypeptide, e.g. from about one to about ten amino acid substitutions, and preferably from about one to about five amino acid substitutions in a native sequence Fc region or in the Fc region of the parent polypeptide. The variant Fc region herein will preferably possess at least about 80% homology with a native sequence Fc region and/or with an Fc region of a parent polypeptide, and most preferably at least about 90% homology therewith, more preferably at least about 95% homology therewith.

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII, and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (“ITAM”) in its cytoplasmic domain. Inhibiting receptor FcγRIIB contains an immunoreceptor tyrosine-based inhibition motif (“ITIM”) in its cytoplasmic domain. Other FcRs, including those to be identified in the future, are encompassed by the term “FcR” herein. FcRs can also increase the serum half-life of antibodies.

As used herein, “percent (%) amino acid sequence identity” and “homology” with respect to a peptide, polypeptide or antibody sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms known in the art needed to achieve maximal alignment over the full length of the sequences being compared.

An “isolated” cell is a cell that is identified and separated from at least one contaminant cell with which it is ordinarily associated in the environment in which it was produced. In some embodiments, the isolated cell is free of association with all components associated with the production environment. The isolated cell is in a form other than in the form or setting in which it is found in nature. Isolated cells are distinguished from cells existing naturally in tissues, organs, or individuals. In some embodiments, the isolated cell is a host cell of the present disclosure.

An “isolated” nucleic acid molecule encoding an antibody, such as an anti-Sortilin antibody of the present disclosure, is a nucleic acid molecule that is identified and separated from at least one contaminant nucleic acid molecule with which it is ordinarily associated in the environment in which it was produced. Preferably, the isolated nucleic acid is free of association with all components associated with the production environment. The isolated nucleic acid molecules encoding the polypeptides and antibodies herein is in a form other than in the form or setting in which it is found in nature. Isolated nucleic acid molecules therefore are distinguished from nucleic acids encoding the polypeptides and antibodies herein existing naturally in cells.

The term “vector,” as used herein, is intended to refer to a nucleic acid molecule capable of transporting another nucleic acid to which it has been linked. One type of vector is a “plasmid,” which refers to a circular double stranded DNA into which additional DNA segments may be ligated. Another type of vector is a phage vector. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors,” or simply, “expression vectors.” In general, expression vectors of utility in recombinant DNA techniques are often in the form of plasmids. In the present specification, “plasmid” and “vector” may be used interchangeably as the plasmid is the most commonly used form of vector.

“Polynucleotide,” or “nucleic acid,” as used interchangeably herein, refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase or by a synthetic reaction.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of the present disclosure.

“Carriers” as used herein include pharmaceutically acceptable carriers, excipients, or stabilizers that are nontoxic to the cell or mammal being exposed thereto at the dosages and concentrations employed.

The term “about” as used herein refers to the usual error range for the respective value readily known to the skilled person in this technical field. Reference to “about” a value or parameter herein includes (and describes) embodiments that are directed to that value or parameter per se.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

It is understood that aspects and embodiments of the present disclosure described herein include “comprising,” “consisting,” and “consisting essentially of” aspects and embodiments.

### Overview

The present disclosure relates to methods of treating and/or delaying the progression of a disease or injury in an individual by administering an anti-Sortilin antibody to the individual. Non-limiting examples of diseases or injuries that may be treated or delayed include frontotemporal dementia (FTD), progressive supranuclear palsy, Alzheimer's disease, vascular dementia, seizures, retinal dystrophy, amyotrophic lateral sclerosis, traumatic brain injury, a spinal cord injury, dementia, stroke, Parkinson's disease, limbic-predominant age-related TDP43 encephalopathy (LATE), acute disseminated encephalomyelitis, retinal degeneration, age related macular degeneration, glaucoma, multiple sclerosis, septic shock, bacterial infection, arthritis, and osteoarthritis. In some embodiments, the disease or injury to be treated or delayed is a neurodegenerative disease, such as FTD.

Patients with neurodegenerative diseases, such as FTD, are affected by the diseases for long periods of time and thus require regular treatment over the course of many years. As intravenous administration of therapeutics cannot be done at home, patients must be transported to infusion centers, which is a burden on both the patient and caregiver. In addition, the memory loss, mood swings, aggression, and other behavioral symptoms of these diseases make patient compliance difficult.

Advantageously, the present disclosure provides methods of treating and/or delaying the progression of FTD in an individual by administering to the individual an anti-Sortilin antibody intravenously at a dose of about 60 mg/kg about once every four weeks (see, e.g., Examples 1 and 2). The relatively infrequent administration of an anti-Sortilin antibody according to the methods described herein (i.e., about once every four weeks) meets the need in the art for identifying methods of treating patients with the correct dose of an anti-Sortilin antibody in ways that ease patient compliance. Moreover, advantageously, the methods of treating and/or delaying the progression of FTD in an individual described herein result in restoration of PGRN to normal levels in plasma and cerebrospinal fluid as compared to controls; time-dependent and durable reduction of biomarkers of FTD disease in cerebrospinal fluid, e.g., lysosomal and inflammatory biomarkers, as compared to controls; stabilization of neurofilament light chain levels in plasma and cerebrospinal fluid; a reduction of brain ventricle enlargement as compared to controls; and a delay in FTD disease progression, as compared to controls. See, e.g., Example 2.

All references cited herein, including patents, patent applications and publications, are hereby incorporated by reference in their entirety.

### Therapeutic Uses

The present disclosure provides methods of treating and/or delaying the progression of a disease or injury in an individual, comprising administering to the individual an anti-Sortilin antibody, wherein the antibody comprises a heavy chain variable region and a light chain variable region, wherein the heavy chain variable region comprises an HVR-H1 comprising the amino acid sequence of SEQ ID NO: 1; an HVR-H2 comprising an amino acid sequence selected from the group consisting of SEQ ID NOs: 2-3; and an HVR-H3 comprising an amino acid sequence selected from the group consisting of SEQ ID NOs: 5-6; and the light chain variable region comprises: an HVR-L1 comprising an amino acid sequence selected from the group consisting of SEQ ID NOs: 8-27; an HVR-L2 comprising an amino acid sequence selected from the group consisting of SEQ ID NOs: 29-30; and an HVR-L3 comprising the amino acid sequence of SEQ ID NO: 32 or 33.

As disclosed herein, anti-Sortilin antibodies of the present disclosure may be used for treating and/or delaying progression of frontotemporal dementia, progressive supranuclear palsy, Alzheimer's disease, vascular dementia, seizures, retinal dystrophy, amyotrophic lateral sclerosis (ALS), traumatic brain injury, a spinal cord injury, dementia, stroke, Parkinson's disease, limbic-predominant age-related TDP43 encephalopathy (LATE), acute disseminated encephalomyelitis, retinal degeneration, age related macular degeneration, glaucoma, multiple sclerosis, septic shock, bacterial infection, arthritis, or osteoarthritis. In some embodiments, the disease or injury is frontotemporal dementia (FTD). In some embodiments, anti-Sortilin antibodies of the present disclosure may be used for treating or alleviating TDP43 pathologies, including but not limited to TDP43 pathologies associated with dementia, C9orf72 associated diseases, FTD, Alzheimer's disease, ALS, LATE, and Parkinson's disease.

In some embodiments, a method of the present disclosure includes an anti-Sortilin antibody comprising two or more anti-Sortilin antibodies.

Dementia

Dementia is a non-specific syndrome (i.e., a set of signs and symptoms) that presents as a serious loss of global cognitive ability in a previously unimpaired person, beyond what might be expected from normal ageing. Dementia may be static as the result of a unique global brain injury. Alternatively, dementia may be progressive, resulting in long-term decline due to damage or disease in the body. While dementia is much more common in the geriatric population, it can also occur before the age of 65. Cognitive areas affected by dementia include, without limitation, memory, attention span, language, and problem solving. Generally, symptoms must be present for at least six months before an individual is diagnosed with dementia.

Exemplary forms of dementia include, without limitation, frontotemporal dementia, Alzheimer's disease, vascular dementia, semantic dementia, and dementia with Lewy bodies.

Without wishing to be bound by theory, it is believed that administering an anti-Sortilin antibody of the present disclosure can treat and/or delay the progression of dementia. In some embodiments, administering an anti-Sortilin antibody may modulate one or more Sortilin activities in an individual having dementia. In some embodiments, administering an anti-Sortilin antibody may induce one or more Progranulin activities in an individual having dementia. In some embodiments, administering an anti-Sortilin antibody may inhibit one or more activities of Sortilin in an individual having dementia. In some embodiments, administering an anti-Sortilin antibody may decrease cellular levels of Sortilin in an individual having dementia. In some embodiments, administering an anti-Sortilin antibody may increase Progranulin levels in an individual having dementia. In some embodiments, administering an anti-Sortilin antibody may inhibit the interaction (e.g., binding) between Progranulin and Sortilin in an individual having dementia. In some embodiments, administering an anti-Sortilin antibody may decrease expression or secretion of pro-inflammatory mediators in an individual having dementia. In some embodiments, administering an anti-Sortilin antibody may inhibit interaction (e.g., binding) between Sortilin and one or more of pro-neurotrophins, neurotrophins, neurotensin, p75, Sortilin propeptide (Sort-pro), amyloid precursor protein (APP), A beta peptide, lipoprotein lipase (LpL), apolipoprotein AV (APOA5), apolipoprotein E (APOE), and receptor associated protein (RAP) in an individual having dementia. In some embodiments, administering an anti-Sortilin antibody may decrease secretion of PCSK9 in an individual having dementia. In some embodiments, administering an anti-Sortilin antibody may decrease production of beta amyloid peptide in an individual having dementia.

### Frontotemporal Dementia

Frontotemporal lobar degeneration (FTLD) or frontotemporal dementia (FTD) is a pathologically and clinically heterogeneous neurodegenerative syndrome characterized by progressive decline in behavior, language, executive skills, and motor function, with atrophy in the frontal and temporal lobes (Rabinovici and Miller, CNS Drugs (2010) 24(5):375-98). Other clinical features of FTD include memory deficits and personality changes (Cruts, M. & Van Broeckhoven, C., Trends Genet. 24:186-194 (2008); Neary, D., et al., Neurology 51:1546-1554 (1998); Ratnavalli, E., Brayne, C., Dawson, K. & Hodges, J. R., Neurology 58:1615-1621 (2002)). Second only to Alzheimer's disease (AD) in prevalence, FTD accounts for about 20% of pre-senile dementia cases. FTD progresses rapidly, with survival after symptom onset of 3 to 14 years (Onyike and Diehl-Schmid, Int Rev Psychiatry (2013) 25(2):130-7).

Classifications of FTD are based on genotype, brain pathology, and phenotype. Individuals with FTD can present with various clinical symptoms, including marked changes in personality, speech, or executive function. Behavioral variant FTD (bvFTD) is characterized by changes in personality and behavior, with persons displaying a mixture of apathy, disinhibition, and lack of insight. Primary progressive aphasia (PPA) is characterized by a gradual, progressive impairment of language capabilities. The two principal language variants, progressive non-fluent aphasia and semantic dementia, present with impaired speech production or impaired comprehension of grammar (agrammatism) and semantic memory, respectively. These progressive changes can result in disrupted relationships with family and friends, loss of decision-making ability and future planning, memory impairment, and loss of independence (Tatton N. FTD Research and Drug Development Landscape. The Association for Frontotemporal Degeneration, 2014. Available from: www.theaftd.org/wp-content/uploads/2014/05/FTD-Research-and-Drug-Development-Landscape.pdf).

A substantial portion of FTD cases are inherited in an autosomal dominant fashion, but even in one family, symptoms can span a spectrum from FTD with behavioral disturbances, to PPA, to Cortico-Basal Ganglionic Degeneration. FTD can be characterized by the pathological presence of specific protein aggregates in the diseased brain. Historically, the first descriptions of FTD recognized the presence of intraneuronal accumulations of hyperphosphorylated Tau protein in neurofibrillary tangles or Pick bodies. A causal role for the microtubule-associated Tau protein was supported by the identification of mutations in the gene encoding the Tau protein in several families (Hutton, M., et al., Nature 393:702-705 (1998). However, the majority of FTD brains show no accumulation of hyperphosphorylated Tau but do exhibit immunoreactivity to ubiquitin (Ub) and TAR DNA binding protein (TDP43) (Neumann, M., et al., Arch. Neurol. 64:1388-1394 (2007)). A majority of FTD cases with Ub inclusions (FTD-U) were shown to carry mutations in the Progranulin gene (GRN).

GRN and C9orf72 Mutations

Progranulin gene (GRN) mutations account for approximately 20% of all familial FTD cases and are caused by a loss-of-function mutation in one allele of GRN (Gass et al., Hum Mol Genet (2006) 15(20):2988-3001; Rademakers et al., Nat Rev Neurol (2012) 8(8):423-34; Rohlfing and Tu, AJNR Am J Neuroradiol (2017) 38(1):10-1; Cruts et al., Hum Mutat (2012) 33(9):1340-4). Heterozygous loss-of-function GRN deficiency almost invariably leads to development of FTD, making GRN a causal gene for the disease (Boxer et al., Alzheimers Dement (2013) 9(2):176-88; Boxer et al., Alzheimers Dement (2013) 9(2):189-98), and suggesting that in healthy individuals, Progranulin expression plays a dose-dependent, critical role in protecting healthy individuals from the development of FTD.

Known GRN mutations include over 77 different mutations in more than 240 unrelated families, which accounts for up to 16% of families worldwide carrying a neurodegenerative disease-causing mutation that encodes for the secreted glycoprotein Progranulin (PGRN) (Ghidoni et al., Brain Res (2012) 1476:172-82; Rademakers et al., Nat Rev Neurol (2012) 8(8):423-34). FTD patients carrying GRN mutations can have reductions in plasma and cerebrospinal fluid (CSF) levels of PGRN (Meeter et al., Dement Geriatr Cogn Dis Extra (2016) 6(2): 330-40). PGRN is associated with many cellular processes that include, but are not limited to, embryogenesis, inflammation, wound repair, neurodegeneration, and lysosome function (Chitramuthu et al., PLoS One (2017) 12(3):e0174784). In the brain, PGRN promotes neurite outgrowth (Gass et al., Mol Neurodegener (2012) 7:33) and enhances the survival of motor and cortical neurons (De Muynck et al., Neurobiol Aging (2013) 34(11):2541-7). The predominant clinical presentation of GRN mutation patients is bvFTD or PPA.

C9orf72 hexanucleotide repeat expansions are also a significant contributor to FTD pathology. Expansion of a non-coding hexanucleotide repeat in C9orf72 is the most common single cause of FTD, representing approximately 25% of familial cases and 6% of sporadic FTD cases (Ng et al., Ann N Y Acad Sci (2015) 1338(1):71-93). The predominant clinical presentation (>80%) of C9orf72 repeat carriers is bvFTD, with motor neuron disease (FTD-MND) or without motor neuron disease (Ng et al., Ann N Y Acad Sci (2015) 1338(1):71-93).

FTD patients with GRN or C9orf72 mutations exhibit a common pathology in frontotemporal degeneration associated with TDP-43 protein-related accumulation. Overlapping functional associations between GRN and C9orf72 proteins include processing of TDP-43, and abnormal glial activation in patients with FTD (Ayala et al., J Cell Sci (2008) 121(22):3778-85; Zhang et al., Proc Natl Acad Sci USA (2009) 106(18):7607-12; Mann and Snowden, Brain Pathol (2017) 27(6):723-36; Valdez et al., Hum Mol Genet (2017) 26(24):4861-72; Busch et al., Hum Mol Genet (2016) 25(13):2681-97; Zhang et al., J Neurosci (2007) 27(39):10530-4; Tanaka et al., Neuroscience (2013) 250:8-19; and O'Rourke et al., Science (2016) 351(6279):1324-29). Moreover, studies have suggested that TMEM106b may be a genetic modifier of both C9orf72 and GRN (Gallagher et al., Acta Neuropathol (2014) 127(3):407-18; van Blitterswijk et al., Acta Neuropathol (2014) 127(3):397-406; and Pottier et al., Lancet Neurol (2018) 17(6):548-58), suggesting that these genes may share a common pathway. In addition, PGRN deficiency has been associated with decreased survival after onset of FTD caused by C9orf72 mutations (van Blitterswijk et al. Mol Neurodegener (2014) 9:38).

In summary, human genetics data and other results support a protective function for Progranulin in FTD. Accordingly, increasing levels of Progranulin, e.g., by inhibiting the interaction between Sortilin and Progranulin, can treat and/or delay the progression of FTD, e.g., in patients with GRN or C9orf72 mutations.

Neurofilament Light Chain Levels

Neurofilaments (Nf) are highly specific major structural proteins of axons, which predominantly consist of Nf-light chain (NfL), Nf-medium chain, Nf-heavy chain, and alpha-internexin. Neurofilament levels are significantly elevated following neuronal degeneration, as disruption of axonal membranes releases Nf into the interstitial fluid and eventually into CSF and blood. Due to this relationship, blood Nf levels may be an effective tool for predicting and monitoring disease progression and for measuring efficacy of neuroprotective treatments (Gaiottino et al., PLoS One (2013) 8(9): e75091).

Increased NfL levels have been observed in FTD (Rohrer et al., Neurology (2016) 87(13):1329-36). A study in 174 subjects identified NfL in both serum and CSF as a potential biomarker for FTD onset (Meeter et al., Ann Clin Transl Neurol (2016) 3(8):623-36). CSF NfL levels in symptomatic and FTD-associated mutation carriers were significantly elevated compared to levels in pre-symptomatic carriers. Serum NfL was elevated in FTD patients as well, and correlated highly with CSF NfL levels. In addition, in bvFTD patient serum, NfL is correlated with functional clinical scores and with atrophy of several brain regions, including the frontal lobes and the white matter underlying these lobes (Steinacker et al., Neurology (2018) 91(15):e1390-401). Serum NfL is now considered a global marker of neurodegeneration (Ashton et al., Acta Neuropathol Commun (2019) 7:5; Lewczuk et al., World J Biol Psychiatry (2018) 19(4):244-328). In addition, it has been shown that an elevated serum NfL level predicts clinical progression and may identify individuals at risk for developing symptoms of FTD (Rojas J C et al., Plasma Neurofilament light chain predicts disease progression in asymptomatic familial frontotemporal lobar degeneration. Poster session presented at: American Academy of Neurology 2019 Annual Meeting; May 4-10; Philadelphia, PA; Rojas et al., Neurology (2021) 96(18):e2296-e2312).

In summary, results from studies in humans suggest that NfL levels in blood (e.g., serum or plasma) and/or CSF, may be useful in identifying individuals who are at risk for developing symptoms of FTD.

In some embodiments, administering an anti-Sortilin antibody of the present disclosure to an individual having symptomatic FTD or at risk for developing symptomatic FTD can treat and/or delay progression of FTD. Accordingly, in some aspects, provided herein are methods of treating and/or delaying the progression of FTD, comprising administering to an individual having symptomatic FTD or at risk for developing symptomatic FTD an anti-Sortilin antibody of the present disclosure.

In some embodiments, administering an anti-Sortilin antibody may modulate one or more Sortilin activities in an individual having symptomatic FTD or at risk for developing symptomatic FTD. In some embodiments, administering an anti-Sortilin antibody may induce one or more Progranulin activities in an individual having symptomatic FTD or at risk for developing symptomatic FTD. In some embodiments, administering an anti-Sortilin antibody may inhibit one or more activities of Sortilin in an individual having symptomatic FTD or at risk for developing symptomatic FTD. In some embodiments, administering an anti-Sortilin antibody may decrease cellular levels of Sortilin in an individual having symptomatic FTD or at risk for developing symptomatic FTD. In some embodiments, administering an anti-Sortilin antibody may increase Progranulin levels in an individual having symptomatic FTD or at risk for developing symptomatic FTD. In some embodiments, administering an anti-Sortilin antibody may inhibit the interaction (e.g., binding) between Progranulin and Sortilin in an individual having symptomatic FTD or at risk for developing symptomatic FTD. In some embodiments, administering an anti-Sortilin antibody may decrease expression or secretion of pro-inflammatory mediators in an individual having symptomatic FTD or at risk for developing symptomatic FTD. In some embodiments, administering an anti-Sortilin antibody may inhibit interaction (e.g., binding) between Sortilin and one or more of pro-neurotrophins, neurotrophins, neurotensin, p75, Sortilin propeptide (Sort-pro), amyloid precursor protein (APP), A beta peptide, lipoprotein lipase (LpL), apolipoprotein AV (APOA5), apolipoprotein E (APOE), and receptor associated protein (RAP) in an individual having symptomatic FTD or at risk for developing symptomatic FTD. In some embodiments, administering an anti-Sortilin antibody may decrease secretion of PCSK9 in an individual having symptomatic FTD or at risk for developing symptomatic FTD. In some embodiments, administering an anti-Sortilin antibody may decrease production of beta amyloid peptide in an individual having symptomatic FTD or at risk for developing symptomatic FTD. In some embodiments, administering an anti-Sortilin antibody may decrease NfL levels in blood (e.g., serum or plasma) and/or CSF in an individual having symptomatic FTD or at risk for developing symptomatic FTD.

In some embodiments, an individual treated according to any of the methods of treating and/or delaying progression of FTD provided herein is at risk for developing symptomatic FTD or has FTD (i.e., has symptomatic FTD) prior to the start of treatment according to the methods provided herein. In some embodiments, the individual is heterozygous for a loss-of-function mutation in GRN (the Granulin gene). In some embodiments, the loss-of-function mutation in GRN is causative of FTD. In some embodiments, the individual is at risk for developing symptomatic FTD due to a heterozygous loss-of-function mutation in GRN. In some embodiments, the individual has symptomatic FTD due to a heterozygous loss-of-function mutation in GRN. In some embodiments, the individual has a global Clinical Dementia Rating Dementia Staging Instrument PLUS National Alzheimer's Disease Coordinating Center Frontotemporal Lobar Degeneration Behavior and Language Domains (CDR® plus NACC FTLD) score of between 0 and 2, e.g., any of 0, 0.5, 1, or 2, prior to the start of treatment according to the methods provided herein.

In some embodiments, an individual treated according to any of the methods of treating and/or delaying progression of FTD provided herein is heterozygous for a C9orf72 mutation. In some embodiments, the C9orf72 mutation is a hexanucleotide repeat expansion. In some embodiments, the C9orf72 mutation is causative of FTD. In some embodiments, the individual has symptomatic FTD. In some embodiments, the individual is asymptomatic but has a C9orf72 mutation. In some embodiments, the individual has a global Clinical Dementia Rating Dementia Staging Instrument PLUS National Alzheimer's Disease Coordinating Center Frontotemporal Lobar Degeneration Behavior and Language Domains (CDR® plus NACC FTLD) score of between 0 and 2, e.g., any of 0, 0.5, 1, or 2, prior to the start of treatment according to the methods provided herein.

In some embodiments, the methods provided herein comprise assessing the individual for the presence of one or more GRN mutations prior to administration of the anti-Sortilin antibody. In some embodiments, the presence of mutations in GRN, e.g. in an individual or in a sample from an individual, is determined by any method known in the art. Non-limiting examples of methods that may be used to determine the presence of mutations in GRN include DNA sequencing, DNA hybridization, polymerase chain reaction (PCR), multiplex PCR, nested PCR, real-time PCR, quantitative PCR, semi-quantitative PCR, DNA microarrays, multiplex ligation-dependent probe amplification, single strand conformation polymorphism analysis, denaturing gradient gel electrophoresis, heteroduplex analysis, Southern blotting, genetic linkage analysis (e.g., using short tandem repeats and/or variable number tandem repeats), fluorescence in situ hybridization, comparative genomic hybridization, allele-specific amplification, and/or restriction enzyme digestion methods (e.g., restriction-fragment length polymorphism analysis) (Mahdieh et al., Iran J Pediatr (2013) 23(4):375-388). In some embodiments, the presence of mutations in GRN is determined by DNA sequencing (Chang et al., (2010) Arch Neurol 67(2):161-170). In some embodiments, the presence of mutations in GRN is determined by DNA sequencing and genotyping (Chang et al., (2010) Arch Neurol 67(2):161-170). In some embodiments, low blood (e.g., plasma or serum) Progranulin levels predicts the presence of mutations in GRN in an individual (Schofield et al., (2010) J Alzheimers Dis 22(3):981-4).

In some embodiments, the individual is at risk for developing symptomatic FTD prior to the start of treatment according to the methods provided herein. In some embodiments, the individual does not show symptoms of FTD prior to the start of treatment according to the methods provided herein. In some embodiments, an individual at risk for developing symptomatic FTD has a CDR® plus NACC FTLD-SB score≤0.5. In some embodiments, an individual at risk for developing symptomatic FTD has an elevated level of serum NfL prior to the start of treatment according to the methods provided herein. In some embodiments, an individual at risk for developing symptomatic FTD has a CDR® plus NACC FTLD-SB score≤0.5 and an elevated level of serum NfL prior to the start of treatment according to the methods provided herein. In some embodiments, an individual with an elevated level of serum NfL prior to the start of treatment according to the methods provided herein has a serum NfL level of at least about 13.6 pg/mL, e.g., as determined in a sample of serum obtained from the individual. In some embodiments, an individual with an elevated level of serum NfL prior to the start of treatment according to the methods provided herein has a serum NfL level of at least about 19.8 pg/mL, e.g., as determined in a sample of serum obtained from the individual. See, e.g., Rojas et al., Neurology (2021) 96(18):e2296-e2312; Rojas J C et al., Plasma Neurofilament light chain predicts disease progression in asymptomatic familial frontotemporal lobar degeneration. Poster session presented at: American Academy of Neurology 2019 Annual Meeting; May 4-10; Philadelphia, PA. In some embodiments, the individual has a serum NfL level of between about 13.6 pg/mL and about 20 pg/mL, between about 20 pg/mL and about 30 pg/mL, between about 30 pg/mL and about 40 pg/mL, between about 40 pg/mL and about 50 pg/mL, between about 50 pg/mL and about 60 pg/mL, between about 60 pg/mL and about 70 pg/mL, between about 70 pg/mL and about 80 pg/mL, between about 80 pg/mL and about 90 pg/mL, between about 90 pg/mL and about 100 pg/mL, between about 100 pg/mL and about 110 pg/mL, between about 110 pg/mL and about 120 pg/mL, between about 120 pg/mL and about 130 pg/mL, between about 130 pg/mL and about 140 pg/mL, between about 140 pg/mL and about 150 pg/mL, between about 150 pg/mL and about 160 pg/mL, between about 160 pg/mL and about 170 pg/mL, between about 170 pg/mL and about 180 pg/mL, between about 180 pg/mL and about 190 pg/mL, between about 190 pg/mL and about 200 pg/mL, between about 200 pg/mL and about 210 pg/mL, between about 210 pg/mL and about 220 pg/mL, between about 220 pg/mL and about 230 pg/mL, between about 230 pg/mL and about 240 pg/mL, between about 240 pg/mL and about 250 pg/mL, between about 250 pg/mL and about 260 pg/mL, between about 260 pg/mL and about 270 pg/mL, between about 270 pg/mL and about 280 pg/mL, between about 280 pg/mL and about 290 pg/mL, between about 290 pg/mL and about 300 pg/mL, between about 300 pg/mL and about 310 pg/mL, between about 310 pg/mL and about 320 pg/mL, between about 320 pg/mL and about 330 pg/mL, between about 330 pg/mL and about 340 pg/mL, between about 340 pg/mL and about 350 pg/mL, between about 350 pg/mL and about 360 pg/mL, between about 360 pg/mL and about 370 pg/mL, between about 370 pg/mL and about 380 pg/mL, between about 380 pg/mL and about 390 pg/mL, between about 390 pg/mL and about 400 pg/mL, between about 400 pg/mL and about 410 pg/mL, between about 410 pg/mL and about 420 pg/mL, between about 420 pg/mL and about 430 pg/mL, between about 430 pg/mL and about 440 pg/mL, between about 440 pg/mL and about 450 pg/mL, between about 450 pg/mL and about 460 pg/mL, between about 460 pg/mL and about 470 pg/mL, between about 470 pg/mL and about 480 pg/mL, between about 480 pg/mL and about 490 pg/mL, between about 490 pg/mL and about 500 pg/mL, between about 500 pg/mL and about 510 pg/mL, between about 510 pg/mL and about 520 pg/mL, between about 520 pg/mL and about 530 pg/mL, between about 530 pg/mL and about 540 pg/mL, between about 540 pg/mL and about 550 pg/mL, between about 550 pg/mL and about 560 pg/mL, between about 560 pg/mL and about 570 pg/mL, between about 570 pg/mL and about 580 pg/mL, between about 580 pg/mL and about 590 pg/mL, or between about 590 pg/mL and about 600 pg/mL. In some embodiments, the individual has a serum NfL level of any of at least about 13.6 pg/mL, at least about 15 pg/mL, at least about 19.8 pg/mL, at least about 20 pg/mL, at least about 30 pg/mL, at least about 40 pg/mL, at least about 50 pg/mL, at least about 60 pg/mL, at least about 70 pg/mL, at least about 80 pg/mL, at least about 90 pg/mL, at least about 100 pg/mL, at least about 110 pg/mL, at least about 120 pg/mL, at least about 130 pg/mL, at least about 140 pg/mL, at least about 150 pg/mL, at least about 160 pg/mL, at least about 170 pg/mL, at least about 180 pg/mL, at least about 190 pg/mL, at least about 200 pg/mL, at least about 210 pg/mL, at least about 220 pg/mL, at least about 230 pg/mL, at least about 240 pg/mL, at least about 250 pg/mL, at least about 260 pg/mL, at least about 270 pg/mL, at least about 280 pg/mL, at least about 290 pg/mL, at least about 300 pg/mL, at least about 310 pg/mL, at least about 320 pg/mL, at least about 330 pg/mL, at least about 340 pg/mL, at least about 350 pg/mL, at least about 360 pg/mL, at least about 370 pg/mL, at least about 380 pg/mL, at least about 390 pg/mL, at least about 400 pg/mL, at least about 410 pg/mL, at least about 420 pg/mL, at least about 430 pg/mL, at least about 440 pg/mL, at least about 450 pg/mL, at least about 460 pg/mL, at least about 470 pg/mL, at least about 480 pg/mL, at least about 490 pg/mL, at least about 500 pg/mL, at least about 510 pg/mL, at least about 520 pg/mL, at least about 530 pg/mL, at least about 540 pg/mL, at least about 550 pg/mL, at least about 560 pg/mL, at least about 570 pg/mL, at least about 580 pg/mL, at least about 590 pg/mL, or at least about 600 pg/mL.

In some embodiments, the methods provided herein comprise assessing the individual for the presence of an elevated level of NfL prior to administration of the anti-Sortilin antibody to the individual, e.g., in a sample of serum from the individual. In some embodiments, the methods provided herein comprise acquiring knowledge of the presence or absence of an elevated level of NfL in an individual (e.g., in a sample of serum from the individual) prior to administration of the anti-Sortilin antibody to the individual. In some embodiments, an anti-Sortilin antibody of the disclosure is administered to an individual according to any of the methods described herein responsive to acquiring knowledge of the presence of an elevated level of NfL in the individual (e.g., in a sample of serum from the individual). In some embodiments, knowledge of the presence or absence of an elevated level of NfL in an individual is acquired directly, e.g., by measuring the level of NfL in sample from the individual (e.g., in a sample of serum from the individual). In some embodiments, knowledge of the presence or absence of an elevated level of NfL in an individual (e.g., in a sample of serum from the individual) is acquired indirectly, e.g., from a third party, such as, without limitation, a clinician, a caregiver, a laboratory, a hospital, a clinic, a nursing home, a third-party payer, an medical insurance company, a government, and the like.

NfL levels in a sample obtained from an individual, e.g., in a serum sample, may be measured by any known methods in the art including, without limitation, immunoassays, a single-molecule array technology (Simoa) assay (e.g., using commercially available kits, such as the NF-light digital immunoassay kit or Simoa HD-1 assay from Quanterix, Billerica, MA; or a Neurology 4-Plex A kit, see, e.g., Heller et al., J Neurol Neurosurg Psychiatry (2020) 91(3):263-270), ELISA, or using other assays from Quanterix or Roche Diagnostics.

In some embodiments, the individual has symptomatic FTD prior to the start of treatment according to the methods provided herein. In some embodiments, an individual with symptomatic FTD has a CDR® plus NACC FTLD-SB score of >0.5, and 1 or more of the 6 behavioral/cognitive symptoms required for a diagnosis of possible behavioral variant FTD (bvFTD), i.e., disinhibition, apathy/inertia, loss of sympathy/empathy, perseverative/compulsive behaviors, hyperorality, and dysexecutive neuropsychological profile (Rascovsky et al., Brain (2011) 134(Pt 9):2456-77). In some embodiments, an individual with symptomatic FTD has a CDR® plus NACC FTLD-SB score of >0.5 and a diagnosis of primary progressive aphasia (PPA; Gorno-Tempini et al., Neurology (2011) 76(11):1006-14). In some embodiments, an individual with symptomatic FTD has one or more of the 6 behavioral/cognitive symptoms required for a diagnosis of possible bvFTD. In some embodiments, an individual with symptomatic FTD has a diagnosis of PPA. In some embodiments, an individual with symptomatic FTD has a CDR® plus NACC FTLD-global score of 0.5, 1, or 2, and one or more of the 6 behavioral/cognitive symptoms required for a diagnosis of possible bvFTD or a diagnosis of PPA. In some embodiments, an individual treated according to the methods provided herein does not have a CDR® plus NACC FTLD global score of greater than 2.

In some embodiments, an individual treated according to the methods provided herein is a human. In some embodiments, an individual treated according to the methods provided herein is a human adult.

In some embodiments, an individual at risk for developing symptomatic FTD or having symptomatic FTD prior to the start of treatment according to the methods provided herein does not have dementia due to a condition other than FTD, including, but not limited to, Alzheimer's disease, Parkinson's disease, dementia with Lewy bodies, Huntington disease, or vascular dementia. In some embodiments, the individual does not have one or more mutations causative of neurodegenerative disorder(s) other than heterozygous loss-of-function GRN mutations causative of FTD prior to the start of treatment according to the methods provided herein. In some embodiments, the individual does not have history of severe allergic, anaphylactic, or other hypersensitivity reactions to chimeric, human, or humanized antibodies or fusion proteins prior to the start of treatment according to the methods provided herein. In some embodiments, the individual does not have signs or symptoms of progressive supranuclear palsy or bulbar dysfunction, such as postural instability, eye problems, and swallowing difficulties, prior to the start of treatment according to the methods provided herein. In some embodiments, the individual does not have history of moderate or severe substance use disorder within the past 2 years prior to the start of treatment according to the methods provided herein, with the exception of nicotine, as defined by the Diagnostic and Statistical Manual of Mental Disorders, fifth edition criteria (American Psychiatric Association 2013). In some embodiments, the individual does not have or has not had an acute illness that requires or required systemic antibiotics within 30 days prior to the start of treatment according to the methods provided herein. In some embodiments, the individual does not have clinically significant vitamin B12 or folate deficiency prior to the start of treatment according to the methods provided herein. In some embodiments, the individual is on a stable regimen for treating vitamin B12 or folate deficiency for at least 3 months prior to the start of treatment according to the methods provided herein. In some embodiments, the individual does not have untreated hypothyroidism prior to the start of treatment according to the methods provided herein. In some embodiments, the individual is treated with a stable thyroid supplementation dose for at least 3 months with a normal thyroid-stimulating hormone level prior to the start of treatment according to the methods provided herein. In some embodiments, the individual does not have insufficiently controlled diabetes mellitus (e.g., hemoglobin A1C≥8%). In some embodiments, the individual has not had any surgery (major or emergent) or hospitalization within 30 days prior to the start of treatment according to the methods provided herein. In some embodiments, the individual does not have history of cancer within the last 5 years prior to the start of treatment according to the methods provided herein, except for basal cell or squamous cell carcinoma. In some embodiments, the individual is not positive for hepatitis B surface antigen, human immunodeficiency virus-1 or -2 antibodies or antigen, and/or does not have history of spirochetal infection of the central nervous system (e.g., syphilis, borreliosis, or Lyme disease) prior to the start of treatment according to the methods provided herein. In some embodiments, the individual is positive for hepatitis C virus antibody and is negative for hepatitis C RNA prior to the start of treatment according to the methods provided herein. In some embodiments, the individual does not have significant kidney disease prior to the start of treatment according to the methods provided herein. In some embodiments, significant kidney disease includes an estimated glomerular filtration rate (eGFR)<30 mL/min/1.73 m², according to the re-expressed abbreviated (four-variable) Modification of Diet in Renal Disease (MDRD) Study equation. The MDRD equation is as follows: eGFR (mL/min/1.73 m²)=175×(standardized serum creatinine)^{-1.154}×(Age)^{-0.203}×(0.742 if female)×(1.212 if black). In some embodiments, significant kidney disease includes a creatinine≥2 mg/dL. In some embodiments, the individual does not have impaired hepatic function prior to the start of treatment according to the methods provided herein, e.g., as indicated by aspartate aminotransferase (AST) or alanine aminotransferase (ALT)≥2.5×the upper limit of normal (ULN), or total bilirubin≥1.5×ULN. In some embodiments, the individual has Gilbert's syndrome prior to the start of treatment according to the methods provided herein. In some embodiments, the individual does not have hematologic abnormalities prior to the start of treatment according to the methods provided herein, as indicated by hemoglobin≤10 g/dL; white blood cells (WBC)≤3 000/mm³; absolute neutrophil count≤1 1,000/mm³; or platelet count≤150,000/mm³. In some embodiments, the individual does not have or has not had unstable or clinically significant cardiovascular disease (e.g., myocardial infarction, angina pectoris, New York Heart Association Class III or IV cardiac failure) within the past 2 years prior to the start of treatment according to the methods provided herein. In some embodiments, the individual does not have uncontrolled hypertension (e.g., repeated supine diastolic blood pressure [BP]>95 mm Hg or systolic BP>150 mm Hg) prior to the start of treatment according to the methods provided herein. In some embodiments, the individual does not have history or presence of an abnormal electrocardiogram that is clinically significant prior to the start of treatment according to the methods provided herein, including complete left bundle branch block, second- or third-degree atrioventricular block, or evidence of acute or subacute myocardial infarction or ischemia. In some embodiments, the individual does not have history of ventricular dysrhythmias or risk factors for ventricular dysrhythmias such as structural heart disease (e.g., severe left ventricular systolic dysfunction, left ventricular hypertrophy) or clinically significant electrolyte abnormalities (e.g., hypokalemia, hypomagnesemia, hypocalcemia) prior to the start of treatment according to the methods provided herein. In some embodiments, the individual has premature ventricular contractions. In some embodiments, the individual does not have history or presence of clinically evident vascular disease potentially affecting the brain prior to the start of treatment according to the methods provided herein (e.g., clinically significant carotid or vertebral artery stenosis or plaque; cerebral hemorrhage or infarct greater than 1 cm³; 3 or more lacunar infarcts in any location; cerebral contusion; encephalomalacia; intracranial aneurysm; arteriovenous malformation; subdural hematoma); hydrocephalus; space-occupying lesions (e.g., abscess or brain tumor such as meningioma) that have the potential to affect cognitive function; or intracranial tumor that is clinically relevant (e.g., glioma, cerebral metastasis). In some embodiments, the individual does not have history of a clinically significant, persistent neurologic deficit, structural brain damage, or CNS trauma prior to the start of treatment according to the methods provided herein.

In some embodiments, the individual has not taken a cannabinoid within at least 90 days prior to the start of treatment according to the methods provided herein. In some embodiments, the individual has not taken any benzodiazepines and tricyclic antidepressants at least 90 days prior to the start of treatment according to the methods provided herein. In some embodiments, the individual has not taken any stimulant medication (e.g., amphetamine, dextroamphetamine, dexmethylphenidate, lisdexamfetamine, methylphenidate) unless prescribed as a stable regimen for at least 90 days prior to the start of treatment according to the methods provided herein. In some embodiments, the individual has not taken any passive immunotherapy (e.g., immunoglobulin) or other long-acting biologic agent to prevent or postpone cognitive decline within 1 year prior to the start of treatment according to the methods provided herein. In some embodiments, the individual has not taken a typical (first-generation) antipsychotic or neuroleptic medication within 6 months prior to the start of treatment according to the methods provided herein, except as needed for brief treatment of a nonpsychiatric indication (e.g., emesis). In some embodiments, the individual has taken an atypical (second-generation) antipsychotic medication or pimavanserin on a stable regimen for at least 90 days prior to the start of treatment according to the methods provided herein. In some embodiments, the individual has not taken anticoagulation medications (e.g., coumadin, heparinoids, apixaban) within 90 days prior to the start of treatment according to the methods provided herein. In some embodiments, the individual has taken aspirin or antiplatelet medication prior to the start of treatment according to the methods provided herein. In some embodiments, the individual has not taken a systemic immunosuppressive therapy prior to the start of treatment according to the methods provided herein. In some embodiments, the individual has taken a stable regimen of prednisone≤10 mg/day or an equivalent corticosteroid for at least 90 days prior to the start of treatment according to the methods provided herein, and the individual has hemoglobin>9 g/dL, WBC count>3 000/mm³, absolute neutrophil count>1 500/mm³, and platelet count>100 000/mm³. In some embodiments, the individual has not had chronic use of opioids (including long-acting opioid medication) within 90 days prior to the start of treatment according to the methods provided herein. In some embodiments, the individual has not had chronic use of barbiturates or hypnotics starting from 3 months prior to the start of treatment according to the methods provided herein.

Assessments and Biomarkers

In some embodiments, treatment and/or delay of FTD progression is assessed based on one or more neurocognitive, functional, or quality of life tests or assessments (i.e., clinical outcome assessments) after the start of treatment according to the methods provided herein, e.g., as compared to the one or more clinical outcome assessments prior to the start of treatment according to the methods provided herein. In some embodiments, the methods of treating or delaying progression of FTD comprise performing one or more clinical outcome assessments on the individual before and after the individual has received one or more doses of the anti-Sortilin antibody. Non-limiting examples of clinical outcome assessments that may be used to evaluate the treatment and/or delay of FTD progression include the Frontotemporal Dementia Clinical Rating Scale (FCRS), the Frontotemporal Dementia Rating Scale (FRS), the Clinical Global Impression-Improvement (CGI-I) assessment, the Neuropsychiatric Inventory (NPI) assessment, the Color Trails Test (CTT) Part 2, the Repeatable Battery for the Assessment of Neuropsychological Status (RBANS), the Delis-Kaplan Executive Function System Color-Word Interference Test, the Interpersonal Reactivity Index, the Winterlight Lab Speech Assessment (WLA), the Summerlight Lab Speech Assessment (SLA), the Sheehan-Suicidality Tracking Scale (Sheehan-STS), the Clinical Global Impression-Severity (CGI-S), the Clinical Dementia Rating Dementia Staging Instrument PLUS National Alzheimer's Disease Coordinating Center frontotemporal lobar degeneration Behavior and Language Domains (CDR® plus NACC FTLD), the European Quality of Life-5 Dimensions (EQ-5D), the Clinical Dementia Rating Dementia Staging Instrument PLUS National Alzheimer's Disease Coordinating Center frontotemporal lobar degeneration Behavior and Language Domains Sum of Boxes (CDR® plus NACC FTLD SB), CDR® plus NACC FTLD-global score, or the Zarit Burden Interview (ZBI). In some embodiments, treatment and/or delay of FTD progression is assessed based on one clinical outcome assessment. In some embodiments, treatment and/or delay of FTD progression is assessed based on more than one clinical outcome assessments (e.g., 2, 3, 4, 5, 6, 7, 8, 9 or more clinical outcome assessments). In some embodiments, the clinical outcome assessment is a pharmacoeconomic assessment. In some embodiments, treatment and/or delay of FTD progression is assessed based on one or more pharmacoeconomic assessments, such as the Resource Utilization in Dementia-Lite Version (RUD-Lite) assessment, after the start of treatment according to the methods provided herein, e.g., as compared to the one or more pharmacoeconomic assessments prior to the start of treatment according to the methods provided herein.

In some embodiments, the clinical outcome assessment is the CDR® plus NACC FTLD assessment. The CDR® plus NACC FTLD assessment is the Clinical Dementia Rating Scale (CDR®) from Washington University plus the behavior and language domains from the NACC FTLD module. The CDR® characterizes 6 domains of cognitive and functional performance applicable to Alzheimer's disease and related dementias: memory, orientation, judgment and problem solving, community affairs, home and hobbies, and personal care. The necessary information to make each rating is obtained through a semi-structured interview of the participant and a reliable informant or collateral source (e.g., a caregiver). See, e.g., knightadre.wustl.edu/cdr/cdr.htm. The sum of boxes (SB) of the CDR® is a detailed quantitative general index that provides more information than the CDR®-global score (GS) in participants with mild dementia (O'Bryant et al., Arch Neurol (2010) 67(6):746-9). The NACC FTLD module from the National Alzheimer's Coordinating Center (NACC) is a standard clinical evaluation for FTLD, see, e.g., naccdata.org/data-collection/forms-documentation/ftld-3. In some embodiments, the clinical outcome assessment is the CDR® plus NACC FTLD-SB assessment. The CDR® plus NACC FTLD-SB includes the CDR®-SB and the behavior and language domains from the NACC FTLD module. Additional information about the CDR® plus NACC FTLD and CDR® plus NACC FTLD-SB assessments may be found in, e.g., Miyagawa et al., Alzheimers Dement (2020) 16(1):79-90; and Miyagawa et al., Alzheimers Dement (2020) 16(1):106-117.

In some embodiments, the clinical outcome assessment is the CGI-S assessment. The CGI-S is a 7-point Likert scale that is used by a clinician to rate the severity of a participant's disease relative to the clinician's past experience with patients who have the same diagnosis. In some embodiments, the clinical outcome assessment is the CGI-I assessment. The CGI-I is a 7-point Likert scale that is used by a clinician to rate how much a participant's disease has improved or worsened relative to baseline. The CGI-I may be administered to assess (a) total improvement/worsening; (b) total improvement/worsening of behavior, motivation, and social cognition symptoms; and/or (c) total improvement/worsening of language abilities. Additional information about the CGI-S and CGI-I assessments may be found in, e.g., Busner and Targum, Psychiatry (Edgmont) (2007) 4(7):28-37.

In some embodiments, the clinical outcome assessment is the RBANS assessment. The RBANS is a collection of 12 subtests representing 5 neurocognitive domains: Immediate Memory, Visuospatial/Constructional, Language, Attention, and Delayed Memory. The raw scores from each subtest within a domain are converted to a summary score, or Index Score, for the domain by consulting normative data tables. The RBANS also provides an overall Index Score that summarizes the patient's overall level of performance on this measure. Additional information about the RBANS assessment may be found in, e.g., Randolph et al., J Clin Exp Neuropsychol (1998) 20(3):310-9.

In some embodiments, the clinical outcome assessment is the FRS assessment. The FRS is a 30-item scale designed to assess the frequency of problematic behaviors and difficulties with activities of daily living such as shopping, chores, telephone use, management of finances and medications, meal preparation and eating, self-care, and mobility. Additional information about the FRS assessment may be found in, e.g., Mioshi et al., Neurology (2010) 74(20):1591-7.

In some embodiments, the clinical outcome assessment is the EQ-5D assessment. The EQ-5D is a standardized instrument developed by the EuroQol Group and consists of 2 pages: the EQ-5D descriptive system and the EQ visual analogue scale. The EQ-5D descriptive system is comprised of 5 dimensions: Mobility, Self-Care, Usual Activities, Pain/Discomfort, and Anxiety/Depression. In the EQ visual analogue scale, an informant or collateral source (e.g., a caregiver) is asked to rate the individual's health-related quality of life in their opinion on a vertical visual analogue scale, which can be used as a quantitative measure of the individual's health outcome. The EQ-5D is available at euroqol.org/eq-5d-instruments. Additional information about the EQ-5D assessment may be found in, e.g., Rabin and de Charro, Ann Med (2001) 33(5):337-43.

In some embodiments, the clinical outcome assessment is the ZBI. The ZBI is a 29-item questionnaire (22 items in the revised questionnaire) that an informant or collateral source (e.g., a caregiver) completes using a 5-point scale (0, never; 4, near always) to determine the degree of burden that the individual's care places on the informant or collateral source (e.g., a caregiver). A sample ZBI can be found in dementiapathways.ie/_filecache/edd/c3c/89-zarit_burden_interview.pdf.

In some embodiments, the clinical outcome assessment is the RUD or RUD-LITE. The RUD is a standardized instrument to compare economic costs and resource utilization in dementia across different countries. The RUD is available as the full Version (RUD) or an abbreviated version (RUD-lite) (see, rudinstrument.com). In some embodiments, the clinical outcome assessment is the RUD-LITE. Additional information about the RUD-LITE assessment may be found in, e.g., Wimo and Winblad, Brain Aging (2003) 3:48-59.

In some embodiments, the clinical outcome assessment is the Sheehan-STS. The Sheehan-STS is a brief scale designed to assess and monitor over time the core phenomena of suicidality. The Sheehan-STS is a sensitive psychometric tool to prospectively assess for treatment-emergent suicidal thoughts and behaviors. The Sheehan-STS is a 16-item scale that may be administered either by a clinician or through a self-report. Each item in the Sheehan-STS is scored on a 5-point Likert scale (0=not at all, 1=a little, 2=moderately, 3=very, and 4=extremely). Additional information about the Sheehan-STS assessment may be found in, e.g., Sheehan et al., Innov Clin Neurosci (2014) 11(9-10):93-140.

In some embodiments, the clinical outcome assessment is the WLA assessment. The WLA evaluates speech, language, and cognition using short samples of speech. Software decomposes a speech sample into over 500 individual markers. These markers quantify both the acoustic and linguistic properties of the speech. Acoustic markers describe properties of the sound wave itself such as tone, speaking rate, pausing (both filled and unfilled), pitch, and spectral power. Linguistic markers are extracted from the content of speech (e.g., transcripts) and include the frequency of different parts of speech (such as nouns, verbs, pronouns, and prepositions) as well as more global measures of discourse coherence and the complexity of syntax and grammar. Additional information about the WLA assessment may be found in, e.g., winterlightlabs.com.

In some embodiments, treatment with an anti-Sortilin antibody of the present disclosure reduces or delays FTD disease progression, assessed based on one or more clinical outcome assessments described herein. In some embodiments, treatment with an anti-Sortilin antibody of the present disclosure reduces or delays FTD disease progression, assessed using the CDR® plus NACC FTLD-SB assessment, as compared to disease progression in a corresponding individual not treated with the anti-Sortilin antibody. In some embodiments, treatment with an anti-Sortilin antibody of the present disclosure results in a reduction or delay of FTD disease progression of at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 60%, at least about 65%, at least about 70%, at least about 75%, at least about 80%, at least about 85%, at least about 90%, at least about 95%, or 100%, e.g., as compared to disease progression in a corresponding individual not treated with the anti-Sortilin antibody. In some embodiments, FTD disease progression is assessed using the CDR® plus NACC FTLD-SB assessment. In some embodiments, the reduction or delay of FTD disease progression is assessed at any of at least about 1 month, at least about 3 months, at least about 6 months, at least about 9 months, at least about 12 months, at least about 15 months, at least about 18 months, at least about 21 months, at least about 24 months, or more, after the start of treatment according to the methods provided herein. In some embodiments, the reduction or delay of FTD disease progression is assessed at about 12 months after the start of treatment according to the methods provided herein.

In some aspects, provided herein are methods of treating and/or delaying the progression of FTD, comprising administering to an individual an anti-Sortilin antibody of the present disclosure, wherein administration of the anti-Sortilin antibody results in a reduction or delay of FTD disease progression of at least about 30%, at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 60%, at least about 65%, at least about 70%, at least about 75%, at least about 80%, at least about 85%, at least about 90%, at least about 95%, or 100%, e.g., as compared to disease progression in a corresponding individual not treated with the anti-Sortilin antibody. In some embodiments, FTD disease progression is assessed using the CDR® plus NACC FTLD-SB assessment. In some embodiments, the anti-Sortilin antibody is administered according to any of the methods for treating and/or delaying the progression of FTD provided herein. In some embodiments, the reduction or delay of FTD disease progression is assessed at any of at least about 1 month, at least about 3 months, at least about 6 months, at least about 9 months, at least about 12 months, at least about 15 months, at least about 18 months, at least about 21 months, at least about 24 months, or more, after the start of treatment according to the methods provided herein. In some embodiments, the reduction or delay of FTD disease progression is assessed at about 12 months after the start of treatment according to the methods provided herein. In some embodiments, the individual has symptomatic FTD. In some embodiments, the individual has one or more Granulin mutations causative of FTD. In some embodiments, the individual has one or more Granulin mutations causative of FTD and is asymptomatic. In some embodiments, the individual has a C9orf72 mutation. In some embodiments, the individual is heterozygous for a C9orf72 mutation. In some embodiments, the C9orf72 mutation is a hexanucleotide repeat expansion. In some embodiments, the C9orf72 mutation is causative of FTD.

In some embodiments, treatment and/or delay of FTD progression is assessed based on the level of Progranulin protein in the plasma of the individual after the start of treatment according to the methods provided herein, e.g., as compared to the level of Progranulin protein in the plasma of the individual prior to the start of treatment according to the methods provided herein. In some embodiments, treatment and/or delay of FTD progression is assessed based on the level of Progranulin protein in the cerebrospinal fluid of the individual after the start of treatment according to the methods provided herein, e.g., as compared to the level of Progranulin protein in the cerebrospinal fluid of the individual prior to the start of treatment according to the methods provided herein. In some embodiments, the methods of treating or delaying progression of FTD comprise measuring the level of Progranulin protein in a sample of blood plasma obtained from the individual before and after the individual has received one or more doses of the anti-Sortilin antibody. In some embodiments, the methods of treating or delaying progression of FTD comprise measuring the level of Progranulin protein in a sample of cerebrospinal fluid obtained from the individual before and after the individual has received one or more doses of the anti-Sortilin antibody. In some embodiments, treatment with an anti-Sortilin antibody of the present disclosure increases Progranulin protein levels in plasma and/or cerebrospinal fluid of the individual by any of at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, or more, e.g., as compared to prior to the start of treatment according to the methods provided herein. In some embodiments, treatment with an anti-Sortilin antibody of the present disclosure increases Progranulin protein levels in plasma and/or cerebrospinal fluid of the individual by any of at least about 100%, at least about 110%, at least about 115%, at least about 120%, at least about 125%, at least about 130%, at least about 135%, at least about 140%, at least about 145%, at least about 150%, or more, e.g., as compared to prior to the start of treatment according to the methods provided herein. In some embodiments, treatment with an anti-Sortilin antibody of the present disclosure increases Progranulin protein levels in plasma and/or cerebrospinal fluid of the individual by any of at least about 150%, at least about 175%, at least about 200%, at least about 225%, at least about 250%, at least about 275%, at least about 300%, at least about 400%, or more, e.g., as compared to prior to the start of treatment according to the methods provided herein.

In some embodiments, an individual treated according to the methods of the disclosure has one or more GRN mutations, and before receiving one or more doses of an anti-Sortilin antibody of the disclosure, the individual has a Progranulin protein plasma or cerebrospinal fluid level that is lower than normal Progranulin protein plasma or cerebrospinal fluid levels, e.g., as observed in controls, such as age-matched procured controls. In some embodiments, treatment with an anti-Sortilin antibody of the present disclosure results in the individual having a Progranulin protein plasma or cerebrospinal fluid level that is elevated compared to the plasma or cerebrospinal fluid level of Progranulin protein prior to administration of the anti-Sortilin antibody. In some embodiments, treatment with an anti-Sortilin antibody of the present disclosure results in the individual having a Progranulin protein plasma or cerebrospinal fluid level that is within the range of normal Progranulin protein plasma or cerebrospinal fluid levels observed in controls, such as age-matched procured controls.

In some embodiments, an individual treated according to the methods of the disclosure has a hexanucleotide repeat expansion C9orf72 mutation. In some embodiments, treatment with an anti-Sortilin antibody of the present disclosure results in the individual having a Progranulin protein plasma or cerebrospinal fluid level that is elevated compared to the plasma or cerebrospinal fluid level of Progranulin protein prior to administration of the anti-Sortilin antibody.

In some embodiments, treatment with an anti-Sortilin antibody of the present disclosure increases Progranulin protein levels in plasma and/or cerebrospinal fluid of the individual to levels that are within the range of Progranulin protein levels of healthy individuals. In some embodiments, a healthy individual is a corresponding individual, e.g., a corresponding human individual, that does not have, or has not been diagnosed with, frontotemporal dementia or symptomatic frontotemporal dementia, and has similar characteristics to the individual treated according to the methods of the disclosure, such as age, sex, genetics, and/or other biomarkers or baseline assessments. In some embodiments, the healthy individual is an age-matched procured control. In some embodiments, treatment with an anti-Sortilin antibody of the present disclosure increases Progranulin protein levels in plasma and/or cerebrospinal fluid of the individual to levels that are within the range of Progranulin protein levels for age-matched procured controls. In some embodiments, the increase in Progranulin protein levels in plasma and/or cerebrospinal fluid of the individual is present at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, at least about 15 weeks, at least about 16 weeks, at least about 17 weeks, at least about 18 weeks, at least about 19 weeks, at least about 20 weeks, at least about 21 weeks, at least about 22 weeks, at least about 23 weeks, at least about 24 weeks, at least about 25 weeks, at least about 26 weeks, at least about 27 weeks, at least about 28 weeks, at least about 29 weeks, at least about 30 weeks, at least about 31 weeks, at least about 32 weeks, at least about 33 weeks, at least about 34 weeks, at least about 35 weeks, at least about 36 weeks, at least about 37 weeks, at least about 38 weeks, at least about 39 weeks, at least about 40 weeks, at least about 41 weeks, at least about 42 weeks, at least about 43 weeks, at least about 44 weeks, at least about 45 weeks, at least about 46 weeks, at least about 47 weeks, at least about 48 weeks, at least about 49 weeks, at least about 50 weeks, at least about 51 weeks, at least about 52 weeks, or more, after the start of treatment according to the methods provided herein.

Non-limiting examples of methods that may be used to measure the levels of Progranulin protein in a sample obtained from the individual, e.g., in a plasma or cerebrospinal fluid sample, include SOMASCAN assay (see, e.g., Candia et al. (2017) Sci Rep 7, 14248), Western blots, mass spectrometry, flow cytometry, and enzyme-linked immunosorbent assay (ELISA) assays.

In some embodiments, treatment and/or delay of FTD progression is assessed based on the level of NfL in the serum or plasma of the individual after the start of treatment according to the methods provided herein, e.g., as compared to the level of NfL in the serum of the individual prior to the start of treatment according to the methods provided herein. In some embodiments, treatment and/or delay of FTD progression is assessed based on the level of NfL in the cerebrospinal fluid of the individual after the start of treatment according to the methods provided herein, e.g., as compared to the level of NfL in cerebrospinal fluid of the individual prior to the start of treatment according to the methods provided herein. In some embodiments, the methods of treating or delaying progression of FTD comprise measuring the level of NfL in a sample of serum or plasma obtained from the individual before and after the individual has received one or more doses of the anti-Sortilin antibody. In some embodiments, the methods of treating or delaying progression of FTD comprise measuring the level of NfL in a sample of cerebrospinal fluid obtained from the individual before and after the individual has received one or more doses of the anti-Sortilin antibody. In some embodiments, treatment with an anti-Sortilin antibody of the present disclosure reduces NfL levels in serum, plasma and/or cerebrospinal fluid by any of at least about 1%, at least about 5%, at least about 10%, at least about 15%, at least about 20%, at least about 25%, at least about 30%, at least about 35%, at least about 40%, at least about 45%, at least about 50%, or more, e.g., as compared to prior to the start of treatment according to the methods provided herein. NfL levels in a sample obtained from the individual, e.g., in a serum or cerebrospinal fluid sample, may be measured by any known methods in the art including, without limitation, immunoassays, a single-molecule array technology (Simoa) assay (e.g., using commercially available kits, such as the NF-light digital immunoassay kit or Simoa HD-1 assay from Quanterix, Lexinton, M A; or a Neurology 4-Plex A kit, see, e.g., Heller et al., J Neurol Neurosurg Psychiatry (2020) 91(3):263-270), ELISA, or using other assays from Quanterix or Roche Diagnostics.

In some embodiments, treatment and/or delay of FTD progression is assessed based on one or more imaging assessments (e.g., one or more of global and regional brain volumes, or volume of white matter hyperintensities, e.g., measured by structural volumetric magnetic resonance imaging (MRI); brain perfusion, e.g., measured by arterial spin labeling MRI; and fractional anisotropy, mean diffusivity, axial diffusivity, and radial diffusivity, e.g., measured by diffusion-tensor imaging) after the start of treatment according to the methods provided herein, e.g., as compared to the one or more imaging assessments prior to the start of treatment according to the methods provided herein. In some embodiments, the methods of treating or delaying progression of FTD comprise assessing global and regional brain volumes in the individual before and after the individual has received one or more doses of the anti-Sortilin antibody. In some embodiments, the methods of treating or delaying progression of FTD comprise assessing volume of white matter hyperintensities in the individual before and after the individual has received one or more doses of the anti-Sortilin antibody. In some embodiments, the methods of treating or delaying progression of FTD comprise assessing brain perfusion in the individual before and after the individual has received one or more doses of the anti-Sortilin antibody. In some embodiments, the methods of treating or delaying progression of FTD comprise assessing fractional anisotropy, mean diffusivity, axial diffusivity, and/or radial diffusivity in the individual before and after the individual has received one or more doses of the anti-Sortilin antibody.

In some embodiments, the methods of treating or delaying progression of FTD provided herein comprise assessing brain ventricles, whole brain, and/or frontotemporal cortex in the individual by volumetric MRI before and after the individual has received one or more doses of the anti-Sortilin antibody. In some embodiments, the methods of treating or delaying progression of FTD provided herein comprise assessing brain atrophy (e.g., in the brain ventricles, whole brain, and/or frontotemporal cortex) in the individual, e.g., by volumetric MRI, before and after the individual has received one or more doses of the anti-Sortilin antibody. In some embodiments, treatment with an anti-Sortilin antibody of the present disclosure results in reduced brain atrophy (e.g., in the brain ventricles, whole brain, and/or frontotemporal cortex) in an individual, e.g., as compared to the rate of brain atrophy that the individual would have experienced if untreated, or compared to a corresponding individual with FTD but not treated with the anti-Sortilin antibody. In some embodiments, a corresponding individual with FTD may be an individual having the same or similar disease severity (e.g., as measured by clinical outcome assessments), age, gender, genetics, and/or other biomarkers or baseline assessments, such as Nfl levels in CSF or plasma.

In some embodiments, treatment with an anti-Sortilin antibody of the present disclosure results in reduced enlargement of the ventricles in an individual, e.g., as compared to a corresponding individual with FTD but not treated with the anti-Sortilin antibody. In some embodiments, treatment with an anti-Sortilin antibody of the present disclosure results in a reduction of brain ventricle enlargement in an individual of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, e.g., as compared to a corresponding individual not treated with the anti-Sortilin antibody. In some embodiments, treatment with an anti-Sortilin antibody of the present disclosure results in a reduction of brain ventricle enlargement in an individual of at least about 50%, e.g., as compared to a corresponding individual with FTD but not treated with the anti-Sortilin antibody. In some embodiments, treatment with an anti-Sortilin antibody of the present disclosure results in a reduction in the annualized rate of change of the brain ventricles in the individual that is at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% lower than the annualized rate of change of the brain ventricles in a corresponding individual not treated with the anti-Sortilin antibody. In some embodiments, enlargement of the brain ventricles is assessed by volumetric MRI.

In some aspects, provided herein are methods of treating and/or delaying the progression of FTD, comprising administering to an individual an anti-Sortilin antibody of the present disclosure, wherein administration of the anti-Sortilin antibody results in a reduction in brain ventricle enlargement in the individual of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100%, e.g., as compared to a corresponding individual with FTD but not treated with the anti-Sortilin antibody. In some embodiments, administration of the anti-Sortilin antibody results in a reduction in brain ventricle enlargement in the individual of at least about 50%, e.g., as compared to a corresponding individual with FTD but not treated with the anti-Sortilin antibody. In some embodiments, brain ventricle enlargement is assessed by volumetric MRJ. In some embodiments, the anti-Sortilin antibody is administered according to any of the methods for treating and/or delaying the progression of FTD provided herein. In some embodiments, the individual has symptomatic FTD. In some embodiments, the individual has one or more Granulin mutations causative of FTD.

In some aspects, provided herein are methods of treating and/or delaying the progression of FTD, comprising administering to an individual an anti-Sortilin antibody of the present disclosure, wherein administration of the anti-Sortilin antibody results in a reduction in the annualized rate of change of the brain ventricles in the individual that is at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% lower than the annualized rate of change of the brain ventricles in a corresponding individual not treated with the anti-Sortilin antibody. In some embodiments, the annualized rate of change of the brain ventricles is assessed by volumetric MRI.

In some aspects, provided herein are methods of treating and/or delaying the progression of FTD, comprising administering to an individual an anti-Sortilin antibody of the present disclosure, wherein administration of the anti-Sortilin antibody results in a reduction in the change of volume of the brain ventricles in the individual over a specified period of time that is at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or 100% lower than the change in volume of the brain ventricles over the same period of time in a corresponding individual not treated with the anti-Sortilin antibody. In some embodiments, the period of time is 6 months, 12 months, 18 months, or longer. In some embodiments, the anti-Sortilin antibody is administered according to any of the methods for treating and/or delaying the progression of FTD provided herein. In some embodiments, the individual has symptomatic FTD. In some embodiments, the individual has one or more Granulin mutations causative of FTD.

In some embodiments, treatment and/or delay of FTD progression is assessed based on the level of one or more biomarkers of neurodegeneration in whole blood, plasma, and/or cerebrospinal fluid after the start of treatment according to the methods provided herein, e.g., as compared to the level of the one or more biomarkers of neurodegeneration prior to the start of treatment according to the methods provided herein. In some embodiments, the methods of treating or delaying progression of FTD comprise measuring the level of one or more biomarkers of neurodegeneration in a sample of whole blood, plasma, or cerebrospinal fluid obtained from the individual before and after the individual has received one or more doses of the anti-Sortilin antibody. Biomarkers of neurodegeneration may include, without limitation, NfL, Tau, and/or pTau. In some embodiments, treatment with an anti-Sortilin antibody of the present disclosure reduces NfL levels in whole blood, plasma and/or cerebrospinal fluid by any of at least about 1%, at least about 5%, at least about 10%, at least about 15%, at least about 20%, at least about 25%, at least about 30%, at least about 35%, at least about 40%, at least about 45%, at least about 50%, or more, e.g., as compared to prior to the start of treatment according to the methods provided herein. In some embodiments, treatment with an anti-Sortilin antibody of the present disclosure stabilizes NfL levels in whole blood, plasma and/or cerebrospinal fluid of the individual with FTD as compared to prior to start of treatment according to the methods provided herein. NfL levels in a sample obtained from the individual, e.g., in a whole blood, plasma and/or cerebrospinal fluid sample, may be measured by any known methods in the art including, without limitation, immunoassays, a single-molecule array technology (Simoa) assay (e.g., using commercially available kits, such as the NF-light digital immunoassay kit or Simoa HD-1 assay from Quanterix, Lexinton, MA; or a Neurology 4-Plex A kit, see, e.g., Heller et al., J Neurol Neurosurg Psychiatry (2020) 91(3):263-270), ELISA, or using other assays from Quanterix or Roche Diagnostics. Non-limiting examples of methods that may be used to measure the levels of the one or more biomarkers of neurodegeneration in a sample obtained from the individual, e.g., in a whole blood, plasma, and/or cerebrospinal fluid sample, include SOMASCAN assay (see, e.g., Candia et al. (2017) Sci Rep 7, 14248), Western blots, mass spectrometry, flow cytometry, and enzyme-linked immunosorbent assay (ELISA) assays.

In some embodiments, treatment and/or delay of FTD progression is assessed based on the level of one or more biomarkers of lysosomal function in whole blood, plasma, and/or cerebrospinal fluid after the start of treatment according to the methods provided herein, e.g., as compared to the level of the one or more biomarkers of lysosomal function prior to the start of treatment according to the methods provided herein. In some embodiments, the methods of treating or delaying progression of FTD comprise measuring the level of one or more biomarkers of lysosomal function in a sample of whole blood, plasma, or cerebrospinal fluid obtained from the individual before and after the individual has received one or more doses of the anti-Sortilin antibody.

Biomarkers of lysosomal function may include, without limitation, N-acetylglucosamine kinase (NAGK) or one or more cathepsins, such as cathepsin B (CTSB). Accordingly, in some embodiments, treatment with an anti-Sortilin antibody of the present disclosure increases the level of certain biomarkers of lysosomal function in whole blood, plasma, and/or cerebrospinal fluid of the individual by any of at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, or more, e.g., as compared to prior to the start of treatment according to the methods provided herein.

Other biomarkers of lysosomal function may include biomarkers that are overexpressed when PGRN is deficient. For example, cathepsin D (CTSD) and Lamp1 are overexpressed in PGRN-deficient mice (GRN knockout mice), and are increased in the brains of FTD-GRN patients. See, Huang et al. (2020) Acta Neuropath Comm 8:163; and Götzl et al., (2014) Acta Neuropathol 127(6):845-60. Restoration of PGRN function may therefore decrease expression of biomarkers of lysosomal function that are increased when PGRN is deficient. Accordingly, in some embodiments, treatment with an anti-Sortilin antibody of the present disclosure decreases the level of certain biomarkers of lysosomal function, such as CTSD and/or Lamp1, in whole blood, plasma, and/or cerebrospinal fluid of the individual by any of at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, or more, e.g., as compared to prior to the start of treatment according to the methods provided herein. In some embodiments, treatment with an anti-Sortilin antibody of the present disclosure decreases the level of certain biomarkers of lysosomal function, such as CTSD and/or Lamp1, in cerebrospinal fluid of the individual by at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, or more, e.g., as compared to prior to the start of treatment according to the methods provided herein. In some embodiments, the decrease in the level of certain biomarkers of lysosomal function, such as CTSD and/or Lamp1, in cerebrospinal fluid of the individual is present at least about 6 months after the start of treatment according to the methods provided herein. In some embodiments, the decrease in the level of certain biomarkers of lysosomal function, such as CTSD and/or Lamp1, in cerebrospinal fluid of the individual is present at least about 12 months after the start of treatment according to the methods provided herein. In some embodiments, treatment with an anti-Sortilin antibody of the present disclosure decreases the level of certain biomarkers of lysosomal function, such as one or more cathepsins (e.g., CTSD) and/or Lamp1, in whole blood, plasma, and/or cerebrospinal fluid of an individual to normal levels, e.g., as observed in controls, such as age-matched procured controls.

Certain biomarkers of complement function show overexpression when PGRN is deficient. For example, Clqb and Clqc (subunits that make up the complement protein Clq) show increased levels in PGRN-deficient (GRNknockout) mice, and levels of Clqa (a subunit of the Clq protein) increase as cognitive function declines in FTD-GRN carriers. See, Huang et al. (2020) Acta Neuropath Comm 8:163; and Lui et al., (2016) Cell 165:921-935. Restoration of PGRN function may therefore decrease expression of biomarkers of complement function that are increased when PGRN is deficient. Accordingly, in some embodiments, treatment with an anti-Sortilin antibody of the present disclosure decreases the level of certain biomarkers of complement function, such as Clqb and/or Clqc, in whole blood, plasma, and/or cerebrospinal fluid of the individual by any of at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, or more, e.g., as compared to prior to the start of treatment according to the methods provided herein. In some embodiments, treatment with an anti-Sortilin antibody of the present disclosure decreases the level of certain biomarkers of complement function, such as Clqb and/or Clqc, in cerebrospinal fluid of the individual by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, or more, e.g., as compared to prior to the start of treatment according to the methods provided herein. In some embodiments, the decrease in the level of certain biomarkers of complement function, such as Clqb and/or Clqc, in cerebrospinal fluid of the individual is present at least about 6 months after the start of treatment according to the methods provided herein. In some embodiments, the decrease in the level of certain biomarkers of complement function, such as Clqb and/or Clqc, in cerebrospinal fluid of the individual is present at least about 12 months after the start of treatment according to the methods provided herein. In some embodiments, treatment with an anti-Sortilin antibody of the present disclosure decreases the level of certain biomarkers of complement function, such as Clqb and/or Clqc, in whole blood, plasma, and/or cerebrospinal fluid of an individual to normal levels, e.g., as observed in controls, such as age-matched procured controls. In some embodiments, treatment with an anti-Sortilin antibody of the present disclosure decreases the level of biomarkers of lysosomal function, such as cathepsins (e.g., CTSD) and/or Lamp1, and decreases the level of certain biomarkers of complement function, such as Clqb and/or Clqc, in whole blood, plasma, and/or cerebrospinal fluid of an individual to normal levels, e.g., as observed in controls, such as age-matched procured controls. In some embodiments, treatment with an anti-Sortilin antibody of the present disclosure decreases the level of biomarkers of lysosomal function, e.g., cathepsins (e.g., CTSD) and/or Lamp1, and decreases the level of biomarkers of complement function, e.g., Clqb, in whole blood, plasma, and/or cerebrospinal fluid of an individual to normal levels, e.g., as observed in controls, such as age-matched procured controls. In some embodiments, treatment with an anti-Sortilin antibody of the present disclosure decreases the level of biomarkers of lysosomal function, e.g., cathepsins (e.g., CTSD) and/or Lamp1, and decreases the level of biomarkers of complement function, e.g., Clqb, in cerebrospinal fluid of an individual to normal levels, e.g., as observed in controls, such as age-matched procured controls.

Non-limiting examples of methods that may be used to measure the levels of the one or more biomarkers of lysosomal function or complement function in a sample obtained from the individual, e.g., in a whole blood, plasma, and/or cerebrospinal fluid sample, include SOMASCAN assay (see, e.g., Candia et al. (2017) Sci Rep 7, 14248), Western blots, mass spectrometry (e.g., Multiple Reaction Monitoring Liquid Chromatography-Mass Spectrometry), flow cytometry, and enzyme-linked immunosorbent assay (ELISA) assays.

Astrogliosis is an abnormal proliferation of astrocytes due to neuronal damage. Certain biomarkers of astrogliosis, such as glial fibrillary acidic protein (GFAP), are elevated in frontotemporal dementia patients, including in FTD-GRN patients. In addition, elevated GFAP levels have been correlated with faster rates of atrophy in the temporal lobe of symptomatic FTD-GRN patients. See, Heller et al. J. Neurol Neurosurg Psychiatry 2020; 91:263-270. Restoration of PGRN function may therefore decrease expression of biomarkers of astrogliosis, such as GFAP, that are elevated in certain frontotemporal dementia patients, including in FTD-GRN patients. Accordingly, in some embodiments, treatment with an anti-Sortilin antibody of the present disclosure decreases the level of certain biomarkers of astrogliosis, such as GFAP, in whole blood, plasma, and/or cerebrospinal fluid of the individual by any of at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or about 100%, e.g., as compared to prior to the start of treatment according to the methods provided herein. In some embodiments, the decrease in the level of certain biomarkers of astrogliosis, such as GFAP, in whole blood, plasma, and/or cerebrospinal fluid of the individual is present at least about 1 week, at least about 2 weeks, at least about 5 weeks, at least about 9 weeks, at least about 13 weeks, at least about 17 weeks, at least about 21 weeks, at least about 25 weeks, at least about 29 weeks, at least about 33 weeks, at least about 37 weeks, at least about 41 weeks, at least about 45 weeks, at least about 49 weeks, or more, after the start of treatment according to the methods provided herein. In some embodiments, the decrease in the level of certain biomarkers of astrogliosis, such as GFAP, in whole blood, plasma, and/or cerebrospinal fluid of the individual is present at least about 6 months after the start of treatment according to the methods provided herein. In some embodiments, the decrease in the level of certain biomarkers of astrogliosis, such as GFAP, in whole blood, plasma, and/or cerebrospinal fluid of the individual is present at least about 12 months after the start of treatment according to the methods provided herein.

Non-limiting examples of methods that may be used to measure the levels of the one or more biomarkers of astrogliosis, e.g., GFAP, in a sample obtained from the individual, e.g., in a whole blood, plasma, and/or cerebrospinal fluid sample, include SOMASCAN assay (see, e.g., Candia et al. (2017) Sci Rep 7, 14248), Western blots, mass spectrometry (e.g., Multiple Reaction Monitoring Liquid Chromatography-Mass Spectrometry), flow cytometry, a single molecule array based-assay (e.g., a Simoa assay by Quanterix; see, e.g., the website: www.quanterix.com/simoa-technology/), and enzyme-linked immunosorbent assay (ELISA) assays.

In some embodiments, treatment and/or delay of FTD progression is assessed based on the level of one or more biomarkers of glial activity in whole blood, plasma, and/or cerebrospinal fluid after the start of treatment according to the methods provided herein, e.g., as compared to the level of the one or more biomarkers of glial activity prior to the start of treatment according to the methods provided herein. In some embodiments, the methods of treating or delaying progression of FTD comprise measuring the level of one or more biomarkers of glial activity in a sample of whole blood, plasma, or cerebrospinal fluid obtained from the individual before and after the individual has received one or more doses of the anti-Sortilin antibody. Biomarkers of glial activity may include, without limitation, YKL40 and IL-6. Non-limiting examples of methods that may be used to measure the levels of the one or more biomarkers of glial activity in a sample obtained from the individual, e.g., in a whole blood, plasma, and/or cerebrospinal fluid sample, include SOMASCAN assay (see, e.g., Candia et al. (2017) Sci Rep 7, 14248), Western blots, mass spectrometry, flow cytometry, and enzyme-linked immunosorbent assay (ELISA) assays.

In some embodiments, treatment and/or delay of FTD progression is assessed based on the level of one or more biomarkers of neuroinflammation in whole blood, plasma, and/or cerebrospinal fluid after the start of treatment according to the methods provided herein, e.g., as compared to the level of the one or more biomarkers of neuroinflammation prior to the start of treatment according to the methods provided herein. In some embodiments, the methods of treating or delaying progression of FTD comprise measuring the level of one or more biomarkers of neuroinflammation in a sample of whole blood, plasma, or cerebrospinal fluid obtained from the individual before and after the individual has received one or more doses of the anti-Sortilin antibody. Biomarkers of neuroinflammation may include, without limitation, macrophage migration inhibitory factor (MIF). MIF is a pleiotropic pro-inflammatory cytokine highly and widely expressed in human neural tissues, including neurons, microglia, astrocytes, and ependymal cells. MIF can promote the secretion of other inflammatory response mediators including IL6, and TNF-α, and can also activate the inflammasome. In addition, elevated CSF levels of MIF have been observed in Alzheimer's disease patients compared to age-matched controls (see, e.g., Zhang et al., Alzheimers Res Ther. 2019; 11(1):54). Furthermore, as disclosed in Example 2 herein, Applicant discovered that the levels of MIF protein are elevated in the CSF of frontotemporal dementia patients, including in FTD-GRN and FTD-C9orf72 patients. Restoration of progranulin (PGRN) function may therefore decrease the levels of biomarkers of neuroinflammation, such as MIF, that are elevated in certain frontotemporal dementia (FTD) patients, including in FTD-GRN and FTD-C9orf72 patients. Accordingly, in some embodiments, treatment with an anti-Sortilin antibody of the present disclosure decreases the level of certain biomarkers of neuroinflammation, such as MIF, in whole blood, plasma, and/or cerebrospinal fluid of the individual by any of at least about 1%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or about 100%, e.g., as compared to prior to the start of treatment according to the methods provided herein. In some embodiments, the decrease in the level of certain biomarkers of neuroinflammation, such as MIF, in whole blood, plasma, and/or cerebrospinal fluid of the individual is present at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 12 months, or more, after the start of treatment according to the methods provided herein. In some embodiments, the decrease in the level of certain biomarkers of neuroinflammation, such as MIF, in whole blood, plasma, and/or cerebrospinal fluid of the individual is present at least about 6 months after the start of treatment according to the methods provided herein. In some embodiments, the decrease in the level of certain biomarkers of neuroinflammation, such as MIF, in whole blood, plasma, and/or cerebrospinal fluid of the individual is present at least about 12 months after the start of treatment according to the methods provided herein. In some embodiments, treatment with an anti-Sortilin antibody of the present disclosure decreases the level of certain biomarkers of neuroinflammation, such as MIF, in whole blood, plasma, and/or cerebrospinal fluid of the individual by at least about 15% at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months after the start of treatment with an anti-Sortilin antibody, as compared to the level prior to the start of treatment. In some embodiments, treatment with an anti-Sortilin antibody of the present disclosure decreases the level of certain biomarkers of neuroinflammation, such as MIF, in whole blood, plasma, and/or cerebrospinal fluid of the individual by at least about 15% at least about 6 months or at least about 12 months after the start of treatment with an anti-Sortilin antibody, as compared to the level prior to the start of treatment. In some embodiments, treatment with an anti-Sortilin antibody of the present disclosure decreases the level of certain biomarkers of neuroinflammation, such as MIF, in whole blood, plasma, and/or cerebrospinal fluid of the individual by at least about 15% at least about 12 months after the start of treatment with an anti-Sortilin antibody, as compared to the level prior to the start of treatment. In some embodiments, the decrease in the level of certain biomarkers of neuroinflammation, such as MIF, in whole blood, plasma, and/or cerebrospinal fluid of the individual is present at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 15 days, at least about 16 days, at least about 17 days, at least about 18 days, at least about 19 days, at least about 20 days, at least about 21 days, at least about 22 days, at least about 23 days, at least about 24 days, at least about 25 days, at least about 26 days, at least about 27 days, at least about 28 days, at least about 29 days, at least about 30 days, at least about 31 days, at least about 32 days, at least about 33 days, at least about 34 days, at least about 35 days, at least about 36 days, at least about 37 days, at least about 38 days, at least about 39 days, at least about 40 days, at least about 41 days, at least about 42 days, at least about 43 days, at least about 44 days, at least about 45 days, at least about 46 days, at least about 47 days, at least about 48 days, at least about 49 days, at least about 50 days, at least about 51 days, at least about 52 days, at least about 53 days, at least about 54 days, at least about 55 days, at least about 56 days, at least about 57 days, or more, after the start of treatment according to the methods provided herein. Non-limiting examples of methods that may be used to measure the levels of the one or more biomarkers of neuroinflammation (e.g., MIF) in a sample obtained from the individual, e.g., in a whole blood, plasma, and/or cerebrospinal fluid sample, include SOMASCAN assay (see, e.g., Candia et al. (2017) Sci Rep 7, 14248), Western blots, mass spectrometry, flow cytometry, and enzyme-linked immunosorbent assay (ELISA). In some embodiments, the levels of MIF protein in a sample of cerebrospinal fluid obtained from the individual are measured using an ELISA method, such as the sandwich Enzyme-Linked Immunosorbent Assay described herein in Example 2.

### Alzheimer's Disease

Alzheimer's disease (AD) is the most common form of dementia. There is no cure for the disease, which worsens as it progresses, and eventually leads to death. Most often, AD is diagnosed in people over 65 years of age. However, the less-prevalent early-onset Alzheimer's can occur much earlier.

Common symptoms of Alzheimer's disease include, behavioral symptoms, difficulty in remembering recent events, cognitive symptoms, confusion, irritability and aggression, mood swings, trouble with language, and long-term memory loss. As the disease progresses bodily functions are lost, ultimately leading to death. Alzheimer's disease develops for an unknown and variable amount of time before becoming fully apparent, and it can progress undiagnosed for years.

It has been shown that Sortilin binds to amyloid precursor protein (APP) and the APP processing enzyme BACE1. Without wishing to be bound by theory, it is believed that these interactions are involved in Alzheimer's disease. Accordingly, and without wishing to be bound by theory, it is believed that anti-Sortilin antibodies of the present disclosure can be utilized to inhibit such interactions and prevent, reduce the risk of, or treat Alzheimer's disease in individuals in need thereof.

In some embodiments, and without wishing to be bound by theory, it is believed that anti-Sortilin antibodies of the present disclosure that inhibit the interaction between Sortilin and neurotrophins of the present disclosure (e.g., pro-neurotrophins, pro-neurotrophin-3, pro-neurotrophin-4/5, pro-NGF, pro-BDNF, neurotrophin-3, neurotrophin-4/5, NGF, BDNF, etc.), p75, amyloid precursor protein (APP), and/or the A beta peptide, or that inhibit one or more activities of Sortilin can be utilized to treat and/or delay the progression of Alzheimer's disease in individuals in need thereof.

In some embodiments, administering an anti-Sortilin antibody of the present disclosure can treat and/or delay the progression of Alzheimer's disease. In some embodiments, administering an anti-Sortilin antibody may modulate one or more Sortilin activities in an individual having Alzheimer's disease. In some embodiments, administering an anti-Sortilin antibody may modulate one or more Sortilin activities in an individual having Alzheimer's disease. In some embodiments, administering an anti-Sortilin antibody may induce one or more Progranulin activities in an individual having Alzheimer's disease. In some embodiments, administering an anti-Sortilin antibody may inhibit one or more activities of Sortilin in an individual having Alzheimer's disease. In some embodiments, administering an anti-Sortilin antibody may decrease cellular levels of Sortilin in an individual having Alzheimer's disease. In some embodiments, administering an anti-Sortilin antibody may increase Progranulin levels in an individual having Alzheimer's disease. In some embodiments, administering an anti-Sortilin antibody may inhibit the interaction (e.g., binding) between Progranulin and Sortilin in an individual having Alzheimer's disease. In some embodiments, administering an anti-Sortilin antibody may decrease expression or secretion of pro-inflammatory mediators in an individual having Alzheimer's disease. In some embodiments, administering an anti-Sortilin antibody may inhibit interaction (e.g., binding) between Sortilin and one or more of pro-neurotrophins, neurotrophins, neurotensin, p75, Sortilin propeptide (Sort-pro), amyloid precursor protein (APP), A beta peptide, lipoprotein lipase (LpL), apolipoprotein AV (APOA5), apolipoprotein E (APOE), and receptor associated protein (RAP) in an individual having Alzheimer's disease. In some embodiments, administering an anti-Sortilin antibody may decrease secretion of PCSK9 in an individual having Alzheimer's disease. In some embodiments, administering an anti-Sortilin antibody may decrease production of beta amyloid peptide in an individual having Alzheimer's disease.

Vascular Dementia

Vascular dementia (VaD) is a subtly progressive worsening of memory and other cognitive functions that is believed to be due to cerebrovascular disease (vascular disease within the brain). Cerebrovascular disease is the progressive change in blood vessels (vasculature) in the brain (cerebrum). The most common vascular change associated with age is the accumulation of cholesterol and other substances in the blood vessel walls. This results in the thickening and hardening of the walls, as well as narrowing of the vessels, which can result in a reduction or even a complete stopping of blood flow to brain regions supplied by the affected artery. Vascular dementia patients often present with similar symptoms to Alzheimer's disease (AD) patients. However, the related changes in the brain are not due to AD pathology but to chronic reduced blood flow in the brain, eventually resulting in dementia. VaD is considered one of the most common types of dementia in older adults. Symptoms of VaD include difficulties with memory, difficulty with organization and solving complex problems, slowed thinking, distraction or “absent mindedness,” difficulty retrieving words from memory, changes in mood or behavior such as depression, irritability, or apathy, and hallucinations or delusions.

Without wishing to be bound by theory, it is believed that one or more activities of Sortilin, or one or more interactions between Sortilin and Progranulin, neurotrophins of the present disclosure (e.g., pro-neurotrophins, pro-neurotrophin-3, pro-neurotrophin-4/5, pro-NGF, pro-BDNF, neurotrophin-3, neurotrophin-4/5, NGF, BDNF, etc.), neurotensin, lipoprotein lipase, apolipoprotein AV, and/or receptor-associated protein are involved in vascular dementia. Accordingly, and without wishing to be bound by theory, it is believed that anti-Sortilin antibodies of the present disclosure that inhibit the interaction between Sortilin and neurotrophins of the present disclosure (e.g., pro-neurotrophins, pro-neurotrophin-3, pro-neurotrophin-4/5, pro-NGF, pro-BDNF, neurotrophin-3, neurotrophin-4/5, NGF, BDNF, etc.), neurotensin, p75, Sortilin propeptide (Sort-pro), amyloid precursor protein (APP), the A beta peptide, lipoprotein lipase (LpL), apolipoprotein AV (APOA5), apolipoprotein E (APOE), and/or receptor associated protein (RAP); or that inhibit one or more activities of Sortilin can be utilized to prevent, reduce the risk of, or treat vascular dementia in individuals in need thereof.

In some embodiments, administering an anti-Sortilin antibody of the present disclosure can treat and/or delay the progression of VaD. In some embodiments, administering an anti-Sortilin antibody may modulate one or more Sortilin activities in an individual having VaD. In some embodiments, administering an anti-Sortilin antibody may induce one or more Progranulin activities in an individual having VaD. In some embodiments, administering an anti-Sortilin antibody may inhibit one or more activities of Sortilin in an individual having VaD. In some embodiments, administering an anti-Sortilin antibody may decrease cellular levels of Sortilin in an individual having VaD. In some embodiments, administering an anti-Sortilin antibody may increase Progranulin levels in an individual having VaD. In some embodiments, administering an anti-Sortilin antibody may inhibit the interaction (e.g., binding) between Progranulin and Sortilin in an individual having VaD. In some embodiments, administering an anti-Sortilin antibody may decrease expression or secretion of pro-inflammatory mediators in an individual having VaD. In some embodiments, administering an anti-Sortilin antibody may inhibit interaction (e.g., binding) between Sortilin and one or more of pro-neurotrophins, neurotrophins, neurotensin, p75, Sortilin propeptide (Sort-pro), amyloid precursor protein (APP), A beta peptide, lipoprotein lipase (LpL), apolipoprotein AV (APOA5), apolipoprotein E (APOE), and receptor associated protein (RAP) in an individual having VaD. In some embodiments, administering an anti-Sortilin antibody may decrease secretion of PCSK9 in an individual having VaD. In some embodiments, administering an anti-Sortilin antibody may decrease production of beta amyloid peptide in an individual having VaD.

### Seizures, Retinal Dystrophy, Traumatic Brain Injuries, and Spinal Cord Injuries

As used herein, retinal dystrophy refers to any disease or condition that involves the degeneration of the retina. Such diseases or conditions may lead to loss of vision or complete blindness.

As used herein, seizures also include epileptic seizures, and refer to a transient symptom of abnormal excessive or synchronous neuronal activity in the brain. The outward effect can be as dramatic as a wild thrashing movement or as mild as a brief loss of awareness. Seizures can manifest as an alteration in mental state, tonic or clonic movements, convulsions, and various other psychic symptoms.

Traumatic brain injuries (TBI), may also be known as intracranial injuries. Traumatic brain injuries occur when an external force traumatically injures the brain. Traumatic brain injuries can be classified based on severity, mechanism (closed or penetrating head injury), or other features (e.g., occurring in a specific location or over a widespread area).

Spinal cord injuries (SCI) include any injury to the spinal cord that is caused by trauma instead of disease. Depending on where the spinal cord and nerve roots are damaged, the symptoms can vary widely, from pain to paralysis to incontinence. Spinal cord injuries are described at various levels of “incomplete”, which can vary from having no effect on the patient to a “complete” injury which means a total loss of function.

It has been shown that pro-neurotrophins (e.g., pro-neurotrophin-4/5, neurotrophin-4/5, pro-NGF, pro-BDNF, etc.) play a role in seizures, retinal dystrophy, traumatic brain injury, and spinal cord injury.

Accordingly, and without wishing to be bound by theory, it is believed that anti-Sortilin antibodies of the present disclosure that inhibit the interaction between Sortilin and neurotrophins of the present disclosure (e.g., pro-neurotrophins, pro-neurotrophin-3, pro-neurotrophin-4/5, pro-NGF, pro-BDNF, neurotrophin-3, neurotrophin-4/5, NGF, BDNF, etc.); or that inhibit one or more activities of Sortilin can be utilized to prevent, reduce the risk of, or treat seizures, retinal dystrophy, traumatic brain injuries, and/or spinal cord injuries in individuals in need thereof.

In some embodiments, administering an anti-Sortilin antibody of the present disclosure can treat and/or delay the progression of seizures, retinal dystrophy, traumatic brain injuries, and/or spinal cord injuries. In some embodiments, administering an anti-Sortilin antibody may modulate one or more Sortilin activities in an individual having seizures, retinal dystrophy, traumatic brain injuries, and/or spinal cord injuries. In some embodiments, administering an anti-Sortilin antibody may induce one or more Progranulin activities in an individual having seizures, retinal dystrophy, traumatic brain injuries, and/or spinal cord injuries. In some embodiments, administering an anti-Sortilin antibody may inhibit one or more activities of Sortilin in an individual having seizures, retinal dystrophy, traumatic brain injuries, and/or spinal cord injuries. In some embodiments, administering an anti-Sortilin antibody may decrease cellular levels of Sortilin in an individual having seizures, retinal dystrophy, traumatic brain injuries, and/or spinal cord injuries. In some embodiments, administering an anti-Sortilin antibody may increase Progranulin levels in an individual having seizures, retinal dystrophy, traumatic brain injuries, and/or spinal cord injuries. In some embodiments, administering an anti-Sortilin antibody may inhibit the interaction (e.g., binding) between Progranulin and Sortilin in an individual having seizures, retinal dystrophy, traumatic brain injuries, and/or spinal cord injuries. In some embodiments, administering an anti-Sortilin antibody may decrease expression or secretion of pro-inflammatory mediators in an individual having seizures, retinal dystrophy, traumatic brain injuries, and/or spinal cord injuries. In some embodiments, administering an anti-Sortilin antibody may inhibit interaction (e.g., binding) between Sortilin and one or more of pro-neurotrophins, neurotrophins, neurotensin, p75, Sortilin propeptide (Sort-pro), amyloid precursor protein (APP), A beta peptide, lipoprotein lipase (LpL), apolipoprotein AV (APOA5), apolipoprotein E (APOE), and receptor associated protein (RAP) in an individual having seizures, retinal dystrophy, traumatic brain injuries, and/or spinal cord injuries. In some embodiments, administering an anti-Sortilin antibody may decrease secretion of PCSK9 in an individual having seizures, retinal dystrophy, traumatic brain injuries, and/or spinal cord injuries. In some embodiments, administering an anti-Sortilin antibody may decrease production of beta amyloid peptide in an individual having seizures, retinal dystrophy, traumatic brain injuries, and/or spinal cord injuries.

Undesirable Symptoms of Aging

As used herein, undesirable symptoms of aging include, without limitation, memory loss, behavioral changes, dementia, Alzheimer's disease, retinal degeneration, atherosclerotic vascular diseases, hearing loss, and cellular break-down.

In some embodiments, and without wishing to be bound by theory, it is believed that anti-Sortilin antibodies of the present disclosure that inhibit the interaction between Sortilin and Progranulin, neurotrophins of the present disclosure (e.g., pro-neurotrophins, pro-neurotrophin-3, pro-neurotrophin-4/5, pro-NGF, pro-BDNF, neurotrophin-3, neurotrophin-4/5, NGF, BDNF, etc.), neurotensin, p75, lipoprotein lipase (LpL), apolipoprotein AV (APOA5), and/or receptor associated protein (RAP); or that inhibit one or more activities of Sortilin can be utilized to prevent, reduce the risk of, or treat one or more undesirable symptoms of aging.

In some embodiments, administering an anti-Sortilin antibody of the present disclosure can treat and/or delay the progression of one or more undesirable symptoms of aging. In some embodiments, administering an anti-Sortilin antibody may modulate one or more Sortilin activities in an individual having one or more undesirable symptoms of aging. In some embodiments, administering an anti-Sortilin antibody may induce one or more Progranulin activities in an individual having one or more undesirable symptoms of aging. In some embodiments, administering an anti-Sortilin antibody may inhibit one or more activities of Sortilin in an individual having one or more undesirable symptoms of aging. In some embodiments, administering an anti-Sortilin antibody may decrease cellular levels of Sortilin in an individual having one or more undesirable symptoms of aging. In some embodiments, administering an anti-Sortilin antibody may increase Progranulin levels in an individual having one or more undesirable symptoms of aging. In some embodiments, administering an anti-Sortilin antibody may inhibit the interaction (e.g., binding) between Progranulin and Sortilin in an individual having one or more undesirable symptoms of aging. In some embodiments, administering an anti-Sortilin antibody may decrease expression or secretion of pro-inflammatory mediators in an individual having one or more undesirable symptoms of aging. In some embodiments, administering an anti-Sortilin antibody may inhibit interaction (e.g., binding) between Sortilin and one or more of pro-neurotrophins, neurotrophins, neurotensin, p75, Sortilin propeptide (Sort-pro), amyloid precursor protein (APP), A beta peptide, lipoprotein lipase (LpL), apolipoprotein AV (APOA5), apolipoprotein E (APOE), and receptor associated protein (RAP) in an individual having one or more undesirable symptoms of aging. In some embodiments, administering an anti-Sortilin antibody may decrease secretion of PCSK9 in an individual having one or more undesirable symptoms of aging. In some embodiments, administering an anti-Sortilin antibody may decrease production of beta amyloid peptide in an individual having one or more undesirable symptoms of aging.

Amyotrophic Lateral Sclerosis (ALS)

As used herein, amyotrophic lateral sclerosis (ALS), motor neuron disease, or Lou Gehrig's disease are used interchangeably and refer to a debilitating disease with varied etiology characterized by rapidly progressive weakness, muscle atrophy and fasciculations, muscle spasticity, difficulty speaking (dysarthria), difficulty swallowing (dysphagia), and difficulty breathing (dyspnea).

Progranulin haploinsufficiency due to heterozygous loss-of-function mutations in the GRN gene results in a reduction of cerebrospinal fluid Progranulin levels and is causal for the development of frontotemporal dementia (FTD) with TDP-43 pathology (Sleegers et al., (2009) Ann Neurol 65:603; Smith et al., (2012) Am J Hum Genet 90:1102). TDP-43 has also been identified as a major pathological protein in ALS, suggesting a similarity between ALS and FTD.

For example, over twenty dominant mutations in TDP-43 have been identified in sporadic and familial ALS patients (Lagier-Tourenne et al., (2009) Cell 136:1001) and TDP-43 positive aggregates are found in approximately 95% of ALS cases (Prasad et al., (2019) Front Mol Neurosci 12:25). Furthermore, ALS risk genes, such as MOBP, C90RF72, MOBKL2B, NSF and FUS, can also cause FTD (Karch et al., (2018) JAMA Neurol 75:860). In addition, both GRN and C90RF72 mutations are associated with abnormal microglial activation, which appears to be another common pathology of FTD and ALS (Haukedal et al., (2019) J Mol Biol 431:1818). Other evidence also suggests that ALS and FTD are closely related conditions with overlapping genetic, neuropathological, and clinical features (Weishaupt et al., (2016) Trends Mol Med 22:769; McCauley et al., (2018) Acta Neuropathol 137:715). Taken together, these results suggest that both diseases could benefit from shared treatments and that GRN genetic variability acts as a modifier of the course of ALS.

Moreover, aside from demonstrations that loss of Progranulin is detrimental in multiple models of acute and chronic neurodegeneration (Boddaert et al., (2018) Methods Mol Biol 1806:233), overexpression of Progranulin has been found to be protective in many animal models of ALS (Laird et al., (2010) PLoS One 5:e13368; Tauffenberger et al., (2013) Hum Mol Genet 22:782; Beel et al., (2018) Mol Neurodegener 13:55; Chang et al., (2017) J Exp Med 214:2611). In addition, common variants in GRN are significantly associated with a reduction in age at onset and a shorter survival after onset in ALS patients (Sleegers et al., (2008) Neurology 71:253).

In summary, both human genetics and data from disease models support a protective function for Progranulin in reducing pathology in ALS patients that are associated with TDP-43 pathology.

In some embodiments, and without wishing to be bound by theory, it is believed that anti-Sortilin antibodies of the present disclosure that inhibit the interaction between Sortilin and Progranulin, neurotrophins of the present disclosure (e.g., pro-neurotrophins, pro-neurotrophin-3, pro-neurotrophin-4/5, pro-NGF, pro-BDNF, neurotrophin-3, neurotrophin-4/5, NGF, BDNF, etc.), neurotensin, p75, lipoprotein lipase (LpL), apolipoprotein AV (APOA5), and/or receptor associated protein (RAP); or that inhibit one or more activities of Sortilin can be utilized to prevent, or treat one or more undesirable symptoms of ALS.

In some embodiments, administering an anti-Sortilin antibody of the present disclosure can treat and/or delay the progression of ALS. In some embodiments, administering an anti-Sortilin antibody may modulate one or more Sortilin activities in an individual having ALS. In some embodiments, administering an anti-Sortilin antibody may induce one or more Progranulin activities in an individual having ALS. In some embodiments, administering an anti-Sortilin antibody may inhibit one or more activities of Sortilin in an individual having ALS. In some embodiments, administering an anti-Sortilin antibody may decrease cellular levels of Sortilin in an individual having ALS. In some embodiments, administering an anti-Sortilin antibody may increase Progranulin levels in an individual having ALS. In some embodiments, administering an anti-Sortilin antibody may inhibit the interaction (e.g., binding) between Progranulin and Sortilin in an individual having ALS. In some embodiments, administering an anti-Sortilin antibody may decrease expression or secretion of pro-inflammatory mediators in an individual having ALS. In some embodiments, administering an anti-Sortilin antibody may inhibit interaction (e.g., binding) between Sortilin and one or more of pro-neurotrophins, neurotrophins, neurotensin, p75, Sortilin propeptide (Sort-pro), amyloid precursor protein (APP), A beta peptide, lipoprotein lipase (LpL), apolipoprotein AV (APOA5), apolipoprotein E (APOE), and receptor associated protein (RAP) in an individual having ALS. In some embodiments, administering an anti-Sortilin antibody may decrease secretion of PCSK9 in an individual having ALS. In some embodiments, administering an anti-Sortilin antibody may decrease production of beta amyloid peptide in an individual having ALS.

In some embodiments, an individual with ALS is heterozygous for a C9orf72 hexanucleotide repeat expansion.

In some embodiments, treatment and/or delay of ALS progression is determined by a change from baseline in brain atrophy, brain connectivity, brain free water and/or brain inflammation. Any method known in the art including, without limitation, MRI, may be used to measure brain atrophy, brain connectivity, brain free water and/or brain inflammation. In certain embodiments, brain atrophy is measured using structural MRI. In certain embodiments, brain free water and/or brain inflammation are measured using diffusion tensor imaging (DTI).

In some embodiments, treatment and/or delay of ALS progression is determined by a change from baseline in Progranulin, markers of neurodegeneration, markers of glial activation, and/or markers of TDP-43 pathology. In certain embodiments, Progranulin is measured using an Adipogen immunoassay. In certain embodiments, markers of neurodegeneration include, without limitation, NfL. NfL may be measured by any known methods in the art including, without limitation, assays from Quanterix and/or Roche Diagnostics. In certain embodiments, markers of glial activation include, without limitation, YKL-40 (CHI3L), IL-6, and/or GFAP. GFAP may be measured using any methods known in the art including, without limitation, assays from Roche Diagnostics.

Parkinson's Disease

Parkinson's disease, which may be referred to as idiopathic or primary parkinsonism, hypokinetic rigid syndrome (HRS), or paralysis agitans, is a neurodegenerative brain disorder that affects motor system control. The progressive death of dopamine-producing cells in the brain leads to the major symptoms of Parkinson's. Most often, Parkinson's disease is diagnosed in people over 50 years of age. Parkinson's disease is idiopathic (having no known cause) in most people. However, genetic factors also play a role in the disease.

Symptoms of Parkinson's disease include, without limitation, tremors of the hands, arms, legs, jaw, and face, muscle rigidity in the limbs and trunk, slowness of movement (bradykinesia), postural instability, difficulty walking, neuropsychiatric problems, changes in speech or behavior, depression, anxiety, pain, psychosis, dementia, hallucinations, and sleep problems.

In some embodiments, administering an anti-Sortilin antibody of the present disclosure can treat and/or delay the progression of Parkinson's disease. In some embodiments, administering an anti-Sortilin antibody may modulate one or more Sortilin activities in an individual having Parkinson's disease. In some embodiments, administering an anti-Sortilin antibody may induce one or more Progranulin activities in an individual having Parkinson's disease. In some embodiments, administering an anti-Sortilin antibody may inhibit one or more activities of Sortilin in an individual having Parkinson's disease. In some embodiments, administering an anti-Sortilin antibody may decrease cellular levels of Sortilin in an individual having Parkinson's disease. In some embodiments, administering an anti-Sortilin antibody may increase Progranulin levels in an individual having Parkinson's disease. In some embodiments, administering an anti-Sortilin antibody may inhibit the interaction (e.g., binding) between Progranulin and Sortilin in an individual having Parkinson's disease. In some embodiments, administering an anti-Sortilin antibody may decrease expression or secretion of pro-inflammatory mediators in an individual having Parkinson's disease. In some embodiments, administering an anti-Sortilin antibody may inhibit interaction (e.g., binding) between Sortilin and one or more of pro-neurotrophins, neurotrophins, neurotensin, p75, Sortilin propeptide (Sort-pro), amyloid precursor protein (APP), A beta peptide, lipoprotein lipase (LpL), apolipoprotein AV (APOA5), apolipoprotein E (APOE), and receptor associated protein (RAP) in an individual having Parkinson's disease. In some embodiments, administering an anti-Sortilin antibody may decrease secretion of PCSK9 in an individual having Parkinson's disease. In some embodiments, administering an anti-Sortilin antibody may decrease production of beta amyloid peptide in an individual having Parkinson's disease.

### Multiple Sclerosis

Multiple sclerosis (MS) can also be referred to as disseminated sclerosis or encephalomyelitis disseminata. MS is an inflammatory disease in which the fatty myelin sheaths around the axons of the brain and spinal cord are damaged, leading to demyelination and scarring as well as a broad spectrum of signs and symptoms. See, e.g., www.ninds.nih.gov/Disorders/Patient-Caregiver-Education/Hope-Through-Research/Multiple-Sclerosis-Hope-Through-Research.

Symptoms of MS include, without limitation, changes in sensation, such as loss of sensitivity or tingling; pricking or numbness, such as hypoesthesia and paresthesia; muscle weakness; clonus; muscle spasms; difficulty in moving; difficulties with coordination and balance, such as ataxia; problems in speech, such as dysarthria, or in swallowing, such as dysphagia; visual problems, such as nystagmus, optic neuritis including phosphenes, and diplopia; fatigue; acute or chronic pain; and bladder and bowel difficulties; cognitive impairment of varying degrees; emotional symptoms of depression or unstable mood; Uhthoffs phenomenon, which is an exacerbation of extant symptoms due to an exposure to higher than usual ambient temperatures; and Lhermitte's sign, which is an electrical sensation that runs down the back when bending the neck.

In some embodiments, administering an anti-Sortilin antibody of the present disclosure can treat and/or delay the progression of multiple sclerosis. In some embodiments, administering an anti-Sortilin antibody may modulate one or more Sortilin activities in an individual having multiple sclerosis. In some embodiments, administering an anti-Sortilin antibody may induce one or more Progranulin activities in an individual having multiple sclerosis. In some embodiments, administering an anti-Sortilin antibody may inhibit one or more activities of Sortilin in an individual having multiple sclerosis. In some embodiments, administering an anti-Sortilin antibody may decrease cellular levels of Sortilin in an individual having multiple sclerosis. In some embodiments, administering an anti-Sortilin antibody may increase Progranulin levels in an individual having multiple sclerosis. In some embodiments, administering an anti-Sortilin antibody may inhibit the interaction (e.g., binding) between Progranulin and Sortilin in an individual having multiple sclerosis. In some embodiments, administering an anti-Sortilin antibody may decrease expression or secretion of pro-inflammatory mediators in an individual having multiple sclerosis. In some embodiments, administering an anti-Sortilin antibody may inhibit interaction (e.g., binding) between Sortilin and one or more of pro-neurotrophins, neurotrophins, neurotensin, p75, Sortilin propeptide (Sort-pro), amyloid precursor protein (APP), A beta peptide, lipoprotein lipase (LpL), apolipoprotein AV (APOA5), apolipoprotein E (APOE), and receptor associated protein (RAP) in an individual having multiple sclerosis. In some embodiments, administering an anti-Sortilin antibody may decrease secretion of PCSK9 in an individual having multiple sclerosis. In some embodiments, administering an anti-Sortilin antibody may decrease production of beta amyloid peptide in an individual having multiple sclerosis.

### Glaucoma and Macular Degeneration

Glaucoma describes, without limitation, a group of diseases that are characterized by a damaged optic nerve, resulting in vision loss and blindness. Glaucoma is usually caused by increased fluid pressure (e.g., intraocular pressure) in the anterior chamber underneath the cornea. Glaucoma results in the successive loss of retinal ganglion cells that are important for vision. Age-related macular degeneration usually affects older people and primarily causes loss of vision in the macula, the central field of vision. Macular degeneration causes, without limitation, drusen, pigmentary changes, distorted vision, hemorrhages of the eye, atrophy, reduced visual acuity, blurred vision, central scotomas, reduced color vision and reduced contrast sensitivity.

Without wishing to be bound by theory, it is believed that administering an anti-Sortilin antibody of the present disclosure can treat and/or delay the progression of glaucoma and macular degeneration. In some embodiments, administering an anti-Sortilin antibody may modulate one or more Sortilin activities in an individual having glaucoma or macular degeneration. In some embodiments, administering an anti-Sortilin antibody may induce one or more Progranulin activities in an individual having glaucoma or macular degeneration. In some embodiments, administering an anti-Sortilin antibody may inhibit one or more activities of Sortilin in an individual having glaucoma or macular degeneration. In some embodiments, administering an anti-Sortilin antibody may decrease cellular levels of Sortilin in an individual having glaucoma or macular degeneration. In some embodiments, administering an anti-Sortilin antibody may increase Progranulin levels in an individual having glaucoma or macular degeneration. In some embodiments, administering an anti-Sortilin antibody may inhibit the interaction (e.g., binding) between Progranulin and Sortilin in an individual having glaucoma or macular degeneration. In some embodiments, administering an anti-Sortilin antibody may decrease expression or secretion of pro-inflammatory mediators in an individual having glaucoma or macular degeneration. In some embodiments, administering an anti-Sortilin antibody may inhibit interaction (e.g., binding) between Sortilin and one or more of pro-neurotrophins, neurotrophins, neurotensin, p75, Sortilin propeptide (Sort-pro), amyloid precursor protein (APP), A beta peptide, lipoprotein lipase (LpL), apolipoprotein AV (APOA5), apolipoprotein E (APOE), and receptor associated protein (RAP) in an individual having glaucoma or macular degeneration. In some embodiments, administering an anti-Sortilin antibody may decrease secretion of PCSK9 in an individual having glaucoma or macular degeneration. In some embodiments, administering an anti-Sortilin antibody may decrease production of beta amyloid peptide in an individual having glaucoma or macular degeneration.

### Pharmaceutical Dosages

An antibody provided herein (and any additional therapeutic agent) can be administered by any suitable means, including parenteral, intrapulmonary, intranasal, intralesional, intracerobrospinal, intracranial, intraspinal, intrasynovial, intrathecal, oral, topical, or inhalation routes. Parenteral infusions include intramuscular, intravenous administration as a bolus or by continuous infusion over a period of time, intraarterial, intra-articular, intraperitoneal, or subcutaneous administration. In some embodiments, the administration is intravenous. In some embodiments, the administration is subcutaneous. Dosing can be by any suitable route, e.g. by injections, such as intravenous or subcutaneous injections, depending in part on whether the administration is brief or chronic. Various dosing schedules including, but not limited to, single or multiple administrations over various time-points, bolus administration, and pulse infusion are contemplated herein.

Antibodies provided herein would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

Dosages for a particular anti-Sortilin antibody may be determined empirically in individuals who have been given one or more administrations of the anti-Sortilin antibody. Individuals are given incremental doses of an anti-Sortilin antibody. To assess efficacy of an anti-Sortilin antibody, a clinical symptom of any of the diseases, disorders, or conditions of the present disclosure (e.g., frontotemporal dementia, Alzheimer's disease, vascular dementia, seizures, retinal dystrophy, a traumatic brain injury, a spinal cord injury, long-term depression, atherosclerotic vascular diseases, and undesirable symptoms of normal aging) can be monitored.

For the prevention or treatment of disease, the appropriate dosage of an antibody of the disclosure (when used alone or in combination with one or more additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments.

Depending on the type and severity of the disease, about 1 μg/kg to 15 mg/kg (e.g., 0.1 mg/kg-10 mg/kg) of antibody can be an initial candidate dosage for administration to the individual, whether, for example, by one or more separate administrations, or by continuous infusion. One daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 15 mg/kg to about 70 mg/kg. Thus, one or more doses of about 15 mg/kg, 20 mg/kg, 25 mg/kg, 30 mg/kg, 35 mg/kg, 40 mg/kg, 45 mg/kg, 50 mg/kg, 55 mg/kg, 60 mg/kg, 65 mg/kg, or 70 mg/kg (or any combination thereof) may be administered to the individual. Another exemplary dosage of the antibody would be in the range from about 30 mg/kg to about 60 mg/kg. Thus, one or more doses of about 30 mg/kg, 35 mg/kg, 40 mg/kg, 45 mg/kg, 50 mg/kg, 55 mg/kg, or 60 mg/kg (or any combination thereof) may be administered to the individual.

In some embodiments, the methods of the present disclosure comprise administering to the individual an anti-Sortilin antibody intravenously at a dose of at least about 30 mg/kg. In some embodiments, the dose is at least about 35 mg/kg, at least about 40 mg/kg, at least about 45 mg/kg, at least about 50 mg/kg, at least about 55 mg/kg, or at least about 60 mg/kg. In some embodiments, the dose is between about 30 mg/kg and about 60 mg/kg. In some embodiments, the dose is about 35 mg/kg, about 40 mg/kg, about 45 mg/kg, about 50 mg/kg, about 55 mg/kg, or about 60 mg/kg. In some embodiments, the dose is about 60 mg/kg. In some embodiments, the dose is 60 mg/kg.

Such doses may be administered intermittently. In certain embodiments, dosing frequency is three times per day, twice per day, once per day, once every other day, once weekly, once every two weeks, once every three weeks, once every four weeks, once every five weeks, once every six weeks, once every seven weeks, once every eight weeks, once every nine weeks, once every ten weeks, or once monthly, once every two months, once every three months, or less frequently. In some embodiments, doses are administered about once every four weeks (q4w). In some embodiments, doses are administered once every four weeks (q4w).

In some embodiments, the anti-Sortilin antibody is administered to the individual intravenously at a dose of 60 mg/kg once every four weeks.

In certain embodiments, the anti-Sortilin antibody is administered to the individual intravenously over about 60 minutes. In certain embodiments, the anti-Sortilin antibody is administered to the individual intravenously at a dose of 60 mg/kg over at least 60 minutes.

In certain embodiments, at least 1 dose, at least 2 doses, at least 3 doses, at least 4 doses, at least 5 doses, at least 6 doses, at least 7 doses, at least 8 doses, at least 9 doses, at least 10 doses, at least 11 doses, at least 12 doses, at least 13 doses, at least 14 doses, at least 15 doses, at least 16 doses, at least 17 doses, at least 18 doses, at least 19 doses, at least 20 doses, at least 21 doses, at least 22 doses, at least 23 doses, at least 24 doses, or at least 25 doses of the anti-Sortilin antibody are administered to the individual. In certain embodiments, a total of 25 doses of the anti-Sortilin antibody are administered to the individual. In certain embodiments, at least 25 doses, at least 26 doses, at least 27 doses, at least 28 doses, at least 29 doses, at least 30 doses, at least 31 doses, at least 32 doses, at least 33 doses, at least 34 doses, at least 35 doses, at least 36 doses, at least 37 doses, at least 38 doses, at least 39 doses, at least 40 doses, at least 41 doses, at least 42 doses, at least 43 doses, at least 44 doses, at least 45 doses, at least 46 doses, at least 47 doses, at least 48 doses, at least 49 doses, or at least 50 doses of the anti-Sortilin antibody are administered to the individual. In certain embodiments, a total of 50 doses of the anti-Sortilin antibody are administered to the individual.

In some embodiments, the individual is treated for a treatment period of at least about 4 weeks, at least about 8 weeks, at least about 12 weeks, at least about 16 weeks, at least about 20 weeks, at least about 24 weeks, at least about 28 weeks, at least about 32 weeks, at least about 36 weeks, at least about 40 weeks, at least about 44 weeks, at least about 48 weeks, at least about 52 weeks, at least about 56 weeks, at least about 60 weeks, at least about 64 weeks, at least about 68 weeks, at least about 72 weeks, at least about 76 weeks, at least about 80 weeks, at least about 84 weeks, at least about 88 weeks, at least about 92 weeks, or at least about 96 weeks. In some embodiments, the individual is treated for a treatment period of 96 weeks. In some embodiments, the individual is treated for a treatment period of at least about 96 weeks, at least about 100 weeks, at least about 104 weeks, at least about 108 weeks, at least about 112 weeks, at least about 116 weeks, at least about 120 weeks, at least about 124 weeks, at least about 128 weeks, at least about 132 weeks, at least about 136 weeks, at least about 140 weeks, at least about 144 weeks, at least about 148 weeks, at least about 152 weeks, at least about 156 weeks, at least about 160 weeks, at least about 164 weeks, at least about 168 weeks, at least about 172 weeks, at least about 176 weeks, at least about 180 weeks, at least about 184 weeks, at least about 188 weeks, or at least about 192 weeks. In some embodiments, the individual is treated for a treatment period of 192 weeks. In some embodiments, the individual is treated during a first treatment period of 96 weeks and during a second treatment period of 96 weeks after the first treatment period.

In some embodiments, administration of the anti-Sortilin antibody occurs on the first day of the treatment period and every four weeks thereafter.

Other dosage regimens may be useful. An initial higher loading dose, followed by one or more lower doses may be administered. The progress of this therapy is easily monitored by conventional techniques and assays.

### Diagnostic Uses

The isolated antibodies of the present disclosure (e.g., an anti-Sortilin antibody described herein) also have diagnostic utility. This disclosure therefore provides for methods of using the antibodies of this disclosure, or functional fragments thereof, for diagnostic purposes, such as the detection of a Sortilin protein in an individual or in tissue samples derived from an individual.

In some embodiments, the individual is a human. In some embodiments, the individual is a human patient suffering from, or at risk for developing a disease, disorder, or injury of the present disclosure. In some embodiments, the diagnostic methods involve detecting a Sortilin protein in a biological sample, such as a biopsy specimen, a tissue, or a cell. An anti-Sortilin antibody described herein is contacted with the biological sample and antigen-bound antibody is detected. For example, a biopsy specimen may be stained with an anti-Sortilin antibody described herein in order to detect and/or quantify disease-associated cells. The detection method may involve quantification of the antigen-bound antibody. Antibody detection in biological samples may occur with any method known in the art, including immunofluorescence microscopy, immunocytochemistry, immunohistochemistry, ELISA, FACS analysis, immunoprecipitation, or micro-positron emission tomography. In certain embodiments, the antibody is radiolabeled, for example with ¹⁸F and subsequently detected utilizing micro-positron emission tomography analysis. Antibody-binding may also be quantified in an individual by non-invasive techniques such as positron emission tomography (PET), X-ray computed tomography, single-photon emission computed tomography (SPECT), computed tomography (CT), and computed axial tomography (CAT).

In other embodiments, an isolated antibody of the present disclosure (e.g., an anti-Sortilin antibody described herein) may be used to detect and/or quantify, for example, microglia in a brain specimen taken from a preclinical disease model (e.g., a non-human disease model). As such, an isolated antibody of the present disclosure (e.g., an anti-Sortilin antibody described herein) may be useful in evaluating therapeutic response after treatment in a model for a nervous system disease or injury such as frontotemporal dementia, Alzheimer's disease, vascular dementia, amyotrophic lateral sclerosis, Parkinson's disease, seizures, retinal dystrophy, atherosclerotic vascular diseases, Nasu-Hakola disease, or multiple sclerosis, as compared to a control.

### Sortilin Antibodies

Certain aspects of the present disclosure relate to anti-Sortilin antibodies comprising one or more improved and/or enhanced functional characteristics. In some embodiments, anti-Sortilin antibodies of the present disclosure comprise one or more improved and/or enhanced functional characteristics relative to an anti-Sortilin antibody, S-60, having a heavy chain variable region and a light chain variable region as described in WO2016164637. In some embodiments, anti-Sortilin antibodies of the present disclosure have an affinity for Sortilin (e.g., human Sortilin) that is higher than that of a control anti-Sortilin antibody (e.g., a control anti-Sortilin antibody comprising a heavy chain variable region and a light chain variable region corresponding to S-60). In some embodiments, anti-Sortilin antibodies of the present disclosure decrease cellular levels (e.g., cell surface levels) of Sortilin to a greater degree and with a half-maximal effective concentration (EC₅₀) that is lower than that of a control antibody (e.g., a control anti-Sortilin antibody comprising a heavy chain variable region and a light chain variable region corresponding to S-60). In some embodiments, anti-Sortilin antibodies of the present disclosure improve the maximal reduction of cell surface levels of Sortilin relative to an anti-Sortilin antibody comprising a heavy chain variable region and a light chain variable region corresponding to S-60. In some embodiments, anti-Sortilin antibodies of the present disclosure increase the secretion of extracellular Progranulin (PGRN) relative to an anti-Sortilin antibody comprising a heavy chain variable region and a light chain variable region corresponding to S-60. In some embodiments, anti-Sortilin antibodies of the present disclosure block binding of PGRN to Sortilin to a greater degree and with a half-maximal effective concentration (EC₅₀) that is lower than that of a control antibody (e.g., a control anti-Sortilin antibody comprising a heavy chain variable region and a light chain variable region corresponding to S-60). In some embodiments, anti-Sortilin antibodies of the present disclosure improve the maximal blocking of PGRN binding to Sortilin relative to an anti-Sortilin antibody comprising a heavy chain variable region and a light chain variable region corresponding to S-60.

Also contemplated herein are anti-Sortilin antibodies with different Fc variants that exhibit one or more improved and/or enhanced functional characteristics relative to an anti-Sortilin antibody comprising a heavy chain variable region and a light chain variable region corresponding to S-60, including decreasing the half-maximal effective concentration (EC₅₀) to reduce cell surface levels of Sortilin, improving the maximal reduction of cell surface levels of Sortilin, increasing extracellular secretion of PGRN, decreasing the half-maximal effective concentration (EC₅₀) to block PGRN binding to Sortilin, and improving the maximal blocking of PGRN binding to Sortilin.

In some embodiments, an anti-Sortilin antibody of the present disclosure is a human antibody, a bispecific antibody, a monoclonal antibody, a multivalent antibody, a conjugated antibody, or a chimeric antibody

In a preferred embodiment, an anti-Sortilin antibody of the present disclosure is a monoclonal antibody.

### Anti-Sortilin Antibody Heavy Chain and Light Chain Variable Regions

A. Heavy Chain HVRs

In some embodiments, anti-Sortilin antibodies of the present disclosure include a heavy chain variable region comprising one or more (e.g., one or more, two or more, or all three) HVRs selected from HVR-H1, HVR-H2, and HVR-H3 (as shown in Tables 1-3). In some embodiments, the heavy chain variable region comprises an HVR-H1, an HVR-H2, and an HVR-H3 (as shown in Tables 1-3).

In some embodiments, the HVR-H1 comprises a sequence of YSISSGYYWG (SEQ ID NO: 1). In some embodiments, the HVR-H2 comprises a sequence according to Formula I: TIYHSGSTYYNPSLX₁S (SEQ ID NO: 4), wherein X₁ is K or E. In some embodiments, the HVR-H2 comprises a sequence selected from SEQ ID NOs: 2-3. In some embodiments, the HVR-H3 comprises a sequence according to Formula II: ARQGSIX₁QGYYGMDV (SEQ ID NO: 7). In some embodiments, the HVR-H3 comprises a sequence selected from SEQ ID NOs: 5-6.

In some embodiments, the HVR-H1 comprises an amino acid sequence with at least about 90%, at least about 91%, at least about 92%, at least about 93%, at least about 94%, at least about 95%, at least about 96%, at least about 97%, at least about 98%, at least about 99%, or 100% identity to an amino acid sequence of SEQ ID NO: 1. In some embodiments, the HVR-H1 comprises an amino acid sequence containing substitutions (e.g., conservative substitutions, insertions, or deletions relative to an amino acid sequence of SEQ ID NO: 1), but retains the ability to bind to Sortilin. In certain embodiments, up to 1, up to 2, up to 3, up to 4, or up to 5 amino acids been substituted, inserted, and/or deleted in the HVR-H1 amino acid sequence of SEQ ID NO: 1. In some embodiments, the HVR-H2 comprises an amino acid sequence with at least about 90%, at least about 91%, at least about 92%, at least about 93%, at least about 94%, at least about 95%, at least about 96%, at least about 97%, at least about 98%, at least about 99%, or 100% identity to an amino acid sequence selected from SEQ ID NOs: 2-3. In some embodiments, the HVR-H2 comprises an amino acid sequence containing substitutions (e.g., conservative substitutions, insertions, or deletions relative to an amino acid sequence selected from SEQ ID NOs: 2-3), but retains the ability to bind to Sortilin. In certain embodiments, up to 1, up to 2, up to 3, up to 4, or up to 5 amino acids been substituted, inserted, and/or deleted in the HVR-H2 amino acid sequence selected from SEQ ID NOs: 2-3. In some embodiments, the HVR-H3 comprises an amino acid sequence with at least about 90%, at least about 91%, at least about 92%, at least about 93%, at least about 94%, at least about 95%, at least about 96%, at least about 97%, at least about 98%, at least about 99%, or 100% identity to an amino acid sequence selected from SEQ ID NOs: 5-6. In some embodiments, the HVR-H3 comprises an amino acid sequence containing substitutions (e.g., conservative substitutions, insertions, or deletions relative to an amino acid sequence selected from SEQ ID NOs: 5-6), but retains the ability to bind to Sortilin. In certain embodiments, up to 1, up to 2, up to 3, up to 4, or up to 5 amino acids been substituted, inserted, and/or deleted in the HVR-H3 amino acid sequence selected from SEQ ID NOs: 5-6.

In some embodiments, the heavy chain variable region comprises an HVR-H1 comprising a sequence of YSISSGYYWG (SEQ ID NO: 1), an HVR-H2 comprising a sequence according to Formula I, and an HVR-H3 comprising a sequence according to Formula II.

In some embodiments, the heavy chain variable region comprises an HVR-H1 comprising a sequence of SEQ ID NO: 1, an HVR-H2 comprising a sequence selected from SEQ ID NOs: 2-3, and an HVR-H3 comprising a sequence selected from SEQ ID NOs: 5-6.

In some embodiments, the heavy chain variable region comprises the HVR-H1, HVR-H2, and HVR-H3 of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N331], S-60-15.14 [N33V], S-60-15.15 [N33A], S-60-15.16 [N33M], S-60-15.17 [N33L], S-60-16, S-60-18, S-60-19, S-60-24, or any combination thereof (as shown in Tables 1-3).

In some embodiments, anti-Sortilin antibodies of the present disclosure include a heavy chain variable region, wherein the heavy chain variable region comprises one or more of: (a) an HVR-H1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an HVR-H1 amino acid sequence of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16, S-60-18, S-60-19, or S-60-24; (b) an HVR-H2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an HVR-H2 amino acid sequence of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16, S-60-18, S-60-19, or S-60-24; and (c) an HVR-H3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an HVR-H3 amino acid sequence of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16, S-60-18, S-60-19, or S-60-24.

In some embodiments, anti-Sortilin antibodies of the present disclosure comprise an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6).

B. Light Chain HVRs

In some embodiments, anti-Sortilin antibodies of the present disclosure comprise a light chain variable region comprising one or more (e.g., one or more, two or more, or all three) HVRs selected from HVR-L1, HVR-L2, and HVR-L3 (as shown in Tables 4-6). In some embodiments, the light chain variable region comprises an HVR-L1, an HVR-L2, and an HVR-L3 (as shown in Tables 4-6).

In some embodiments, the HVR-L1 comprises a sequence according to Formula III: RSSQX₁LLX₂SX₃GYNYLD (SEQ ID NO: 28), wherein X₁ is S or G, X₂ is R or H, and X₃ is N, T, S, G, R, D, H, K, Q, Y, E, W, F, I, V, A, M, or L. In some embodiments, the HVR-L1 comprises a sequence selected from SEQ ID NOs: 8-27. In some embodiments, the HVR-L1 comprises a sequence of RSSQSLLRSNGYNYLD (SEQ ID NO:8), RSSQSLLRSTGYNYLD (SEQ ID NO:9), RSSQS LLRSSGYNYLD (SEQ ID NO: 10), RSSQSLLRSGGYNYLD (SEQ ID NO:11), RSSQSLLRSRG YNYLD (SEQ ID NO:12), RSSQSLLRSDGYNYLD (SEQ ID NO:13), RSSQSLLRSHGYNYLD (SEQ ID NO: 14), RSSQSLLRSKGYNYLD (SEQ ID NO:15), RSSQSLLRSQGYNYLD (SEQ ID NO:16), RSSQSLLRSYGYNYLD (SEQ ID NO:17), RSSQSLLRSEGYNYLD (SEQ ID NO: 18), RSSQSLLRSWGYNYLD (SEQ ID NO:19), RSSQSLLRSFGYNYLD (SEQ ID NO:20), RSSQSL LRSIGYNYLD (SEQ ID NO:21), RSSQSLLRSVGYNYLD (SEQ ID NO:22), RSSQSLLRSAG YNYLD (SEQ ID NO:23), RSSQSLLRSMGYNYLD (SEQ ID NO:24), RSSQSLLRSLGYNYLD (SEQ ID NO:25), RSSQSLLHSNGYNYLD (SEQ ID NO:26), or RSSQGLLRSNGYNYLD (SEQ ID NO:27). In one specific embodiment, the HVR-L1 comprises a sequence of RSSQSLLRSNGYNYLD (SEQ ID NO:8). In another specific embodiment, the HVR-L1 comprises a sequence of RSSQSLLRSTGYNYLD (SEQ ID NO:9) (as shown in Table 4).

In some embodiments, the HVR-L2 comprises a sequence according to Formula IV: LGSNRX1S (SEQ ID NO: 31), wherein X1 is A or V. In some embodiments, the HVR-L2 comprises a sequence selected from SEQ ID NOs: 29-30.

In some embodiments, the HVR-L3 comprises a sequence according to Formula V: MQQQEX1PLT (SEQ ID NO: 34), wherein X1 is A or T. In some embodiments, the HVR-L3 comprises a sequence selected from SEQ ID NOs: 32-33.

In some embodiments, the HVR-L1 comprises an amino acid sequence with at least about 90%, at least about 91%, at least about 92%, at least about 93%, at least about 94%, at least about 95%, at least about 96%, at least about 97%, at least about 98%, at least about 99%, or 100% identity to an amino acid sequence selected from SEQ ID NOs: 8-27. In some embodiments, the HVR-L1 comprises an amino acid sequence containing substitutions (e.g., conservative substitutions, insertions, or deletions relative to an amino acid sequence selected from SEQ ID NOs: 8-27), but retains the ability to bind to Sortilin. In certain embodiments, up to 1, up to 2, up to 3, up to 4, or up to 5 amino acids been substituted, inserted, and/or deleted in the HVR-L1 amino acid sequence selected from SEQ ID NOs: 8-27. In some embodiments, the HVR-L2 comprises an amino acid sequence with at least about 90%, at least about 91%, at least about 92%, at least about 93%, at least about 94%, at least about 95%, at least about 96%, at least about 97%, at least about 98%, at least about 99%, or 100% identity to an amino acid sequence selected from SEQ ID NOs: 29-30. In some embodiments, the HVR-L2 comprises an amino acid sequence containing substitutions (e.g., conservative substitutions, insertions, or deletions relative to an amino acid sequence selected from SEQ ID NOs: 29-30), but retains the ability to bind to Sortilin. In certain embodiments, up to 1, up to 2, up to 3, up to 4, or up to 5 amino acids been substituted, inserted, and/or deleted in the HVR-L2 amino acid sequence selected from SEQ ID NOs: 29-30. In some embodiments, the HVR-L3 comprises an amino acid sequence with at least about 90%, at least about 91%, at least about 92%, at least about 93%, at least about 94%, at least about 95%, at least about 96%, at least about 97%, at least about 98%, at least about 99%, or 100% identity to an amino acid sequence selected from SEQ ID NOs: 32-33. In some embodiments, the HVR-L3 comprises an amino acid sequence containing substitutions (e.g., conservative substitutions, insertions, or deletions relative to an amino acid sequence selected from SEQ ID NOs: 32-33), but retains the ability to bind to Sortilin. In certain embodiments, up to 1, up to 2, up to 3, up to 4, or up to 5 amino acids been substituted, inserted, and/or deleted in the HVR-L3 amino acid sequence selected from SEQ ID NOs: 32-33.

In some embodiments, the light chain variable region comprises an HVR-L1 comprising a sequence according to Formula III, an HVR-L2 comprising a sequence according to Formula IV, and an HVR-L3 comprising a sequence according to Formula V. In some embodiments, the light chain variable region comprises an HVR-L1 comprising a sequence selected from SEQ ID NOs: 8-27, an HVR-L2 comprising a sequence selected from SEQ ID NOs: 29-30, and an HVR-L3 comprising a sequence selected from SEQ ID NOs: 32-33.

In some embodiments, the light chain variable region comprises the HVR-L1, HVR-L2, and HVR-L3 of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16, S-60-18, S-60-19, S-60-24, or any combination thereof (as shown in Tables 4-6).

In some embodiments, anti-Sortilin antibodies of the present disclosure include a light chain variable region, wherein the light chain variable region comprises one or more of: (a) an HVR-L1 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an HVR-L1 amino acid sequence of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16, S-60-18, S-60-19, or S-60-24; (b) an HVR-L2 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an HVR-L2 amino acid sequence of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16, S-60-18, S-60-19, or S-60-24; and (c) an HVR-L3 comprising an amino acid sequence with at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identity to an HVR-L3 amino acid sequence of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16, S-60-18, S-60-19, or S-60-24.

In some embodiments, anti-Sortilin antibodies of the present disclosure comprise an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32).

In some embodiments, anti-Sortilin antibodies of the present disclosure comprise an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32).

C. Heavy Chain HVRs and Light Chain HVRs

In some embodiments, anti-Sortilin antibodies of the present disclosure include a heavy chain variable region comprising one or more (e.g., one or more, two or more, or all three) HVRs selected from HVR-H1, HVR-H2, and HVR-H3 (as shown in Tables 1-3), and a light chain variable region comprising one or more (e.g., one or more, two or more, or all three) HVRs selected from HVR-L1, HVR-L2, and HVR-L3 (as shown in Tables 4-6). In some embodiments, the heavy chain variable region comprises an HVR-H1, an HVR-H2, and an HVR-H3 (as shown in Tables 1-3), and the light chain variable region comprises an HVR-L1, an HVR-L2, and an HVR-L3 (as shown in Tables 4-6).

In some embodiments, the heavy chain variable region comprises an HVR-H1 comprising a sequence of YSISSGYYWG (SEQ ID NO: 1), an HVR-H2 comprising a sequence according to Formula I, and an HVR-H3 comprising a sequence according to Formula II, and the light chain variable region comprises an HVR-L1 comprising a sequence according to Formula III, an HVR-L2 comprising a sequence according to Formula IV, and an HVR-L3 comprising a sequence according to Formula V. In some embodiments, the heavy chain variable region comprises an HVR-H1 comprising a sequence of SEQ ID NO: 1, an HVR-H2 comprising a sequence selected from SEQ ID NOs: 2-3, and an HVR-H3 comprising a sequence selected from SEQ ID NOs: 5-6, and the light chain variable region comprises an HVR-L1 comprising a sequence selected from SEQ ID NOs: 8-27, an HVR-L2 comprising a sequence selected from SEQ ID NOs: 29-30, and an HVR-L3 comprising a sequence selected from SEQ ID NOs: 32-33.

In some aspects, the heavy chain variable region comprises an HVR-H1 comprising a sequence of SEQ ID NO: 1, an HVR-H2 comprising a sequence selected from SEQ ID NOs: 2-3, and an HVR-H3 comprising a sequence selected from SEQ ID NOs: 5-6, and the light chain variable region comprises an HVR-L1 comprising a sequence selected from SEQ ID NOs: 8-27, an HVR-L2 comprising a sequence selected from SEQ ID NOs: 29-30, and an HVR-L3 comprising a sequence of SEQ ID NO: 32.

In some embodiments, anti-Sortilin antibodies of the present disclosure comprise a heavy chain variable region comprising the HVR-H1, HVR-H2, and HVR-H3 of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16, S-60-18, S-60-19, S-60-24, or any combination thereof (as shown in Tables 1-3); and a light chain variable region comprising the HVR-L1, HVR-L2, and HVR-L3 of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16, S-60-18, S-60-19, S-60-24, or any combination thereof (as shown in Tables 4-6).

In some embodiments, anti-Sortilin antibodies of the present disclosure comprise a heavy chain variable region comprising an HVR-H1, HVR-H2, and HVR-H3 and a light chain variable region comprising an HVR-L1, HVR-L2, and HVR-L3, wherein the antibody comprises the HVR-H1, HVR-H2, HVR-H3, HVR-L1, HVR-L2, and HVR-L3 of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16; S-60-18, S-60-19, or S-60-24 (as shown in Tables 1-6).

In some embodiments, anti-Sortilin antibodies of the present disclosure comprise a heavy chain variable region and a light chain variable region, wherein the heavy chain variable region comprises one or more of: (a) an HVR-H1 comprising an amino acid sequence with at least 90%, at least 91%, at least 92%, at least 93%, at least 94%, at least 95%, at least 96%, at least 97%, at least 98%, at least 99%, or 100% identity to an HVR-H1 amino acid sequence of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16. S-60-18, S-60-19, or S-60-24; (b) an HVR-H2 comprising an amino acid sequence with at least 90%, at least 91%, at least 92%, at least 93%, at least 94%, at least 95%, at least 96%, at least 97%, at least 98%, at least 99%, or 100% identity to an HVR-H2 amino acid sequence of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16, S-60-18, S-60-19, or S-60-24; and (c) an HVR-H3 comprising an amino acid sequence with at least 90%, at least 91%, at least 92%, at least 93%, at least 94%, at least 95%, at least 96%, at least 97%, at least 98%, at least 99%, or 100% identity to an HVR-H3 amino acid sequence of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16, S-60-18, S-60-19, or S-60-24; and wherein the light chain variable region comprises one or more of: (a) an HVR-L1 comprising an amino acid sequence with at least 90%, at least 91%, at least 92%, at least 93%, at least 94%, at least 95%, at least 96%, at least 97%, at least 98%, at least 99%, or 100% identity to an HVR-L1 amino acid sequence of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16, S-60-18, S-60-19, or S-60-24; (b) an HVR-L2 comprising an amino acid sequence with at least 90%, at least 91%, at least 92%, at least 93%, at least 94%, at least 95%, at least 96%, at least 97%, at least 98%, at least 99%, or 100% identity to an HVR-L2 amino acid sequence of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16, S-60-18, S-60-19, or S-60-24; and (c) an HVR-L3 comprising an amino acid sequence with at least 90%, at least 91%, at least 92%, at least 93%, at least 94%, at least 95%, at least 96%, at least 97%, at least 98%, at least 99%, or 100% identity to an HVR-L3 amino acid sequence of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16, S-60-18, S-60-19, or S-60-24.

In some embodiments, an anti-Sortilin antibody of the present disclosure comprises a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIQQGYYGMDV (SEQ ID NO: 5); and a light chain variable region comprising the HVR-L1 comprising the amino acid sequence RSSQSLLRSNGYNYLD (SEQ ID NO: 8), the HVR-L2 comprising the amino acid sequence LGSNRAS (SEQ ID NO: 29), and the HVR-L3 comprising the amino acid sequence MQQQEAPLT (SEQ ID NO: 32).

In some embodiments, an anti-Sortilin antibody of the present disclosure comprises a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIQQGYYGMDV (SEQ ID NO: 5); and a light chain variable region comprising the HVR-L1 comprising the amino acid sequence RSSQSLLRSNGYNYLD (SEQ ID NO: 8), the HVR-L2 comprising the amino acid sequence LGSNRVS (SEQ ID NO: 30), and the HVR-L3 comprising the amino acid sequence MQQQETPLT (SEQ ID NO: 33).

In some embodiments, an anti-Sortilin antibody of the present disclosure comprises a heavy chain variable region comprising the HVR-H1 comprising the amino acid sequence YSISSGYYWG (SEQ ID NO: 1), the HVR-H2 comprising the amino acid sequence TIYHSGSTYYNPSLES (SEQ ID NO: 3), the HVR-H3 comprising the amino acid sequence ARQGSIQQGYYGMDV (SEQ ID NO: 5); and a light chain variable region comprising the HVR-L1 comprising the amino acid sequence RSSQSLLRSNGYNYLD (SEQ ID NO: 8), the HVR-L2 comprising the amino acid sequence LGSNRAS (SEQ ID NO: 29), and the HVR-L3 comprising the amino acid sequence MQQQEAPLT (SEQ ID NO: 32).

In some aspects, an anti-Sortilin antibody of the present disclosure comprises a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIKQGYYGMDV (SEQ ID NO: 6); and a light chain variable region comprising the HVR-L1 comprising the amino acid sequence RSSQSLLRSNGYNYLD (SEQ ID NO: 8), the HVR-L2 comprising the amino acid sequence LGSNRAS (SEQ ID NO: 29), and the HVR-L3 comprising the amino acid sequence MQQQEAPLT (SEQ ID NO: 32).

In some aspects, an anti-Sortilin antibody of the present disclosure comprises a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIKQGYYGMDV (SEQ ID NO: 6); and a light chain variable region comprising the HVR-L1 comprising the amino acid sequence RSSQSLLRSTGYNYLD (SEQ ID NO: 9), the HVR-L2 comprising the amino acid sequence LGSNRAS (SEQ ID NO: 29), and the HVR-L3 comprising the amino acid sequence MQQQEAPLT (SEQ ID NO: 32).

In some embodiments, an anti-Sortilin antibody of the present disclosure comprises a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIKQGYYGMDV (SEQ ID NO: 6); and a light chain variable region comprising the HVR-L1 comprising the amino acid sequence RSSQSLLRSNGYNYLD (SEQ ID NO: 8), the HVR-L2 comprising the amino acid sequence LGSNRAS (SEQ ID NO: 29), and the HVR-L3 comprising the amino acid sequence MQQQETPLT (SEQ ID NO: 33).

In some embodiments, an anti-Sortilin antibody of the present disclosure comprises a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIQQGYYGMDV (SEQ ID NO: 5); and a light chain variable region comprising the HVR-L1 comprising the amino acid sequence RSSQSLLHSNGYNYLD (SEQ ID NO: 26), the HVR-L2 comprising the amino acid sequence LGSNRAS (SEQ ID NO: 29), and the HVR-L3 comprising the amino acid sequence MQQQETPLT (SEQ ID NO: 33).

In some embodiments, an anti-Sortilin antibody of the present disclosure comprises a heavy chain variable region comprising the HVR-H1 comprising the amino acid sequence YSISSGYYWG (SEQ ID NO: 1), the HVR-H2 comprising the amino acid sequence TIYHSGSTYYNPSLKS (SEQ ID NO: 2), the HVR-H3 comprising the amino acid sequence ARQGSIKQGYYGMDV (SEQ ID NO: 6); and a light chain variable region comprising the HVR-L1 comprising the amino acid sequence RSSQGLLRSNGYNYLD (SEQ ID NO: 27), the HVR-L2 comprising the amino acid sequence LGSNRAS (SEQ ID NO: 29), and the HVR-L3 comprising the amino acid sequence MQQQEAPLT (SEQ ID NO: 32).

D. Heavy Chain Variable Regions

In some embodiments, anti-Sortilin antibodies of the present disclosure include a heavy chain variable region comprising an amino acid sequence selected from SEQ ID NOs: 54-56. In some embodiments, the heavy chain variable region comprises an amino acid sequence with at least about 90%, at least about 91%, at least about 92%, at least about 93%, at least about 94%, at least about 95%, at least about 96%, at least about 97%, at least about 98%, at least about 99%, or 100% identity to an amino acid sequence selected from SEQ ID NOs: 54-56. In some embodiments, the heavy chain variable region comprises an amino acid sequence containing substitutions (e.g., conservative substitutions, insertions, or deletions relative to an amino acid sequence selected from SEQ ID NOs: 54-56), but retains the ability to bind to Sortilin. In certain embodiments, up to 1, up to 2, up to 3, up to 4, up to 5, up to 6, up to 7, up to 8, up to 9, or up to 10 amino acids been substituted, inserted, and/or deleted in the heavy chain variable region amino acid sequence selected from SEQ ID NOs: 54-56.

In some embodiments, the heavy chain variable region comprises the amino acid sequence of SEQ ID NO: 56.

In some embodiments, anti-Sortilin antibodies of the present disclosure include a heavy chain variable region of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16, S-60-18, S-60-19, or S-60-24 (as shown in Table 15).

In some embodiments, anti-Sortilin antibodies of the present disclosure include a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6).

E. Light Chain Variable Regions

In some embodiments, anti-Sortilin antibodies of the present disclosure include a light chain variable region comprising an amino acid sequence selected from SEQ ID NOs: 57-80. In some embodiments, the light chain variable region comprises an amino acid sequence with at least about 90%, at least about 91%, at least about 92%, at least about 93%, at least about 94%, at least about 95%, at least about 96%, at least about 97%, at least about 98%, at least about 99%, or 100% identity to an amino acid sequence selected from SEQ ID NOs: 57-80. In some embodiments, the light chain variable region comprises an amino acid sequence containing substitutions (e.g., conservative substitutions, insertions, or deletions relative to an amino acid sequence selected from SEQ ID NOs: 57-80), but retains the ability to bind to Sortilin. In certain embodiments, up to 1, up to 2, up to 3, up to 4, up to 5, up to 6, up to 7, up to 8, up to 9, or up to 10 amino acids been substituted, inserted, and/or deleted in the light chain variable region amino acid sequence selected from SEQ ID NOs: 57-80.

In some embodiments, the light chain variable region includes the amino acid sequence of SEQ ID NO: 57. In some embodiments, the light chain variable region includes the amino acid sequence of SEQ ID NO: 60.

In some embodiments, anti-Sortilin antibodies of the present disclosure comprise a light chain variable region of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16, S-60-18, S-60-19, or S-60-24 (as shown in Table 16).

In some embodiments, anti-Sortilin antibodies of the present disclosure include a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32).

In some embodiments, anti-Sortilin antibodies of the present disclosure include a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32).

F. Heavy Chain Variable Regions and Light Chain Variable Regions

In some aspects, an anti-Sortilin antibody of the present disclosure includes a heavy chain variable region comprising an amino acid sequence selected from the group consisting of SEQ ID NOs: 54-56; and/or a light chain variable region comprising an amino acid sequence selected from the group consisting of SEQ ID NOs: 57-80. In some embodiments, the heavy chain variable region comprises an amino acid sequence with at least about 90%, at least about 91%, at least about 92%, at least about 93%, at least about 94%, at least about 95%, at least about 96%, at least about 97%, at least about 98%, at least about 99%, or 100% identity to an amino acid sequence selected from SEQ ID NOs: 54-56, and the light chain variable region comprises an amino acid sequence with at least about 90%, at least about 91%, at least about 92%, at least about 93%, at least about 94%, at least about 95%, at least about 96%, at least about 97%, at least about 98%, at least about 99%, or 100% identity to an amino acid sequence selected from SEQ ID NOs: 57-80. In some embodiments, an anti-Sortilin antibody of the present disclosure includes a heavy chain variable region comprising an amino acid sequence containing substitutions (e.g., conservative substitutions, insertions, or deletions relative to an amino acid sequence selected from SEQ ID NOs: 54-56), and a light chain variable region comprising an amino acid sequence containing substitutions (e.g., conservative substitutions, insertions, or deletions relative to an amino acid sequence selected from SEQ ID NOs: 57-80), but retains the ability to bind to Sortilin. In certain embodiments, up to 1, up to 2, up to 3, up to 4, up to 5, up to 6, up to 7, up to 8, up to 9, or up to 10 amino acids been substituted, inserted, and/or deleted in the heavy chain variable region amino acid sequence selected from SEQ ID NOs: 54-56; and up to 1, up to 2, up to 3, up to 4, up to 5, up to 6, up to 7, up to 8, up to 9, or up to 10 amino acids been substituted, inserted, and/or deleted in the light chain variable region amino acid sequence selected from SEQ ID NOs: 57-80.

In some aspects, an anti-Sortilin antibody of the present disclosure includes a heavy chain variable region comprising an amino acid sequence selected from the group consisting of SEQ ID NOs: 54-56; and/or a light chain variable region comprising an amino acid sequence selected from the group consisting of SEQ ID NOs: 57-58, 60-78, and 80.

In some embodiments, an anti-Sortilin antibody of the present disclosure binds to a Sortilin protein, wherein the antibody includes a heavy chain variable region comprising the amino acid sequence of SEQ ID NO: 54, and a light chain variable region comprising the amino acid sequence of SEQ ID NO: 57; a heavy chain variable region comprising the amino acid sequence of SEQ ID NO: 54, and a light chain variable region comprising the amino acid sequence of SEQ ID NO: 58; a heavy chain variable region comprising the amino acid sequence of SEQ ID NO: 54, and a light chain variable region comprising the amino acid sequence of SEQ ID NO: 59; a heavy chain variable region comprising the amino acid sequence of SEQ ID NO: 55, and a light chain variable region comprising the amino acid sequence of SEQ ID NO: 57; a heavy chain variable region comprising the amino acid sequence of SEQ ID NO: 55, and a light chain variable region comprising the amino acid sequence of SEQ ID NO: 58; a heavy chain variable region comprising the amino acid sequence of SEQ ID NO: 56, and a light chain variable region comprising the amino acid sequence of SEQ ID NO: 57; a heavy chain variable region comprising the amino acid sequence of SEQ ID NO: 56, and a light chain variable region comprising the amino acid sequence of SEQ ID NO: 77; a heavy chain variable region comprising the amino acid sequence of SEQ ID NO: 56, and a light chain variable region comprising the amino acid sequence of SEQ ID NO: 78; a heavy chain variable region comprising the amino acid sequence of SEQ ID NO: 54, and a light chain variable region comprising the amino acid sequence of SEQ ID NO: 79; or a heavy chain variable region comprising the amino acid sequence of SEQ ID NO: 56, and a light chain variable region comprising the amino acid sequence of SEQ ID NO: 80.

In one aspect, an anti-Sortilin antibody of the present disclosure includes a heavy chain variable region having the amino acid sequence of SEQ ID NO: 56, and a light chain variable region having the amino acid sequence of SEQ ID NO: 57.

In one aspect, an anti-Sortilin antibody of the present disclosure includes a heavy chain variable region having the amino acid sequence of SEQ ID NO: 56, and a light chain variable region having the amino acid sequence of SEQ ID NO: 60.

In some embodiments, anti-Sortilin antibodies of the present disclosure comprise a heavy chain variable region of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16, S-60-18, S-60-19, or S-60-24 (as shown in Table 15), and a light chain variable region of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16, S-60-18, S-60-19, or S-60-24 (as shown in Table 16).

In some embodiments, anti-Sortilin antibodies of the present disclosure comprise a heavy chain variable region comprising the amino acid sequence of SEQ ID NO: 56, and a light chain variable region comprising an amino acid sequence selected from SEQ ID NOs: 57 and 60. In some embodiments, the antibody comprises a heavy chain variable region of antibody S-60-15 [N33 (wt)](as shown in Table 15), and a light chain variable region of antibody S-60-15 [N33 (wt)] (as shown in Table 16). In some embodiments, the antibody comprises a heavy chain variable region of antibody S-60-15.1 [N33T] (as shown in Table 15), and a light chain variable region of antibody S-60-15.1 [N33T] (as shown in Table 16).

### Exemplary Anti-Sortilin Antibodies

In some embodiments, the anti-Sortilin antibody is an anti-Sortilin monoclonal antibody comprising the heavy chain variable region and the light chain variable region of an antibody selected from S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16, S-60-18, S-60-19, or S-60-24. In some embodiments, the anti-Sortilin antibody is an anti-Sortilin monoclonal antibody comprising the heavy chain and the light chain of an antibody selected from S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16, S-60-18, S-60-19, or S-60-24.

(1) S-60-10

In some embodiments, anti-Sortilin antibodies of the present disclosure comprise a heavy chain variable domain and a light chain variable domain, wherein the heavy chain variable domain comprises an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-10 or to the amino acid sequence of SEQ ID NO: 54; and/or the light chain variable domain comprises an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-10 or to the amino acid sequence of SEQ ID NO: 57. In some embodiments, anti-Sortilin antibodies of the present disclosure comprise a heavy chain variable domain comprising an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-10 or to the amino acid sequence of SEQ ID NO: 54, wherein the heavy chain variable domain comprises the HVR-H1, HVR-H2, and HVR-H3 amino acid sequences of antibody S-60-10. In some embodiments, anti-Sortilin antibodies of the present disclosure comprise a light chain variable domain comprising an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-10 or to the amino acid sequence of SEQ ID NO: 57, wherein the light chain variable domain comprises the HVR-L1, HVR-L2, and HVR-L3 amino acid sequences of antibody S-60-10. In some embodiments, the anti-Sortilin antibody comprises a heavy chain variable domain (V_{H}) sequence having at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-10 or to the amino acid sequence of SEQ ID NO: 54 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the heavy chain variable domain amino acid sequence of antibody S-60-10 or the amino acid sequence of SEQ ID NO: 54. In certain embodiments, a total of 1 to 5 amino acids have been substituted, inserted and/or deleted in the heavy chain variable domain amino acid sequence of antibody S-60-10 or the amino acid sequence of SEQ ID NO: 54. In certain embodiments, substitutions, insertions, or deletions occur in regions outside the HVRs (i.e., in the FR regions). In some embodiments, the substitutions, insertions, or deletions occur in the FR regions. Optionally, the anti-Sortilin antibody comprises the V_{H} sequence of antibody S-60-10 or of SEQ ID NO: 54, including post-translational modifications of that sequence. In a particular embodiment, the V_{H} comprises one, two or three HVRs selected from: (a) the HVR-H1 amino acid sequence of antibody S-60-10, (b) the HVR-H2 amino acid sequence of antibody S-60-10, and (c) the HVR-H3 amino acid sequence of antibody S-60-10. In some embodiments, anti-Sortilin antibodies of the present disclosure comprise a light chain variable domain (V_{L}) sequence having at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-10 or to the amino acid sequence of SEQ ID NO: 57 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the light chain variable domain amino acid sequence of antibody S-60-10 or the amino acid sequence of SEQ ID NO: 57. In certain embodiments, a total of 1 to 5 amino acids have been substituted, inserted and/or deleted in the light chain variable domain amino acid sequence of antibody S-60-10 or the amino acid sequence of SEQ ID NO: 57. In certain embodiments, substitutions, insertions, or deletions occur in regions outside the HVRs (i.e., in the FR regions). In some embodiments, the substitutions, insertions, or deletions occur in the FR regions. Optionally, the anti-Sortilin antibody comprises the V_{L} sequence of antibody S-60-10 or of SEQ ID NO: 57, including post-translational modifications of that sequence. In a particular embodiment, the V_{L} comprises one, two or three HVRs selected from: (a) the HVR-L1 amino acid sequence of antibody S-60-10, (b) the HVR-L2 amino acid sequence of antibody S-60-10, and (c) the HVR-L3 amino acid sequence of antibody S-60-10.

In some embodiments, anti-Sortilin antibodies of the present disclosure comprise a heavy chain comprising the amino acid sequence of SEQ ID NO: 86 or SEQ ID NO: 87. In some embodiments, anti-Sortilin antibodies of the present disclosure comprise a light chain comprising the amino acid sequence of SEQ ID NO: 92. In some embodiments, anti-Sortilin antibodies of the present disclosure comprise a heavy chain comprising the amino acid sequence of SEQ ID NO: 86 or SEQ ID NO: 87 and a light chain comprising the amino acid sequence of SEQ ID NO: 92.

(2) S-60-11

In some embodiments, anti-Sortilin antibodies of the present disclosure comprise a heavy chain variable domain and a light chain variable domain, wherein the heavy chain variable domain comprises an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-11 or to the amino acid sequence of SEQ ID NO: 54; and/or the light chain variable domain comprises an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-11 or to the amino acid sequence of SEQ ID NO: 58. In some embodiments, anti-Sortilin antibodies of the present disclosure comprise a heavy chain variable domain comprising an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-11 or to the amino acid sequence of SEQ ID NO: 54, wherein the heavy chain variable domain comprises the HVR-H1, HVR-H2, and HVR-H3 amino acid sequences of antibody S-60-11. In some embodiments, anti-Sortilin antibodies of the present disclosure comprise a light chain variable domain comprising an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-11 or to the amino acid sequence of SEQ ID NO: 58, wherein the light chain variable domain comprises the HVR-L1, HVR-L2, and HVR-L3 amino acid sequences of antibody S-60-11. In some embodiments, the anti-Sortilin antibody comprises a heavy chain variable domain (V_{H}) sequence having at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-11 or to the amino acid sequence of SEQ ID NO: 54 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the heavy chain variable domain amino acid sequence of antibody S-60-11 or the amino acid sequence of SEQ ID NO: 54. In certain embodiments, a total of 1 to 5 amino acids have been substituted, inserted and/or deleted in the heavy chain variable domain amino acid sequence of antibody S-60-11 or the amino acid sequence of SEQ ID NO: 54. In certain embodiments, substitutions, insertions, or deletions occur in regions outside the HVRs (i.e., in the FR regions). In some embodiments, the substitutions, insertions, or deletions occur in the FR regions. Optionally, the anti-Sortilin antibody comprises the V_{H} sequence of antibody S-60-11 or of SEQ ID NO: 54, including post-translational modifications of that sequence. In a particular embodiment, the V_{H} comprises one, two or three HVRs selected from: (a) the HVR-H1 amino acid sequence of antibody S-60-11, (b) the HVR-H2 amino acid sequence of antibody S-60-11, and (c) the HVR-H3 amino acid sequence of antibody S-60-11. In some embodiments, anti-Sortilin antibodies of the present disclosure comprise a light chain variable domain (V_{L}) sequence having at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-11 or to the amino acid sequence of SEQ ID NO: 58 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the light chain variable domain amino acid sequence of antibody S-60-11 or the amino acid sequence of SEQ ID NO: 58. In certain embodiments, a total of 1 to 5 amino acids have been substituted, inserted and/or deleted in the light chain variable domain amino acid sequence of antibody S-60-11 or the amino acid sequence of SEQ ID NO: 58. In certain embodiments, substitutions, insertions, or deletions occur in regions outside the HVRs (i.e., in the FR regions). In some embodiments, the substitutions, insertions, or deletions occur in the FR regions. Optionally, the anti-Sortilin antibody comprises the V_{L} sequence of antibody S-60-11 or of SEQ ID NO: 58, including post-translational modifications of that sequence. In a particular embodiment, the V_{L} comprises one, two or three HVRs selected from: (a) the HVR-L1 amino acid sequence of antibody S-60-11, (b) the HVR-L2 amino acid sequence of antibody S-60-11, and (c) the HVR-L3 amino acid sequence of antibody S-60-11.

In some embodiments, anti-Sortilin antibodies of the present disclosure comprise a heavy chain comprising the amino acid sequence of SEQ ID NO: 86 or SEQ ID NO: 87. In some embodiments, anti-Sortilin antibodies of the present disclosure comprise a light chain comprising the amino acid sequence of SEQ ID NO: 93. In some embodiments, anti-Sortilin antibodies of the present disclosure comprise a heavy chain comprising the amino acid sequence of SEQ ID NO: 86 or SEQ ID NO: 87 and a light chain comprising the amino acid sequence of SEQ ID NO: 93.

(3) S-60-12

In some embodiments, anti-Sortilin antibodies of the present disclosure comprise a heavy chain variable domain and a light chain variable domain, wherein the heavy chain variable domain comprises an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-12 or to the amino acid sequence of SEQ ID NO: 54; and/or the light chain variable domain comprises an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-12 or to the amino acid sequence of SEQ ID NO: 59. In some embodiments, anti-Sortilin antibodies of the present disclosure comprise a heavy chain variable domain comprising an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-12 or to the amino acid sequence of SEQ ID NO: 54, wherein the heavy chain variable domain comprises the HVR-H1, HVR-H2, and HVR-H3 amino acid sequences of antibody S-60-12. In some embodiments, anti-Sortilin antibodies of the present disclosure comprise a light chain variable domain comprising an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-12 or to the amino acid sequence of SEQ ID NO: 59, wherein the light chain variable domain comprises the HVR-L1, HVR-L2, and HVR-L3 amino acid sequences of antibody S-60-12. In some embodiments, the anti-Sortilin antibody comprises a heavy chain variable domain (VH) sequence having at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-12 or to the amino acid sequence of SEQ ID NO: 54 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the heavy chain variable domain amino acid sequence of antibody S-60-12 or the amino acid sequence of SEQ ID NO: 54. In certain embodiments, a total of 1 to 5 amino acids have been substituted, inserted and/or deleted in the heavy chain variable domain amino acid sequence of antibody S-60-12 or the amino acid sequence of SEQ ID NO: 54. In certain embodiments, substitutions, insertions, or deletions occur in regions outside the HVRs (i.e., in the FR regions). In some embodiments, the substitutions, insertions, or deletions occur in the FR regions. Optionally, the anti-Sortilin antibody comprises the VH sequence of antibody S-60-12 or of SEQ ID NO: 54, including post-translational modifications of that sequence. In a particular embodiment, the VH comprises one, two or three HVRs selected from: (a) the HVR-H1 amino acid sequence of antibody S-60-12, (b) the HVR-H2 amino acid sequence of antibody S-60-12, and (c) the HVR-H3 amino acid sequence of antibody S-60-12. In some embodiments, anti-Sortilin antibodies of the present disclosure comprise a light chain variable domain (VL) sequence having at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-12 or to the amino acid sequence of SEQ ID NO: 59 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the light chain variable domain amino acid sequence of antibody S-60-12 or the amino acid sequence of SEQ ID NO: 59. In certain embodiments, a total of 1 to 5 amino acids have been substituted, inserted and/or deleted in the light chain variable domain amino acid sequence of antibody S-60-12 or the amino acid sequence of SEQ ID NO: 59. In certain embodiments, substitutions, insertions, or deletions occur in regions outside the HVRs (i.e., in the FR regions). In some embodiments, the substitutions, insertions, or deletions occur in the FR regions. Optionally, the anti-Sortilin antibody comprises the VL sequence of antibody S-60-12 or of SEQ ID NO: 59, including post-translational modifications of that sequence. In a particular embodiment, the VL comprises one, two or three HVRs selected from: (a) the HVR-L1 amino acid sequence of antibody S-60-12, (b) the HVR-L2 amino acid sequence of antibody S-60-12, and (c) the HVR-L3 amino acid sequence of antibody S-60-12.

In some embodiments, anti-Sortilin antibodies of the present disclosure comprise a heavy chain comprising the amino acid sequence of SEQ ID NO: 86 or SEQ ID NO: 87. In some embodiments, anti-Sortilin antibodies of the present disclosure comprise a light chain comprising the amino acid sequence of SEQ ID NO: 94. In some embodiments, anti-Sortilin antibodies of the present disclosure comprise a heavy chain comprising the amino acid sequence of SEQ ID NO: 86 or SEQ ID NO: 87 and a light chain comprising the amino acid sequence of SEQ ID NO: 94.

(4) S-60-13

In some embodiments, anti-Sortilin antibodies of the present disclosure comprise a heavy chain variable domain and a light chain variable domain, wherein the heavy chain variable domain comprises an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-13 or to the amino acid sequence of SEQ ID NO: 55; and/or the light chain variable domain comprises an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-13 or to the amino acid sequence of SEQ ID NO: 57. In some embodiments, anti-Sortilin antibodies of the present disclosure comprise a heavy chain variable domain comprising an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-13 or to the amino acid sequence of SEQ ID NO: 55, wherein the heavy chain variable domain comprises the HVR-H1, HVR-H2, and HVR-H3 amino acid sequences of antibody S-60-13. In some embodiments, anti-Sortilin antibodies of the present disclosure comprise a light chain variable domain comprising an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-13 or to the amino acid sequence of SEQ ID NO: 57, wherein the light chain variable domain comprises the HVR-L1, HVR-L2, and HVR-L3 amino acid sequences of antibody S-60-13. In some embodiments, the anti-Sortilin antibody comprises a heavy chain variable domain (VH) sequence having at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-13 or to the amino acid sequence of SEQ ID NO: 55 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the heavy chain variable domain amino acid sequence of antibody S-60-13 or the amino acid sequence of SEQ ID NO: 55. In certain embodiments, a total of 1 to 5 amino acids have been substituted, inserted and/or deleted in the heavy chain variable domain amino acid sequence of antibody S-60-13 or the amino acid sequence of SEQ ID NO: 55. In certain embodiments, substitutions, insertions, or deletions occur in regions outside the HVRs (i.e., in the FR regions). In some embodiments, the substitutions, insertions, or deletions occur in the FR regions. Optionally, the anti-Sortilin antibody comprises the VH sequence of antibody S-60-13 or of SEQ ID NO: 55, including post-translational modifications of that sequence. In a particular embodiment, the V_{H} comprises one, two or three HVRs selected from: (a) the HVR-H1 amino acid sequence of antibody S-60-13, (b) the HVR-H2 amino acid sequence of antibody S-60-13, and (c) the HVR-H3 amino acid sequence of antibody S-60-13. In some embodiments, anti-Sortilin antibodies of the present disclosure comprise a light chain variable domain (VL) sequence having at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-13 or to the amino acid sequence of SEQ ID NO: 57 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the light chain variable domain amino acid sequence of antibody S-60-13 or the amino acid sequence of SEQ ID NO: 57. In certain embodiments, a total of 1 to 5 amino acids have been substituted, inserted and/or deleted in the light chain variable domain amino acid sequence of antibody S-60-13 or the amino acid sequence of SEQ ID NO: 57. In certain embodiments, substitutions, insertions, or deletions occur in regions outside the HVRs (i.e., in the FR regions). In some embodiments, the substitutions, insertions, or deletions occur in the FR regions. Optionally, the anti-Sortilin antibody comprises the VL sequence of antibody S-60-13 or of SEQ ID NO: 57, including post-translational modifications of that sequence. In a particular embodiment, the VL comprises one, two or three HVRs selected from: (a) the HVR-L1 amino acid sequence of antibody S-60-13, (b) the HVR-L2 amino acid sequence of antibody S-60-13, and (c) the HVR-L3 amino acid sequence of antibody S-60-13.

In some embodiments, anti-Sortilin antibodies of the present disclosure comprise a heavy chain comprising the amino acid sequence of SEQ ID NO: 88 or SEQ ID NO: 89. In some embodiments, anti-Sortilin antibodies of the present disclosure comprise a light chain comprising the amino acid sequence of SEQ ID NO: 92. In some embodiments, anti-Sortilin antibodies of the present disclosure comprise a heavy chain comprising the amino acid sequence of SEQ ID NO: 88 or SEQ ID NO: 89 and a light chain comprising the amino acid sequence of SEQ ID NO: 92.

(5) S-60-14

In some embodiments, anti-Sortilin antibodies of the present disclosure comprise a heavy chain variable domain and a light chain variable domain, wherein the heavy chain variable domain comprises an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-14 or to the amino acid sequence of SEQ ID NO: 55; and/or the light chain variable domain comprises an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-14 or to the amino acid sequence of SEQ ID NO: 58. In some embodiments, anti-Sortilin antibodies of the present disclosure comprise a heavy chain variable domain comprising an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-14 or to the amino acid sequence of SEQ ID NO: 55, wherein the heavy chain variable domain comprises the HVR-H1, HVR-H2, and HVR-H3 amino acid sequences of antibody S-60-14. In some embodiments, anti-Sortilin antibodies of the present disclosure comprise a light chain variable domain comprising an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-14 or to the amino acid sequence of SEQ ID NO: 58, wherein the light chain variable domain comprises the HVR-L1, HVR-L2, and HVR-L3 amino acid sequences of antibody S-60-14. In some embodiments, the anti-Sortilin antibody comprises a heavy chain variable domain (VH) sequence having at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-14 or to the amino acid sequence of SEQ ID NO: 55 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the heavy chain variable domain amino acid sequence of antibody S-60-14 or the amino acid sequence of SEQ ID NO: 55. In certain embodiments, a total of 1 to 5 amino acids have been substituted, inserted and/or deleted in the heavy chain variable domain amino acid sequence of antibody S-60-14 or the amino acid sequence of SEQ ID NO: 55. In certain embodiments, substitutions, insertions, or deletions occur in regions outside the HVRs (i.e., in the FR regions). In some embodiments, the substitutions, insertions, or deletions occur in the FR regions. Optionally, the anti-Sortilin antibody comprises the VH sequence of antibody S-60-14 or of SEQ ID NO: 55, including post-translational modifications of that sequence. In a particular embodiment, the V_{H} comprises one, two or three HVRs selected from: (a) the HVR-H1 amino acid sequence of antibody S-60-14, (b) the HVR-H2 amino acid sequence of antibody S-60-14, and (c) the HVR-H3 amino acid sequence of antibody S-60-14. In some embodiments, anti-Sortilin antibodies of the present disclosure comprise a light chain variable domain (VL) sequence having at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-14 or to the amino acid sequence of SEQ ID NO: 58 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the light chain variable domain amino acid sequence of antibody S-60-14 or the amino acid sequence of SEQ ID NO: 58. In certain embodiments, a total of 1 to 5 amino acids have been substituted, inserted and/or deleted in the light chain variable domain amino acid sequence of antibody S-60-14 or the amino acid sequence of SEQ ID NO: 58. In certain embodiments, substitutions, insertions, or deletions occur in regions outside the HVRs (i.e., in the FR regions). In some embodiments, the substitutions, insertions, or deletions occur in the FR regions. Optionally, the anti-Sortilin antibody comprises the VL sequence of antibody S-60-14 or of SEQ ID NO: 58, including post-translational modifications of that sequence. In a particular embodiment, the VL comprises one, two or three HVRs selected from: (a) the HVR-L1 amino acid sequence of antibody S-60-14, (b) the HVR-L2 amino acid sequence of antibody S-60-14, and (c) the HVR-L3 amino acid sequence of antibody S-60-14.

In some embodiments, anti-Sortilin antibodies of the present disclosure comprise a heavy chain comprising the amino acid sequence of SEQ ID NO: 88 or SEQ ID NO: 89. In some embodiments, anti-Sortilin antibodies of the present disclosure comprise a light chain comprising the amino acid sequence of SEQ ID NO: 93. In some embodiments, anti-Sortilin antibodies of the present disclosure comprise a heavy chain comprising the amino acid sequence of SEQ ID NO: 88 or SEQ ID NO: 89 and a light chain comprising the amino acid sequence of SEQ ID NO: 93.

(6) S-60-15

In some embodiments, anti-Sortilin antibodies of the present disclosure comprise a heavy chain variable domain and a light chain variable domain, wherein the heavy chain variable domain comprises an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-15 or to the amino acid sequence of SEQ ID NO: 56; and/or the light chain variable domain comprises an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-15 or to the amino acid sequence of SEQ ID NO: 57. In some embodiments, anti-Sortilin antibodies of the present disclosure comprise a heavy chain variable domain comprising an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-15 or to the amino acid sequence of SEQ ID NO: 56, wherein the heavy chain variable domain comprises the HVR-H1, HVR-H2, and HVR-H3 amino acid sequences of antibody S-60-15. In some embodiments, anti-Sortilin antibodies of the present disclosure comprise a light chain variable domain comprising an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-15 or to the amino acid sequence of SEQ ID NO: 57, wherein the light chain variable domain comprises the HVR-L1, HVR-L2, and HVR-L3 amino acid sequences of antibody S-60-15. In some embodiments, the anti-Sortilin antibody comprises a heavy chain variable domain (VH) sequence having at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-15 or to the amino acid sequence of SEQ ID NO: 56 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the heavy chain variable domain amino acid sequence of antibody S-60-15 or the amino acid sequence of SEQ ID NO: 56. In certain embodiments, a total of 1 to 5 amino acids have been substituted, inserted and/or deleted in the heavy chain variable domain amino acid sequence of antibody S-60-15 or the amino acid sequence of SEQ ID NO: 56. In certain embodiments, substitutions, insertions, or deletions occur in regions outside the HVRs (i.e., in the FR regions). In some embodiments, the substitutions, insertions, or deletions occur in the FR regions. Optionally, the anti-Sortilin antibody comprises the VH sequence of antibody S-60-15 or of SEQ ID NO: 56, including post-translational modifications of that sequence. In a particular embodiment, the V_{H} comprises one, two or three HVRs selected from: (a) the HVR-H1 amino acid sequence of antibody S-60-15, (b) the HVR-H2 amino acid sequence of antibody S-60-15, and (c) the HVR-H3 amino acid sequence of antibody S-60-15. In some embodiments, anti-Sortilin antibodies of the present disclosure comprise a light chain variable domain (VL) sequence having at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-15 or to the amino acid sequence of SEQ ID NO: 57 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the light chain variable domain amino acid sequence of antibody S-60-15 or the amino acid sequence of SEQ ID NO: 57. In certain embodiments, a total of 1 to 5 amino acids have been substituted, inserted and/or deleted in the light chain variable domain amino acid sequence of antibody S-60-15 or the amino acid sequence of SEQ ID NO: 57. In certain embodiments, substitutions, insertions, or deletions occur in regions outside the HVRs (i.e., in the FR regions). In some embodiments, the substitutions, insertions, or deletions occur in the FR regions. Optionally, the anti-Sortilin antibody comprises the VL sequence of antibody S-60-15 or of SEQ ID NO: 57, including post-translational modifications of that sequence. In a particular embodiment, the VL comprises one, two or three HVRs selected from: (a) the HVR-L1 amino acid sequence of antibody S-60-15, (b) the HVR-L2 amino acid sequence of antibody S-60-15, and (c) the HVR-L3 amino acid sequence of antibody S-60-15.

In some embodiments, anti-Sortilin antibodies of the present disclosure comprise a heavy chain comprising the amino acid sequence of SEQ ID NO: 90 or SEQ ID NO: 91. In some embodiments, anti-Sortilin antibodies of the present disclosure comprise a light chain comprising the amino acid sequence of SEQ ID NO: 92. In some embodiments, anti-Sortilin antibodies of the present disclosure comprise a heavy chain comprising the amino acid sequence of SEQ ID NO: 90 or SEQ ID NO: 91 and a light chain comprising the amino acid sequence of SEQ ID NO: 92.

(7) S-60-15.1

In some embodiments, anti-Sortilin antibodies of the present disclosure comprise a heavy chain variable domain and a light chain variable domain, wherein the heavy chain variable domain comprises an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-15.1 or to the amino acid sequence of SEQ ID NO: 56; and/or the light chain variable domain comprises an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-15.1 or to the amino acid sequence of SEQ ID NO: 60. In some embodiments, anti-Sortilin antibodies of the present disclosure comprise a heavy chain variable domain comprising an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-15.1 or to the amino acid sequence of SEQ ID NO: 56, wherein the heavy chain variable domain comprises the HVR-H1, HVR-H2, and HVR-H3 amino acid sequences of antibody S-60-15.1. In some embodiments, anti-Sortilin antibodies of the present disclosure comprise a light chain variable domain comprising an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-15.1 or to the amino acid sequence of SEQ ID NO: 60, wherein the light chain variable domain comprises the HVR-L1, HVR-L2, and HVR-L3 amino acid sequences of antibody S-60-15.1. In some embodiments, the anti-Sortilin antibody comprises a heavy chain variable domain (VH) sequence having at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-15.1 or to the amino acid sequence of SEQ ID NO: 56 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the heavy chain variable domain amino acid sequence of antibody S-60-15.1 or the amino acid sequence of SEQ ID NO: 56. In certain embodiments, a total of 1 to 5 amino acids have been substituted, inserted and/or deleted in the heavy chain variable domain amino acid sequence of antibody S-60-15.1 or the amino acid sequence of SEQ ID NO: 56. In certain embodiments, substitutions, insertions, or deletions occur in regions outside the HVRs (i.e., in the FR regions). In some embodiments, the substitutions, insertions, or deletions occur in the FR regions. Optionally, the anti-Sortilin antibody comprises the VH sequence of antibody S-60-15.1 or of SEQ ID NO: 56, including post-translational modifications of that sequence. In a particular embodiment, the VH comprises one, two or three HVRs selected from: (a) the HVR-H1 amino acid sequence of antibody S-60-15.1, (b) the HVR-H2 amino acid sequence of antibody S-60-15.1, and (c) the HVR-H3 amino acid sequence of antibody S-60-15.1. In some embodiments, anti-Sortilin antibodies of the present disclosure comprise a light chain variable domain (VL) sequence having at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-15.1 or to the amino acid sequence of SEQ ID NO: 60 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the light chain variable domain amino acid sequence of antibody S-60-15.1 or the amino acid sequence of SEQ ID NO: 60. In certain embodiments, a total of 1 to 5 amino acids have been substituted, inserted and/or deleted in the light chain variable domain amino acid sequence of antibody S-60-15.1 or the amino acid sequence of SEQ ID NO: 60. In certain embodiments, substitutions, insertions, or deletions occur in regions outside the HVRs (i.e., in the FR regions). In some embodiments, the substitutions, insertions, or deletions occur in the FR regions. Optionally, the anti-Sortilin antibody comprises the VL sequence of antibody S-60-15.1 or of SEQ ID NO: 60, including post-translational modifications of that sequence. In a particular embodiment, the VL comprises one, two or three HVRs selected from: (a) the HVR-L1 amino acid sequence of antibody S-60-15.1, (b) the HVR-L2 amino acid sequence of antibody S-60-15.1, and (c) the HVR-L3 amino acid sequence of antibody S-60-15.1.

In some embodiments, anti-Sortilin antibodies of the present disclosure comprise a heavy chain comprising the amino acid sequence of SEQ ID NO: 90 or SEQ ID NO: 91. In some embodiments, anti-Sortilin antibodies of the present disclosure comprise a light chain comprising the amino acid sequence of SEQ ID NO: 95. In some embodiments, anti-Sortilin antibodies of the present disclosure comprise a heavy chain comprising the amino acid sequence of SEQ ID NO: 90 or SEQ ID NO: 91 and a light chain comprising the amino acid sequence of SEQ ID NO: 95.

(8) S-60-16

In some embodiments, anti-Sortilin antibodies of the present disclosure comprise a heavy chain variable domain and a light chain variable domain, wherein the heavy chain variable domain comprises an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-16 or to the amino acid sequence of SEQ ID NO: 56; and/or the light chain variable domain comprises an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-16 or to the amino acid sequence of SEQ ID NO: 77. In some embodiments, anti-Sortilin antibodies of the present disclosure comprise a heavy chain variable domain comprising an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-16 or to the amino acid sequence of SEQ ID NO: 56, wherein the heavy chain variable domain comprises the HVR-H1, HVR-H2, and HVR-H3 amino acid sequences of antibody S-60-16. In some embodiments, anti-Sortilin antibodies of the present disclosure comprise a light chain variable domain comprising an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-16 or to the amino acid sequence of SEQ ID NO: 77, wherein the light chain variable domain comprises the HVR-L1, HVR-L2, and HVR-L3 amino acid sequences of antibody S-60-16. In some embodiments, the anti-Sortilin antibody comprises a heavy chain variable domain (VH) sequence having at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-16 or to the amino acid sequence of SEQ ID NO: 56 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the heavy chain variable domain amino acid sequence of antibody S-60-16 or the amino acid sequence of SEQ ID NO: 56. In certain embodiments, a total of 1 to 5 amino acids have been substituted, inserted and/or deleted in the heavy chain variable domain amino acid sequence of antibody S-60-16 or the amino acid sequence of SEQ ID NO: 56. In certain embodiments, substitutions, insertions, or deletions occur in regions outside the HVRs (i.e., in the FR regions). In some embodiments, the substitutions, insertions, or deletions occur in the FR regions. Optionally, the anti-Sortilin antibody comprises the VH sequence of antibody S-60-16 or of SEQ ID NO: 56, including post-translational modifications of that sequence. In a particular embodiment, the VH comprises one, two or three HVRs selected from: (a) the HVR-H1 amino acid sequence of antibody S-60-16, (b) the HVR-H2 amino acid sequence of antibody S-60-16, and (c) the HVR-H3 amino acid sequence of antibody S-60-16. In some embodiments, anti-Sortilin antibodies of the present disclosure comprise a light chain variable domain (VL) sequence having at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-16 or to the amino acid sequence of SEQ ID NO: 77 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the light chain variable domain amino acid sequence of antibody S-60-16 or the amino acid sequence of SEQ ID NO: 77. In certain embodiments, a total of 1 to 5 amino acids have been substituted, inserted and/or deleted in the light chain variable domain amino acid sequence of antibody S-60-16 or the amino acid sequence of SEQ ID NO: 77. In certain embodiments, substitutions, insertions, or deletions occur in regions outside the HVRs (i.e., in the FR regions). In some embodiments, the substitutions, insertions, or deletions occur in the FR regions. Optionally, the anti-Sortilin antibody comprises the VL sequence of antibody S-60-16 or of SEQ ID NO: 77, including post-translational modifications of that sequence. In a particular embodiment, the VL comprises one, two or three HVRs selected from: (a) the HVR-L1 amino acid sequence of antibody S-60-16, (b) the HVR-L2 amino acid sequence of antibody S-60-16, and (c) the HVR-L3 amino acid sequence of antibody S-60-16.

In some embodiments, anti-Sortilin antibodies of the present disclosure comprise a heavy chain comprising the amino acid sequence of SEQ ID NO: 90 or SEQ ID NO: 91. In some embodiments, anti-Sortilin antibodies of the present disclosure comprise a light chain comprising the amino acid sequence of SEQ ID NO: 112. In some embodiments, anti-Sortilin antibodies of the present disclosure comprise a heavy chain comprising the amino acid sequence of SEQ ID NO: 90 or SEQ ID NO: 91 and a light chain comprising the amino acid sequence of SEQ ID NO: 112.

(9) S-60-18

In some embodiments, anti-Sortilin antibodies of the present disclosure comprise a heavy chain variable domain and a light chain variable domain, wherein the heavy chain variable domain comprises an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-18 or to the amino acid sequence of SEQ ID NO: 56; and/or the light chain variable domain comprises an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-18 or to the amino acid sequence of SEQ ID NO: 78. In some embodiments, anti-Sortilin antibodies of the present disclosure comprise a heavy chain variable domain comprising an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-18 or to the amino acid sequence of SEQ ID NO: 56, wherein the heavy chain variable domain comprises the HVR-H1, HVR-H2, and HVR-H3 amino acid sequences of antibody S-60-18. In some embodiments, anti-Sortilin antibodies of the present disclosure comprise a light chain variable domain comprising an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-18 or to the amino acid sequence of SEQ ID NO: 78, wherein the light chain variable domain comprises the HVR-L1, HVR-L2, and HVR-L3 amino acid sequences of antibody S-60-18. In some embodiments, the anti-Sortilin antibody comprises a heavy chain variable domain (VH) sequence having at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-18 or to the amino acid sequence of SEQ ID NO: 56 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the heavy chain variable domain amino acid sequence of antibody S-60-18 or the amino acid sequence of SEQ ID NO: 56. In certain embodiments, a total of 1 to 5 amino acids have been substituted, inserted and/or deleted in the heavy chain variable domain amino acid sequence of antibody S-60-18 or the amino acid sequence of SEQ ID NO: 56. In certain embodiments, substitutions, insertions, or deletions occur in regions outside the HVRs (i.e., in the FR regions). In some embodiments, the substitutions, insertions, or deletions occur in the FR regions. Optionally, the anti-Sortilin antibody comprises the VH sequence of antibody S-60-18 or of SEQ ID NO: 56, including post-translational modifications of that sequence. In a particular embodiment, the VH comprises one, two or three HVRs selected from: (a) the HVR-H1 amino acid sequence of antibody S-60-18, (b) the HVR-H2 amino acid sequence of antibody S-60-18, and (c) the HVR-H3 amino acid sequence of antibody S-60-18. In some embodiments, anti-Sortilin antibodies of the present disclosure comprise a light chain variable domain (VL) sequence having at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-18 or to the amino acid sequence of SEQ ID NO: 78 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the light chain variable domain amino acid sequence of antibody S-60-18 or the amino acid sequence of SEQ ID NO: 78. In certain embodiments, a total of 1 to 5 amino acids have been substituted, inserted and/or deleted in the light chain variable domain amino acid sequence of antibody S-60-18 or the amino acid sequence of SEQ ID NO: 78. In certain embodiments, substitutions, insertions, or deletions occur in regions outside the HVRs (i.e., in the FR regions). In some embodiments, the substitutions, insertions, or deletions occur in the FR regions. Optionally, the anti-Sortilin antibody comprises the VL sequence of antibody S-60-18 or of SEQ ID NO: 78, including post-translational modifications of that sequence. In a particular embodiment, the VL comprises one, two or three HVRs selected from: (a) the HVR-L1 amino acid sequence of antibody S-60-18, (b) the HVR-L2 amino acid sequence of antibody S-60-18, and (c) the HVR-L3 amino acid sequence of antibody S-60-18.

In some embodiments, anti-Sortilin antibodies of the present disclosure comprise a heavy chain comprising the amino acid sequence of SEQ ID NO: 90 or SEQ ID NO: 91. In some embodiments, anti-Sortilin antibodies of the present disclosure comprise a light chain comprising the amino acid sequence of SEQ ID NO: 113. In some embodiments, anti-Sortilin antibodies of the present disclosure comprise a heavy chain comprising the amino acid sequence of SEQ ID NO: 90 or SEQ ID NO: 91 and a light chain comprising the amino acid sequence of SEQ ID NO: 113.

(10) S-60-19

In some embodiments, anti-Sortilin antibodies of the present disclosure comprise a heavy chain variable domain and a light chain variable domain, wherein the heavy chain variable domain comprises an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-19 or to the amino acid sequence of SEQ ID NO: 54; and/or the light chain variable domain comprises an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-19 or to the amino acid sequence of SEQ ID NO: 79. In some embodiments, anti-Sortilin antibodies of the present disclosure comprise a heavy chain variable domain comprising an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-19 or to the amino acid sequence of SEQ ID NO: 54, wherein the heavy chain variable domain comprises the HVR-H1, HVR-H2, and HVR-H3 amino acid sequences of antibody S-60-19. In some embodiments, anti-Sortilin antibodies of the present disclosure comprise a light chain variable domain comprising an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-19 or to the amino acid sequence of SEQ ID NO: 79, wherein the light chain variable domain comprises the HVR-L1, HVR-L2, and HVR-L3 amino acid sequences of antibody S-60-19. In some embodiments, the anti-Sortilin antibody comprises a heavy chain variable domain (VH) sequence having at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-19 or to the amino acid sequence of SEQ ID NO: 54 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the heavy chain variable domain amino acid sequence of antibody S-60-19 or the amino acid sequence of SEQ ID NO: 54. In certain embodiments, a total of 1 to 5 amino acids have been substituted, inserted and/or deleted in the heavy chain variable domain amino acid sequence of antibody S-60-19 or the amino acid sequence of SEQ ID NO: 54. In certain embodiments, substitutions, insertions, or deletions occur in regions outside the HVRs (i.e., in the FR regions). In some embodiments, the substitutions, insertions, or deletions occur in the FR regions. Optionally, the anti-Sortilin antibody comprises the VH sequence of antibody S-60-19 or of SEQ ID NO: 54, including post-translational modifications of that sequence. In a particular embodiment, the VH comprises one, two or three HVRs selected from: (a) the HVR-H1 amino acid sequence of antibody S-60-19, (b) the HVR-H2 amino acid sequence of antibody S-60-19, and (c) the HVR-H3 amino acid sequence of antibody S-60-19. In some embodiments, anti-Sortilin antibodies of the present disclosure comprise a light chain variable domain (VL) sequence having at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-19 or to the amino acid sequence of SEQ ID NO: 79 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the light chain variable domain amino acid sequence of antibody S-60-19 or the amino acid sequence of SEQ ID NO: 79. In certain embodiments, a total of 1 to 5 amino acids have been substituted, inserted and/or deleted in the light chain variable domain amino acid sequence of antibody S-60-19 or the amino acid sequence of SEQ ID NO: 79. In certain embodiments, substitutions, insertions, or deletions occur in regions outside the HVRs (i.e., in the FR regions). In some embodiments, the substitutions, insertions, or deletions occur in the FR regions. Optionally, the anti-Sortilin antibody comprises the VL sequence of antibody S-60-19 or of SEQ ID NO: 79, including post-translational modifications of that sequence. In a particular embodiment, the VL comprises one, two or three HVRs selected from: (a) the HVR-L1 amino acid sequence of antibody S-60-19, (b) the HVR-L2 amino acid sequence of antibody S-60-19, and (c) the HVR-L3 amino acid sequence of antibody S-60-19.

In some embodiments, anti-Sortilin antibodies of the present disclosure comprise a heavy chain comprising the amino acid sequence of SEQ ID NO: 86 or SEQ ID NO: 87. In some embodiments, anti-Sortilin antibodies of the present disclosure comprise a light chain comprising the amino acid sequence of SEQ ID NO: 114. In some embodiments, anti-Sortilin antibodies of the present disclosure comprise a heavy chain comprising the amino acid sequence of SEQ ID NO: 86 or SEQ ID NO: 87 and a light chain comprising the amino acid sequence of SEQ ID NO: 114.

(11) S-60-24

In some embodiments, anti-Sortilin antibodies of the present disclosure comprise a heavy chain variable domain and a light chain variable domain, wherein the heavy chain variable domain comprises an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-24 or to the amino acid sequence of SEQ ID NO: 56; and/or the light chain variable domain comprises an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-24 or to the amino acid sequence of SEQ ID NO: 80. In some embodiments, anti-Sortilin antibodies of the present disclosure comprise a heavy chain variable domain comprising an amino acid sequence with at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-24 or to the amino acid sequence of SEQ ID NO: 56, wherein the heavy chain variable domain comprises the HVR-H1, HVR-H2, and HVR-H3 amino acid sequences of antibody S-60-24. In some embodiments, anti-Sortilin antibodies of the present disclosure comprise a light chain variable domain comprising an amino acid sequence with at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-24 or to the amino acid sequence of SEQ ID NO: 80, wherein the light chain variable domain comprises the HVR-L1, HVR-L2, and HVR-L3 amino acid sequences of antibody S-60-24. In some embodiments, the anti-Sortilin antibody comprises a heavy chain variable domain (VH) sequence having at least 90%, at least 91%, at least 92%, at least 93%, at least 94%, at least 95%, at least 96%, at least 97%, at least 98%, at least 99%, or 100% identity to a heavy chain variable domain amino acid sequence of antibody S-60-24 or to the amino acid sequence of SEQ ID NO: 56 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the heavy chain variable domain amino acid sequence of antibody S-60-24 or the amino acid sequence of SEQ ID NO: 56. In certain embodiments, a total of 1 to 5 amino acids have been substituted, inserted and/or deleted in the heavy chain variable domain amino acid sequence of antibody S-60-24 or the amino acid sequence of SEQ ID NO: 56. In certain embodiments, substitutions, insertions, or deletions occur in regions outside the HVRs (i.e., in the FR regions). In some embodiments, the substitutions, insertions, or deletions occur in the FR regions. Optionally, the anti-Sortilin antibody comprises the V_{H} sequence of antibody S-60-24 or of SEQ ID NO: 56, including post-translational modifications of that sequence. In a particular embodiment, the V_{H} comprises one, two or three HVRs selected from: (a) the HVR-H1 amino acid sequence of antibody S-60-24, (b) the HVR-H2 amino acid sequence of antibody S-60-24, and (c) the HVR-H3 amino acid sequence of antibody S-60-24. In some embodiments, anti-Sortilin antibodies of the present disclosure comprise a light chain variable domain (V_{L}) sequence having at least 90%, at least 91%, at least 92%, at least 93%, at least 94%, at least 95%, at least 96%, at least 97%, at least 98%, at least 99%, or 100% identity to a light chain variable domain amino acid sequence of antibody S-60-24 or to the amino acid sequence of SEQ ID NO: 80 and contains substitutions (e.g., conservative substitutions, insertions, or deletions relative to the reference sequence), but the anti-Sortilin antibody comprising that sequence retains the ability to bind to Sortilin. In certain embodiments, a total of 1 to 10 amino acids have been substituted, inserted, and/or deleted in the light chain variable domain amino acid sequence of antibody S-60-24 or the amino acid sequence of SEQ ID NO: 80. In certain embodiments, a total of 1 to 5 amino acids have been substituted, inserted and/or deleted in the light chain variable domain amino acid sequence of antibody S-60-24 or the amino acid sequence of SEQ ID NO: 80. In certain embodiments, substitutions, insertions, or deletions occur in regions outside the HVRs (i.e., in the FR regions). In some embodiments, the substitutions, insertions, or deletions occur in the FR regions. Optionally, the anti-Sortilin antibody comprises the VL sequence of antibody S-60-24 or of SEQ ID NO: 80, including post-translational modifications of that sequence. In a particular embodiment, the VL comprises one, two or three HVRs selected from: (a) the HVR-L1 amino acid sequence of antibody S-60-24, (b) the HVR-L2 amino acid sequence of antibody S-60-24, and (c) the HVR-L3 amino acid sequence of antibody S-60-24.

In some embodiments, anti-Sortilin antibodies of the present disclosure comprise a heavy chain comprising the amino acid sequence of SEQ ID NO: 90 or SEQ ID NO: 91. In some embodiments, anti-Sortilin antibodies of the present disclosure comprise a light chain comprising the amino acid sequence of SEQ ID NO: 115. In some embodiments, anti-Sortilin antibodies of the present disclosure comprise a heavy chain comprising the amino acid sequence of SEQ ID NO: 90 or SEQ ID NO: 91 and a light chain comprising the amino acid sequence of SEQ ID NO: 115.

In some embodiments, an anti-Sortilin antibody of the present disclosure binds essentially the same Sortilin epitope as an antibody comprising the heavy chain variable domain and the light chain variable domain of an antibody selected from the group consisting of S-60-10, S-60-11, S-60-12, S-60-13, S-60-14, S-60-15 [N33 (wt)], S-60-15.1 [N33T], S-60-16, S-60-18, S-60-19, and S-60-24.

In some embodiments, the anti-Sortilin antibody is anti-Sortilin monoclonal antibody S-60-10. In some embodiments, the anti-Sortilin antibody is an isolated antibody which binds essentially the same Sortilin epitope as S-60-10. In some embodiments, the anti-Sortilin antibody is an isolated antibody comprising the heavy chain variable region of monoclonal antibody S-60-10. In some embodiments, the anti-Sortilin antibody is an isolated antibody comprising the light chain variable region of monoclonal antibody S-60-10. In some embodiments, the anti-Sortilin antibody is an isolated antibody comprising the heavy chain variable region and the light chain variable region of monoclonal antibody S-60-10.

In some embodiments, the anti-Sortilin antibody is anti-Sortilin monoclonal antibody S-60-11. In some embodiments, the anti-Sortilin antibody is an isolated antibody which binds essentially the same Sortilin epitope as S-60-11. In some embodiments, the anti-Sortilin antibody is an isolated antibody comprising the heavy chain variable region of monoclonal antibody S-60-11. In some embodiments, the anti-Sortilin antibody is an isolated antibody comprising the light chain variable region of monoclonal antibody S-60-11. In some embodiments, the anti-Sortilin antibody is an isolated antibody comprising the heavy chain variable region and the light chain variable region of monoclonal antibody S-60-11.

In some embodiments, the anti-Sortilin antibody is anti-Sortilin monoclonal antibody S-60-12. In some embodiments, the anti-Sortilin antibody is an isolated antibody which binds essentially the same Sortilin epitope as S-60-12. In some embodiments, the anti-Sortilin antibody is an isolated antibody comprising the heavy chain variable region of monoclonal antibody S-60-12. In some embodiments, the anti-Sortilin antibody is an isolated antibody comprising the light chain variable region of monoclonal antibody S-60-12. In some embodiments, the anti-Sortilin antibody is an isolated antibody comprising the heavy chain variable region and the light chain variable region of monoclonal antibody S-60-12.

In some embodiments, the anti-Sortilin antibody is anti-Sortilin monoclonal antibody S-60-13. In some embodiments, the anti-Sortilin antibody is an isolated antibody which binds essentially the same Sortilin epitope as S-60-13. In some embodiments, the anti-Sortilin antibody is an isolated antibody comprising the heavy chain variable region of monoclonal antibody S-60-13. In some embodiments, the anti-Sortilin antibody is an isolated antibody comprising the light chain variable region of monoclonal antibody S-60-13. In some embodiments, the anti-Sortilin antibody is an isolated antibody comprising the heavy chain variable region and the light chain variable region of monoclonal antibody S-60-13.

In some embodiments, the anti-Sortilin antibody is anti-Sortilin monoclonal antibody S-60-14. In some embodiments, the anti-Sortilin antibody is an isolated antibody which binds essentially the same Sortilin epitope as S-60-14. In some embodiments, the anti-Sortilin antibody is an isolated antibody comprising the heavy chain variable region of monoclonal antibody S-60-14. In some embodiments, the anti-Sortilin antibody is an isolated antibody comprising the light chain variable region of monoclonal antibody S-60-14. In some embodiments, the anti-Sortilin antibody is an isolated antibody comprising the heavy chain variable region and the light chain variable region of monoclonal antibody S-60-14.

In some embodiments, the anti-Sortilin antibody is anti-Sortilin monoclonal antibody S-60-15. In some embodiments, the anti-Sortilin antibody is an isolated antibody which binds essentially the same Sortilin epitope as S-60-15. In some embodiments, the anti-Sortilin antibody is an isolated antibody comprising the heavy chain variable region of monoclonal antibody S-60-15. In some embodiments, the anti-Sortilin antibody is an isolated antibody comprising the light chain variable region of monoclonal antibody S-60-15. In some embodiments, the anti-Sortilin antibody is an isolated antibody comprising the heavy chain variable region and the light chain variable region of monoclonal antibody S-60-15.

In some embodiments, the anti-Sortilin antibody is anti-Sortilin monoclonal antibody S-60-15.1. In some embodiments, the anti-Sortilin antibody is an isolated antibody which binds essentially the same Sortilin epitope as S-60-15.1. In some embodiments, the anti-Sortilin antibody is an isolated antibody comprising the heavy chain variable region of monoclonal antibody S-60-15.1. In some embodiments, the anti-Sortilin antibody is an isolated antibody comprising the light chain variable region of monoclonal antibody S-60-15.1. In some embodiments, the anti-Sortilin antibody is an isolated antibody comprising the heavy chain variable region and the light chain variable region of monoclonal antibody S-60-15.1.

In some embodiments, the anti-Sortilin antibody is anti-Sortilin monoclonal antibody S-60-16. In some embodiments, the anti-Sortilin antibody is an isolated antibody which binds essentially the same Sortilin epitope as S-60-16. In some embodiments, the anti-Sortilin antibody is an isolated antibody comprising the heavy chain variable region of monoclonal antibody S-60-16. In some embodiments, the anti-Sortilin antibody is an isolated antibody comprising the light chain variable region of monoclonal antibody S-60-16. In some embodiments, the anti-Sortilin antibody is an isolated antibody comprising the heavy chain variable region and the light chain variable region of monoclonal antibody S-60-16.

In some embodiments, the anti-Sortilin antibody is anti-Sortilin monoclonal antibody S-60-18. In some embodiments, the anti-Sortilin antibody is an isolated antibody which binds essentially the same Sortilin epitope as S-60-18. In some embodiments, the anti-Sortilin antibody is an isolated antibody comprising the heavy chain variable region of monoclonal antibody S-60-18. In some embodiments, the anti-Sortilin antibody is an isolated antibody comprising the light chain variable region of monoclonal antibody S-60-18. In some embodiments, the anti-Sortilin antibody is an isolated antibody comprising the heavy chain variable region and the light chain variable region of monoclonal antibody S-60-18.

In some embodiments, the anti-Sortilin antibody is anti-Sortilin monoclonal antibody S-60-19. In some embodiments, the anti-Sortilin antibody is an isolated antibody which binds essentially the same Sortilin epitope as S-60-19. In some embodiments, the anti-Sortilin antibody is an isolated antibody comprising the heavy chain variable region of monoclonal antibody S-60-19. In some embodiments, the anti-Sortilin antibody is an isolated antibody comprising the light chain variable region of monoclonal antibody S-60-19. In some embodiments, the anti-Sortilin antibody is an isolated antibody comprising the heavy chain variable region and the light chain variable region of monoclonal antibody S-60-19.

In some embodiments, the anti-Sortilin antibody is anti-Sortilin monoclonal antibody S-60-24. In some embodiments, the anti-Sortilin antibody is an isolated antibody which binds essentially the same Sortilin epitope as S-60-24. In some embodiments, the anti-Sortilin antibody is an isolated antibody comprising the heavy chain variable region of monoclonal antibody S-60-24. In some embodiments, the anti-Sortilin antibody is an isolated antibody comprising the light chain variable region of monoclonal antibody S-60-24. In some embodiments, the anti-Sortilin antibody is an isolated antibody comprising the heavy chain variable region and the light chain variable region of monoclonal antibody S-60-24.

In certain embodiments, the anti-Sortilin antibody is an antagonist antibody. In certain embodiments, the anti-Sortilin antibody is an agonist antibody. In some embodiments, anti-Sortilin antibodies of the present disclosure are of the IgG class the IgM class, or the IgA class. In some embodiments, anti-Sortilin antibodies of the present disclosure are of the IgG class and have an IgG1, IgG2, IgG3, or IgG4 isotype.

Additional anti-Sortilin antibodies, e.g., antibodies that specifically bind to a Sortilin protein of the present disclosure, may be identified, screened, and/or characterized for their physical/chemical properties and/or biological activities by various assays known in the art.

Certain aspects of the present disclosure relate to the use of two or more anti-Sortilin antibodies that when utilized together display additive or synergistic effects, as compared to utilization of a corresponding single anti-Sortilin antibody.

In some embodiments, an anti-Sortilin antibody of the present disclosure is an antibody fragment that binds to a human Sortilin protein.

In some embodiments, an anti-Sortilin antibody of the present disclosure is an antibody fragment that binds to one or more human proteins selected from the group consisting of human Sortilin, a naturally occurring variant of human Sortilin, and a disease variant of human Sortilin.

In some embodiments, an anti-Sortilin antibody of the present disclosure is an antibody fragment, wherein the antibody fragment is an Fab, Fab′, Fab′-SH, F(ab′)2, Fv, or scFv fragment.

### Antibody Frameworks

In some embodiments, anti-Sortilin antibodies of the present disclosure comprise a heavy chain variable region comprising one or more (e.g., one or more, two or more, three or more, or all four) framework regions selected from VH FR1, VH FR2, VH FR3, and VH FR4 (as shown in Tables 7-10). In some embodiments, the VH FR1 comprises a sequence of QVQLQESGPGLVKPSETLSL TCAVSG (SEQ ID NO: 35). In some embodiments, the VH FR2 comprises a sequence of WIRQPPGKGLEWIG (SEQ ID NO: 36). In some embodiments, the VH FR3 comprises the sequence according to Formula VI: X₁VTISVDTSKNQFSLX₂LSSVTAADTAVYYC (SEQ ID NO: 39), wherein X₁ is Q or R, and X₂ is E or K. In some embodiments, VH FR3 comprises a sequence selected from the group consisting of SEQ ID NOs: 37-38. In some embodiments, VH FR4 comprises a sequence of WGQGTTVTVSS (SEQ ID NO: 40). In some embodiments, an antibody comprises a heavy chain variable region comprising a VH FR1 comprising the sequence of SEQ ID NO: 35, a VH FR2 comprising the sequence of SEQ ID NO: 36, a VH FR3 according to Formula VI, and a VH FR4 comprising the sequence of SEQ ID NO: 40.

In some embodiments, an antibody comprises a heavy chain variable region comprising a VH FR1 comprising the sequence of SEQ ID NO: 35, a VH FR2 comprising the sequence of SEQ ID NO: 36, a VH FR3 comprising the sequence selected from SEQ ID NOs: 37-38, and a VH FR4 comprising the sequence of SEQ ID NO: 40.

In some embodiments, anti-Sortilin antibodies of the present disclosure comprise a heavy chain variable region comprising a VH FR1, a VH FR2, a VH FR3, and VH FR4 of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16, S-60-18, S-60-19, or S-60-24 (as shown in Tables 7-10).

In some embodiments, anti-Sortilin antibodies of the present disclosure comprise a light chain variable region comprising one or more (e.g., one or more, two or more, three or more, or all four) framework regions selected from VL FR1, VL FR2, VL FR3, and VL FR4 (as shown in Tables 11-14). In some embodiments, the VL FR1 comprises a sequence according to Formula VII: DIVMTQSPLSLPVTPGX₁X₂ASISC (SEQ ID NO: 44), wherein X₁ is E or G, and X₂ is P or S. In some embodiments, VL FR1 comprises a sequence selected from the group consisting of SEQ ID NOs: 41-43. In some embodiments, the VL FR2 comprises a sequence according to Formula VIII: WYLQKPGQX₁PQLLIY (SEQ ID NO: 47), wherein X₁ is S or P. In some embodiments, VL FR2 comprises a sequence selected from the group consisting of SEQ ID NOs: 45-46. In some embodiments, the VL FR3 comprises a sequence according to Formula IX: GVPDRX₁SGSGSGT DFTLKISRX₂EAEDVGX₃YYC (SEQ ID NO: 52), wherein X₁ is F or L, X₂ is A or V, and X₃ is V or A. In some embodiments, VL FR3 comprises a sequence selected from the group consisting of SEQ ID NOs: 48-51. In some embodiments, the VL FR4 comprises a sequence of FGGGTKVEIK (SEQ ID NO: 53). In some embodiments, anti-Sortilin antibodies of the present disclosure comprise a light chain variable region comprising a VL FR1 comprising the sequence according to Formula VII, a V_{L} FR2 comprising the sequence according to Formula VIII, a VL FR3 comprising the sequence according to Formula IX, and a VL FR4 comprising the sequence of SEQ ID NO: 53.

In some embodiments, anti-Sortilin antibodies of the present disclosure comprise a light chain variable region comprising a VL FR1 comprising the sequence selected from SEQ ID NOs: 41-43, a VL FR2 comprising the sequence selected from SEQ ID NOs: 45-46, a VL FR3 comprising the sequence selected from SEQ ID NOs: 48-51, and a VL FR4 comprising the sequence of SEQ ID NO: 53.

In some embodiments, anti-Sortilin antibodies of the present disclosure comprise a light chain variable region comprising a VL FR1, a VL FR2, a VL FR3, and VL FR4 of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16, S-60-18, S-60-19, or S-60-24 (as shown in Tables 11-14).

In some embodiments, anti-Sortilin antibodies of the present disclosure comprise a heavy chain variable region comprising one or more (e.g., one or more, two or more, three or more, or all four) framework regions selected from VH FR1, VH FR2, VH FR3, and VH FR4 (as shown in Tables 7-10), and a light chain variable region comprising one or more (e.g., one or more, two or more, three or more, or all four) framework regions selected from VL FR1, VL FR2, VL FR3, and VL FR4 (as shown in Tables 11-14). In some embodiments, anti-Sortilin antibodies of the present disclosure comprise a heavy chain variable region comprising a VH FR1 comprising the sequence of SEQ ID NO: 35, a VH FR2 comprising the sequence of SEQ ID NO: 36, a VH FR3 according to Formula VI, and a VH FR4 comprising the sequence of SEQ ID NO: 40; and a light chain variable region comprising a VL FR1 comprising the sequence according to Formula VII, a VL FR2 comprising the sequence according to Formula VIII, a VL FR3 comprising the sequence according to Formula IX, and a VL FR4 comprising the sequence of SEQ ID NO: 53. In some embodiments, anti-Sortilin antibodies of the present disclosure comprise a heavy chain variable region comprising a VH FR1 comprising the sequence of SEQ ID NO: 35, a VH FR2 comprising the sequence of SEQ ID NO: 36, a VH FR3 comprising the sequence selected from SEQ ID NOs: 37-38, and a VH FR4 comprising the sequence of SEQ ID NO: 40; a light chain variable region comprising a VL FR1 comprising the sequence selected from SEQ ID NOs: 41-43, a VL FR2 comprising the sequence selected from SEQ ID NOs: 45-46, a VL FR3 comprising the sequence selected from SEQ ID NOs: 48-51, and a VL FR4 comprising the sequence of SEQ ID NO: 53.

In some embodiments, anti-Sortilin antibodies of the present disclosure comprise a heavy chain variable region comprising a VH FR1, a VH FR2, a VH FR3, and VH FR4 of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16, S-60-18, S-60-19, or S-60-24 (as shown in Tables 7-10), and a light chain variable region comprising a VL FR1, a VL FR2, a VL FR3, and VL FR4 of antibody S-60-10, S-60-11, S-60-12, S-60-13, S-60-14, S-60-15 [N33 (wt)], S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], S-60-15.17 [N33L], S-60-16, S-60-18, S-60-19, or S-60-24 (as shown in Tables 11-14).

### Anti-Sortilin Antibody Activities

In certain aspects, any of the anti-Sortilin antibodies of the present disclosure can inhibit one or more activities of a Sortilin protein, including, but not limited to, decreasing cellular levels of Sortilin (e.g., cell surface levels of Sortilin, intracellular levels of Sortilin, and/or total levels of Sortilin); increasing Progranulin levels (e.g., extracellular levels of Progranulin and/or cellular levels of Progranulin); and inhibiting the interaction (e.g., binding) between Progranulin and Sortilin. As contemplated herein, anti-Sortilin antibodies of the present disclosure may inhibit additional activities of a Sortilin protein, including but not limited to inhibiting interaction (e.g., binding) with one or more of pro-neurotrophins of the present disclosure (pro-neurotrophin-3, pro-neurotrophin-4/5, pro-NGF, pro-BDNF, etc.), neurotrophins of the present disclosure (neurotrophin-3, neurotrophin-4/5, NGF, BDNF, etc.), neurotensin, p75, Sortilin propeptide (Sort-pro), amyloid precursor protein (APP), the A beta peptide, lipoprotein lipase (LpL), apolipoprotein AV (APOA5), apolipoprotein E (APOE), and receptor associated protein (RAP); decreasing secretion of PCSK9; and/or decreasing production of beta amyloid peptide.

In certain embodiments, the present disclosure provides an anti-Sortilin antibody, wherein (a) the anti-Sortilin antibody increases extracellular levels of Progranulin, decreases cellular levels of Sortilin, inhibits interaction between Sortilin and Progranulin, or any combination thereof; (b) the anti-Sortilin antibody decreases cell surface levels of Sortilin, increases extracellular levels of Progranulin, inhibits interaction between Sortilin and Progranulin, or any combination thereof; (c) the anti-Sortilin antibody decreases cell surface levels of Sortilin, decreases intracellular levels of Sortilin, decreases total levels of Sortilin, or any combination thereof; (d) the anti-Sortilin antibody induces Sortilin degradation, Sortilin cleavage, Sortilin internalization, Sortilin down regulation, or any combination thereof; (e) the anti-Sortilin antibody decreases cellular levels of Sortilin and inhibits the interaction between Sortilin and Progranulin; (f) the anti-Sortilin antibody decreases cellular levels of Sortilin and increases cellular levels of Progranulin; and/or (g) the anti-Sortilin antibody increases the effective concentration of Progranulin.

In certain embodiments, the present disclosure provides an anti-Sortilin antibody, wherein the anti-Sortilin antibody decreases cell surface levels of Sortilin, increases extracellular levels of Progranulin, inhibits interaction between Sortilin and Progranulin, or any combination thereof.

In some embodiments, an anti-Sortilin antibody of the present disclosure (a) reduces cell surface levels of Sortilin with a half maximal effective concentration (EC₅₀) that is less than 150 μM, as measured by flow cytometry; (b) reduces cell surface levels of Sortilin by more than about 50% at 1.25 nM IgG, by more than about 80% at 0.63 nM IgG, or by more than about 69% at 150 nM IgG relative to control, as measured by flow cytometry; (c) increases Progranulin secretion by more than about 1.13 fold over control at 0.63 nM IgG, or by more than about 1.22 fold over control at 50 nM IgG, as measured by standard ELISA; (d) blocks binding of Progranulin to Sortilin with a half maximal effective concentration (EC₅₀) that is less than 0.325 nM, as measured by flow cytometry; (e) blocks binding of Progranulin to Sortilin by more than about 88% at 50 nM IgG, or by more than about 27.5% at 150 nM IgG relative to control, as measured by flow cytometry; or (f) any combination thereof.

In some embodiments, an anti-Sortilin antibody of the present disclosure (a) reduces cell surface levels of Sortilin with a half maximal effective concentration (EC₅₀) that is less than 681 μM, as measured by flow cytometry; (b) reduces cell surface levels of Sortilin by more than about 40% at 1.25 nM IgG, by more than about 29% at 0.6 nM IgG, or by more than about 62% at 150 nM IgG relative to control, as measured by flow cytometry; (c) increases Progranulin secretion by more than about 1.11 fold over control at 0.63 nM IgG, or by more than about 1.75 fold over control at 50 nM IgG, as measured by standard ELISA; (d) blocks binding of Progranulin to Sortilin with a half maximal effective concentration (EC₅₀) that is less than 0.751 nM, as measured by flow cytometry; (e) blocks binding of Progranulin to Sortilin by more than about 90% at 50 nM IgG, or by more than about 95% at 150 nM IgG relative to control, as measured by flow cytometry; or (f) any combination thereof.

### Decreasing Sortilin Levels

In some embodiments, anti-Sortilin antibodies of the present disclosure bind to a Sortilin protein of the present disclosure expressed on the surface of a cell and modulate (e.g., induce or inhibit) one or more Sortilin activities of the present disclosure after binding to the surface-expressed Sortilin protein.

In some embodiments, anti-Sortilin antibodies of the present disclosure decrease cellular levels of Sortilin in vitro. In some embodiments, anti-Sortilin antibodies of the present disclosure may decrease cellular levels of Sortilin in vivo (e.g., in the brain, and/or peripheral organs of an individual). In some embodiments, a decrease in cellular levels of Sortilin comprises a decrease in cell surface levels of Sortilin. As used herein, an anti-Sortilin antibody decreases cell surface levels of Sortilin if it induces a decrease at saturating antibody concentrations (e.g., 0.6 nM, 0.63 nM, 1.25 nM, 50 nM or 150 nM) and/or relative to a control antibody (e.g. an anti-Sortilin antibody having a heavy chain variable region and a light chain variable region corresponding to S-60) in cell surface levels of Sortilin as measured by any in vitro cell-based assays or suitable in vivo model described herein or known in the art. In some embodiments, a decrease in cellular levels of Sortilin comprises a decrease in intracellular levels of Sortilin. As contemplated herein, an anti-Sortilin antibody decreases intracellular levels of Sortilin if it induces a decrease at saturating antibody concentrations and/or relative to a control antibody (e.g. an anti-Sortilin antibody having a heavy chain variable region and a light chain variable region corresponding to S-60) in intracellular levels of Sortilin as measured by any in vitro cell-based assays or suitable in vivo model described herein or known in the art. In some embodiments, a decrease in cellular levels of Sortilin comprises a decrease in total levels of Sortilin. As contemplated herein, an anti-Sortilin antibody decreases total levels of Sortilin if it induces a decrease at saturating antibody concentrations and/or relative to a control antibody (e.g. an anti-Sortilin antibody having a heavy chain variable region and a light chain variable region corresponding to S-60) in total levels of Sortilin as measured by any in vitro cell-based assays or suitable in vivo model described herein or known in the art.

As used herein, levels of Sortilin may refer to expression levels of the gene encoding Sortilin; to expression levels of one or more transcripts encoding Sortilin; to expression levels of Sortilin protein; and/or to the amount of Sortilin protein present within cells and/or on the cell surface. Any methods known in the art for measuring levels of gene expression, transcription, translation, and/or protein abundance or localization may be used to determine the levels of Sortilin.

Cellular levels of Sortilin may refer to, without limitation, cell surface levels of Sortilin, intracellular levels of Sortilin, and total levels of Sortilin. In some embodiments, a decrease in cellular levels of Sortilin comprises decrease in cell surface levels of Sortilin. In some embodiments, anti-Sortilin antibodies of the present disclosure that decrease cellular levels of Sortilin (e.g., cell surface levels of Sortilin) have one or more of the following characteristics: (1) inhibits or reduces one or more Sortilin activities; (2) the ability to inhibit or reduce binding of a Sortilin to one or more of its ligands; (3) the ability to reduce Sortilin expression in Sortilin-expressing cells; (4) the ability to interact, bind, or recognize a Sortilin protein; (5) the ability to specifically interact with or bind to a Sortilin protein; and (6) the ability to treat, ameliorate, or prevent any aspect of a disease or disorder described or contemplated herein.

In some embodiments, an isolated anti-Sortilin antibody of the present disclosure induces downregulation of Sortilin. In some embodiments, an isolated anti-Sortilin antibody of the present disclosure induces cleavage of Sortilin. In some embodiments, an isolated anti-Sortilin antibody of the present disclosure induces internalization of Sortilin. In some embodiments, an isolated anti-Sortilin antibody of the present disclosure induces shedding of Sortilin. In some embodiments, an isolated anti-Sortilin antibody of the present disclosure induces degradation of Sortilin. In some embodiments, an isolated anti-Sortilin antibody of the present disclosure induces desensitization of Sortilin. In some embodiments, an isolated anti-Sortilin antibody of the present disclosure acts as a ligand mimetic to transiently activate Sortilin. In some embodiments, an isolated anti-Sortilin antibody of the present disclosure acts as a ligand mimetic and transiently activates Sortilin before inducing a decrease in cellular levels of Sortilin and/or inhibition of interaction (e.g., binding) between Sortilin and one or more Sortilin ligands. In some embodiments, an isolated anti-Sortilin antibody of the present disclosure acts as a ligand mimetic and transiently activates Sortilin before inducing degradation of Sortilin. In some embodiments, an isolated anti-Sortilin antibody of the present disclosure acts as a ligand mimetic and transiently activates Sortilin before inducing cleavage of Sortilin. In some embodiments, an isolated anti-Sortilin antibody of the present disclosure acts as a ligand mimetic and transiently activates Sortilin before inducing internalization of Sortilin. In some embodiments, an isolated anti-Sortilin antibody of the present disclosure acts as a ligand mimetic and transiently activates Sortilin before inducing shedding of Sortilin. In some embodiments, an isolated anti-Sortilin antibody of the present disclosure acts as a ligand mimetic and transiently activates Sortilin before inducing downregulation of Sortilin expression. In some embodiments, an isolated anti-Sortilin antibody of the present disclosure acts as a ligand mimetic and transiently activates Sortilin before inducing desensitization of Sortilin.

In certain embodiments, anti-Sortilin antibodies of the present disclosure may decrease cellular levels of Sortilin (e.g., cell surface levels of Sortilin, intracellular levels of Sortilin, and/or total levels of Sortilin) by inducing Sortilin degradation. Accordingly, in some embodiments, anti-Sortilin antibodies of the present disclosure induce Sortilin degradation.

Anti-Sortilin antibodies of the present disclosure may decrease cellular levels (e.g., cell surface levels) of Sortilin with a half-maximal effective concentration (EC₅₀) (e.g., when measured in vitro) in the picomolar range. In certain embodiments, the EC₅₀ of the antibody is less than about 680.9 pM. In certain embodiments, the EC₅₀ of the antibody is about 72.58 pM to about 680.9 nM. In certain embodiments, the EC₅₀ of the antibody is about 103.6 pM to about 680.9 nM. In certain embodiments, the EC₅₀ of the antibody is less than about 600 pM, 500 pM, 400 pM, 300 pM, 200 pM, 100 pM, 50 pM, 40 pM, 30 pM, 20 pM, 10 pM, 1 pM, or 0.5 pM.

In some embodiments, the EC₅₀ of the antibody is less than about or equal to about 675 pM, 650 pM, 625 pM, 600 pM, 575 pM, 550 pM, 525 pM, 500 pM, 475 pM, 450 pM, 425 pM, 400 pM, 375 pM, 350 pM, 325 pM, 300 pM, 275 pM, 250 pM, 225 pM, 200 pM, 175 pM, 150 pM, 125 pM, 100 pM, 90 pM, 80 pM, 70 pM, 60 pM, 50 pM, 40 pM, 30 pM, 20 pM, 10 pM, 9 pM, 8 pM, 7 pM, 6 pM, 5 pM, 4 pM, 3 pM, 2 pM, 1 pM, or 0.5 pM.

In some embodiments, the EC₅₀ of the antibody is less than about 680.9 pM. In some embodiments, the EC₅₀ of the antibody is greater than about or equal to about 0.1 pM, 0.5 pM, 1 pM, 10 pM, 20 pM, 30 pM, 40 pM, 50 pM, 60 pM, 70 pM, 80 pM, 90 pM, 100 pM, 125 pM, 150 pM, 175 pM, 200 pM, 225 pM, 250 pM, 275 pM, 300 pM, 325 pM, 350 pM, 375 pM, 400 pM, 425 pM, 450 pM, 475 pM, 500 pM, 525 pM, 550 pM, 575 pM, 600 pM, 625 pM, 650 pM, 675 pM. That is, the EC₅₀ of the antibody can be any of a range having an upper limit of about 675 pM, 650 nM, 650 pM, 625 pM, 600 pM, 575 pM, 550 pM, 525 pM, 500 pM, 475 pM, 450 pM, 425 pM, 400 pM, 375 pM, 350 pM, 325 pM, 300 pM, 275 pM, 250 pM, 225 pM, 200 pM, 175 pM, 150 pM, 125 pM, 100 pM, 90 pM, 80 pM, 70 pM, 60 pM, 50 pM, 40 pM, 30 pM, 20 pM, 10 pM, 1 pM, or 0.5 pM, and an independently selected lower limit of about 0.1 pM, 0.5 pM, 1 pM, 10 pM, 20 pM, 30 pM, 40 pM, 50 pM, 60 pM, 70 pM, 80 pM, 90 pM, 100 pM, 125 pM, 150 pM, 175 pM, 200 pM, 225 pM, 250 pM, 275 pM, 300 pM, 325 pM, 350 pM, 375 pM, 400 pM, 425 pM, 450 pM, 475 pM, 500 pM, 525 pM, 550 pM, 575 pM, 600 pM, 625 pM, 650 pM, or 675 pM, wherein the lower limit is less than the upper limit. In some embodiments, the EC₅₀ of the antibody is any of about 1 pM, 2 pM, 3 pM, 4 pM, 5 pM, 6 pM, 7 pM, 8 pM, 9 pM, 10 pM, 15 pM, 20 pM, 25 pM, 30 pM, 35 pM, 40 pM, 45 pM, 50 pM, 55 pM, 60 pM, 65 pM, 70 pM, 75 pM, 80 pM, 85 pM, 90 pM, 95 pM, 100 pM, 105 pM, 110 pM, 115 pM, 120 pM, 125 pM, 130 pM, 135 pM, 140 pM, 145 pM, 150 pM, 155 pM, 160 pM, 165 pM, 170 pM, 175 pM, 180 pM, 185 pM, 190 pM, 195 pM, or 200 pM.

In some embodiments, an anti-Sortilin antibody of the present disclosure reduces cell surface levels of Sortilin with a half maximal effective concentration (EC₅₀) that is less than 150 pM, as measured by flow cytometry. In some embodiments, the EC₅₀ of an anti-Sortilin antibody of the present disclosure is about 103.6 pM. In some embodiments, the EC₅₀ of an anti-Sortilin antibody of the present disclosure is about 72.58 pM.

In some embodiments, an anti-Sortilin antibody of the present disclosure reduces cell surface levels of Sortilin by more than about 40% at 1.25 nM IgG or by more than about 80% at 0.63 nM IgG, as measured by flow cytometry. In some embodiments, an anti-Sortilin antibody of the present disclosure reduces cell surface levels of Sortilin by about 60.92% at 1.25 nM IgG, as measured by flow cytometry. In some embodiments, an anti-Sortilin antibody of the present disclosure reduces cell surface levels of Sortilin by about 69.3% at 150 nM IgG, as measured by flow cytometry. In some embodiments, an anti-Sortilin antibody of the present disclosure reduces cell surface levels of Sortilin by about 70.3% at 150 nM IgG, as measured by flow cytometry.

Various methods of measuring antibody EC₅₀ values are known in the art, including, for example, by flow cytometry. In some embodiments, the EC₅₀ is measured in vitro using cells engineered to express human Sortilin. In some embodiments, the EC₅₀ is measured at a temperature of approximately 4° C. In some embodiments, the EC₅₀ is measured at a temperature of approximately 25° C. In some embodiments, the EC₅₀ is measured at a temperature of approximately 35° C. In some embodiments, the EC₅₀ is measured at a temperature of approximately 37° C. In some embodiments, the EC₅₀ is determined using a monovalent antibody (e.g., a Fab) or a full-length antibody in a monovalent form. In some embodiments, the EC₅₀ is determined using antibodies containing constant regions that demonstrate enhanced Fc receptor binding. In some embodiments, the EC₅₀ is determined using antibodies containing constant regions that demonstrate reduced Fc receptor binding.

In some embodiments, anti-Sortilin antibodies of the present disclosure have higher potencies in reducing cell surface levels of Sortilin relative to a control antibody (e.g. an anti-Sortilin antibody having a heavy chain variable region and a light chain variable region corresponding to S-60). In some embodiments, anti-Sortilin antibodies of the present disclosure decrease cellular levels (e.g., cell surface levels) of Sortilin with a lower EC₅₀ (e.g., as measured in vitro) than a control antibody (e.g. an anti-Sortilin antibody having a heavy chain variable region and a light chain variable region corresponding to S-60). In some embodiments, anti-Sortilin antibodies of the present disclosure decrease cellular levels (e.g., cell surface levels) of Sortilin with an EC₅₀ that is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% lower than the EC₅₀ of a control antibody (e.g. an anti-Sortilin antibody having a heavy chain variable region and a light chain variable region corresponding to S-60). In some embodiments, anti-Sortilin antibodies of the present disclosure decrease cellular levels (e.g., cell surface levels) of Sortilin with an EC₅₀ that is at least about 1-fold, at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 12.5-fold, at least about 15-fold, at least about 17.5-fold, at least about 20-fold, at least about 22.5-fold, at least about 25-fold, at least about 27.5-fold, at least about 30-fold, at least about 50-fold, or at least about 100-fold lower than the EC₅₀ of a control antibody (e.g. an anti-Sortilin antibody having a heavy chain variable region and a light chain variable region corresponding to S-60).

In some embodiments, anti-Sortilin antibodies of the present disclosure have an EC₅₀ that is at least 1.5-fold lower than control antibody (e.g. an anti-Sortilin antibody having a heavy chain variable region and a light chain variable region corresponding to S-60). In some embodiments, anti-Sortilin antibodies of the present disclosure have an EC₅₀ that is at least 1.1-fold lower than control antibody (e.g. an anti-Sortilin antibody having a heavy chain variable region and a light chain variable region corresponding to S-60).

In some embodiments, an anti-Sortilin antibody of the present disclosure (a) reduces cell surface levels of Sortilin with a half maximal effective concentration (EC₅₀) that is less than 681 pM, as measured by flow cytometry; (b) reduces cell surface levels of Sortilin by more than about 40% at 1.25 nM IgG, by more than about 29% at 0.6 nM IgG, or by more than about 62% at 150 nM IgG relative to control, as measured by flow cytometry; (c) increases Progranulin secretion by more than about 1.11 fold over control at 0.63 nM IgG, or by more than about 1.75 fold over control at 50 nM IgG, as measured by standard ELISA; (d) blocks binding of Progranulin to Sortilin with a half maximal effective concentration (EC₅₀) that is less than 0.751 nM, as measured by flow cytometry; (e) blocks binding of Progranulin to Sortilin by more than about 90% at 50 nM IgG, or by more than about 95% at 150 nM IgG relative to control, as measured by flow cytometry; or (f) any combination thereof.

### Increasing Progranulin Levels

In some embodiments, anti-Sortilin antibodies of the present disclosure increase extracellular levels of Progranulin in vitro. In some embodiments, anti-Sortilin antibodies of the present disclosure may increase cellular levels of Progranulin in vitro or in vivo (e.g., in the brain, blood, and/or peripheral organs of an individual). As used herein, an anti-Sortilin antibody increases extracellular levels of Progranulin if it induces an increase at saturating antibody concentrations (e.g., 0.6 nM, 0.63 nM, 1.25 nM, 50 nM or 150 nM) and/or relative to a control antibody (e.g. an anti-Sortilin antibody having a heavy chain variable region and a light chain variable region corresponding to S-60) in extracellular levels of Progranulin as measured by any in vitro cell-based assays or in tissue-based (such as brain tissue-based) assays described herein or known in the art. As contemplated herein, an anti-Sortilin antibody increases cellular levels of Progranulin if it induces an increase at saturating antibody concentrations (e.g., 0.6 nM, 0.63 nM, 1.25 nM, 50 nM or 150 nM) and/or relative to a control antibody (e.g. an anti-Sortilin antibody having a heavy chain variable region and a light chain variable region corresponding to S-60) in cellular levels of Progranulin as measured by any in vitro cell-based assays or in tissue-based (such as brain tissue-based) assays described herein or known in the art.

As used herein, levels of Progranulin may refer to expression levels of the gene encoding Progranulin; to expression levels of one or more transcripts encoding Progranulin; to expression levels of Progranulin protein; and/or to the amount of Progranulin protein secreted from cells and/or present within cells. Any methods known in the art for measuring levels of gene expression, transcription, translation, protein abundance, protein secretion, and/or protein localization may used to determine the levels of Progranulin.

As used herein, Progranulin levels may refer to, without limitation, extracellular levels of Progranulin, intracellular levels of Progranulin, and total levels of Progranulin. In some embodiments, an increase in levels of Progranulin comprises an increase in extracellular levels of Progranulin.

In some embodiments, an anti-Sortilin antibody of the present disclosure increases Progranulin secretion by more than about 1.11 fold over control at 0.63 nM IgG, as measured by standard ELISA. In some embodiments, an anti-Sortilin antibody of the present disclosure increases Progranulin secretion by about 1.42 fold over control at 0.63 nM IgG, as measured by standard ELISA. In some embodiments, an anti-Sortilin antibody of the present disclosure increases Progranulin secretion by more than about 1.75 fold over control at 50 nM IgG, as measured by standard ELISA. In some embodiments, an anti-Sortilin antibody of the present disclosure increases Progranulin secretion by about 1.97 fold over control at 50 nM IgG, as measured by standard ELISA. In some embodiments, an anti-Sortilin antibody of the present disclosure increases Progranulin secretion by about 2.29 fold over control at 50 nM IgG, as measured by standard ELISA.

Various methods of measuring Progranulin secretion are known in the art, including, for example, by ELISA. In some embodiments, the EC₅₀ is measured in vitro using cells expressing human Sortilin. In some embodiments, Progranulin secretion is determined using a monovalent antibody (e.g., a Fab) or a full-length antibody in a monovalent form. In some embodiments, Progranulin secretion is determined using antibodies containing constant regions that demonstrate enhanced Fc receptor binding. In some embodiments, Progranulin secretion is determined using antibodies containing constant regions that demonstrate reduced Fc receptor binding.

In some embodiments, anti-Sortilin antibodies of the present disclosure have higher potencies in increasing levels of Progranulin relative to a control antibody (e.g. an anti-Sortilin antibody having a heavy chain variable region and a light chain variable region corresponding to S-60). In some embodiments, anti-Sortilin antibodies of the present disclosure increase levels (e.g., extracellular levels) of Progranulin with a lower EC₅₀ (e.g., as measured in vitro) than a control antibody (e.g. an anti-Sortilin antibody having a heavy chain variable region and a light chain variable region corresponding to S-60). In some embodiments, anti-Sortilin antibodies of the present disclosure increase levels (e.g., extracellular levels) of Progranulin by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% greater than a control antibody (e.g. an anti-Sortilin antibody having a heavy chain variable region and a light chain variable region corresponding to S-60). In some embodiments, anti-Sortilin antibodies of the present disclosure increase levels (e.g., extracellular levels) of Progranulin by at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 12.5-fold, at least about 15-fold, at least about 17.5-fold, at least about 20-fold, at least about 22.5-fold, at least about 25-fold, at least about 27.5-fold, at least about 30-fold, at least about 50-fold, or at least about 100-fold higher than a control antibody (e.g. an anti-Sortilin antibody having a heavy chain variable region and a light chain variable region corresponding to S-60).

In some embodiments, anti-Sortilin antibodies of the present disclosure increase Progranulin levels by about 1.1-fold higher than a control antibody (e.g. an anti-Sortilin antibody having a heavy chain variable region and a light chain variable region corresponding to S-60). In some embodiments, anti-Sortilin antibodies of the present disclosure increase Progranulin levels by about 1.3-fold higher than a control antibody (e.g. an anti-Sortilin antibody having a heavy chain variable region and a light chain variable region corresponding to S-60).

In some embodiments, anti-Sortilin antibodies of the present disclosure increase the effective concentration of Progranulin. The effective concentration of Progranulin refers to the concentration of Progranulin in plasma or cerebrospinal fluid. In some embodiments, an increase in the effective concentration of Progranulin is an increase of greater than 1.5 fold. In some embodiments, the effective concentration of Progranulin is increased for 7-28 days.

Decreasing Interaction between Sortilin and Progranulin

In some embodiments, anti-Sortilin antibodies of the present disclosure increase Progranulin levels and/or decrease cellular levels of Sortilin while blocking (e.g. inhibiting) the interaction (e.g., binding) between Sortilin and Progranulin. Accordingly, in some embodiments, anti-Sortilin antibodies of the present disclosure block the interaction (e.g., binding) between Sortilin and Progranulin. As used herein, an anti-Sortilin antibody blocks the interaction (e.g., binding) between Sortilin and Progranulin if it decreases Progranulin binding to Sortilin relative to a control antibody (e.g. an anti-Sortilin antibody having a heavy chain variable region and a light chain variable region corresponding to S-60) at saturating antibody concentrations (e.g., 0.6 nM, 0.63 nM, 1.25 nM, 50 nM or 150 nM) in any in vitro assay or cell-based culture assay described herein or known in the art.

Anti-Sortilin antibodies of the present disclosure may decrease Progranulin binding to Sortilin with a half-maximal effective concentration (EC₅₀) (e.g., when measured in vitro) in the picomolar range. In certain embodiments, the EC₅₀ of the antibody is less than about 2.2 nM. In certain embodiments, the EC₅₀ of the antibody is less than about 1.22 nM. In certain embodiments, the EC₅₀ of the antibody is less than about 751 pM. In certain embodiments, the EC₅₀ of the antibody is about 325 pM to about 751 nM. In certain embodiments, the EC₅₀ of the antibody is about 405 pM to about 751 nM. In certain embodiments, the EC₅₀ of the antibody is about 588 pM to about 751 nM. In certain embodiments, the EC₅₀ of the antibody is less than about 2.2 nM, 2.1 nM, 2.0 nM, 1.9 nM, 1.8 nM, 1.7 nM, 1.6 nM, 1.5 nM, 1.4 nM, 1.3 nM, 1.2 nM, 1.1 nM, 1.0 nM, 900 pM, 800 pM, 700 pM, 600 pM, 500 pM, 400 pM, 300 pM, 200 pM, 100 pM, 50 pM, 40 pM, 30 pM, 20 pM, 10 pM, 1 pM, or 0.5 pM.

In some embodiments, the EC₅₀ of the antibody for decreasing Progranulin binding to Sortilin is less than about or equal to about 2.2 nM, 2.1 nM, 2.0 nM, 1.9 nM, 1.8 nM, 1.7 nM, 1.6 nM, 1.5 nM, 1.4 nM, 1.3 nM, 1.2 nM, 1.1 nM, 1.0 nM, 900 pM, 800 pM, 700 pM, 600 pM, 500 pM, 475 pM, 450 pM, 425 pM, 400 pM, 375 pM, 350 pM, 325 pM, 300 pM, 275 pM, 250 pM, 225 pM, 200 pM, 175 pM, 150 pM, 125 pM, 100 pM, 90 pM, 80 pM, 70 pM, 60 pM, 50 pM, 40 pM, 30 pM, 20 pM, 10 pM, 9 pM, 8 pM, 7 pM, 6 pM, 5 pM, 4 pM, 3 pM, 2 pM, 1 pM, or 0.5 pM.

In some embodiments, the EC₅₀ of an anti-Sortilin antibody of the present disclosure is about 1.22 nM. In some embodiments, the EC₅₀ of an anti-Sortilin antibody of the present disclosure is about 588 pM. In some embodiments, the EC₅₀ of an anti-Sortilin antibody of the present disclosure is about 405 pM. In some embodiments, the EC₅₀ of an anti-Sortilin antibody of the present disclosure is about 325 pM.

Various methods of measuring antibody EC₅₀ values are known in the art, including, for example, by flow cytometry. In some embodiments, the EC₅₀ for decreasing Progranulin binding to Sortilin is measured in vitro using cells expressing human Sortilin. In some embodiments, the EC₅₀ is measured at a temperature of approximately 4° C. In some embodiments, the EC₅₀ is measured at a temperature of approximately 25° C. In some embodiments, the EC₅₀ is measured at a temperature of approximately 35° C. In some embodiments, the EC₅₀ is measured at a temperature of approximately 37° C. In some embodiments, the EC₅₀ for decreasing Progranulin binding to Sortilin is determined using a monovalent antibody (e.g., a Fab) or a full-length antibody in a monovalent form. In some embodiments, the EC₅₀ is determined using antibodies containing constant regions that demonstrate enhanced Fc receptor binding. In some embodiments, the EC₅₀ for decreasing Progranulin binding to Sortilin is determined using antibodies containing constant regions that demonstrate reduced Fc receptor binding.

In some embodiments, anti-Sortilin antibodies of the present disclosure have higher potencies in reducing Progranulin binding to Sortilin relative to a control antibody (e.g. an anti-Sortilin antibody having a heavy chain variable region and a light chain variable region corresponding to S-60). In some embodiments, anti-Sortilin antibodies of the present disclosure decrease Progranulin binding to Sortilin with a lower EC₅₀ (e.g., as measured in vitro) than a control antibody (e.g. an anti-Sortilin antibody having a heavy chain variable region and a light chain variable region corresponding to S-60). In some embodiments, anti-Sortilin antibodies of the present disclosure decrease Progranulin binding to Sortilin with an EC₅₀ that is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% lower than the EC₅₀ of a control antibody (e.g. an anti-Sortilin antibody having a heavy chain variable region and a light chain variable region corresponding to S-60). In some embodiments, anti-Sortilin antibodies of the present disclosure decrease Progranulin binding to Sortilin with an EC₅₀ that is at least about 1-fold, at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 12.5-fold, at least about 15-fold, at least about 17.5-fold, at least about 20-fold, at least about 22.5-fold, at least about 25-fold, at least about 27.5-fold, at least about 30-fold, at least about 50-fold, or at least about 100-fold lower than the EC₅₀ of a control antibody (e.g. an anti-Sortilin antibody having a heavy chain variable region and a light chain variable region corresponding to S-60).

In some embodiments, anti-Sortilin antibodies of the present disclosure have an EC₅₀ that is at least 1.3-fold lower than control antibody (e.g. an anti-Sortilin antibody having a heavy chain variable region and a light chain variable region corresponding to S-60). In some embodiments, anti-Sortilin antibodies of the present disclosure have an EC₅₀ that is at least 1.8-fold lower than control antibody (e.g. an anti-Sortilin antibody having a heavy chain variable region and a light chain variable region corresponding to S-60). In some embodiments, anti-Sortilin antibodies of the present disclosure have an EC₅₀ that is at least 1.9-fold lower than control antibody (e.g. an anti-Sortilin antibody having a heavy chain variable region and a light chain variable region corresponding to S-60). In some embodiments, anti-Sortilin antibodies of the present disclosure have an EC₅₀ that is at least 2.3-fold lower than control antibody (e.g. an anti-Sortilin antibody having a heavy chain variable region and a light chain variable region corresponding to S-60).

Any in vitro cell-based assays or suitable in vivo model described herein or known in the art may be used to measure inhibition or reduction of interaction (e.g., binding) between Sortilin and one or more Sortilin ligands, e.g., Progranulin. In some embodiments, anti-Sortilin antibodies of the present disclosure inhibit or reduce interaction (e.g., binding) between Sortilin and one or more Sortilin ligands, e.g., Progranulin, by reducing Sortilin expression (e.g., by reducing cell surface levels of Sortilin). In some embodiments, anti-Sortilin antibodies of the present disclosure inhibit or reduce interaction (e.g., binding) between Sortilin and one or more Sortilin ligands, e.g., Progranulin, by at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more at saturating antibody concentrations utilizing any in vitro assay or cell-based culture assay described herein or known in the art.

In some embodiments, an anti-Sortilin antibody of the present disclosure blocks Progranulin binding to Sortilin by more than about 90% at 50 nM IgG or by more than about 96% at 150 nM IgG, as measured by flow cytometry. In some embodiments, an anti-Sortilin antibody of the present disclosure blocks Progranulin binding to Sortilin by about 90.74% at 50 nM IgG, as measured by flow cytometry. In some embodiments, an anti-Sortilin antibody of the present disclosure blocks Progranulin binding to Sortilin by about 96.5% at 150 nM IgG, as measured by flow cytometry. In some embodiments, an anti-Sortilin antibody of the present disclosure blocks Progranulin binding to Sortilin by about 96.9% at 150 nM IgG, as measured by flow cytometry.

### Decreasing Expression of Pro-Inflammatory Mediators

In some embodiments, anti-Sortilin antibodies of the present disclosure may decrease the expression of pro-inflammatory mediators after binding to a Sortilin protein expressed in a cell.

As used herein, pro-inflammatory mediators are proteins involved either directly or indirectly (e.g., by way of pro-inflammatory signaling pathways) in a mechanism that induces, activates, promotes, or otherwise increases an inflammatory response, such as neuroinflammation. Any method known in the art for identifying and characterizing pro-inflammatory mediators may be used.

Examples of pro-inflammatory mediators include, without limitation, cytokines, such as type I and II interferons, IL-6, IL12p70, IL12p40, IL-1β, TNF-α, IL-8, CRP, IL-20 family members, IL-33, LIF, OSM, CNTF, GM-CSF, IL-11, IL-12, IL-17, IL-18, and CRP. Further examples of pro-inflammatory mediators include, without limitation, chemokines, such as CXCL1, CCL2, CCL3, CCL4, and CCL5. Yet another example of a pro-inflammatory mediator is macrophage migration inhibitory factor (MIF), which is a pleiotropic pro-inflammatory cytokine that is highly and widely expressed in human neural tissues, including neurons, microglia, astrocytes, and ependymal cells.

In some embodiments, the anti-Sortilin antibodies of the present disclosure may decrease functional expression and/or secretion of pro-inflammatory mediators, e.g., IL-6, IL12p70, IL12p40, IL-1β, TNF-α, CXCL1, CCL2, CCL3, CCL4, CCL5, and/or MIF. In certain embodiments, decreased expression of the pro-inflammatory mediators occurs in neurons, astrocytes, ependymal cells, macrophages, dendritic cells, monocytes, osteoclasts, Langerhans cells of skin, Kupffer cells, T cells, and/or microglial cells. Decreased expression may include, without limitation, a decrease in gene expression, a decrease in transcriptional expression, or a decrease in protein expression. In certain embodiments, decreased expression of a pro-inflammatory mediator refers to a decrease in transcript (e.g., mRNA) or protein levels of the pro-inflammatory mediator in blood (e.g., whole blood, plasma or serum), or in cerebrospinal fluid of an individual. Any method known in the art for determining gene, transcript (e.g., mRNA), and/or protein expression may be used. For example, Northern blot analysis may be used to determine pro-inflammatory mediator gene expression levels, RT-PCR may be used to determine the level of pro-inflammatory mediator transcription, and Western blot analysis, SOMASCAN assays (see, e.g., Candia et al. (2017) Sci Rep 7, 14248), or enzyme-linked immunosorbent assays (ELISA) may be used to determine pro-inflammatory mediator protein levels.

As used herein, a pro-inflammatory mediator may have decreased expression if its expression in one or more cells of a subject treated with a Sortilin agent, such as an anti-Sortilin antibody of the present disclosure, is less than the expression of the same pro-inflammatory mediator expressed in one or more cells of a corresponding subject that is not treated with the anti-Sortilin antibody. In some embodiments, the anti-Sortilin antibody of the present disclosure may decrease pro-inflammatory mediator expression in one or more cells of a subject by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, for example, as compared to pro-inflammatory mediator expression in one or more cells of a corresponding subject that is not treated with the anti-Sortilin antibody. In other embodiments, the anti-Sortilin antibody may decrease pro-inflammatory mediator expression in one or more cells of a subject by at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to pro-inflammatory mediator expression in one or more cells of a corresponding subject that is not treated with the anti-Sortilin antibody.

In some embodiments, a pro-inflammatory mediator may have decreased transcript or protein levels in blood (e.g., whole blood, plasma or serum) or in cerebrospinal fluid if the transcript or protein levels of the pro-inflammatory mediator in a subject treated with a Sortilin agent, such as an anti-Sortilin antibody of the present disclosure, are less than the levels of the same pro-inflammatory mediator in the blood (e.g., whole blood, plasma or serum) or in the cerebrospinal fluid of the subject prior to administration of the Sortilin agent, such as an anti-Sortilin antibody of the present disclosure. In some embodiments, an anti-Sortilin antibody of the present disclosure may decrease transcript or protein levels of a pro-inflammatory mediator in the blood (e.g., whole blood, plasma or serum) or in the cerebrospinal fluid of a subject administered the anti-Sortilin antibody by any of at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or 100%, for example, as compared to pro-inflammatory mediator transcript or protein levels in the blood (e.g., whole blood, plasma or serum) or in the cerebrospinal fluid of the subject prior to administration of the anti-Sortilin antibody, or as compared to pro-inflammatory mediator transcript or protein levels in the blood (e.g., whole blood, plasma or serum) or in the cerebrospinal fluid of a corresponding subject that is not treated with the anti-Sortilin antibody. In some embodiments, an anti-Sortilin antibody of the present disclosure may decrease transcript or protein levels of a pro-inflammatory mediator in the blood (e.g., whole blood, plasma or serum) or in the cerebrospinal fluid of a subject administered the anti-Sortilin antibody by any of at least 1.5 fold, at least 1.6 fold, at least 1.7 fold, at least 1.8 fold, at least 1.9 fold, at least 2.0 fold, at least 2.1 fold, at least 2.15 fold, at least 2.2 fold, at least 2.25 fold, at least 2.3 fold, at least 2.35 fold, at least 2.4 fold, at least 2.45 fold, at least 2.5 fold, at least 2.55 fold, at least 3.0 fold, at least 3.5 fold, at least 4.0 fold, at least 4.5 fold, at least 5.0 fold, at least 5.5 fold, at least 6.0 fold, at least 6.5 fold, at least 7.0 fold, at least 7.5 fold, at least 8.0 fold, at least 8.5 fold, at least 9.0 fold, at least 9.5 fold, or at least 10 fold, for example, as compared to pro-inflammatory mediator transcript or protein levels in the blood (e.g., whole blood, plasma or serum) or in the cerebrospinal fluid of the subject prior to administration of the anti-Sortilin antibody, or as compared to pro-inflammatory mediator transcript or protein levels in the blood (e.g., whole blood, plasma or serum) or in the cerebrospinal fluid of a corresponding subject that is not treated with the anti-Sortilin antibody.

In some embodiments, an anti-Sortilin antibody according to any of the above embodiments may incorporate any of the features, singly or in combination, as described in Sections 1-8 below:

### (1) Anti-Sortilin Antibody Binding Affinity

In some embodiments of any of the antibodies provided herein, the antibody has a dissociation constant (Kd) of <1 μM, <100 nM, <10 nM, <1 nM, <0.1 nM, <0.01 nM, or <0.001 nM (e.g., 10⁻⁸ M or less, e.g., from 10⁻⁸ M to 10⁻¹³ M, e.g., from 10⁻⁹ M to 10⁻¹³ M).

Anti-Sortilin antibodies of the present disclosure may have nanomolar or even picomolar affinities for the target antigen (e.g., human Sortilin or mammalian Sortilin). In certain embodiments, the binding affinity of an anti-Sortilin antibody of the present disclosure for target antigen (e.g., human Sortilin or mammalian Sortilin) is measured by the dissociation constant, K_{D}. Dissociation constants may be determined through any analytical technique, including any biochemical or biophysical technique such as fluorescent activated cell sorting (FACS), flow cytometry, enzyme-linked immunosorbent assay (ELISA), surface plasmon resonance (SPR), BioLayer interferometry (see, e.g., Octet System by ForteBio), meso scale discover (see, e.g., MSD-SET), isothermal titration calorimetry (ITC), differential scanning calorimetry (DSC), circular dichroism (CD), stopped-flow analysis, and colorimetric or fluorescent protein melting analyses; or a cell binding assay. In some embodiments, the K_{D} for Sortilin is determined at a temperature of approximately 25° C. In some embodiments, the dissociation constant (K_{D}) may be measured at 4° C. or room temperature utilizing, for example, FACS or BioLlayer interferometry assay.

In some embodiments, the K_{D} for Sortilin is determined at a temperature of approximately 4° C. In some embodiments, the K_{D} is determined using a monovalent antibody (e.g., a Fab) or a full-length antibody in a monovalent form. In some embodiments, the K_{D} is determined using a bivalent antibody and monomeric recombinant Sortilin protein.

In certain embodiments, the K_{D} of an anti-Sortilin antibody of the present disclosure for human Sortilin, mammalian Sortilin, or both, is measured using FACS. In certain embodiments, the K_{D} of an anti-Sortilin antibody of the present disclosure for human Sortilin, mammalian Sortilin, or both, is measured using BioLayer Interferometry.

In some embodiments, the anti-Sortilin antibody has a dissociation constant (K_{D}) for human Sortilin that is up to 2.5-fold lower than an anti-Sortilin antibody comprising a heavy chain variable region comprising the sequence of SEQ ID NO: 56 and a light chain variable region comprising the sequence of SEQ ID NO: 79, wherein the K_{D} is determined by FACS. In some embodiments, the anti-Sortilin antibody has a dissociation constant (K_{D}) for human Sortilin that ranges from about 1.10E-8 M to about 4.68E-10 M wherein the K_{D} is determined by FACS, or about 270 to about 2910 μM wherein the K_{D} is determined by Bio-layer interferometry.

In certain embodiments, the K_{D} of an anti-Sortilin antibody of the present disclosure for human Sortilin, mammalian Sortilin, or both, may be less than 100 nM, less than 90 nM, less than 80 nM, less than 70 nM, less than 60 nM, less than 50 nM, less than 40 nM, less than 30 nM, less than 20 nM, less than 10 nM, less than 9 nM, less than 8 nM, less than 7 nM, less than 6 nM, less than 5 nM, less than 4 nM, less than 3 nM, less than 2 nM, less than 1 nM, less than 0.5 nM, less than 0.1 nM, less than 0.09 nM, less than 0.08 nM, less than 0.07 nM, less than 0.06 nM, less than 0.05 nM, less than 0.04 nM, less than 0.03 nM, less than 0.02 nM, less than 0.01 nM, less than 0.009 nM, less than 0.008 nM, less than 0.007 nM, less than 0.006 nM, less than 0.005 nM, less than 0.004 nM, less than 0.003 nM, less than 0.002 nM, less than 0.001 nM, or less than 0.001 nM.

The dissociation constants (K_{D}) of anti-Sortilin antibodies for human Sortilin, mammalian Sortilin, or both, may be less than 10 nM, less than 9.5 nM, less than 9 nM, less than 8.5 nM, less than 8 nM, less than 7.5 nM, less than 7 nM, less than 6.9 nM, less than 6.8 nM, less than 6.7 nM, less than 6.6 nM, less than 6.5 nM, less than 6.4 nM, less than 6.3 nM, less than 6.2 nM, less than 6.1 nM, less than 6 nM, less than 5.5 nM, less than 5 nM, less than 4.5 nM, less than 4 nM, less than 3.5 nM, less than 3 nM, less than 2.5 nM, less than 2 nM, less than 1.5 nM, less than 1 nM, less than 0.95 nM, less than 0.9 nM, less than 0.89 nM, less than 0.88 nM, less than 0.87 nM, less than 0.86 nM, less than 0.85 nM, less than 0.84 nM, less than 0.83 nM, less than 0.82 nM, less than 0.81 nM, less than 0.8 nM, less than 0.75 nM, less than 0.7 nM, less than 0.65 nM, less than 0.64 nM, less than 0.63 nM, less than 0.62 nM, less than 0.61 nM, less than 0.6 nM, less than 0.55 nM, less than 0.5 nM, less than 0.45 nM, less than 0.4 nM, less than 0.35 nM, less than 0.3 nM, less than 0.29 nM, less than 0.28 nM, less than 0.27 nM, less than 0.26 nM, less than 0.25 nM, less than 0.24 nM, less than 0.23 nM, less than 0.22 nM, less than 0.21 nM, less than 0.2 nM, less than 0.15 nM, less than 0.1 nM, less than 0.09 nM, less than 0.08 nM, less than 0.07 nM, less than 0.06 nM, less than 0.05 nM, less than 0.04 nM, less than 0.03 nM, less than 0.02 nM, less than 0.01 nM, less than 0.009 nM, less than 0.008 nM, less than 0.007 nM, less than 0.006 nM, less than 0.005 nM, less than 0.004 nM, less than 0.003 nM, less than 0.002 nM, or less than 0.001 nM.

In certain embodiments, the dissociation constant (K_{D}) of the antibody for Sortilin is from about 0.560 nM to about 1.63 nM, for example when the K_{D} is determined by FACS. In certain embodiments, the dissociation constant (K_{D}) of the antibody for Sortilin is from about 0.270 nM to about 2.910 nM, for example when the K_{D} is determined by BioLayer Interferometry. In some embodiments, the antibody has a dissociation constant (K_{D}) for human Sortilin, mouse Sortilin, or both, that ranges from about 0.36 nM to about 0.43 nM, or less than 1.02 nM. In some embodiments, the dissociation constant is less than 1.02 nM. In some embodiments, an anti-Sortilin antibody of the present disclosure has a dissociation constant for human Sortilin of 0.560 nM or less.

In one specific embodiment, an anti-Sortilin antibody of the present disclosure has a dissociation constant for human Sortilin of about 0.560 nM. In one specific embodiment, an anti-Sortilin antibody of the present disclosure has a dissociation constant for human Sortilin of about 0.423 nM. In one specific embodiment, an anti-Sortilin antibody of the present disclosure has a dissociation constant for human Sortilin of about 0.365 nM. In one specific embodiment, an anti-Sortilin antibody of the present disclosure has a dissociation constant for human Sortilin of about 0.344 nM. In one specific embodiment, an anti-Sortilin antibody of the present disclosure has a dissociation constant for human Sortilin of about 0.298 nM. In one specific embodiment, an anti-Sortilin antibody of the present disclosure has a dissociation constant for human Sortilin of about 0.270 nM. In another specific embodiment, an anti-Sortilin antibody of the present disclosure has a dissociation constant for human Sortilin of about 0.260 nM.

In some embodiments, anti-Sortilin antibodies of the present disclosure have a lower dissociation constant (K_{D}) for Sortilin than a control anti-Sortilin antibody (e.g., a control anti-Sortilin antibody comprising a heavy chain variable region and a light chain variable region corresponding to S-60). In some embodiments, anti-Sortilin antibodies of the present disclosure have a K_{D} for a target (e.g., human Sortilin) that is at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or at least about 99% lower than the K_{D} of a control anti-Sortilin antibody for the target (e.g., a control anti-Sortilin antibody comprising a heavy chain variable region and a light chain variable region corresponding to S-60). In some embodiments, anti-Sortilin antibodies of the present disclosure have a K_{D} for a target (e.g., human Sortilin) that is at least about 1-fold, at least about 1.1-fold, at least about 1.5-fold, at least about 2-fold, at least about 3-fold, at least about 4-fold, at least about 5-fold, at least about 6-fold, at least about 7-fold, at least about 8-fold, at least about 9-fold, at least about 10-fold, at least about 12.5-fold, at least about 15-fold, at least about 17.5-fold, at least about 20-fold, at least about 22.5-fold, at least about 25-fold, at least about 27.5-fold, at least about 30-fold, at least about 50-fold, at least about 100-fold, at least about 200-fold, at least about 300-fold, at least about 400-fold, at least about 500-fold, at least about 600-fold, at least about 700-fold, at least about 800-fold, at least about 900-fold, or at least about 1000-fold lower than the K_{D} of a control anti-Sortilin antibody for the target (e.g., a control anti-Sortilin antibody comprising a heavy chain variable region and a light chain variable region corresponding to S-60).

In some embodiments, anti-Sortilin antibodies of the present disclosure have a K_{D} for human Sortilin that is at least 100-fold lower than an anti-Sortilin antibody having a heavy chain variable region and a light chain variable region corresponding to S-60. In some embodiments, anti-Sortilin antibodies of the present disclosure have a K_{D} for human Sortilin that is at least 50-fold lower than an anti-Sortilin antibody having a heavy chain variable region and a light chain variable region corresponding to S-60. In some embodiments, anti-Sortilin antibodies of the present disclosure have a K_{D} for human Sortilin that is at least 10-fold lower than an anti-Sortilin antibody having a heavy chain variable region and a light chain variable region corresponding to S-60. In some embodiments, anti-Sortilin antibodies of the present disclosure have a K_{D} for human Sortilin that is at least 5-fold lower than an anti-Sortilin antibody having a heavy chain variable region and a light chain variable region corresponding to S-60. In some embodiments, anti-Sortilin antibodies of the present disclosure have a K_{D} for human Sortilin that is at least 2-fold lower than an anti-Sortilin antibody having a heavy chain variable region and a light chain variable region corresponding to S-60.

In a specific embodiment, an anti-Sortilin antibody of the present disclosure has a K_{D} for human Sortilin that is about 2.79-fold lower than an anti-Sortilin antibody having a heavy chain variable region and a light chain variable region corresponding to S-60. In another specific embodiment, an anti-Sortilin antibody of the present disclosure has a K_{D} for human Sortilin that is about 2.05-fold lower than an anti-Sortilin antibody having a heavy chain variable region and a light chain variable region corresponding to S-60.

### (2) Antibody Fragments

In some embodiments of any of the antibodies provided herein, the antibody is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP404097; WO 1993/01161; Hudson et al. Nat. Med. 9:129-134 (2003). Triabodies and tetrabodies are also described in Hudson et al. Nat. Med. 9:129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (see, e.g., U.S. Pat. No. 6,248,516).

Antibody fragments can be made by various techniques, including but not limited to proteolytic digestion of an intact antibody as well as production by recombinant host cells (e.g., E. coli or phage), as described herein.

In some embodiments, the antibody fragment is used in combination with a second Sortilin antibody and/or with one or more antibodies that specifically bind a disease-causing protein selected from: amyloid beta or fragments thereof, Tau, IAPP, alpha-synuclein, TDP-43, FUS protein, prion protein, PrPSc, huntingtin, calcitonin, superoxide dismutase, ataxin, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, proline-arginine (PR) repeat peptides, and any combination thereof.

### (3) Chimeric and Humanized Antibodies

In some embodiments of any of the antibodies provided herein, the antibody is a chimeric antibody. Certain chimeric antibodies are described, e.g., in U.S. Pat. No. 4,816,567. In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In some embodiments of any of the antibodies provided herein, the antibody is a humanized antibody. Typically, a non-human antibody is humanized to reduce immunogenicity to humans, while retaining the specificity and affinity of the parental non-human antibody. In certain embodiments, a humanized antibody is substantially non-immunogenic in humans. In certain embodiments, a humanized antibody has substantially the same affinity for a target as an antibody from another species from which the humanized antibody is derived. See, e.g., U.S. Pat. Nos. 5,530,101, 5,693,761; 5,693,762; and 5,585,089. In certain embodiments, amino acids of an antibody variable domain that can be modified without diminishing the native affinity of the antigen binding domain while reducing its immunogenicity are identified. See, e.g., U.S. Pat. Nos. 5,766,886 and 5,869,619. Generally, a humanized antibody comprises one or more variable domains in which HVRs (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), for example, to restore or improve antibody specificity or affinity.

Humanized antibodies and methods of making them are reviewed, for example, in Almagro et al. Front. Biosci. 13:161 9-1633 (2008), and are further described, e.g., in U.S. Pat. Nos. 5,821,337, 7,527,791, 6,982,321, and 7,087,409. Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA 89:4285 (1992); and Presta et al., J. Immunol. 151:2623 (1993)); human mature (somatically mutated) framework regions or human germline framework regions (see, e.g., Almagro and Fransson Front. Biosci. 13:1619-1633 (2008)); and framework regions derived from screening FR libraries (see, e.g., Baca et al. J. Biol. Chem. 272:10678-10684 (1997) and Rosok et al. J. Biol. Chem. 271:22611-22618 (1996)).

### (4) Human Antibodies

In some embodiments of any of the antibodies provided herein, the antibody is a human antibody. Human antibodies can be produced using various techniques known in the art. Human antibodies are described generally in van Dijk et al. Curr. Opin. Pharmacol. 5:368-74 (2001) and Lonberg Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. One can engineer mouse strains deficient in mouse antibody production with large fragments of the human Ig loci in anticipation that such mice would produce human antibodies in the absence of mouse antibodies. Large human Ig fragments can preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains can yield high affinity fully human antibodies against any antigen of interest, including human antigens. Using the hybridoma technology, antigen-specific human MAbs with the desired specificity can be produced and selected. Certain exemplary methods are described in U.S. Pat. No. 5,545,807, EP 546073, and EP 546073. See also, for example, U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology. Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol. 133:3001 (1984) and Boerner et al. J. Immunol. 147:86 (1991)). Human antibodies generated via human B-cell hybridoma technology are also described in Li et al. Proc. Natl. Acad. Sci. USA, 1 03:3557-3562 (2006). Additional methods include those described, for example, in U.S. Pat. No. 7,189,826 (describing production of monoclonal human IgM antibodies from hybridoma cell lines). Human hybridoma technology (Trioma technology) is also described in Vollmers et al. Histology and Histopathology 20(3):927-937 (2005) and Vollmers et al. Methods and Findings in Experimental and Clinical Pharmacology 27(3):185-91 (2005). Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

In some embodiments of any of the antibodies provided herein, the antibody is a human antibody isolated by in vitro methods and/or screening combinatorial libraries for antibodies with the desired activity or activities. Suitable examples include but are not limited to phage display (CAT, Morphosys, Dyax, Biosite/Medarex, Xoma, Symphogen, Alexion (formerly Proliferon), Affimed), ribosome display (CAT), yeast-based platforms (Adimab), and the like. In certain phage display methods, repertoires of VH and VL genes are separately cloned by polymerase chain reaction (PCR) and recombined randomly in phage libraries, which can then be screened for antigen-binding phage as described in Winter et al. Ann. Rev. Immunol. 12: 433-455 (1994). For example, a variety of methods are known in the art for generating phage display libraries and screening such libraries for antibodies possessing the desired binding characteristics. See also Sidhu et al. J. Mol. Biol. 338(2): 299-310, 2004; Lee et al. J. Mol. Biol. 340(5): 1073-1093, 2004; Fellouse Proc. Natl. Acad. Sci. USA 101(34):12467-12472 (2004); and Lee et al. J. Immunol. Methods 284(−2):1 19-132 (2004). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self-antigens without any immunization as described by Griffiths et al. EMBO J. 12: 725-734 (1993). Finally, naive libraries can also be made synthetically by cloning unrearranged V-gene segments from stem cells, and using PCR primers comprising random sequence to encode the highly variable HVR3 regions and to accomplish rearrangement in vitro, as described by Hoogenboom et al. J. Mol. Biol., 227: 381-388, 1992. Patent publications describing human antibody phage libraries include, for example: U.S. Pat. No. 5,750,373, and US Patent Publication Nos. 2007/0292936 and 2009/0002360. Antibodies isolated from human antibody libraries are considered human antibodies or human antibody fragments herein.

(5) Constant Regions Including Fc regions

In some embodiments of any of the antibodies provided herein, the antibody comprises an Fc. In some embodiments, the Fc is a human IgG1, IgG2, IgG3, and/or IgG4 isotype. In some embodiments, the antibody is of the IgG class, the IgM class, or the IgA class.

In certain embodiments of any of the antibodies provided herein, the antibody has an IgG2 isotype. In some embodiments, the antibody contains a human IgG2 constant region. In some embodiments, the human IgG2 constant region includes an Fc region. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has an IgG1 isotype. In some embodiments, the antibody contains a mouse IgG1 constant region. In some embodiments, the antibody contains a human IgG1 constant region. In some embodiments, the human IgG1 constant region includes an Fc region. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has an IgG4 isotype. In some embodiments, the antibody contains a human IgG4 constant region. In some embodiments, the human IgG4 constant region includes an Fc region. In some embodiments, the antibody binds an inhibitory Fc receptor. In certain embodiments, the inhibitory Fc receptor is inhibitory Fc-gamma receptor IIB (FcγIIB).

In certain embodiments of any of the antibodies provided herein, the antibody has a hybrid IgG2/4 isotype. In some embodiments, the antibody includes an amino acid sequence comprising amino acids 118 to 260 according to EU numbering of human IgG2 and amino acids 261-447 according to EU numbering of human IgG4 (WO 1997/11971; WO 2007/106585).

In some embodiments, the Fc region increases clustering without activating complement as compared to a corresponding antibody comprising an Fc region that does not comprise the amino acid substitutions. In some embodiments, the antibody induces one or more activities of a target specifically bound by the antibody. In some embodiments, the antibody binds to Sortilin.

It may also be desirable to modify an anti-Sortilin antibody of the present disclosure to modify effector function and/or to increase serum half-life of the antibody. For example, the Fc receptor binding site on the constant region may be modified or mutated to remove or reduce binding affinity to certain Fc receptors, such as FcγRI, FcγRII, and/or FcγRIII to reduce antibody-dependent cell-mediated cytotoxicity. In some embodiments, the effector function is impaired by removing N-glycosylation of the Fc region (e.g., in the CH2 domain of IgG) of the antibody. In some embodiments, the effector function is impaired by modifying regions such as 233-236, 297, and/or 327-331 of human IgG as described in WO 99/58572 and Armour et al. Molecular Immunology 40: 585-593 (2003); Reddy et al. J. Immunology 164:1925-1933 (2000). In other embodiments, it may also be desirable to modify an anti-Sortilin antibody of the present disclosure to modify effector function to increase binding selectivity toward the ITIM-containing FcgRIIb (CD32b) to increase clustering of Sortilin antibodies on adjacent cells without activating humoral responses including antibody-dependent cell-mediated cytotoxicity and antibody-dependent cellular phagocytosis.

To increase the serum half-life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

### (6) Multispecific Antibodies

Multispecific antibodies are antibodies that have binding specificities for at least two different epitopes, including those on the same or another polypeptide (e.g., one or more Sortilin polypeptides of the present disclosure). In some embodiments, the multispecific antibody can be a bispecific antibody. In some embodiments, the multispecific antibody can be a trispecific antibody. In some embodiments, the multispecific antibody can be a tetraspecific antibody. Such antibodies can be derived from full-length antibodies or antibody fragments (e.g., F(ab′)₂ bispecific antibodies). In some embodiments, the multispecific antibody comprises a first antigen binding region which binds to a first site on Sortilin and comprises a second antigen binding region which binds to a second site on Sortilin. In some embodiments, the multispecific antibodies comprise a first antigen binding region which binds to Sortilin and a second antigen binding region that binds to a second polypeptide.

Provided herein are multispecific antibodies comprising a first antigen binding region, wherein the first antigen binding region comprises the six HVRs of an antibody described herein, which binds to Sortilin, and a second antigen binding region that binds to a second polypeptide. In some embodiments, the first antigen binding region comprises the V_{H} or V_{L} of an antibody described herein.

In some embodiments of any of the multispecific antibodies, the second polypeptide is a) an antigen facilitating transport across the blood-brain-barrier; (b) an antigen facilitating transport across the blood-brain-barrier selected from transferrin receptor (TR), insulin receptor (HIR), insulin-like growth factor receptor (IGFR), low-density lipoprotein receptor related proteins 1 and 2 (LPR-1 and 2), diphtheria toxin receptor, CRM197, a llama single domain antibody, TMEM 30(A), a protein transduction domain, TAT, Syn-B, penetratin, a poly-arginine peptide, an angiopep peptide, and ANG1005; (c) a disease-causing protein selected from amyloid beta, oligomeric amyloid beta, amyloid beta plaques, amyloid precursor protein or fragments thereof, Tau, IAPP, alpha-synuclein, TDP-43, FUS protein, C9orf72 (chromosome 9 open reading frame 72), c9RAN protein, prion protein, PrPSc, huntingtin, calcitonin, superoxide dismutase, ataxin, ataxin 1, ataxin 2, ataxin 3, ataxin 7, ataxin 8, ataxin 10, Lewy body, atrial natriuretic factor, islet amyloid polypeptide, insulin, apolipoprotein AI, serum amyloid A, medin, prolactin, transthyretin, lysozyme, beta 2 microglobulin, gelsolin, keratoepithelin, cystatin, immunoglobulin light chain AL, S-IBM protein, Repeat-associated non-ATG (RAN) translation products, DiPeptide repeat (DPR) peptides, glycine-alanine (GA) repeat peptides, glycine-proline (GP) repeat peptides, glycine-arginine (GR) repeat peptides, proline-alanine (PA) repeat peptides, ubiquitin, and proline-arginine (PR) repeat peptides; (d) ligands and/or proteins expressed on immune cells, wherein the ligands and/or proteins are selected from CD40, OX40, ICOS, CD28, CD137/4-1BB, CD27, GITR, PD-L1, CTLA-4, PD-L2, PD-1, B7-H3, B7-H4, HVEM, BTLA, KIR, GAL9, TIM3, A2AR, LAG-3, and phosphatidylserine; and/or (e) a protein, lipid, polysaccharide, or glycolipid expressed on one or more tumor cells; and any combination thereof.

Numerous antigens are known in the art that facilitate transport across the blood-brain barrier (see, e.g., Gabathuler R. Neurobiol. Dis. 37:48-57 (2010)). Such second antigens include, without limitation, transferrin receptor (TR), insulin receptor (HIR), Insulin-like growth factor receptor (IGFR), low-density lipoprotein receptor related proteins 1 and 2 (LPR-1 and 2), diphtheria toxin receptor, including CRM197 (a non-toxic mutant of diphtheria toxin), llama single domain antibodies such as TMEM 30(A) (Flippase), protein transduction domains such as TAT, Syn-B, or penetratin, poly-arginine or generally positively charged peptides, Angiopep peptides such as ANG1005 (see, e.g., Gabathuler, 2010), and other cell surface proteins that are enriched on blood-brain barrier endothelial cells (see, e.g., Daneman et al. PLoS One 5(10):e13741 (2010)).

The multivalent antibodies may recognize the Sortilin antigen as well as additional antigens, such as, without limitation, Aβ peptide antigen; an α-synuclein protein antigen; Tau protein antigen; TDP-43 protein antigen; prion protein antigen; huntingtin protein antigen; a RAN translation products antigen, including the DiPeptide Repeats (DPR peptides) composed of glycine-alanine (GA), glycine-proline (GP), glycine-arginine (GR), proline-alanine (PA), or proline-arginine (PR); Insulin receptor; insulin like growth factor receptor; Transferrin receptor; or any other antigen that facilitates antibody transfer across the blood brain barrier. In some embodiments, the second antigen is transferrin. In some embodiments, the second antigen is Tau. In some embodiments, the second antigen is Aβ. In some embodiments, the second antigen is TREM2. In some embodiments, the second antigen is α-synuclein.

The multivalent antibody contains at least one polypeptide chain (and preferably two polypeptide chains), wherein the polypeptide chain or chains comprise two or more variable domains. For instance, the polypeptide chain or chains may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. Similarly, the polypeptide chain or chains may comprise V_{H}-C_{H}1-flexible linker-V_{H}-C_{H}1-Fc region chain; or V_{H}-C_{H}1-V_{H}-C_{H}1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated herein comprise a light chain variable domain and, optionally, further comprise a CL domain.

Techniques for making multispecific antibodies include, but are not limited to, recombinant co-expression of two immunoglobulin heavy chain-light chain pairs having different specificities (see Milstein and Cuello Nature 305: 537 (1983), WO 93/08829, and Traunecker et al. EMBO J. 10:3655 (1991)), and “knob-in-hole” engineering (see, e.g., U.S. Pat. No. 5,731,168). See also WO 2013/026833 (CrossMab). Multi-specific antibodies may also be made by engineering electrostatic steering effects for making antibody Fc-heterodimeric molecules (WO 2009/089004A1); cross-linking two or more antibodies (see, e.g., U.S. Pat. No. 4,676,980); using leucine; using “diabody” technology for making bispecific antibody fragments (see, e.g., Hollinger et al. Proc. Natl. Acad. Sci. USA 90:6444-6448 (1993)); using single-chain Fv (scFv) dimers (see, e.g., Gruber et al. J. Immunol. 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g., US 2006/0025576). The antibody herein also includes a “Dual Acting FAb” or “DAF” comprising an antigen binding site that binds to multiple Sortilin antigens (see, US 2008/0069820, for example).

(7) Antibodies with Improved Stability

Amino acid sequence modifications of anti-Sortilin antibodies of the present disclosure, or antibody fragments thereof, to improve stability during manufacturing, storage, and in vivo administration, are also contemplated. For example, it may be desirable to reduce degradation of the antibodies or antibody fragments of the present disclosure through multiple pathways, including without limitation, oxidation and deamidation. Amino acid sequence variants of the antibodies or antibody fragments are prepared by introducing appropriate nucleotide changes into the nucleic acid encoding the antibodies or antibody fragments, or by peptide synthesis. Such modifications include, for example, deletions from, and/or insertions into and/or substitutions of, residues within the amino acid sequences of the antibody. Any combination of deletion, insertion, and substitution can be made to arrive at the final construct, provided that the final construct possesses the desired characteristics (i.e., reduced susceptibility to degradation).

In some embodiments, the asparagine (N33) site in the HVR-L1 region of an anti-Sortilin antibody of the present disclosure may be susceptible to degradation by means of deamidation. In certain embodiments, the asparagine (N33) site in the HVR-L1 region of S-60-15 (SEQ ID NO:8) may be susceptible to deamidation. Upon deamidation, the asparagine (N33) site in the HVR-L1 region of S-60-15 results in an Asn to Asp/IsoAsp change. In certain embodiments, the asparagine (N33) site in the HVR-L1 region of S-60-15 may be substituted to prevent or reduce deamidation. Non-limiting exemplary amino acid sequence variants of S-60-15 having amino acid substitutions in the asparagine (N33) site of the HVR-L1 region include S-60-15.1 [N33T], S-60-15.2 [N33S], S-60-15.3 [N33G], S-60-15.4 [N33R], S-60-15.5 [N33D], S-60-15.6 [N33H], S-60-15.7 [N33K], S-60-15.8 [N33Q], S-60-15.9 [N33Y], S-60-15.10 [N33E], S-60-15.11 [N33W], S-60-15.12 [N33F], S-60-15.13 [N33I], S-60-15.14 [N33V], S-60-15.15 [N33A], S-60-15.16 [N33M], or S-60-15.17 [N33L].

### (8) Antibody Variants

In some embodiments of any of the antibodies provided herein, amino acid sequence variants of the antibodies are contemplated. For example, it may be desirable to improve the binding affinity and/or other biological properties of the antibody.

(i) Substitution, Insertion, and Deletion Variants

In some embodiments of any of the antibodies provided herein, antibody variants having one or more amino acid substitutions are provided. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody.

TABLE A
Amino Acid Substitutions

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Leu; Val; Ile; Ala; Tyr | Tyr |
| Pro (P) | Ala | Ala |
| Ser (S) | Thr | Thr |
| Thr (T) | Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Substantial modifications in the biological properties of the antibody are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) Hydrophobic: Norleucine, Met, Ala, Val, Leu, Ile;
(2) Neutral hydrophilic: Cys, Ser, Thr, Asn, Gln;
(3) Acidic: Asp, Glu;
(4) Basic: His, Lys, Arg;
(5) Residues that influence chain orientation: Gly, Pro; and
(6) Aromatic: Trp, Tyr, Phe.

For example, non-conservative substitutions can involve the exchange of a member of one of these classes for a member from another class. Such substituted residues can be introduced, for example, into regions of a human antibody that are homologous with non-human antibodies, or into the non-homologous regions of the molecule.

In making changes to the polypeptide or antibody described herein, according to certain embodiments, the hydropathic index of amino acids can be considered. Each amino acid has been assigned a hydropathic index on the basis of its hydrophobicity and charge characteristics. They are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al. J. Mol. Biol., 157:105-131 (1982). It is known that certain amino acids can be substituted for other amino acids having a similar hydropathic index or score and still retain a similar biological activity. In making changes based upon the hydropathic index, in certain embodiments, the substitution of amino acids whose hydropathic indices are within ±2 is included. In certain embodiments, those which are within ±1 are included, and in certain embodiments, those within ±0.5 are included.

It is also understood in the art that the substitution of like amino acids can be made effectively on the basis of hydrophilicity, particularly where the biologically functional protein or peptide thereby created is intended for use in immunological embodiments, as in the present case. In certain embodiments, the greatest local average hydrophilicity of a protein, as governed by the hydrophilicity of its adjacent amino acids, correlates with its immunogenicity and antigenicity, i.e., with a biological property of the protein.

The following hydrophilicity values have been assigned to these amino acid residues: arginine (+3.0); lysine (+3.0±1); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5) and tryptophan (−3.4). In making changes based upon similar hydrophilicity values, in certain embodiments, the substitution of amino acids whose hydrophilicity values are within ±2 is included, in certain embodiments, those which are within ±1 are included, and in certain embodiments, those within ±0.5 are included. One can also identify epitopes from primary amino acid sequences on the basis of hydrophilicity. These regions are also referred to as “epitopic core regions”.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

Amino acid sequence insertions include amino- and/or carboxyl-terminal fusions ranging in length from one residue to polypeptides comprising a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT) or a polypeptide which increases the serum half-life of the antibody.

Any cysteine residue not involved in maintaining the proper conformation of the antibody also may be substituted, generally with serine, to improve the oxidative stability of the molecule and prevent aberrant crosslinking. Conversely, cysteine bond(s) may be added to the antibody to improve its stability (particularly where the antibody is an antibody fragment, such as an Fv fragment).

(ii) Glycosylation Variants

In some embodiments of any of the antibodies provided herein, the antibody is altered to increase or decrease the extent to which the antibody is glycosylated. Addition or deletion of glycosylation sites to an antibody may be conveniently accomplished by altering the amino acid sequence such that one or more glycosylation sites is created or removed.

Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites to the antibody is conveniently accomplished by altering the amino acid sequence such that it contains one or more of the above-described tripeptide sequences (for N-linked glycosylation sites). The alteration may also be made by the addition of, or substitution by, one or more serine or threonine residues to the sequence of the original antibody (for O-linked glycosylation sites).

Where the antibody comprises an Fc region, the carbohydrate attached thereto may be altered. Native antibodies produced by mammalian cells typically comprise a branched, biantennary oligosaccharide that is generally attached by an N-linkage to Asn297 according to Kabat numbering of the CH2 domain of the Fc region. The oligosaccharide may include various carbohydrates, for example, mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. See, e.g., US Patent Publication Nos. 2003/0157108 and 2004/0093621. Examples of publications related to “defucosylated” or “fucose-deficient” antibody variants include: US 2003/0157108; US 2003/0115614; US 2002/0164328; US 2004/0093621; US 2004/0132140; US 2004/0110704; US 2004/0110282; US 2004/0109865; Okazaki et al. J. Mol. Biol. 336:1239-1249 (2004); Yamane-Ohnuki et al. Biotech. Bioeng. 87:614 (2004). Examples of cell lines capable of producing defucosylated antibodies include Led 3 CHO cells deficient in protein fucosylation (Ripka et al. Arch. Biochem. Biophys. 249:533-545 (1986); US 2003/0157108), and knockout cell lines, such as alpha-1,6-fucosyltransferase gene, FUT8, knockout CHO cells (see, e.g., Yamane-Ohnuki et al. Biotech. Bioeng. 87: 614 (2004) and Kanda et al. Biotechnol. Bioeng. 94(4):680-688 (2006)).

(iii) Modified Constant Regions

In some embodiments of any of the antibodies provided herein, the antibody Fc comprises one or more modifications. In some embodiments, the antibody Fc, e.g., comprising one or more modifications, is capable of binding to Fc gamma receptor.

In some embodiments of any of the antibodies provided herein, the modified antibody Fc is an IgG1 modified Fc. In some embodiments, the IgG1 modified Fc comprises one or more modifications. For example, in some embodiments, the IgG1 modified Fc comprises one or more amino acid substitutions (e.g., relative to a wild-type Fc region of the same isotype). In some embodiments, the one or more amino acid substitutions are selected from N297A (Bolt S et al. (1993) Eur J Immunol 23:403-411), D265A (Shields et al. (2001) R. J. Biol. Chem. 276, 6591-6604), L234A, L235A (Hutchins et al. (1995) Proc Natl Acad Sci USA, 92:11980-11984; Alegre et al., (1994) Transplantation 57:1537-1543. 31; Xu et al., (2000) Cell Immunol, 200:16-26), G237A (Alegre et al. (1994) Transplantation 57:1537-1543. 31; Xu et al. (2000) Cell Immunol, 200:16-26), C226S, C229S, E233P, L234V, L234F, L235E (McEarchern et al., (2007) Blood, 109:1185-1192), P331S (Sazinsky et al., (2008) Proc Natl Acad Sci USA 2008, 105:20167-20172), S267E, L328F, A330L, M252Y, S254T, and/or T256E, wherein the amino acid position is according to the EU numbering convention. In some embodiments of any of the antibodies provided herein, the antibody is an IgG1 isotype and the Fc region comprises amino acid substitutions at positions L234A, L235A, and P331S, wherein the numbering of the residue position is according to EU numbering.

In some embodiments of any of the IgG1 modified Fc, the Fc comprises an N297A mutation according to EU numbering. In some embodiments of any of the IgG1 modified Fc, the Fc comprises D265A and N297A mutations according to EU numbering. In some embodiments of any of the IgG1 modified Fc, the Fc comprises a D270A mutation according to EU numbering. In some embodiments, the IgG1 modified Fc comprises L234A and L235A mutations according to EU numbering. In some embodiments of any of the IgG1 modified Fc, the Fc comprises L234A and G237A mutations according to EU numbering. In some embodiments of any of the IgG1 modified Fc, the Fc comprises L234A, L235A and G237A mutations according to EU numbering. In some embodiments of any of the IgG1 modified Fc, the Fc comprises one or more (including all) of P238D, L328E, E233, G237D, H268D, P271G and A330R mutations according to EU numbering. In some embodiments of any of the IgG1 modified Fc, the Fc comprises one or more of S267E/L328F mutations according to EU numbering. In some embodiments of any of the IgG1 modified Fc, the Fc comprises P238D, L328E, E233D, G237D, H268D, P271G and A330R mutations according to EU numbering. In some embodiments of any of the IgG1 modified Fc, the Fc comprises P238D, L328E, G237D, H268D, P271G and A330R mutations according to EU numbering. In some embodiments of any of the IgG1 modified Fc, the Fc comprises P238D, S267E, L328E, E233D, G237D, H268D, P271G and A330R mutations according to EU numbering. In some embodiments of any of the IgG1 modified Fc, the Fc comprises P238D, S267E, L328E, G237D, H268D, P271G and A330R mutations according to EU numbering. In some embodiments of any of the IgG1 modified Fc, the Fc comprises C226S, C229S, E233P, L234V, and L235A mutations according to EU numbering. In some embodiments of any of the IgG1 modified Fc, the Fc comprises L234F, L235E, and P331S mutations according to EU numbering. In some embodiments of any of the IgG1 modified Fc, the Fc comprises S267E and L328F mutations according to EU numbering. In some embodiments of any of the IgG1 modified Fc, the Fc comprises S267E mutations according to EU numbering. In some embodiments of any of the IgG1 modified Fc, the Fc comprises a substitute of the constant heavy 1 (CH1) and hinge region of IgG1 with CH1 and hinge region of IgG2 (amino acids 118-230 of IgG2 according to EU numbering) with a Kappa light chain.

In some embodiments of any of the IgG1 modified Fc, the Fc includes two or more amino acid substitutions that increase antibody clustering without activating complement as compared to a corresponding antibody having an Fc region that does not include the two or more amino acid substitutions. Accordingly, in some embodiments of any of the antibodies comprising an IgG1 modified Fc, the antibody comprises an Fc region, wherein the antibody comprises an amino acid substitution at position E430G and one or more amino acid substitutions in the Fc region at a residue position selected from: L234F, L235A, L235E, S267E, K322A, L328F, A330S, P331S, and any combination thereof according to EU numbering. In some embodiments, the IgG1 modified Fc comprises an amino acid substitution at positions E430G, L243A, L235A, and P331S according to EU numbering. In some embodiments, the IgG1 modified Fc comprises an amino acid substitution at positions E430G and P331S according to EU numbering. In some embodiments, the IgG1 modified Fc comprises an amino acid substitution at positions E430G and K322A according to EU numbering. In some embodiments, the IgG1 modified Fc comprises an amino acid substitution at positions E430G, A330S, and P331S according to EU numbering. In some embodiments, the IgG1 modified Fc comprises an amino acid substitution at positions E430G, K322A, A330S, and P331S according to EU numbering. In some embodiments, the IgG1 modified Fc comprises an amino acid substitution at positions E430G, K322A, and A330S according to EU numbering. In some embodiments, the IgG1 modified Fc comprises an amino acid substitution at positions E430G, K322A, and P331S according to EU numbering.

In some embodiments of any of the IgG1 modified Fc, the IgG1 modified Fc may further comprise an A330L mutation (Lazar et al. Proc Nal Acad Sci USA, 103:4005-4010 (2006)), or one or more of L234F, L235E, and/or P331S mutations (Sazinsky et al. Proc Nal Acad Sci USA, 105:20167-20172 (2008)), according to the EU numbering convention, to eliminate complement activation. In some embodiments of any of the IgG1 modified Fc, the IgG1 modified Fc may further comprise one or more of A330L, A330S, L234F, L235E, and/or P331S according to EU numbering. In some embodiments of any of the IgG1 modified Fc, the IgG1 modified Fc may further comprise one or more mutations to enhance the antibody half-life in human serum (e.g., one or more (including all) of M252Y, S254T, and T256E mutations according to the EU numbering convention). In some embodiments of any of the IgG1 modified Fc, the IgG1 modified Fc may further comprise one or more of E430G, E430S, E430F, E430T, E345K, E345Q, E345R, E345Y, S440Y, and/or S440W according to EU numbering.

Other aspects of the present disclosure relate to antibodies having modified constant regions (i.e., Fc regions). An antibody dependent on binding to FcgR receptor to activate targeted receptors may lose its agonist activity if engineered to eliminate FcgR binding (see, e.g., Wilson et al. Cancer Cell 19:101-113 (2011); Armour at al. Immunology 40:585-593 (2003); and White et al. Cancer Cell 27:138-148 (2015)). As such, it is thought that an anti-Sortilin antibody of the present disclosure with the correct epitope specificity can activate the target antigen, with minimal adverse effects, when the antibody has an Fc domain from a human IgG2 isotype (CH1 and hinge region) or another type of Fc domain that is capable of preferentially binding the inhibitory FcgRIIB receptors, or a variation thereof.

In some embodiments of any of the antibodies provided herein, the modified antibody Fc is an IgG2 modified Fc. In some embodiments, the IgG2 modified Fc comprises one or more modifications. For example, in some embodiments, the IgG2 modified Fc comprises one or more amino acid substitutions (e.g., relative to a wild-type Fc region of the same isotype). In some embodiments of any of the IgG2 modified Fc, the one or more amino acid substitutions are selected from V234A (Alegre et al. Transplantation 57:1537-1543 (1994); Xu et al. Cell Immunol, 200:16-26 (2000)); G237A (Cole et al. Transplantation, 68:563-571 (1999)); H268Q, V309L, A330S, P331S (US 2007/0148167; Armour et al. Eur J Immunol 29: 2613-2624 (1999); Armour et al. The Haematology Journal 1(Suppl. 1):27 (2000); Armour et al. The Haematology Journal 1(Suppl. 1):27 (2000)), C219S, and/or C220S (White et al. Cancer Cell 27, 138-148 (2015)); S267E, L328F (Chu et al. Mol Immunol, 45:3926-3933 (2008)); and M252Y, S254T, and/or T256E according to the EU numbering convention. In some embodiments of any of the IgG2 modified Fc, the Fc comprises an amino acid substitution at positions V234A and G237A according to EU numbering. In some embodiments of any of the IgG2 modified Fc, the Fc comprises an amino acid substitution at positions C219S or C220S according to EU numbering. In some embodiments of any of the IgG2 modified Fc, the Fc comprises an amino acid substitution at positions A330S and P331S according to EU numbering. In some embodiments of any of the IgG2 modified Fc, the Fc comprises an amino acid substitution at positions S267E and L328F according to EU numbering.

In some embodiments of any of the IgG2 modified Fc, the Fc comprises a C127S amino acid substitution according to the EU numbering convention (White et al., (2015) Cancer Cell 27, 138-148; Lightle et al. Protein Sci. 19:753-762 (2010); and WO 2008/079246). In some embodiments of any of the IgG2 modified Fc, the antibody has an IgG2 isotype with a Kappa light chain constant domain that comprises a C214S amino acid substitution according to the EU numbering convention (White et al. Cancer Cell 27:138-148 (2015); Lightle et al. Protein Sci. 19:753-762 (2010); and WO 2008/079246).

In some embodiments of any of the IgG2 modified Fc, the Fc comprises a C220S amino acid substitution according to the EU numbering convention. In some embodiments of any of the IgG2 modified Fc, the antibody has an IgG2 isotype with a Kappa light chain constant domain that comprises a C214S amino acid substitution according to the EU numbering convention.

In some embodiments of any of the IgG2 modified Fc, the Fc comprises a C219S amino acid substitution according to the EU numbering convention. In some embodiments of any of the IgG2 modified Fc, the antibody has an IgG2 isotype with a Kappa light chain constant domain that comprises a C214S amino acid substitution according to the EU numbering convention.

In some embodiments of any of the IgG2 modified Fc, the Fc includes an IgG2 isotype heavy chain constant domain 1(CH1) and hinge region (White et al. Cancer Cell 27:138-148 (2015)). In certain embodiments of any of the IgG2 modified Fc, the IgG2 isotype CH1 and hinge region comprise the amino acid sequence of 118-230 according to EU numbering. In some embodiments of any of the IgG2 modified Fc, the antibody Fc region comprises a S267E amino acid substitution, a L328F amino acid substitution, or both, and/or a N297A or N297Q amino acid substitution according to the EU numbering convention.

In some embodiments of any of the IgG2 modified Fc, the Fc further comprises one or more amino acid substitutions at positions E430G, E430S, E430F, E430T, E345K, E345Q, E345R, E345Y, S440Y, and S440W according to EU numbering. In some embodiments of any of the IgG2 modified Fc, the Fc may further comprise one or more mutations to enhance the antibody half-life in human serum (e.g., one or more (including all) of M252Y, S254T, and T256E mutations according to the EU numbering convention). In some embodiments of any of the IgG2 modified Fc, the Fc may further comprise A330S and P331S mutations.

In some embodiments of any of the IgG2 modified Fc, the Fc is an IgG2/4 hybrid Fc. In some embodiments, the IgG2/4 hybrid Fc comprises IgG2 amino acids 118 to 260 and IgG4 amino acids 261 to 447. In some embodiments of any IgG2 modified Fc, the Fc comprises one or more amino acid substitutions at positions H268Q, V309L, A330S, and P331S according to EU numbering.

In some embodiments of any of the IgG1 and/or IgG2 modified Fc, the Fc comprises one or more additional amino acid substitutions selected from A330L, L234F, L235E, or P331S according to EU numbering; and any combination thereof.

In certain embodiments of any of the IgG1 and/or IgG2 modified Fc, the Fc comprises one or more amino acid substitutions at a residue position selected from C127S, L234A, L234F, L235A, L235E, S267E, K322A, L328F, A330S, P331S, E345R, E430G, S440Y, and any combination thereof according to EU numbering. In some embodiments of any of the IgG1 and/or IgG2 modified Fc, the Fc comprises an amino acid substitution at positions E430G, L243A, L235A, and P331S according to EU numbering. In some embodiments of any of the IgG1 and/or IgG2 modified Fc, the Fc comprises an amino acid substitution at positions E430G and P331S according to EU numbering. In some embodiments of any of the IgG1 and/or IgG2 modified Fc, the Fc comprises an amino acid substitution at positions E430G and K322A according to EU numbering. In some embodiments of any of the IgG1 and/or IgG2 modified Fc, the Fc comprises an amino acid substitution at positions E430G, A330S, and P331S according to EU numbering. In some embodiments of any of the IgG1 and/or IgG2 modified Fc, the Fc comprises an amino acid substitution at positions E430G, K322A, A330S, and P331S according to EU numbering. In some embodiments of any of the IgG1 and/or IgG2 modified Fc, the Fc comprises an amino acid substitution at positions E430G, K322A, and A330S according to EU numbering. In some embodiments of any of the IgG1 and/or IgG2 modified Fc, the Fc comprises an amino acid substitution at positions E430G, K322A, and P331S according to EU numbering. In some embodiments of any of the IgG1 and/or IgG2 modified Fc, the Fc comprises an amino acid substitution at positions S267E and L328F according to EU numbering. In some embodiments of any of the IgG1 and/or IgG2 modified Fc, the Fc comprises an amino acid substitution at position C127S according to EU numbering. In some embodiments of any of the IgG1 and/or IgG2 modified Fc, the Fc comprises an amino acid substitution at positions E345R, E430G and S440Y according to EU numbering.

In some embodiments of any of the antibodies provided herein, the modified antibody Fc is an IgG4 modified Fc. In some embodiments, the IgG4 modified Fc comprises one or more modifications. For example, in some embodiments, the IgG4 modified Fc comprises one or more amino acid substitutions (e.g., relative to a wild-type Fc region of the same isotype). In some embodiments of any of the IgG4 modified Fc, the one or more amino acid substitutions are selected from L235A, G237A, S229P, L236E (Reddy et al. J Immunol 164:1925-1933(2000)), S267E, E318A, L328F, M252Y, S254T, and/or T256E according to the EU numbering convention. In some embodiments of any of the IgG4 modified Fc, the Fc may further comprise L235A, G237A, and E318A amino acid substitutions according to the EU numbering convention. In some embodiments of any of the IgG4 modified Fc, the Fc may further comprise S228P and L235E amino acid substitutions according to the EU numbering convention. In some embodiments of any of the IgG4 modified Fc, the IgG4 modified Fc may further comprise S267E and L328F amino acid substitutions according to the EU numbering convention.

In some embodiments of any of the IgG4 modified Fc, the IgG4 modified Fc comprises an S228P mutation according to the EU numbering convention (Angal et al. Mol Immunol. 30:105-108 (1993)) and/or one or more mutations described in (Peters et al. J Biol Chem. 287(29):24525-33 (2012)) to enhance antibody stabilization.

In some embodiments of any of the IgG4 modified Fc, the IgG4 modified Fc may further comprise one or more mutations to enhance the antibody half-life in human serum (e.g., one or more (including all) of M252Y, S254T, and T256E mutations according to the EU numbering convention).

In some embodiments of any of the IgG4 modified Fc, the Fc comprises an L235E amino acid substitution according to EU numbering. In certain embodiments of any of the IgG4 modified Fc, the Fc comprises one or more amino acid substitutions at a residue position selected from C127S, F234A, L235A, L235E, S267E, K322A, L328F, E345R, E430G, S440Y, and any combination thereof, according to EU numbering. In some embodiments of any of the IgG4 modified Fc, the Fc comprises an amino acid substitution at positions E430G, L243A, L235A, and P331S according to EU numbering. In some embodiments of any of the IgG4 modified Fc, the Fc comprises an amino acid substitution at positions E430G and P331S according to EU numbering. In some embodiments of any of the IgG4 modified Fc, the Fc comprises an amino acid substitution at positions E430G and K322A according to EU numbering. In some embodiments of any of the IgG4 modified Fc, the Fc comprises an amino acid substitution at position E430 according to EU numbering. In some embodiments of any of the IgG4 modified Fc, the Fc region comprises an amino acid substitution at positions E430G and K322A according to EU numbering. In some embodiments of any of the IgG4 modified Fc, the Fc comprises an amino acid substitution at positions S267E and L328F according to EU numbering. In some embodiments of any of the IgG4 modified Fc, the Fc comprises an amino acid substitution at position C127S according to EU numbering. In some embodiments of any of the IgG4 modified Fc, the Fc comprises an amino acid substitution at positions E345R, E430G and S440Y according to EU numbering.

### Nucleic Acids, Vectors, and Host Cells

Anti-Sortilin antibodies of the present disclosure may be produced using recombinant methods and compositions, e.g., as described in U.S. Pat. No. 4,816,567. In some embodiments, isolated nucleic acids having a nucleotide sequence encoding any of the anti-Sortilin antibodies of the present disclosure are provided. Such nucleic acids may encode an amino acid sequence comprising the V_{L} and/or an amino acid sequence comprising the V_{H} of the anti-Sortilin antibody (e.g., the light and/or heavy chains of the antibody). In some embodiments, one or more vectors (e.g., expression vectors) comprising such nucleic acids are provided. In some embodiments, a host cell comprising such nucleic acids or vectors is also provided. In some embodiments, the host cell comprises (e.g., has been transduced with): (1) a vector comprising a nucleic acid that encodes an amino acid sequence comprising the V_{L} of the antibody and an amino acid sequence comprising the V_{H} of the antibody, or (2) a first vector comprising a nucleic acid that encodes an amino acid sequence comprising the V_{L} of the antibody and a second vector comprising a nucleic acid that encodes an amino acid sequence comprising the V_{H} of the antibody. In some embodiments, the host cell is eukaryotic, e.g., a Chinese Hamster Ovary (CHO) cell or lymphoid cell (e.g., Y0, NS0, Sp20 cell). Host cells of the present disclosure also include, without limitation, isolated cells, in vitro cultured cells, and ex vivo cultured cells.

Methods of making an anti-Sortilin antibody of the present disclosure are provided. In some embodiments, the method includes culturing a host cell of the present disclosure comprising a nucleic acid encoding the anti-Sortilin antibody, under conditions suitable for expression of the antibody. In some embodiments, the antibody is subsequently recovered from the host cell (or host cell culture medium).

For recombinant production of an anti-Sortilin antibody of the present disclosure, a nucleic acid encoding the anti-Sortilin antibody is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable vectors comprising a nucleic acid sequence encoding any of the anti-Sortilin antibodies of the present disclosure, or cell-surface expressed fragments or polypeptides thereof (including antibodies) described herein include, without limitation, cloning vectors and expression vectors. Suitable cloning vectors can be constructed according to standard techniques, or may be selected from a large number of cloning vectors available in the art. While the cloning vector selected may vary according to the host cell intended to be used, useful cloning vectors generally have the ability to self-replicate, may possess a single target for a particular restriction endonuclease, and/or may carry genes for a marker that can be used in selecting clones comprising the vector. Suitable examples include plasmids and bacterial viruses, e.g., pUC18, pUC19, Bluescript (e.g., pBS SK+) and its derivatives, mpl8, mpl9, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Suitable host cells for cloning or expression of antibody-encoding vectors include prokaryotic or eukaryotic cells. For example, anti-Sortilin antibodies of the present disclosure may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. After expression, the antibody may be isolated from the bacterial cell paste in a soluble fraction and can be further purified.

In addition to prokaryotes, eukaryotic microorganisms, such as filamentous fungi or yeast, are also suitable cloning or expression hosts for antibody-encoding vectors, including fungi and yeast strains whose glycosylation pathways have been “humanized,” resulting in the production of an antibody with a partially or fully human glycosylation pattern (e.g., Gerngross Nat. Biotech. 22:1409-1414 (2004); and Li et al. Nat. Biotech. 24:210-215 (2006)).

Suitable host cells for the expression of glycosylated antibody can also be derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts (e.g., U.S. Pat. Nos. 5,959,177, 6,040,498, 6,420,548, 7,125,978, and 6,417,429, describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al. J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK); buffalo rat liver cells (BRL 3A); human lung cells (W138); human liver cells (Hep G2); mouse mammary tumor cells (MMT 060562); TRI cells, as described, e.g., in Mather et al. Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al. Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, NJ), pp. 255-268 (2003).

### Methods of Monitoring Treatment

Also provided herein are methods of monitoring the treatment of an individual being administered an anti-Sortilin antibody of the present disclosure.

In some embodiments, the methods comprise measuring the level of Progranulin protein in a sample of plasma from the individual before and after the individual has received one or more doses of an anti-Sortilin antibody. In some embodiments, the methods comprise measuring the level of Progranulin protein in a sample of cerebrospinal fluid from the individual before and after the individual has received one or more doses of an anti-Sortilin antibody. In some embodiments, the method further comprises a step of assessing the activity of the anti-Sortilin antibody in the individual based on the level of Progranulin protein in a sample from the individual. The level of Progranulin protein in a sample, e.g., a plasma or cerebrospinal fluid sample, may be measured using any suitable method known in the art, such as immunoblotting (e.g., Western blots), SOMASCAN assay (see, e.g., Candia et al. (2017) Sci Rep 7, 14248), mass spectrometry, flow cytometry, and enzyme-linked immunosorbent assay (ELISA). In some embodiments, the anti-Sortilin antibody is determined to be active in the individual if the level of Progranulin protein in a sample obtained after the individual has received one or more doses of the anti-Sortilin antibody is increased compared to the level of Progranulin protein in a sample obtained before the individual received one or more doses of the anti-Sortilin antibody.

In some embodiments, the methods comprise measuring the level of NfL in a sample of serum or plasma from the individual before and after the individual has received one or more doses of an anti-Sortilin antibody. In some embodiments, the methods comprise measuring the level of NfL in a sample of cerebrospinal fluid from the individual before and after the individual has received one or more doses of an anti-Sortilin antibody. In some embodiments, the method further comprises a step of assessing the activity of the anti-Sortilin antibody in the individual based on the level of NfL in a sample from the individual. The level of NfL in a sample, e.g., a serum, plasma, or cerebrospinal fluid sample, may be measured using any suitable method known in the art, such as immunoassays, a single-molecule array technology (Simoa) assay (e.g., using commercially available kits, such as the NF-light digital immunoassay kit or Simoa HD-1 assay from Quanterix, Lexinton, MA; or a Neurology 4-Plex A kit, see, e.g., Heller et al., J Neurol Neurosurg Psychiatry (2020) 91(3):263-270), ELISA, or using other assays from Quanterix or Roche Diagnostics. In some embodiments, the anti-Sortilin antibody is determined to be active in the individual if the level of NfL in a sample obtained after the individual has received one or more doses of the anti-Sortilin antibody is decreased compared to the level of NfL light chain in a sample obtained before the individual received one or more doses of the anti-Sortilin antibody.

In some embodiments, the methods comprise measuring the level of one or more biomarkers of neurodegeneration in a sample of whole blood, plasma, or CSF from the individual before and after the individual has received one or more doses of an anti-Sortilin antibody. In some embodiments, the method further comprises a step of assessing the activity of the anti-Sortilin antibody in the individual based on the level of the one or more biomarkers of neurodegeneration in a sample from the individual. Biomarkers of neurodegeneration may include, without limitation, NfL, Tau, and/or phosphorylated tau (pTau). The level of the one or more biomarkers of neurodegeneration in a sample, e.g., a whole blood, plasma, or CSF sample, may be measured using any suitable method known in the art, such as immunoblotting (e.g., Western blots), SOMASCAN assay (see, e.g., Candia et al. (2017) Sci Rep 7, 14248), mass spectrometry, flow cytometry, and ELISA.

In some embodiments, the methods comprise measuring the level of one or more biomarkers of lysosomal function in a sample of whole blood, plasma, or CSF from the individual before and after the individual has received one or more doses of an anti-Sortilin antibody. In some embodiments, the method further comprises a step of assessing the activity of the anti-Sortilin antibody in the individual based on the level of the one or more biomarkers of lysosomal function in a sample from the individual. In some embodiments, the one or more biomarkers of lysosomal function include, without limitation, N-acetylglucosamine kinase (NAGK), LAMP1, or one or more cathepsins, such as cathepsin B (CTSB) and/or cathepsin D. The level of the one or more biomarkers of lysosomal function in a sample, e.g., a whole blood, plasma, or CSF sample, may be measured using any suitable method known in the art, such as immunoblotting (e.g., Western blots), SOMASCAN assay (see, e.g., Candia et al. (2017) Sci Rep 7, 14248), mass spectrometry, flow cytometry, and ELISA.

In some embodiments, the methods comprise measuring the level of one or more biomarkers of complement activation or function in a sample of whole blood, plasma, or CSF from the individual before and after the individual has received one or more doses of an anti-Sortilin antibody. In some embodiments, the method further comprises a step of assessing the activity of the anti-Sortilin antibody in the individual based on the level of the one or more biomarkers of complement activation or function in a sample from the individual. In some embodiments, the one or more biomarkers of complement activation or function comprise Clqb and/or Clqc. The level of the one or more biomarkers of complement activation or function in a sample, e.g., a whole blood, plasma, or CSF sample, may be measured using any suitable method known in the art, such as immunoblotting (e.g., Western blots), SOMASCAN assay (see, e.g., Candia et al. (2017) Sci Rep 7, 14248), mass spectrometry, flow cytometry, and ELISA.

In some embodiments, the methods comprise measuring the level of one or more biomarkers of astrogliosis in a sample of whole blood, plasma, or CSF from the individual before and after the individual has received one or more doses of an anti-Sortilin antibody. In some embodiments, the method further comprises a step of assessing the activity of the anti-Sortilin antibody in the individual based on the level of the one or more biomarkers of astrogliosis in a sample from the individual. In some embodiments, the one or more biomarkers of astrogliosis include, without limitation, glial fibrillary acidic protein (GFAP). Non-limiting examples of methods that may be used to measure the levels of the one or more biomarkers of astrogliosis, e.g., GFAP, in a sample, e.g., in a whole blood, plasma, and/or CSF sample, include SOMASCAN assay (see, e.g., Candia et al. (2017) Sci Rep 7, 14248), Western blots, mass spectrometry (e.g., Multiple Reaction Monitoring Liquid Chromatography-Mass Spectrometry), flow cytometry, a single molecule array based-assay (e.g., a Simoa assay by Quanterix; see, e.g., the website: www.quanterix.com/simoa-technology/), and enzyme-linked immunosorbent assay (ELISA) assays.

In some embodiments, the methods comprise measuring the level of one or more biomarkers of neuroinflammation in a sample of whole blood, plasma, or CSF from the individual before and after the individual has received one or more doses of an anti-Sortilin antibody. In some embodiments, the method further comprises a step of assessing the activity of the anti-Sortilin antibody in the individual based on the level of the one or more biomarkers of neuroinflammation in a sample from the individual. In some embodiments, the one or more biomarkers of neuroinflammation include, without limitation, macrophage migration inhibitory factor (MIF). The level of the one or more biomarkers of neuroinflammation in a sample, e.g., a whole blood, plasma, or CSF sample, may be measured using any suitable method known in the art, such as immunoblotting (e.g., Western blots), SOMASCAN assay (see, e.g., Candia et al. (2017) Sci Rep 7, 14248), mass spectrometry, flow cytometry, and ELISA. In some embodiments, the levels of MIF protein in a CSF sample are measured using a quantitative ELISA method, such as the sandwich Enzyme-Linked Immunosorbent Assay described in Example 2 herein.

In some embodiments, the methods comprise measuring the level of one or more biomarkers of glial activity in a sample of whole blood, plasma, or CSF from the individual before and after the individual has received one or more doses of an anti-Sortilin antibody. In some embodiments, the method further comprises a step of assessing the activity of the anti-Sortilin antibody in the individual based on the level of the one or more biomarkers of glial activity in a sample from the individual. In some embodiments, the one or more biomarkers of glial activity include, without limitation, YKL40 and IL-6. The level of the one or more biomarkers of glial activity in a sample, e.g., a whole blood, plasma, or CSF sample, may be measured using any suitable method known in the art, such as immunoblotting (e.g., Western blots), SOMASCAN assay (see, e.g., Candia et al. (2017) Sci Rep 7, 14248), mass spectrometry, flow cytometry, and ELISA.

In some embodiments, the methods comprise assessing whole, global and/or regional brain volume in the individual before and after the individual has received one or more doses of an anti-Sortilin antibody. In some embodiments, the method further comprises a step of assessing the activity of the anti-Sortilin antibody in the individual based on whole, global and/or regional brain volume in the individual. Whole, global and/or regional brain volume may be assessed using any suitable method known in the art, such as using structural volumetric magnetic resonance imaging (MRI).

In some embodiments, the methods comprise assessing the volume of white matter hyperintensities in the individual before and after the individual has received one or more doses of an anti-Sortilin antibody. In some embodiments, the method further comprises a step of assessing the activity of the anti-Sortilin antibody in the individual based on volume of white matter hyperintensities in the individual. Volume of white matter hyperintensities may be assessed using any suitable method known in the art, such as using volumetric MRI.

In some embodiments, the methods comprise assessing brain perfusion in the individual before and after the individual has received one or more doses of an anti-Sortilin antibody. In some embodiments, the method further comprises a step of assessing the activity of the anti-Sortilin antibody in the individual based on brain perfusion in the individual. Brain perfusion may be assessed using any suitable method known in the art, such as using arterial spin labeling MRI.

In some embodiments, the methods comprise assessing fractional anisotropy, mean diffusivity, axial diffusivity, and/or radial diffusivity in the individual before and after the individual has received one or more doses of an anti-Sortilin antibody. In some embodiments, the method further comprises a step of assessing the activity of the anti-Sortilin antibody in the individual based on fractional anisotropy, mean diffusivity, axial diffusivity, and/or radial diffusivity in the individual. Fractional anisotropy, mean diffusivity, axial diffusivity, and/or radial diffusivity may be assessed using any suitable method known in the art, such as using diffusion-tensor imaging.

In some embodiments, the methods comprise performing one or more clinical outcome assessments on the individual before and after the individual has received one or more doses of an anti-Sortilin antibody. In some embodiments, the method further comprises a step of assessing the activity of the anti-Sortilin antibody in the individual based on a result of the one or more clinical outcome assessments. In some embodiments, the anti-Sortilin antibody is determined to be active in the individual if a result of the one or more clinical outcome assessments improves after the individual has received one or more doses of the anti-Sortilin antibody compared to a corresponding result before the individual received one or more doses of the anti-Sortilin antibody. In some embodiments, the anti-Sortilin antibody is determined to be active in the individual if a result of the one or more clinical outcome assessments remains stable after the individual has received one or more doses of the anti-Sortilin antibody compared to a corresponding result before the individual received one or more doses of the anti-Sortilin antibody. In some embodiments, the anti-Sortilin antibody is determined to be active in the individual if a result of the one or more clinical outcome assessments does not worsen after the individual has received one or more doses of the anti-Sortilin antibody compared to a corresponding result before the individual received one or more doses of the anti-Sortilin antibody. In some embodiments, the one or more clinical outcome assessments comprise the Frontotemporal Dementia Clinical Rating Scale (FCRS), the Frontotemporal Dementia Rating Scale (FRS), the Clinical Global Impression-Improvement (CGI-I) assessment, the Neuropsychiatric Inventory (NPI) assessment, the Color Trails Test (CTT) Part 2, the Repeatable Battery for the Assessment of Neuropsychological Status (RBANS), the Delis-Kaplan Executive Function System Color-Word Interference Test, the Interpersonal Reactivity Index, the Winterlight Lab Speech Assessment (WLA), the Summerlight Lab Speech Assessment (SLA), the Sheehan-Suicidality Tracking Scale (Sheehan-STS), the Clinical Global Impression-Severity (CGI-S), the Clinical Dementia Rating Dementia Staging Instrument PLUS National Alzheimer's Disease Coordinating Center frontotemporal lobar degeneration Behavior and Language Domains (CDR® plus NACC FTLD), the European Quality of Life-5 Dimensions (EQ-5D), the Clinical Dementia Rating Dementia Staging Instrument PLUS National Alzheimer's Disease Coordinating Center frontotemporal lobar degeneration Behavior and Language Domains Sum of Boxes (CDR® plus NACC FTLD SB), and the Zarit Burden Interview (ZBI).

### Pharmaceutical Compositions

Provided herein are pharmaceutical compositions and/or pharmaceutical formulations comprising the anti-Sortilin antibodies of the present disclosure and a pharmaceutically acceptable carrier.

In some embodiments, pharmaceutically acceptable carriers preferably are nontoxic to recipients at the dosages and concentrations employed. The antibodies described herein may be formulated into preparations in solid, semi-solid, liquid or gaseous forms. Examples of such formulations include, without limitation, tablets, capsules, powders, granules, ointments, solutions, suppositories, injections, inhalants, gels, microspheres, and aerosols. Pharmaceutically acceptable carriers can include, depending on the formulation desired, pharmaceutically-acceptable, non-toxic carriers of diluents, which are vehicles commonly used to formulate pharmaceutical compositions for animal or human administration. In certain embodiments, the pharmaceutical composition can comprise formulation materials for modifying, maintaining or preserving, for example, the pH, osmolarity, viscosity, clarity, color, isotonicity, odor, sterility, stability, rate of dissolution or release, adsorption or penetration of the composition.

In certain embodiments, pharmaceutically acceptable carriers include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. Further examples of formulations that are suitable for various types of administration can be found in Remington: The Science and Practice of Pharmacy, Pharmaceutical Press 22nd ed. (2013). For a brief review of methods for drug delivery, see, Langer, Science 249:1527-1533 (1990).

Formulations suitable for parenteral administration include aqueous and non-aqueous, isotonic sterile injection solutions, which can comprise antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

Formulations may be optimized for retention and stabilization in the brain or central nervous system. When the agent is administered into the cranial compartment, it is desirable for the agent to be retained in the compartment, and not to diffuse or otherwise cross the blood brain barrier. Stabilization techniques include cross-linking, multimerizing, or linking to groups such as polyethylene glycol, polyacrylamide, neutral protein carriers, etc., in order to achieve an increase in molecular weight.

Other strategies for increasing retention include the entrapment of the antibody, such as an anti-Sortilin antibody of the present disclosure, in a biodegradable or bioerodible implant. The rate of release of the therapeutically active agent is controlled by the rate of transport through the polymeric matrix, and the biodegradation of the implant. Implants may be particles, sheets, patches, plaques, fibers, microcapsules and the like and may be of any size or shape compatible with the selected site of insertion. Biodegradable polymeric compositions which may be employed may be organic esters or ethers, which when degraded result in physiologically acceptable degradation products, including the monomers. Anhydrides, amides, orthoesters or the like, by themselves or in combination with other monomers, may find use in the implants of the disclosure. The polymers may be condensation polymers. The polymers may be cross-linked or non-cross-linked. Of particular interest are polymers of hydroxyaliphatic carboxylic acids, either homo- or copolymers, and polysaccharides. Included among the polyesters of interest are polymers of D-lactic acid, L-lactic acid, racemic lactic acid, glycolic acid, polycaprolactone, and combinations thereof. Among the polysaccharides of interest are calcium alginate, and functionalized celluloses, particularly carboxymethylcellulose esters characterized by being water insoluble, a molecular weight of about 5 kD to 500 kD, etc. Biodegradable hydrogels may also be employed in the implants of the disclosure. Hydrogels are typically a copolymer material, characterized by the ability to imbibe a liquid.

### Kits/Articles of Manufacture

Provided herein are articles of manufacture (e.g., kits) comprising an anti-Sortilin antibody described herein. An article of manufacture of the disclosure may include one or more containers comprising an antibody described herein. Containers may be any suitable packaging including, but not limited to, vials, bottles, jars, flexible packaging (e.g., sealed Mylar or plastic bags), and the like. The containers may be unit doses, bulk packages (e.g., multi-dose packages) or sub-unit doses.

In some embodiments, the kits may further include a second agent. In some embodiments, the second agent is a pharmaceutically-acceptable buffer or diluting agent including, but not limited to, bacteriostatic water for injection (BWFI), phosphate-buffered saline, Ringer's solution and dextrose solution. In some embodiments, the second agent is a pharmaceutically active agent.

In some embodiments of any of the articles of manufacture, the article of manufacture further includes instructions for use in accordance with the methods of this disclosure. The instructions generally include information as to dosage, dosing schedule, and route of administration for the intended treatment. In some embodiments, these instructions comprise a description of administration of the isolated antibody of the present disclosure (e.g., an anti-Sortilin antibody described herein) to prevent, reduce risk, or treat an individual having a disease, disorder, or injury selected from dementia, frontotemporal dementia, Alzheimer's disease, gauche's disease, vascular dementia, seizures, retinal dystrophy, a traumatic brain injury, a spinal cord injury, atherosclerotic vascular diseases, undesirable symptoms of normal aging, amyotrophic lateral sclerosis (ALS), long-term depression, Parkinson's disease, Huntington's disease, Taupathy disease, multiple sclerosis, age related macular degeneration, glaucoma, degenerative disc disease (DDD), Creutzfeldt-Jakob disease, normal pressure hydrocephalus, Nasu-Hakola disease, stroke, acute trauma, chronic trauma, lupus, acute and chronic colitis, Crohn's disease, inflammatory bowel disease, ulcerative colitis, malaria, essential tremor, central nervous system lupus, Behcet's disease, mixed dementia, dementia with Lewy bodies, multiple system atrophy, Shy-Drager syndrome, progressive supranuclear palsy, cortical basal ganglionic degeneration, acute disseminated encephalomyelitis, granulomatous disorders, sarcoidosis, diseases of aging, retinitis pigmentosa, retinal degeneration, respiratory tract infection, sepsis, eye infection, systemic infection, lupus, arthritis, and wound healing, according to any methods of this disclosure. In some embodiments, the disease, disorder, or injury is frontotemporal dementia. In some embodiments, the instructions include instructions for use of the anti-Sortilin antibody and the second agent (e.g., second pharmaceutically active agent).

### Exemplary Embodiments

The following exemplary embodiments are representative of some aspects of the disclosure:

Exemplary Embodiment 1: A method of treating and/or delaying the progression of a disease or injury in an individual, comprising administering to the individual an anti-Sortilin antibody intravenously at a dose of about 60 mg/kg about once every four weeks, wherein the antibody comprises:
(i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33);
(iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32);
(vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33);
(vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or
(viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32).

Exemplary Embodiment 2: A method of treating and/or delaying the progression of a disease or injury in an individual, comprising administering to the individual an anti-Sortilin antibody intravenously at a dose of about 60 mg/kg about once every four weeks, wherein the antibody comprises:
a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32).

Exemplary Embodiment 3: A method of treating and/or delaying the progression of frontotemporal dementia in an individual at risk for developing symptomatic frontotemporal dementia, comprising administering to the individual an anti-Sortilin antibody intravenously at a dose of about 60 mg/kg about once every four weeks, wherein the individual has an elevated serum neurofilament light chain level, and wherein the antibody comprises:
(i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33);
(iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32);
(vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33);
(vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or
(viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32).

Exemplary Embodiment 4: A method of treating and/or delaying the progression of frontotemporal dementia in an individual at risk for developing symptomatic frontotemporal dementia, comprising administering to the individual an anti-Sortilin antibody intravenously at a dose of about 60 mg/kg about once every four weeks, wherein the individual has an elevated serum neurofilament light chain level, and wherein the antibody comprises:
a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32).

Exemplary Embodiment 5: A method of treating and/or delaying the progression of frontotemporal dementia in an individual, comprising administering to the individual an anti-Sortilin antibody intravenously at a dose of about 60 mg/kg about once every four weeks, wherein the antibody comprises:
(i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33);
(iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32);
(vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33);
(vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or
(viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32); and
wherein administration of the anti-Sortilin antibody to the individual results in a reduction or delay of frontotemporal dementia disease progression of at least about 10%, as compared to disease progression in a corresponding individual not treated with the anti-Sortilin antibody.

Exemplary Embodiment 6: A method of treating and/or delaying the progression of frontotemporal dementia in an individual, comprising administering to the individual an anti-Sortilin antibody intravenously at a dose of about 60 mg/kg about once every four weeks, wherein the antibody comprises:
a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32); and
wherein administration of the anti-Sortilin antibody to the individual results in a reduction or delay of frontotemporal dementia disease progression of at least about 10%, as compared to disease progression in a corresponding individual not treated with the anti-Sortilin antibody.

Exemplary Embodiment 7: An anti-Sortilin antibody for use in a method of treating and/or delaying the progression of a disease or injury in an individual, comprising administering the anti-Sortilin antibody to the individual at a dose of about 60 mg/kg intravenously about once every four weeks, wherein the anti-Sortilin antibody comprises:
(i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33);
(iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32);
(vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33);
(vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or
(viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32).

Exemplary Embodiment 8: An anti-Sortilin antibody for use in a method of treating and/or delaying the progression of a disease or injury in an individual, comprising administering the anti-Sortilin antibody to the individual at a dose of about 60 mg/kg intravenously about once every four weeks, wherein the anti-Sortilin antibody comprises:
a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32).

Exemplary Embodiment 9: An anti-Sortilin antibody for use in a method of treating and/or delaying the progression of frontotemporal dementia in an individual at risk for developing symptomatic frontotemporal dementia, comprising administering the anti-Sortilin antibody to the individual at a dose of about 60 mg/kg intravenously about once every four weeks, wherein the individual has an elevated serum neurofilament light chain level, and wherein the antibody comprises:
(i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33);
(iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32);
(vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33);
(vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or
(viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32).

Exemplary Embodiment 10: An anti-Sortilin antibody for use in a method of treating and/or delaying the progression of frontotemporal dementia in an individual at risk for developing symptomatic frontotemporal dementia, comprising administering the anti-Sortilin antibody to the individual at a dose of about 60 mg/kg intravenously about once every four weeks, wherein the individual has an elevated serum neurofilament light chain level, and wherein the antibody comprises:
a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32).

Exemplary Embodiment 11: An anti-Sortilin antibody for use in a method of treating and/or delaying the progression of frontotemporal dementia in an individual, comprising administering the anti-Sortilin antibody to the individual at a dose of about 60 mg/kg intravenously about once every four weeks, wherein the antibody comprises:
(i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33);
(iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32);
(vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33);
(vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or
(viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32); and
wherein administration of the anti-Sortilin antibody to the individual results in a reduction or delay of frontotemporal dementia disease progression of at least about 10%, as compared to disease progression in a corresponding individual not treated with the anti-Sortilin antibody.

Exemplary Embodiment 12: An anti-Sortilin antibody for use in a method of treating and/or delaying the progression of frontotemporal dementia in an individual, comprising administering the anti-Sortilin antibody to the individual at a dose of about 60 mg/kg intravenously about once every four weeks, wherein the antibody comprises:
a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32); and
wherein administration of the anti-Sortilin antibody to the individual results in a reduction or delay of frontotemporal dementia disease progression of at least about 10%, as compared to disease progression in a corresponding individual not treated with the anti-Sortilin antibody.

Exemplary Embodiment 13: The method of embodiment 5 or 6, or the anti-Sortilin antibody for use of embodiment 11 or 12, wherein frontotemporal dementia disease progression is assessed using the Clinical Dementia Rating Dementia Staging Instrument PLUS National Alzheimer's Disease Coordinating Center frontotemporal lobar degeneration Behavior and Language Domains Sum of Boxes (CDR® plus NACC FTLD-SB) assessment.

Exemplary Embodiment 14: The method of any one of embodiments 1, 3, 5 and 13, or the anti-Sortilin antibody for use of any one of embodiments 7, 9, 11 and 13, wherein the heavy chain variable region comprises an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and the light chain variable region comprises an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32).

Exemplary Embodiment 15: The method of any one of embodiments 1-6 and 13, or the anti-Sortilin antibody for use of any one of embodiments 7-13, wherein the heavy chain variable region comprises an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and the light chain variable region comprises an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32).

Exemplary Embodiment 16: The method of any one of embodiments 1, 3, 5 and 13, or the anti-Sortilin antibody for use of any one of embodiments 7, 9, 11 and 13, wherein the antibody comprises:
a heavy chain variable region comprising the amino acid sequence of SEQ ID NO: 54, and a light chain variable region comprising the amino acid sequence of SEQ ID NO: 57;
a heavy chain variable region comprising the amino acid sequence of SEQ ID NO: 54, and a light chain variable region comprising the amino acid sequence of SEQ ID NO: 58;
a heavy chain variable region comprising the amino acid sequence of SEQ ID NO: 54, and a light chain variable region comprising the amino acid sequence of SEQ ID NO: 59;
a heavy chain variable region comprising the amino acid sequence of SEQ ID NO: 55, and a light chain variable region comprising the amino acid sequence of SEQ ID NO: 57;
a heavy chain variable region comprising the amino acid sequence of SEQ ID NO: 55, and a light chain variable region comprising the amino acid sequence of SEQ ID NO: 58;
a heavy chain variable region comprising the amino acid sequence of SEQ ID NO: 56, and a light chain variable region comprising the amino acid sequence of SEQ ID NO: 57;
a heavy chain variable region comprising the amino acid sequence of SEQ ID NO: 56, and a light chain variable region comprising the amino acid sequence of SEQ ID NO: 60;
a heavy chain variable region comprising the amino acid sequence of SEQ ID NO: 56, and a light chain variable region comprising the amino acid sequence of SEQ ID NO: 77;
a heavy chain variable region comprising the amino acid sequence of SEQ ID NO: 56, and a light chain variable region comprising the amino acid sequence of SEQ ID NO: 78;
a heavy chain variable region comprising the amino acid sequence of SEQ ID NO: 54, and a light chain variable region comprising the amino acid sequence of SEQ ID NO: 79; or
a heavy chain variable region comprising the amino acid sequence of SEQ ID NO: 56, and a light chain variable region comprising the amino acid sequence of SEQ ID NO: 80.

Exemplary Embodiment 17: The method of any one of embodiments 1, 3, 5, and 13, or the anti-Sortilin antibody for use of any one of embodiments 7, 9, 11, and 13, wherein the antibody comprises:
(i) a heavy chain variable region comprising the amino acid sequence of SEQ ID NO: 56, and a light chain variable region comprising the amino acid sequence of SEQ ID NO: 57; or
(ii) a heavy chain variable region comprising the amino acid sequence of SEQ ID NO: 56, and a light chain variable region comprising the amino acid sequence of SEQ ID NO: 60.

Exemplary Embodiment 18: The method of any one of embodiments 1-6 and 13, or the anti-Sortilin antibody for use of any one of embodiments 7-13, wherein the antibody comprises a heavy chain comprising the amino acid sequence of SEQ ID NO: 90 or SEQ ID NO: 91, and a light chain comprising the amino acid sequence of SEQ ID NO: 95.

Exemplary Embodiment 19: The method of any one of embodiments 1-6 and 13-18, or the anti-Sortilin antibody for use of any one of embodiments 7-18, wherein the antibody has an IgG1 isotype and the Fc region comprises amino acid substitutions at positions L234A, L235A, and P331S, wherein the numbering of the residue position is according to EU numbering.

Exemplary Embodiment 20: The method of any one of embodiments 1-2 and 14-19, or the anti-Sortilin antibody for use of any one of embodiments 7-8 and 14-19, wherein the disease or injury is selected from the group consisting of frontotemporal dementia, progressive supranuclear palsy, Alzheimer's disease, vascular dementia, seizures, retinal dystrophy, amyotrophic lateral sclerosis, traumatic brain injury, a spinal cord injury, dementia, stroke, Parkinson's disease, acute disseminated encephalomyelitis, retinal degeneration, age related macular degeneration, glaucoma, multiple sclerosis, septic shock, bacterial infection, arthritis, and osteoarthritis.

Exemplary Embodiment 21: The method of any one of embodiments 1-2 and 14-20, or the anti-Sortilin antibody for use of any one of embodiments 7-8 and 14-20, wherein the disease or injury is frontotemporal dementia.

Exemplary Embodiment 22: The method of any one of embodiments 1-6 and 13-21, or the anti-Sortilin antibody for use of any one of embodiments 7-21, wherein the individual is heterozygous for a mutation in the Progranulin gene (GRN).

Exemplary Embodiment 23: The method or the anti-Sortilin antibody for use of embodiment 22, wherein the GRN mutation is a loss-of-function mutation.

Exemplary Embodiment 24: The method or the anti-Sortilin antibody for use of embodiment 22 or embodiment 23, wherein the GRN mutation is causative of frontotemporal dementia.

Exemplary Embodiment 25: The method of any one of embodiments 1-6 and 13-24, or the anti-Sortilin antibody for use of any one of embodiments 7-24, wherein the individual does not show symptoms of frontotemporal dementia prior to administration of the anti-Sortilin antibody.

Exemplary Embodiment 26: The method of any one of embodiments 5-6, 13-19 and 21-25, or the anti-Sortilin antibody for use of any one of embodiments 11-19 and 21-25, wherein the individual is at risk for developing symptomatic frontotemporal dementia prior to administration of the anti-Sortilin antibody.

Exemplary Embodiment 27: The method or the anti-Sortilin antibody for use of embodiment 26, wherein the individual has an elevated serum neurofilament light chain level prior to administration of the anti-Sortilin antibody.

Exemplary Embodiment 28: The method of any one of embodiments 3-4, 14-19, 22-25, and 27, or the anti-Sortilin antibody for use of any one of embodiments 9-10, 14-19, 22-25, and 27, wherein the elevated serum neurofilament light chain level comprises a serum neurofilament light chain level of at least about 13.6 μg/mL.

Exemplary Embodiment 29: The method of any one of embodiments 3-4, 14-19, 22-25, and 27, or the anti-Sortilin antibody for use of any one of embodiments 9-10, 14-19, 22-25, and 27, wherein the elevated serum neurofilament light chain level comprises a serum neurofilament light chain level of at least about 19.8 μg/mL.

Exemplary Embodiment 30: The method of any one of embodiments 3-6, 13-19, and 21-29, or the anti-Sortilin antibody for use of any one of embodiments 9-19 and 21-29, wherein the individual has a Clinical Dementia Rating Dementia Staging Instrument PLUS National Alzheimer's Disease Coordinating Center Frontotemporal Lobar Degeneration Behavior and Language Domains Sum of Boxes (CDR plus NACC FTLD-SB) score of 0.5 or less prior to administration of the anti-Sortilin antibody.

Exemplary Embodiment 31: The method of any one of embodiments 5-6, 13-19, and 21-24, or the anti-Sortilin antibody for use of any one of embodiments 11-19 and 21-24, wherein the individual has symptomatic frontotemporal dementia prior to administration of the anti-Sortilin antibody.

Exemplary Embodiment 32: The method of any one of embodiments 5-6, 13-19, 21-24, and 31, or the anti-Sortilin antibody for use of any one of embodiments 11-19, 21-24 and 31, wherein the individual has a CDR plus NACC FTLD-SB score greater than 0.5 prior to administration of the anti-Sortilin antibody.

Exemplary Embodiment 33: The method of any one of embodiments 1-6 and 13-21, or the anti-Sortilin antibody for use of any one of embodiments 7-21, wherein the individual is heterozygous for a hexanucleotide repeat expansion C9orf72 mutation.

Exemplary Embodiment 34: The method or the anti-Sortilin antibody for use of embodiment 33, wherein the hexanucleotide repeat expansion C9orf72 mutation is causative of FTD.

Exemplary Embodiment 35: The method or the anti-Sortilin antibody for use of embodiment 33 or embodiment 34, wherein the individual has symptomatic frontotemporal dementia prior to administration of the anti-Sortilin antibody.

Exemplary Embodiment 36: The method of any one of embodiments 5-6, 13-19, 21-24, and 31-35, or the anti-Sortilin antibody for use of any one of embodiments 11-19, 21-24 and 31-35, wherein the individual has one or more symptoms required for a diagnosis of possible behavioral variant frontotemporal dementia (bvFTD) prior to administration of the anti-Sortilin antibody.

Exemplary Embodiment 37: The method or the anti-Sortilin antibody for use of embodiment 36, wherein the one or more symptoms are selected from the group consisting of: disinhibition, apathy or inertia, loss of sympathy or empathy, perseverative or compulsive behaviors, hyperorality, and dysexecutive neuropsychological profile.

Exemplary Embodiment 38: The method of any one of embodiments 5-6, 13-19, 21-24, and 31-35, or the anti-Sortilin antibody for use of any one of embodiments 11-19, 21-24 and 31-35, wherein the individual has a diagnosis of primary progressive aphasia (PPA) prior to administration of the anti-Sortilin antibody.

Exemplary Embodiment 39: The method of any one of embodiments 3-6, 13-19, and 21-38, or the anti-Sortilin antibody for use of any one of embodiments 9-19 and 21-38, wherein the individual has a Clinical Dementia Rating Dementia Staging Instrument PLUS National Alzheimer's Disease Coordinating Center frontotemporal lobar degeneration Behavior and Language Domains (CDR plus NACC FTLD) score of between 0 and 2 prior to administration of the anti-Sortilin antibody.

Exemplary Embodiment 40: The method or the anti-Sortilin antibody for use of embodiment 39, wherein the individual has a CDR plus NACC FTLD score of 0.5, 1, or 2.

Exemplary Embodiment 41: The method of any one of embodiments 1-6 and 13-40, or the anti-Sortilin antibody for use of any one of embodiments 7-40, wherein the individual is treated for a treatment period of 96 weeks.

Exemplary Embodiment 42: The method or the anti-Sortilin antibody for use of embodiment 41, wherein administration of the anti-Sortilin antibody occurs on the first day of the treatment period and every four weeks thereafter.

Exemplary Embodiment 43: The method or the anti-Sortilin antibody for use of embodiment 41 or embodiment 42, wherein a total of 25 doses of the anti-Sortilin antibody are administered to the individual during the treatment period.

Exemplary Embodiment 44: The method or the anti-Sortilin antibody for use of any one of embodiments 41-43, further comprising continuing administration of the anti-Sortilin antibody to the individual once every four weeks after the end of the 96-week treatment period.

Exemplary Embodiment 45: The method or the anti-Sortilin antibody for use of embodiment 44, wherein administration of the anti-Sortilin antibody to the individual continues once every four weeks for up to 96 weeks.

Exemplary Embodiment 46: The method or the anti-Sortilin antibody for use of embodiment 44 or embodiment 45, wherein administration of the anti-Sortilin antibody to the individual continues once every four weeks for up to 25 doses.

Exemplary Embodiment 47: The method of any one of embodiments 1-6 and 13-46, or the anti-Sortilin antibody for use of any one of embodiments 7-46, wherein the individual is a human adult.

Exemplary Embodiment 48: The method of any one of embodiments 1-6 and 13-47, or the anti-Sortilin antibody for use of any one of embodiments 7-47, further comprising assessing the individual for the presence of one or more GRN mutations prior to administration of the anti-Sortilin antibody.

Exemplary Embodiment 49: The method of any one of embodiments 1-6 and 13-48, or the anti-Sortilin antibody for use of any one of embodiments 7-48, further comprising assessing the individual for the presence of a hexanucleotide repeat expansion C9orf72 mutation prior to administration of the anti-Sortilin antibody.

Exemplary Embodiment 50: The method of any one of embodiments 1-6 and 13-49, or the anti-Sortilin antibody for use of any one of embodiments 7-49, further comprising assessing the individual for the presence of an elevated level of neurofilament light chain prior to administration of the anti-Sortilin antibody to the individual, wherein the level of neurofilament light chain is assessed in a sample of serum obtained from the individual.

Exemplary Embodiment 51: The method of any one of embodiments 1-6 and 13-50, or the anti-Sortilin antibody for use of any one of embodiments 7-50, further comprising performing one or more clinical outcome assessments on the individual before and after the individual has received one or more doses of the anti-Sortilin antibody, wherein the one or more clinical outcome assessments are selected from the group consisting of: CDR plus NACC FTLD, CDR plus NACC FTLD-SB, Clinical Global Impression-Severity (CGI-S), Clinical Global Impression Improvement (CGI I), Repeatable Battery for the Assessment of Neuropsychological Status (RBANS), European Quality of Life-5 Dimensions (EQ 5D), Zarit Burden Interview (ZBI), Resource Utilization in Dementia-Lite Version (RUD Lite), Frontotemporal Dementia Rating Scale (FRS), and Winterlight Labs Speech Assessment (WLA).

Exemplary Embodiment 52: The method or the anti-Sortilin antibody for use of embodiment 51, wherein the disease or injury is frontotemporal dementia, and wherein the individual exhibits a reduction or delay of frontotemporal dementia disease progression of at least about 10% after the individual has received one or more doses of the anti-Sortilin antibody, as compared to disease progression in a corresponding individual not treated with the anti-Sortilin antibody, wherein frontotemporal dementia disease progression is assessed using the CDR® plus NACC FTLD-SB clinical outcome assessment.

Exemplary Embodiment 53: The method of any one of embodiments 5, 6 and 52, or the anti-Sortilin antibody for use of any one of embodiments 11, 12 and 52, wherein the reduction or delay of frontotemporal dementia disease progression comprises a reduction or delay of at least about 15%, at least about 20%, at least about 25%, at least about 30%, at least about 35%, at least about 40%, at least about 45%, at least about 50%, or at least about 55%, as compared to disease progression in a corresponding individual not treated with the anti-Sortilin antibody.

Exemplary Embodiment 54: The method of any one of embodiments 5, 6 and 52, or the anti-Sortilin antibody for use of any one of embodiments 11, 12 and 52, wherein the reduction or delay of frontotemporal dementia disease progression comprises a reduction or delay of at least about 47% or at least about 54%, as compared to disease progression in a corresponding individual not treated with the anti-Sortilin antibody.

Exemplary Embodiment 55: The method of any one of embodiments 1-6 and 13-54, or the anti-Sortilin antibody for use of any one of embodiments 7-54, further comprising measuring the level of Progranulin protein (PGRN) in a sample of blood plasma obtained from the individual before and after the individual has received one or more doses of the anti-Sortilin antibody.

Exemplary Embodiment 56: The method or the anti-Sortilin antibody for use of embodiment 55, wherein the individual has one or more GRN mutations, and before receiving one or more doses of the anti-Sortilin antibody, the individual has a PGRN plasma level that is lower than normal PGRN plasma levels observed in controls.

Exemplary Embodiment 57: The method or the anti-Sortilin antibody for use of embodiment 55 or embodiment 56, wherein the individual has one or more GRN mutations, and after receiving one or more doses of the anti-Sortilin antibody, the individual has a PGRN plasma level that is elevated compared to the plasma level of PGRN in the individual prior to administration of the anti-Sortilin antibody.

Exemplary Embodiment 58: The method or the anti-Sortilin antibody for use of embodiment 55 or embodiment 56, wherein the individual has one or more GRN mutations, and wherein administration of one or more doses of the anti-Sortilin antibody to the individual results in restored PGRN levels to normal levels for the duration of treatment with the anti-Sortilin antibody.

Exemplary Embodiment 59: The method or the anti-Sortilin antibody for use of any one of embodiments 55-58, wherein the individual has one or more GRN mutations, and after receiving one or more doses of the anti-Sortilin antibody, the individual has a PGRN plasma level that is within the range of normal PGRN plasma levels observed in controls.

Exemplary Embodiment 60: The method or the anti-Sortilin antibody for use of embodiment 59, wherein the PGRN plasma level that is within the range of normal PGRN plasma levels observed in controls is sustained after two or more doses of the anti-Sortilin antibody.

Exemplary Embodiment 61: The method or the anti-Sortilin antibody for use of embodiment 55, wherein the individual has a hexanucleotide repeat expansion C9orf72 mutation, and after receiving one or more doses of the anti-Sortilin antibody, the individual has a PGRN plasma level that is elevated compared to PGRN plasma levels in the individual prior to administration of the anti-Sortilin antibody.

Exemplary Embodiment 62: The method of any one of embodiments 1-6 and 13-61, or the anti-Sortilin antibody for use of any one of embodiments 7-61, further comprising measuring the level of neurofilament light chain in a sample of serum or plasma obtained from the individual before and after the individual has received one or more doses of the anti-Sortilin antibody.

Exemplary Embodiment 63: The method of any one of embodiments 1-6 and 13-62, or the anti-Sortilin antibody for use of any one of embodiments 7-62, further comprising measuring the level of Progranulin protein (PGRN) in a sample of cerebrospinal fluid (CSF) obtained from the individual before and after the individual has received one or more doses of the anti-Sortilin antibody.

Exemplary Embodiment 64: The method or the anti-Sortilin antibody for use of embodiment 63, wherein the individual has one or more GRN mutations, and before receiving one or more doses of the anti-Sortilin antibody, the individual has a CSF PGRN level that is lower than normal CSF PGRN levels observed in controls.

Exemplary Embodiment 65: The method or the anti-Sortilin antibody for use of embodiment 63 or embodiment 64, wherein the individual has one or more GRN mutations, and after receiving one or more doses of the anti-Sortilin antibody, the individual has a CSF level of PGRN that is elevated compared to the CSF level of PGRN in the individual prior to administration of the anti-Sortilin antibody.

Exemplary Embodiment 66: The method or the anti-Sortilin antibody for use of any one of embodiments 63-65, wherein the individual has one or more GRN mutations, and after receiving one or more doses of the anti-Sortilin antibody, the individual has a CSF level of PGRN that is within the range of normal CSF levels of PGRN observed in controls.

Exemplary Embodiment 67: The method or the anti-Sortilin antibody for use of embodiment 63, wherein the individual has a hexanucleotide repeat expansion C9orf72 mutation, and after receiving one or more doses of the anti-Sortilin antibody, the individual has a CSF level of PGRN that is elevated compared to the CSF level of PGRN in the individual prior to administration of the anti-Sortilin antibody.

Exemplary Embodiment 68: The method of any one of embodiments 1-6 and 13-67, or the anti-Sortilin antibody for use of any one of embodiments 7-67, further comprising measuring the level of neurofilament light chain in a sample of cerebrospinal fluid obtained from the individual before and after the individual has received one or more doses of the anti-Sortilin antibody.

Exemplary Embodiment 69: The method of any one of embodiments 1-6 and 13-68, or the anti-Sortilin antibody for use of any one of embodiments 7-68, further comprising measuring the level of one or more biomarkers of neurodegeneration in a sample of whole blood, plasma, or cerebrospinal fluid obtained from the individual before and after the individual has received one or more doses of the anti-Sortilin antibody.

Exemplary Embodiment 70: The method or the anti-Sortilin antibody for use of embodiment 69, wherein the one or more biomarkers of neurodegeneration comprise tau and phosphorylated tau.

Exemplary Embodiment 71: The method of any one of embodiments 1-6 and 13-70, or the anti-Sortilin antibody for use of any one of embodiments 7-70, further comprising measuring the level of one or more biomarkers of lysosomal function and/or one or more biomarkers of complement activation in a sample of whole blood, plasma, or cerebrospinal fluid obtained from the individual before and after the individual has received one or more doses of the anti-Sortilin antibody.

Exemplary Embodiment 72: The method or the anti-Sortilin antibody for use of embodiment 71, wherein the one or more biomarkers of lysosomal function comprise one or more cathepsins.

Exemplary Embodiment 73: The method or the anti-Sortilin antibody for use of embodiment 71 or 72, wherein the levels of the one or more biomarkers of lysosomal function and/or the one or more biomarkers of complement activation are normalized in the individual after administration of the one or more doses of the anti-Sortilin antibody.

Exemplary Embodiment 74: The method or the anti-Sortilin antibody for use of any one of embodiments 72-73, wherein the one or more cathepsins comprise cathepsin D.

Exemplary Embodiment 75: The method or the anti-Sortilin antibody for use of embodiment 72 or embodiment 74, wherein the levels of the one or more cathepsins are decreased to normal levels in a sample from the individual after administration of one or more doses of the anti-Sortilin antibody as compared to a control.

Exemplary Embodiment 76: The method or the anti-Sortilin antibody for use of embodiment 71, wherein the one or more biomarkers of lysosomal function comprise LAMP1.

Exemplary Embodiment 77: The method or the anti-Sortilin antibody for use of embodiment 76, wherein the levels of LAMP1 are decreased to normal levels in a sample from the individual after administration of one or more doses of the anti-Sortilin antibody as compared to a control.

Exemplary Embodiment 78: The method or the anti-Sortilin antibody for use of embodiment 71, wherein the one or more biomarkers of complement activation comprise C1QB.

Exemplary Embodiment 79: The method or the anti-Sortilin antibody for use of embodiment 78, wherein the levels of C1QB are decreased to normal levels in a sample from the individual after administration of one or more doses of the anti-Sortilin antibody as compared to a control.

Exemplary Embodiment 80: The method of any one of embodiments 1-6 and 13-79, or the anti-Sortilin antibody for use of any one of embodiments 7-79, further comprising measuring the level of one or more biomarkers of glial activity in a sample of whole blood, plasma, or cerebrospinal fluid obtained from the individual before and after the individual has received one or more doses of the anti-Sortilin antibody.

Exemplary Embodiment 81: The method or the anti-Sortilin antibody for use of embodiment 80, wherein the one or more biomarkers of glial activity comprise YKL40 and IL-6.

Exemplary Embodiment 82: The method of any one of embodiments 1-6 and 13-81, or the anti-Sortilin antibody for use of any one of embodiments 7-81, further comprising assessing global and regional brain volumes in the individual before and after the individual has received one or more doses of the anti-Sortilin antibody.

Exemplary Embodiment 83: The method or the anti-Sortilin antibody for use of embodiment 82, wherein the individual exhibits a reduction in ventricle enlargement after receiving one or more doses of the anti-Sortilin antibody.

Exemplary Embodiment 84: The method of any one of embodiments 1-6 and 13-83, or the anti-Sortilin antibody for use of any one of embodiments 7-83, further comprising assessing volume of white matter hyperintensities in the individual before and after the individual has received one or more doses of the anti-Sortilin antibody.

Exemplary Embodiment 85: The method of any one of embodiments 1-6 and 13-84, or the anti-Sortilin antibody for use of any one of embodiments 7-84, further comprising assessing brain perfusion in the individual before and after the individual has received one or more doses of the anti-Sortilin antibody.

Exemplary Embodiment 86: The method of any one of embodiments 1-6 and 13-85, or the anti-Sortilin antibody for use of any one of embodiments 7-85, further comprising assessing fractional anisotropy, mean diffusivity, axial diffusivity, and/or radial diffusivity in the individual before and after the individual has received one or more doses of the anti-Sortilin antibody.

Exemplary Embodiment 87: The method of any one of embodiments 1-6 and 13-86, or the anti-Sortilin antibody for use of any one of embodiments 7-86, further comprising measuring the level of one or more biomarkers of astrogliosis in a sample of whole blood, plasma, or cerebrospinal fluid obtained from the individual before and after the individual has received one or more doses of the anti-Sortilin antibody.

Exemplary Embodiment 88: The method or the anti-Sortilin antibody for use of embodiment 87, wherein the one or more biomarkers of astrogliosis comprise glial fibrillary acidic protein (GFAP).

Exemplary Embodiment 89: The method or the anti-Sortilin antibody for use of embodiment 88, wherein the levels of GFAP are decreased in a sample from the individual after the individual has received one or more doses of the anti-Sortilin antibody, as compared to the levels of GFAP in a sample from the individual before the individual received one or more doses of the anti-Sortilin antibody.

Exemplary Embodiment 90: The method of any one of embodiments 1-6 and 13-89, or the anti-Sortilin antibody for use of any one of embodiments 7-89, further comprising measuring the level of one or more biomarkers of neuroinflammation in a sample of cerebrospinal fluid (CSF) obtained from the individual before and after the individual has received one or more doses of the anti-Sortilin antibody.

Exemplary Embodiment 91: The method or the anti-Sortilin antibody for use of embodiment 90, wherein the one or more biomarkers of neuroinflammation comprise macrophage migration inhibitory factor (MIF).

Exemplary Embodiment 92: The method or the anti-Sortilin antibody for use of embodiment 91, wherein the levels of MIF in a sample of CSF from the individual after the individual has received one or more doses of the anti-Sortilin antibody are decreased, as compared to levels of MIF in a sample of CSF from the individual before the individual received one or more doses of the anti-Sortilin antibody.

Exemplary Embodiment 93: The method of any one of embodiments 1-6 and 13-92, or the anti-Sortilin antibody for use of any one of embodiments 7-92, further comprising measuring the level of the anti-Sortilin antibody in a sample of blood or cerebrospinal fluid obtained from the individual before and after the individual has received one or more doses of the anti-Sortilin antibody.

Exemplary Embodiment 94: A method of monitoring treatment of an individual being administered an anti-Sortilin antibody, comprising performing one or more clinical outcome assessments on the individual before and after the individual has received one or more doses of an anti-Sortilin antibody, wherein the one or more clinical outcome assessments are selected from the group consisting of: CDR plus NACC FTLD, CDR plus NACC FTLD-SB, CGI-S, CGI I, RBANS, EQ 5D, ZBI, RUD Lite, FRS, and WLA.

Exemplary Embodiment 95: The method of embodiment 94, wherein the clinical outcome assessment is the CDR plus NACC FTLD-SB.

Exemplary Embodiment 96: A method of monitoring treatment of an individual being administered an anti-Sortilin antibody, comprising measuring the level of Progranulin protein (PGRN) in a sample obtained from the individual before and after the individual has received one or more doses of an anti-Sortilin antibody.

Exemplary Embodiment 97: The method of embodiment 96, wherein the sample is a blood plasma sample or a cerebrospinal fluid sample.

Exemplary Embodiment 98: A method of monitoring treatment of an individual being administered an anti-Sortilin antibody, comprising measuring the level of neurofilament light chain in a sample obtained from the individual before and after the individual has received one or more doses of an anti-Sortilin antibody.

Exemplary Embodiment 99: The method of embodiment 98, wherein the sample is a serum sample or a cerebrospinal fluid sample.

Exemplary Embodiment 100: A method of monitoring treatment of an individual being administered an anti-Sortilin antibody, comprising measuring the level of one or more biomarkers of neurodegeneration, lysosomal function, complement activation, astrogliosis, neuroinflammation, or glial activity in a sample obtained from the individual before and after the individual has received one or more doses of an anti-Sortilin antibody.

Exemplary Embodiment 101: The method of embodiment 100, wherein the sample is a whole blood, plasma, or cerebrospinal fluid sample.

Exemplary Embodiment 102: The method of any one of embodiments 100-101, wherein the one or more biomarkers of neurodegeneration comprise tau and phosphorylated tau.

Exemplary Embodiment 103: The method of any one of embodiments 100-101, wherein the one or more biomarkers of lysosomal function comprise one or more cathepsins.

Exemplary Embodiment 104: The method of embodiment 103, wherein the one or more cathepsins comprise cathepsin D.

Exemplary Embodiment 105: The method of any one of embodiments 100-101, wherein the one or more biomarkers of glial activity comprise YKL40 and IL-6.

Exemplary Embodiment 106: The method of any one of embodiments 100-101, wherein the one or more biomarkers of astrogliosis comprise GFAP.

Exemplary Embodiment 107: The method of any one of embodiments 100-101, wherein the one or more biomarkers of neuroinflammation comprise macrophage migration inhibitory factor (MIF).

Exemplary Embodiment 108: The method of any one of embodiments 100-101, wherein the one or more biomarkers of complement activation comprise C1QB.

Exemplary Embodiment 109: A method of monitoring treatment of an individual being administered an anti-Sortilin antibody, comprising assessing global and regional brain volumes, volume of white matter hyperintensities, brain perfusion, fractional anisotropy, mean diffusivity, axial diffusivity, and/or radial diffusivity in the individual before and after the individual has received one or more doses of an anti-Sortilin antibody.

Exemplary Embodiment 110: The method of any one of embodiments 94-109, wherein the anti-Sortilin antibody comprises:
a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and
a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32).

Exemplary Embodiment 111: The method of any one of embodiments 94-110, wherein the anti-Sortilin antibody comprises a heavy chain variable region comprising the amino acid sequence of SEQ ID NO: 56, and a light chain variable region comprising the amino acid sequence of SEQ ID NO: 60.

Exemplary Embodiment 112: The method of any one of embodiments 94-111, wherein the antibody comprises a heavy chain comprising the amino acid sequence of SEQ ID NO: 90 or SEQ ID NO: 91, and a light chain comprising the amino acid sequence of SEQ ID NO: 95.

Exemplary Embodiment 113: The method of any one of embodiments 94-112, wherein the antibody has an IgG1 isotype and the Fc region comprises amino acid substitutions at positions L234A, L235A, and P331S, wherein the numbering of the residue position is according to EU numbering.

Exemplary Embodiment 114: An anti-sortilin antibody at a dose of about 60 mg/kg intravenously about once every four weeks for use in a method of treating and/or delaying the progression of a disease or injury in an individual, wherein the antibody comprises:
(i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33);
(iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32);
(vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33);
(vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or
(viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32).

Exemplary Embodiment 115: An anti-sortilin antibody at a dose of about 60 mg/kg intravenously about once every four weeks for use in a method of treating and/or delaying the progression of frontotemporal dementia in an individual at risk for developing symptomatic frontotemporal dementia, wherein the individual has an elevated serum neurofilament light chain level, and wherein the antibody comprises:
(i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33);
(iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32);
(vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33);
(vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or
(viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32).

Exemplary Embodiment 116: A use of an anti-sortilin antibody at a dose of about 60 mg/kg intravenously about once every four weeks in the manufacture of a medicament for treating and/or delaying the progression of a disease or injury in an individual, wherein the antibody comprises:
(i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33);
(iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32);
(vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33);
(vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or
(viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32).

Exemplary Embodiment 117: A use of an anti-sortilin antibody at a dose of about 60 mg/kg intravenously about once every four weeks in the manufacture of a medicament for treating and/or delaying the progression of frontotemporal dementia in an individual at risk for developing symptomatic frontotemporal dementia, wherein the individual has an elevated serum neurofilament light chain level, and wherein the antibody comprises:
(i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33);
(iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32);
(vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33);
(vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or
(viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32).

Exemplary Embodiment 118: A method of treating and/or delaying the progression of a disease or injury in an individual, comprising administering to the individual an anti-Sortilin antibody intravenously at a dose of about 60 mg/kg about once every four weeks, wherein the antibody comprises:
(i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33);
(iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32);
(vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33);
(vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or
(viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32);
wherein the method further comprises measuring the level of macrophage migration inhibitory factor (MIF) in a sample of whole blood, plasma or cerebrospinal fluid (CSF) obtained from the individual before and/or after the individual has received one or more doses of the anti-Sortilin antibody.

Exemplary Embodiment 119: A method of treating and/or delaying the progression of a disease or injury in an individual, comprising administering to the individual an anti-Sortilin antibody intravenously at a dose of about 60 mg/kg about once every four weeks, wherein the antibody comprises:
a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32);
wherein the method further comprises measuring the level of macrophage migration inhibitory factor (MIF) in a sample of whole blood, plasma or cerebrospinal fluid (CSF) obtained from the individual before and/or after the individual has received one or more doses of the anti-Sortilin antibody.

Exemplary Embodiment 120: An anti-Sortilin antibody for use in a method of treating and/or delaying the progression of a disease or injury in an individual, comprising administering the anti-Sortilin antibody to the individual at a dose of about 60 mg/kg intravenously about once every four weeks, wherein the anti-Sortilin antibody comprises:
(i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33);
(iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32);
(vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33);
(vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or
(viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32);
wherein the method further comprises measuring the level of macrophage migration inhibitory factor (MIF) in a sample of whole blood, plasma or cerebrospinal fluid (CSF) obtained from the individual before and/or after the individual has received one or more doses of the anti-Sortilin antibody.

Exemplary Embodiment 121: An anti-Sortilin antibody for use in a method of treating and/or delaying the progression of a disease or injury in an individual, comprising administering the anti-Sortilin antibody to the individual at a dose of about 60 mg/kg intravenously about once every four weeks, wherein the anti-Sortilin antibody comprises: a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32);
wherein the method further comprises measuring the level of macrophage migration inhibitory factor (MIF) in a sample of whole blood, plasma or cerebrospinal fluid (CSF) obtained from the individual before and/or after the individual has received one or more doses of the anti-Sortilin antibody.

Exemplary Embodiment 122: The method of any one of embodiments 118-119, or the anti-Sortilin antibody for use of any one of embodiments 120-121, wherein the sample is a sample of CSF.

Exemplary Embodiment 123: The method or the anti-Sortilin antibody for use of embodiment 122, wherein the levels of MIF in a sample of CSF obtained from the individual after the individual has received one or more doses of the anti-Sortilin antibody are decreased, as compared to levels of MIF in a sample of CSF from the individual before the individual received one or more doses of the anti-Sortilin antibody.

### Sequences of the Disclosure

TABLE 1
Heavy chain HVR H1 sequences of anti-Sortilin antibodies

| Ab(s) | HVR H1 | SEQ ID NO: |
|---|---|---|
| S-60; S-60-10; S-60-11; S-60-12; S-60-13; S-60-14; S-60-15 | YSISSGYYWG | 1 |
| [N33(wt)]; S-60-15.1 [N33T]; S-60-15.2 [N33S]; S-60-15.3 | | |
| [N33G]; S-60-15.4 [N33R]; S-60-15.5 [N33D]; S-60-15.6 | | |
| [N33H]; S-60-15.7 [N33K]; S-60-15.8 [N33Q]; S-60-15.9 | | |
| [N33Y]; S-60-15.10 [N33E]; S-60-15.11 [N33W]; S-60-15.12 | | |
| [N33F]; S-60-15.13 [N33I]; S-60-15.14 [N33V]; S-60-15.15 | | |
| [N33A]; S-60-15.16 [N33M]; S-60-15.17 [N33L]; S-60-16; S- | | |
| 60-18; S-60-19; S-60-24 | | |

TABLE 2
Heavy chain HVR H2 sequences of anti-Sortilin antibodies

| Ab(s) | HVR H2 | SEQ ID NO: |
|---|---|---|
| S-60; S-60-10; S-60-11; S-60-12; S-60-15 [N33(wt)]; S-60- | TIYHSGSTYYNPSL | 2 |
| 15.1 [N33T]; S-60-15.2 [N33S]; S-60-15.3 [N33G]; S-60-15.4 | KS | |
| [N33R]; S-60-15.5 [N33D]; S-60-15.6 [N33H]; S-60-15.7 | | |
| [N33K]; S-60-15.8 [N33Q]; S-60-15.9 [N33Y]; S-60-15.10 | | |
| [N33E]; S-60-15.11 [N33W]; S-60-15.12 [N33F]; S-60-15.13 | | |
| [N33I]; S-60-15.14 [N33V]; S-60-15.15 [N33A]; S-60-15.16 | | |
| [N33M]; S-60-15.17 [N33L]; S-60-16; S-60-18; S-60-19; S-60- | | |
| 24 | | |
| S-60-13; S-60-14 | TIYHSGSTYYNPSL | 3 |
| | ES | |
| Formula I | TIYHSGSTYYNPSL | 4 |
| | X₁S | |
| | X₁ is K or E | |

TABLE 3
Heavy chain HVR H3 sequences of anti-Sortilin antibodies

| Ab(s) | HVR H3 | SEQ ID NO: |
|---|---|---|
| S-60-10; S-60-11; S-60-12; S-60-13; S-60-14; S-60-19 | ARQGSIQQGYYGM | 5 |
| | DV | |
| S-60; S-60-15 [N33(wt)]; S-60-15.1 [N33T]; S-60-15.2 | ARQGSIKQGYYGM | 6 |
| [N33S]; S-60-15.3 [N33G]; S-60-15.4 [N33R]; S-60-15.5 | DV | |
| [N33D]; S-60-15.6 [N33H]; S-60-15.7 [N33K]; S-60-15.8 | | |
| [N33Q]; S-60-15.9 [N33Y]; S-60-15.10 [N33E]; S-60-15.11 | | |
| [N33W]; S-60-15.12 [N33F]; S-60-15.13 [N33I]; S-60-15.14 | | |
| [N33V]; S-60-15.15 [N33A]; S-60-15.16 [N33M]; S-60-15.17 | | |
| [N33L]; S-60-16; S-60-18; S-60-24 | | |
| Formula II | ARQGSIX₁QGYYGM | 7 |
| | DV | |
| | X₁ is Q or K | |

TABLE 4
Light chain HVR L1 sequences of antibodies

| Ab(s) | HVR L1 | SEQ ID NO: |
|---|---|---|
| S-60-10; S-60-11; S-60-12; S-60-13; | RSSQSLLRSNGYNY | 8 |
| S-60-14; S-60-15 [N33(wt)]; S-60-16; | LD | |
| S-60-18 | | |
| S-60-15.1 [N33T] | RSSQSLLRSTGYNYL | 9 |
| | D | |
| S-60-15.2 [N33S] | RSSQSLLRSSGYNYL | 10 |
| | D | |
| S-60-15.3 [N33G] | RSSQSLLRSGGYNY | 11 |
| | LD | |
| S-60-15.4 [N33R] | RSSQSLLRSRGYNY | 12 |
| | LD | |
| S-60-15.5 [N33D] | RSSQSLLRSDGYNY | 13 |
| | LD | |
| S-60-15.6 [N33H] | RSSQSLLRSHGYNY | 14 |
| | LD | |
| S-60-15.7 [N33K] | RSSQSLLRSKGYNY | 15 |
| | LD | |
| S-60-15.8 [N33Q] | RSSQSLLRSQGYNY | 16 |
| | LD | |
| S-60-15.9 [N33Y] | RSSQSLLRSYGYNY | 17 |
| | LD | |
| S-60-15.10 [N33E] | RSSQSLLRSEGYNYL | 18 |
| | D | |
| S-60-15.11 [N33W] | RSSQSLLRSWGYNY | 19 |
| | LD | |
| S-60-15.12 [N33F] | RSSQSLLRSFGYNYL | 20 |
| | D | |
| S-60-15.13 [N33I] | RSSQSLLRSIGYNYL | 21 |
| | D | |
| S-60-15.14 [N33V] | RSSQSLLRSVGYNY | 22 |
| | LD | |
| S-60-15.15 [N33A] | RSSQSLLRSAGYNY | 23 |
| | LD | |
| S-60-15.16 [N33M] | RSSQSLLRSMGYNY | 24 |
| | LD | |
| S-60-15.17 [N33L] | RSSQSLLRSLGYNYL | 25 |
| | D | |
| S-60; S-60-19 | RSSQSLLHSNGYNY | 26 |
| | LD | |
| S-60-24 | RSSQGLLRSNGYNY | 27 |
| | LD | |
| Formula III | RSSQX₁LLX₂SX₃GYN | 28 |
| | YLD | |
| | X₁ is S or G | |
| | X₂ is R or H | |
| | X₃ is N, T, S, G, R, D, | |
| | H, K, Q, Y, E, W, F, I, | |
| | V, A, M, or L | |

TABLE 5
Light chain HVR L2 sequences of anti-Sortilin antibodies

| Ab(s) | HVR L2 | SEQ ID NO: |
|---|---|---|
| S-60; S-60-10; S-60-11; S-60-13; S-60-14; S-60-15 [N33 | LGSNRAS | 29 |
| (wt)]; S-60-15.1 [N33T]; S-60-15.2 [N33S]; S-60-15.3 | | |
| [N33G]; S-60-15.4 [N33R]; S-60-15.5 [N33D]; S-60-15.6 | | |
| [N33H]; S-60-15.7 [N33K]; S-60-15.8 [N33Q]; S-60-15.9 | | |
| [N33Y]; S-60-15.10 [N33E]; S-60-15.11 [N33W]; S-60-15.12 | | |
| [N33F]; S-60-15.13 [N33I]; S-60-15.14 [N33V]; S-60-15.15 | | |
| [N33A]; S-60-15.16 [N33M]; S-60-15.17 [N33L]; S-60-16; S- | | |
| 60-18; S-60-19; S-60-24 | | |
| S-60-12 | LGSNRVS | 30 |
| Formula IV | LGSNRX₁S | 31 |
| | X₁ is A or V | |

TABLE 6
Light chain HVR L3 sequences of anti-Sortilin antibodies

| Ab(s) | HVR L3 | SEQ ID NO: |
|---|---|---|
| S-60-10; S-60-11; S-60-13; S-60-14; S-60-15 [N33(wt)]; S- | MQQQEAPLT | 32 |
| 60-15.1 [N33T]; S-60-15.2 [N33S]; S-60-15.3 [N33G]; S-60- | | |
| 15.4 [N33R]; S-60-15.5 [N33D]; S-60-15.6 [N33H]; S-60-15.7 | | |
| [N33K]; S-60-15.8 [N33Q]; S-60-15.9 [N33Y]; S-60-15.10 | | |
| [N33E]; S-60-15.11 [N33W]; S-60-15.12 [N33F]; S-60-15.13 | | |
| [N33I]; S-60-15.14 [N33V]; S-60-15.15 [N33A]; S-60-15.16 | | |
| [N33M]; S-60-15.17 [N33L]; S-60-16; S-60-24 | | |
| S-60; S-60-12; S-60-18; S-60-19 | MQQQETPLT | 33 |
| Formula V | MQQQEX₁PLT | 34 |
| | X₁ is A or T | |

TABLE 7
Heavy chain framework 1 sequences of anti-Sortilin antibodies

| Ab(s) | VH FR1 | SEQ ID NO: |
|---|---|---|
| S-60-10; S-60-11; S-60-12; S-60-13; S-60-14; S-60-15 [N33 | QVQLQESGPGLVKP | 35 |
| (wt)]; S-60-15.1 [N33T]; S-60-15.2 [N33S]; S-60-15.3 | SETLSLTCAVSG | |
| [N33G]; S-60-15.4 [N33R]; S-60-15.5 [N33D]; S-60-15.6 | | |
| [N33H]; S-60-15.7 [N33K]; S-60-15.8 [N33Q]; S-60-15.9 | | |
| [N33Y]; S-60-15.10 [N33E]; S-60-15.11 [N33W]; S-60-15.12 | | |
| [N33F]; S-60-15.13 [N33I]; S-60-15.14 [N33V]; S-60-15.15 | | |
| [N33A]; S-60-15.16 [N33M]; S-60-15.17 [N33L]; S-60-16; S- | | |
| 60-18; S-60-19; S-60-24 | | |

TABLE 8
Heavy chain framework 2 sequences of anti-Sortilin antibodies

| Ab(s) | VH FR2 | SEQ ID NO: |
|---|---|---|
| S-60-10; S-60-11; S-60-12; S-60-13; S-60-14; S-60-15 [N33 | WIRQPPGKGLEWIG | 36 |
| (wt)]; S-60-15.1 [N33T]; S-60-15.2 [N33S]; S-60-15.3 | | |
| [N33G]; S-60-15.4 [N33R]; S-60-15.5 [N33D]; S-60-15.6 | | |
| [N33H]; S-60-15.7 [N33K]; S-60-15.8 [N33Q]; S-60-15.9 | | |
| [N33Y]; S-60-15.10 [N33E]; S-60-15.11 [N33W]; S-60-15.12 | | |
| [N33F]; S-60-15.13 [N33I]; S-60-15.14 [N33V]; S-60-15.15 | | |
| [N33A]; S-60-15.16 [N33M]; S-60-15.17 [N33L]; S-60-16; S- | | |
| 60-18; S-60-19; S-60-24 | | |

TABLE 9
Heavy chain framework 3 sequences of anti-Sortilin antibodies

| Ab(s) | VH FR3 | SEQ ID NO: |
|---|---|---|
| S-60-10; S-60-11; S-60-12; S-60-19 | QVTISVDTSKNQFSL | 37 |
| | ELSSVTAADTAVYY | |
| | C | |
| S-60-13; S-60-14; S-60-15 [N33(wt)]; S-60-15.1 [N33T]; S- | RVTISVDTSKNQFSL | 38 |
| 60-15.2 [N33S]; S-60-15.3 [N33G]; S-60-15.4 [N33R]; S-60- | KLSSVTAADTAVYY | |
| 15.5 [N33D]; S-60-15.6 [N33H]; S-60-15.7 [N33K]; S-60- | C | |
| 15.8 [N33Q]; S-60-15.9 [N33Y]; S-60-15.10 [N33E]; S-60- | | |
| 15.11 [N33W]; S-60-15.12 [N33F]; S-60-15.13 [N33I]; S-60- | | |
| 15.14 [N33V]; S-60-15.15 [N33A]; S-60-15.16 [N33M]; S-60- | | |
| 15.17 [N33L]; S-60-16; S-60-18; S-60-24 | | |
| Formula VI | X₁VTISVDTSKNQFS | 39 |
| | LX₂LSSVTAADTAVY | |
| | YC | |
| | X₁ is Q or R | |
| | X₂ is E or K | |

TABLE 10
Heavy chain framework 4 sequences of anti-Sortilin antibodies

| Ab(s) | VH FR4 | SEQ ID NO: |
|---|---|---|
| S-60-10; S-60-11; S-60-12; S-60-13; S-60-14; S-60-15 [N33 | WGQGTTVTVSS | 40 |
| (wt)]; S-60-15.1 [N33T]; S-60-15.2 [N33S]; S-60-15.3 | | |
| [N33G]; S-60-15.4 [N33R]; S-60-15.5 [N33D]; S-60-15.6 | | |
| [N33H]; S-60-15.7 [N33K]; S-60-15.8 [N33Q]; S-60-15.9 | | |
| [N33Y]; S-60-15.10 [N33E]; S-60-15.11 [N33W]; S-60-15.12 | | |
| [N33F]; S-60-15.13 [N33I]; S-60-15.14 [N33V]; S-60-15.15 | | |
| [N33A]; S-60-15.16 [N33M]; S-60-15.17 [N33L]; S-60-16; S- | | |
| 60-18; S-60-19; S-60-24 | | |

TABLE 11
Light chain framework 1 sequences of anti-Sortilin antibodies

| Ab(s) | VL FR1 | SEQ ID NO: |
|---|---|---|
| S-60-10; S-60-11; S-60-12; S-60-13; S-60-14; S-60-15 [N33 | DIVMTQSPLSLPVTP | 41 |
| (wt)]; S-60-15.1 [N33T]; S-60-15.2 [N33S]; S-60-15.3 | GEPASISC | |
| [N33G]; S-60-15.4 [N33R]; S-60-15.5 [N33D]; S-60-15.6 | | |
| [N33H]; S-60-15.7 [N33K]; S-60-15.8 [N33Q]; S-60-15.9 | | |
| [N33Y]; S-60-15.10 [N33E]; S-60-15.11 [N33W]; S-60-15.12 | | |
| [N33F]; S-60-15.13 [N33I]; S-60-15.14 [N33V]; S-60-15.15 | | |
| [N33A]; S-60-15.16 [N33M]; S-60-15.17 [N33L]; S-60-16; S- | | |
| 60-19 | | |
| S-60-18 | DIVMTQSPLSLPVTP | 42 |
| | GGPASISC | |
| S-60-24 | DIVMTQSPLSLPVTP | 43 |
| | GESASISC | |
| Formula VII | DIVMTQSPLSLPVTP | 44 |
| | GX₁X₂ASISC | |
| | X₁ is E or G | |
| | X₂ is Por S | |

TABLE 12
Light chain framework 2 sequences of anti-Sortilin antibodies

| Ab(s) | VL FR2 | SEQ ID NO: |
|---|---|---|
| S-60-10; S-60-11; S-60-13; S-60-14; S-60-15 [N33(wt)]; S- | WYLQKPGQSPQLLI | 45 |
| 60-15.1 [N33T]; S-60-15.2 [N33S]; S-60-15.3 [N33G]; S-60- | Y | |
| 15.4 [N33R]; S-60-15.5 [N33D]; S-60-15.6 [N33H]; S-60-15.7 | | |
| [N33K]; S-60-15.8 [N33Q]; S-60-15.9 [N33Y]; S-60-15.10 | | |
| [N33E]; S-60-15.11 [N33W]; S-60-15.12 [N33F]; S-60-15.13 | | |
| [N33I]; S-60-15.14 [N33V]; S-60-15.15 [N33A]; S-60-15.16 | | |
| [N33M]; S-60-15.17 [N33L]; S-60-16; S-60-18; S-60-19; S- | | |
| 60-24 | | |
| S-60-12 | WYLQKPGQPPQLLI | 46 |
| | Y | |
| Formula VIII | WYLQKPGQX₁PQLLI | 47 |
| | Y | |
| | X₁ is S or P | |

TABLE 13
Light chain framework 3 sequences of anti-Sortilin antibodies

| Ab(s) | VL FR3 | SEQ ID NO: |
|---|---|---|
| S-60-10; S-60-13; S-60-15 [N33(wt)]; S-60-15.1 [N33T]; S- | GVPDRFSGSGSGTD | 48 |
| 60-15.2 [N33S]; S-60-15.3 [N33G]; S-60-15.4 [N33R]; S-60- | FTLKISRAEAEDVGV | |
| 15.5 [N33D]; S-60-15.6 [N33H]; S-60-15.7 [N33K]; S-60- | YYC | |
| 15.8 [N33Q]; S-60-15.9 [N33Y]; S-60-15.10 [N33E]; S-60- | | |
| 15.11 [N33W]; S-60-15.12 [N33F]; S-60-15.13 [N33I]; S-60- | | |
| 15.14 [N33V]; S-60-15.15 [N33A]; S-60-15.16 [N33M]; S-60- | | |
| 15.17 [N33L] | | |
| S-60-11; S-60-12; S-60-14; S-60-19; S-60-24 | GVPDRFSGSGSGTD | 49 |
| | FTLKISRVEAEDVGV | |
| | YYC | |
| S-60-16 | GVPDRFSGSGSGTD | 50 |
| | FTLKISRVEAEDVGA | |
| | YYC | |
| S-60-18 | GVPDRLSGSGSGTD | 51 |
| | FTLKISRVEAEDVGV | |
| | YYC | |
| Formula IX | GVPDRX₁SGSGSGTD | 52 |
| | FTLKISRX₂EAEDVG | |
| | X₃ YYC | |
| | X₁ is For L | |
| | X₂ is A or V | |
| | X₃ is V or A | |

TABLE 14
Light chain framework 4 sequences of anti-Sortilin antibodies

| Ab(s) | VL FR4 | SEQ ID NO: |
|---|---|---|
| S-60-10; S-60-11; S-60-12; S-60-13; S-60-14; S-60-15 | FGGGTKVEIK | 53 |
| [N33(wt)]; S-60-15.1 [N33T]; S-60-15.2 [N33S]; S-60- | | |
| 15.3 [N33G]; S-60-15.4 [N33R]; S-60-15.5 [N33D]; S- | | |
| 60-15.6 [N33H]; S-60-15.7 [N33K]; S-60-15.8 [N33Q]; | | |
| S-60-15.9 [N33Y]; S-60-15.10 [N33E]; S-60-15.11 | | |
| [N33W]; S-60-15.12 [N33F]; S-60-15.13 [N33I]; S-60- | | |
| 15.14 [N33V]; S-60-15.15 [N33A]; S-60-15.16 [N33M]; | | |
| S-60-15.17 [N33L]; S-60-16; S-60-18; S-60-19; S-60-24 | | |

TABLE 15
Heavy chain variable region sequences of anti-Sortilin antibodies

| Ab(s) | HCVR | SEQ ID NO: |
|---|---|---|
| S-60-10, S-60-11, S-60-12, S-60-19 | QVQLQESGPGLVKPSETLSLTCAVSG | 54 |
| | YSISSGYYWGWIRQPPGKGLEWIGTIY | |
| | HSGSTYYNPSLKSQVTISVDTSKNQFS | |
| | LELSSVTAADTAVYYCARQGSIQQGY | |
| | YGMDVWGQGTTVTVSS | |
| S-60-13, S-60-14 | QVQLQESGPGLVKPSETLSLTCAVSG | 55 |
| | YSISSGYYWGWIRQPPGKGLEWIGTIY | |
| | HSGSTYYNPSLESRVTISVDTSKNQFS | |
| | LKLSSVTAADTAVYYCARQGSIQQGY | |
| | YGMDVWGQGTTVTVSS | |
| S-60, S-60-15 [N33 (wt)], S-60-15.1 | QVQLQESGPGLVKPSETLSLTCAVSG | 56 |
| [N33T], S-60-15.2 [N33S], S-60-15.3 | YSISSGYYWGWIRQPPGKGLEWIGTIY | |
| [N33G], S-60-15.4 [N33R], S-60-15.5 | HSGSTYYNPSLKSRVTISVDTSKNQFS | |
| [N33D], S-60-15.6 [N33H], S-60-15.7 | LKLSSVTAADTAVYYCARQGSIKQGY | |
| [N33K], S-60-15.8 [N33Q], S-60-15.9 | YGMDVWGQGTTVTVSS | |
| [N33Y], S-60-15.10 [N33E], S-60-15.11 | | |
| [N33W], S-60-15.12 [N33F], S-60-15.13 | | |
| [N33I], S-60-15.14 [N33V], S-60-15.15 | | |
| [N33A], S-60-15.16 [N33M], S-60-15.17 | | |
| [N33L], S-60-16, S-60-18, S-60-24 | | |

TABLE 16
Light chain variable sequences of anti-Sortilin antibodies

| Ab(s) | LCVR | SEQ ID NO: |
|---|---|---|
| S-60-10; S-60-13; S-60-15 [N33(wt)] | DIVMTQSPLSLPVTPGEPASISCRSSQS | 57 |
| | LLRSNGYNYLDWYLQKPGQSPQLLIY | |
| | LGSNRASGVPDRFSGSGSGTDFTLKIS | |
| | RAEAEDVGVYYCMQQQEAPLTFGGG | |
| | TKVEIK | |
| S-60-11; S-60-14 | DIVMTQSPLSLPVTPGEPASISCRSSQS | 58 |
| | LLRSNGYNYLDWYLQKPGQSPQLLIY | |
| | LGSNRASGVPDRFSGSGSGTDFTLKIS | |
| | RVEAEDVGVYYCMQQQEAPLTFGGG | |
| | TKVEIK | |
| S-60-12 | DIVMTQSPLSLPVTPGEPASISCRSSQS | 59 |
| | LLRSNGYNYLDWYLQKPGQPPQLLIY | |
| | LGSNRVSGVPDRFSGSGSGTDFTLKIS | |
| | RVEAEDVGVYYCMQQQETPLTFGGG | |
| | TKVEIK | |
| S-60-15.1 [N33T] | DIVMTQSPLSLPVTPGEPASISCRSSQS | 60 |
| | LLRSTGYNYLDWYLQKPGQSPQLLIY | |
| | LGSNRASGVPDRFSGSGSGTDFTLKIS | |
| | RAEAEDVGVYYCMQQQEAPLTFGGG | |
| | TKVEIK | |
| S-60-15.2 [N33S] | DIVMTQSPLSLPVTPGEPASISCRSSQS | 61 |
| | LLRSSGYNYLDWYLQKPGQSPQLLIY | |
| | LGSNRASGVPDRFSGSGSGTDFTLKIS | |
| | RAEAEDVGVYYCMQQQEAPLTFGGG | |
| | TKVEIK | |
| S-60-15.3 [N33G] | DIVMTQSPLSLPVTPGEPASISCRSSQS | 62 |
| | LLRSGGYNYLDWYLQKPGQSPQLLIY | |
| | LGSNRASGVPDRFSGSGSGTDFTLKIS | |
| | RAEAEDVGVYYCMQQQEAPLTFGGG | |
| | TKVEIK | |
| S-60-15.4 [N33R] | DIVMTQSPLSLPVTPGEPASISCRSSQS | 63 |
| | LLRSRGYNYLDWYLQKPGQSPQLLIY | |
| | LGSNRASGVPDRFSGSGSGTDFTLKIS | |
| | RAEAEDVGVYYCMQQQEAPLTFGGG | |
| | TKVEIK | |
| S-60-15.5 [N33D] | DIVMTQSPLSLPVTPGEPASISCRSSQS | 64 |
| | LLRSDGYNYLDWYLQKPGQSPQLLIY | |
| | LGSNRASGVPDRFSGSGSGTDFTLKIS | |
| | RAEAEDVGVYYCMQQQEAPLTFGGG | |
| | TKVEIK | |
| S-60-15.6 [N33H] | DIVMTQSPLSLPVTPGEPASISCRSSQS | 65 |
| | LLRSHGYNYLDWYLQKPGQSPQLLIY | |
| | LGSNRASGVPDRFSGSGSGTDFTLKIS | |
| | RAEAEDVGVYYCMQQQEAPLTFGGG | |
| | TKVEIK | |
| S-60-15.7 [N33K] | DIVMTQSPLSLPVTPGEPASISCRSSQS | 66 |
| | LLRSKGYNYLDWYLQKPGQSPQLLIY | |
| | LGSNRASGVPDRFSGSGSGTDFTLKIS | |
| | RAEAEDVGVYYCMQQQEAPLTFGGG | |
| | TKVEIK | |
| S-60-15.8 [N33Q] | DIVMTQSPLSLPVTPGEPASISCRSSQS | 67 |
| | LLRSQGYNYLDWYLQKPGQSPQLLIY | |
| | LGSNRASGVPDRFSGSGSGTDFTLKIS | |
| | RAEAEDVGVYYCMQQQEAPLTFGGG | |
| | TKVEIK | |
| S-60-15.9 [N33Y] | DIVMTQSPLSLPVTPGEPASISCRSSQS | 68 |
| | LLRSYGYNYLDWYLQKPGQSPQLLIY | |
| | LGSNRASGVPDRFSGSGSGTDFTLKIS | |
| | RAEAEDVGVYYCMQQQEAPLTFGGG | |
| | TKVEIK | |
| S-60-15.10 [N33E] | DIVMTQSPLSLPVTPGEPASISCRSSQS | 69 |
| | LLRSEGYNYLDWYLQKPGQSPQLLIY | |
| | LGSNRASGVPDRFSGSGSGTDFTLKIS | |
| | RAEAEDVGVYYCMQQQEAPLTFGGG | |
| | TKVEIK | |
| S-60-15.11 [N33W] | DIVMTQSPLSLPVTPGEPASISCRSSQS | 70 |
| | LLRSWGYNYLDWYLQKPGQSPQLLI | |
| | YLGSNRASGVPDRFSGSGSGTDFTLKI | |
| | SRAEAEDVGVYYCMQQQEAPLTFGG | |
| | GTKVEIK | |
| S-60-15.12 [N33F] | DIVMTQSPLSLPVTPGEPASISCRSSQS | 71 |
| | LLRSFGYNYLDWYLQKPGQSPQLLIY | |
| | LGSNRASGVPDRFSGSGSGTDFTLKIS | |
| | RAEAEDVGVYYCMQQQEAPLTFGGG | |
| | TKVEIK | |
| S-60-15.13 [N33] | DIVMTQSPLSLPVTPGEPASISCRSSQS | 72 |
| | LLRSIGYNYLDWYLQKPGQSPQLLIY | |
| | LGSNRASGVPDRFSGSGSGTDFTLKIS | |
| | RAEAEDVGVYYCMQQQEAPLTFGGG | |
| | TKVEIK | |
| S-60-15.14 [N33V] | DIVMTQSPLSLPVTPGEPASISCRSSQS | 73 |
| | LLRSVGYNYLDWYLQKPGQSPQLLIY | |
| | LGSNRASGVPDRFSGSGSGTDFTLKIS | |
| | RAEAEDVGVYYCMQQQEAPLTFGGG | |
| | TKVEIK | |
| S-60-15.15 [N33A] | DIVMTQSPLSLPVTPGEPASISCRSSQS | 74 |
| | LLRSAGYNYLDWYLQKPGQSPQLLIY | |
| | LGSNRASGVPDRFSGSGSGTDFTLKIS | |
| | RAEAEDVGVYYCMQQQEAPLTFGGG | |
| | TKVEIK | |
| S-60-15.16 [N33M] | DIVMTQSPLSLPVTPGEPASISCRSSQS | 75 |
| | LLRSMGYNYLDWYLQKPGQSPQLLIY | |
| | LGSNRASGVPDRFSGSGSGTDFTLKIS | |
| | RAEAEDVGVYYCMQQQEAPLTFGGG | |
| | TKVEIK | |
| S-60-15.17 [N33L] | DIVMTQSPLSLPVTPGEPASISCRSSQS | 76 |
| | LLRSLGYNYLDWYLQKPGQSPQLLIY | |
| | LGSNRASGVPDRFSGSGSGTDFTLKIS | |
| | RAEAEDVGVYYCMQQQEAPLTFGGG | |
| | TKVEIK | |
| S-60-16 | DIVMTQSPLSLPVTPGEPASISCRSSQS | 77 |
| | LLRSNGYNYLDWYLQKPGQSPQLLIY | |
| | LGSNRASGVPDRFSGSGSGTDFTLKIS | |
| | RVEAEDVGAYYCMQQQEAPLTFGGG | |
| | TKVEIK | |
| S-60-18 | DIVMTQSPLSLPVTPGGPASISCRSSQS | 78 |
| | LLRSNGYNYLDWYLQKPGQSPQLLIY | |
| | LGSNRASGVPDRLSGSGSGTDFTLKIS | |
| | RVEAEDVGVYYCMQQQETPLTFGGG | |
| | TKVEIK | |
| S-60, S-60-19 | DIVMTQSPLSLPVTPGEPASISCRSSQS | 79 |
| | LLHSNGYNYLDWYLQKPGQSPQLLIY | |
| | LGSNRASGVPDRFSGSGSGTDFTLKIS | |
| | RVEAEDVGVYYCMQQQETPLTFGGG | |
| | TKVEIK | |
| S-60-24 | DIVMTQSPLSLPVTPGESASISCRSSQG | 80 |
| | LLRSNGYNYLDWYLQKPGQSPQLLIY | |
| | LGSNRASGVPDRFSGSGSGTDFTLKIS | |
| | RVEAEDVGVYYCMQQQEAPLTFGGG | |
| | TKVEIK | |

TABLE 17
Sortilin amino acid sequences

| | | SEQ ID |
|---|---|---|
| Description | Sequence | NO |
| Human | MERPWGAADGLSRWPHGLGLLLLLQLLPPSTLSQDRLDAPPPPA | 81 |
| Sortilin | APLPRWSGPIGVSWGLRAAAAGGAFPRGGRWRRSAPGEDEECG | |
| | RVRDFVAKLANNTHQHVFDDLRGSVSLSWVGDSTGVILVLTTFH | |
| | VPLVIMTFGQSKLYRSEDYGKNFKDITDLINNTFIRTEFGMAIGPE | |
| | NSGKVVLTAEVSGGSRGGRIFRSSDFAKNFVQTDLPFHPLTQMM | |
| | YSPQNSDYLLALSTENGLWVSKNFGGKWEEIHKAVCLAKWGSD | |
| | NTIFFTTYANGSCKADLGALELWRTSDLGKSFKTIGVKIYSFGLG | |
| | GRFLFASVMADKDTTRRIHVSTDQGDTWSMAQLPSVGQEQFYS | |
| | ILAANDDMVFMHVDEPGDTGFGTIFTSDDRGIVYSKSLDRHLYT | |
| | TTGGETDFTNVTSLRGVYITSVLSEDNSIQTMITFDQGGRWTHLR | |
| | KPENSECDATAKNKNECSLHIHASYSISQKLNVPMAPLSEPNAVG | |
| | IVIAHGSVGDAISVMVPDVYISDDGGYSWTKMLEGPHYYTILDS | |
| | GGIIVAIEHSSRPINVIKFSTDEGQCWQTYTFTRDPIYFTGLASEPG | |
| | ARSMNISIWGFTESFLTSQWVSYTIDFKDILERNCEEKDYTIWLA | |
| | HSTDPEDYEDGCILGYKEQFLRLRKSSVCQNGRDYVVTKQPSICL | |
| | CSLEDFLCDFGYYRPENDSKCVEQPELKGHDLEFCLYGREEHLT | |
| | TNGYRKIPGDKCQGGVNPVREVKDLKKKCTSNFLSPEKQNSKSN | |
| | SVPIILAIVGLMLVTVVAGVLIVKKYVCGGRFLVHRYSVLQQHA | |
| | EANGVDGVDALDTASHTNKSGYHDDSDEDLLE | |
| Mouse | MERPRGAADG LLRWPLGLLL LLQLLPPAAV GQDRLDAPPP | 82 |
| Sortilin | PAPPLLRWAG PVGVSWGLRA AAPGGPVPRA GRWRRGAPAE | |
| | DQDCGRLPDF IAKLTNNTHQ HVFDDLSGSV SLSWVGDSTG | |
| | VILVLTTFQV PLVIVSFGQS KLYRSEDYGK NFKDITNLIN | |
| | NTFIRTEFGM AIGPENSGKV ILTAEVSGGS RGGRVFRSSD | |
| | FAKNFVQTDL PFHPLTQMMY SPQNSDYLLA LSTENGLWVS | |
| | KNFGEKWEEI HKAVCLAKWG PNNIIFFTTH VNGSCKADLG | |
| | ALELWRTSDL GKTFKTIGVK IYSFGLGGRF LFASVMADKD | |
| | TTRRIHVSTD QGDTWSMAQL PSVGQEQFYS ILAANEDMVF | |
| | MHVDEPGDTG FGTIFTSDDR GIVYSKSLDR HLYTTTGGET | |
| | DFTNVTSLRG VYITSTLSED NSIQSMITFD QGGRWEHLRK | |
| | PENSKCDATA KNKNECSLHI HASYSISQKL NVPMAPLSEP | |
| | NAVGIVIAHG SVGDAISVMV PDVYISDDGG YSWAKMLEGP | |
| | HYYTILDSGG IIVAIEHSNR PINVIKFSTD EGQCWQSYVF | |
| | TQEPIYFTGL ASEPGARSMN ISIWGFTESF ITRQWVSYTV | |
| | DFKDILERNC EEDDYTTWLA HSTDPGDYKD GCILGYKEQF | |
| | LRLRKSSVCQ NGRDYVVAKQ PSVCPCSLED FLCDFGYFRP | |
| | ENASECVEQP ELKGHELEFC LYGKEEHLTT NGYRKIPGDK | |
| | CQGGMNPARE VKDLKKKCTS NFLNPTKQNS KSNSVPIILA | |
| | IVGLMLVTVV AGVLIVKKYV CGGRFLVHRY SVLQQHAEAD | |
| | GVEALDSTSH AKSGYHDDSD EDLLE | |
| Rat Sortilin | MERPRGAADG LLRWPLGLLL LLQLLPPAAV GQDRLDAPPP | 83 |
| | PAPPLLRWAG PVGVSWGLRA AAPGGPVPRA GRWRRGAPAE | |
| | DQDCGRLPDF IAKLTNNTHQ HVFDDLSGSV SLSWVGDSTG | |
| | VILVLTTFQV PLVIVSFGQS KLYRSEDYGK NFKDITNLIN | |
| | NTFIRTEFGM AIGPENSGKV ILTAEVSGGS RGGRVFRSSD | |
| | FAKNFVQTDL PFHPLTQMMY SPQNSDYLLA LSTENGLWVS | |
| | KNFGEKWEEI HKAVCLAKWG PNNIIFFTTH VNGSCKADLG | |
| | ALELWRTSDL GKTFKTIGVK IYSFGLGGRF LFASVMADKD | |
| | TTRRIHVSTD QGDTWSMAQL PSVGQEQFYS ILAANDDMVF | |
| | MHVDEPGDTG FGTIFTSDDR GIVYSKSLDR HLYTTTGGET | |
| | DFTNVTSLRG VYITSTLSED NSIQSMITFD QGGRWEHLQK | |
| | PENSKCDATA KNKNECSLHI HASYSISQKL NVPMAPLSEP | |
| | NAVGIVIAHG SVGDAISVMV PDVYISDDGG YSWAKMLEGP | |
| | HYYTILDSGG IIVAIEHSNR PINVIKFSTD EGQCWQSYVF | |
| | SQEPVYFTGL ASEPGARSMN ISIWGFTESF LTRQWVSYTI | |
| | DFKDILERNC EENDYTTWLA HSTDPGDYKD GCILGYKEQF | |
| | LRLRKSSVCQ NGRDYVVAKQ PSICPCSLED FLCDFGYFRP | |
| | ENASECVEQP ELKGHELEFC LYGKEEHLTT NGYRKIPGDR | |
| | CQGGMNPARE VKDLKKKCTS NFLNPKKQNS KSSSVPIILA | |
| | IVGLMLVTVV AGVLIVKKYV CGGRFLVHRY SVLQQHAEAD | |
| | GVEALDTASH AKSGYHDDSD EDLLE | |

TABLE 18
Fc domain amino acid sequences

| | | SEQ ID |
|---|---|---|
| Description | Sequence | NO |
| huIgG1 | ASTKGPSVFPLAPSSKSTSGGTAALGCLVKDYFPEPVTVSWNSGALT | 84 |
| LALAPS Fc | SGVHTFPAVLQSSGLYSLSSVVTVPSSSLGTQTYICNVNHKPSNTKV | |
| with C- | DKKVEPKSCDKTHTCPPCPAPEAAGGPSVFLFPPKPKDTLMISRTPE | |
| terminal lysine | VTCVVVDVSHEDPEVKFNWYVDGVEVHNAKTKPREEQYNSTYRV | |
| | VSVLTVLHQDWLNGKEYKCKVSNKALPASIEKTISKAKGQPREPQV | |
| | YTLPPSRDELTKNQVSLTCLVKGFYPSDIAVEWESNGQPENNYKTT | |
| | PPVLDSDGSFFLYSKLTVDKSRWQQGNVFSCSVMHEALHNHYTQK | |
| | SLSLSPGK | |
| huIgG1 | ASTKGPSVFPLAPSSKSTSGGTAALGCLVKDYFPEPVTVSWNSGALT | 85 |
| LALAPS Fc | SGVHTFPAVLQSSGLYSLSSVVTVPSSSLGTQTYICNVNHKPSNTKV | |
| without C- | DKKVEPKSCDKTHTCPPCPAPEAAGGPSVFLFPPKPKDTLMISRTPE | |
| terminal lysine | VTCVVVDVSHEDPEVKFNWYVDGVEVHNAKTKPREEQYNSTYRV | |
| | VSVLTVLHQDWLNGKEYKCKVSNKALPASIEKTISKAKGQPREPQV | |
| | YTLPPSRDELTKNQVSLTCLVKGFYPSDIAVEWESNGQPENNYKTT | |
| | PPVLDSDGSFFLYSKLTVDKSRWQQGNVFSCSVMHEALHNHYTQK | |
| | SLSLSPG | |

TABLE 19
Full-length heavy chain amino acid sequences

| | | SEQ ID |
|---|---|---|
| Description | Sequence | NO |
| S-60-10, S-60-11, S-60-12, S- | QVQLQESGPGLVKPSETLSLTCAVSGYSISSGYYWGW | 86 |
| 60-19 with Fc LALAPS with | IRQPPGKGLEWIGTIYHSGSTYYNPSLKSQVTISVDTS | |
| C-terminal Lysine | KNQFSLELSSVTAADTAVYYCARQGSIQQGYYGMDV | |
| | WGQGTTVTVSSASTKGPSVFPLAPSSKSTSGGTAALG | |
| | CLVKDYFPEPVTVSWNSGALTSGVHTFPAVLQSSGLY | |
| | SLSSVVTVPSSSLGTQTYICNVNHKPSNTKVDKKVEP | |
| | KSCDKTHTCPPCPAPEAAGGPSVFLFPPKPKDTLMISR | |
| | TPEVTCVVVDVSHEDPEVKFNWYVDGVEVHNAKTK | |
| | PREEQYNSTYRVVSVLTVLHQDWLNGKEYKCKVSN | |
| | KALPASIEKTISKAKGQPREPQVYTLPPSRDELTKNQV | |
| | SLTCLVKGFYPSDIAVEWESNGQPENNYKTTPPVLDS | |
| | DGSFFLYSKLTVDKSRWQQGNVFSCSVMHEALHNH | |
| | YTQKSLSLSPGK | |
| S-60-10, S-60-11, S-60-12, S- | QVQLQESGPGLVKPSETLSLTCAVSGYSISSGYYWGW | 87 |
| 60-19 with Fc LALAPS | IRQPPGKGLEWIGTIYHSGSTYYNPSLKSQVTISVDTS | |
| without C-terminal Lysine | KNQFSLELSSVTAADTAVYYCARQGSIQQGYYGMDV | |
| | WGQGTTVTVSSASTKGPSVFPLAPSSKSTSGGTAALG | |
| | CLVKDYFPEPVTVSWNSGALTSGVHTFPAVLQSSGLY | |
| | SLSSVVTVPSSSLGTQTYICNVNHKPSNTKVDKKVEP | |
| | KSCDKTHTCPPCPAPEAAGGPSVFLFPPKPKDTLMISR | |
| | TPEVTCVVVDVSHEDPEVKFNWYVDGVEVHNAKTK | |
| | PREEQYNSTYRVVSVLTVLHQDWLNGKEYKCKVSN | |
| | KALPASIEKTISKAKGQPREPQVYTLPPSRDELTKNQV | |
| | SLTCLVKGFYPSDIAVEWESNGQPENNYKTTPPVLDS | |
| | DGSFFLYSKLTVDKSRWQQGNVFSCSVMHEALHNH | |
| | YTQKSLSLSPG | |
| S-60-13, S-60-14 with Fc | QVQLQESGPGLVKPSETLSLTCAVSGYSISSGYYWGW | 88 |
| LALAPS with C-terminal | IRQPPGKGLEWIGTIYHSGSTYYNPSLESRVTISVDTSK | |
| Lysine | NQFSLKLSSVTAADTAVYYCARQGSIQQGYYGMDV | |
| | WGQGTTVTVSSASTKGPSVFPLAPSSKSTSGGTAALG | |
| | CLVKDYFPEPVTVSWNSGALTSGVHTFPAVLQSSGLY | |
| | SLSSVVTVPSSSLGTQTYICNVNHKPSNTKVDKKVEP | |
| | KSCDKTHTCPPCPAPEAAGGPSVFLFPPKPKDTLMISR | |
| | TPEVTCVVVDVSHEDPEVKFNWYVDGVEVHNAKTK | |
| | PREEQYNSTYRVVSVLTVLHQDWLNGKEYKCKVSN | |
| | KALPASIEKTISKAKGQPREPQVYTLPPSRDELTKNQV | |
| | SLTCLVKGFYPSDIAVEWESNGQPENNYKTTPPVLDS | |
| | DGSFFLYSKLTVDKSRWQQGNVFSCSVMHEALHNH | |
| | YTQKSLSLSPGK | |
| S-60-13, S-60-14 with Fc | QVQLQESGPGLVKPSETLSLTCAVSGYSISSGYYWGW | 89 |
| LALAPS without C- | IRQPPGKGLEWIGTIYHSGSTYYNPSLESRVTISVDTSK | |
| terminal Lysine | NQFSLKLSSVTAADTAVYYCARQGSIQQGYYGMDV | |
| | WGQGTTVTVSSASTKGPSVFPLAPSSKSTSGGTAALG | |
| | CLVKDYFPEPVTVSWNSGALTSGVHTFPAVLQSSGLY | |
| | SLSSVVTVPSSSLGTQTYICNVNHKPSNTKVDKKVEP | |
| | KSCDKTHTCPPCPAPEAAGGPSVFLFPPKPKDTLMISR | |
| | TPEVTCVVVDVSHEDPEVKFNWYVDGVEVHNAKTK | |
| | PREEQYNSTYRVVSVLTVLHQDWLNGKEYKCKVSN | |
| | KALPASIEKTISKAKGQPREPQVYTLPPSRDELTKNQV | |
| | SLTCLVKGFYPSDIAVEWESNGQPENNYKTTPPVLDS | |
| | DGSFFLYSKLTVDKSRWQQGNVFSCSVMHEALHNH | |
| | YTQKSLSLSPG | |
| S-60, S-60-15 [N33(wt)], S- | QVQLQESGPGLVKPSETLSLTCAVSGYSISSGYYWGW | 90 |
| 60-15.1 [N33T], S-60-15.2 | IRQPPGKGLEWIGTIYHSGSTYYNPSLKSRVTISVDTS | |
| [N33S], S-60-15.3 [N33G], | KNQFSLKLSSVTAADTAVYYCARQGSIKQGYYGMD | |
| S-60-15.4 [N33R], S-60-15.5 | VWGQGTTVTVSSASTKGPSVFPLAPSSKSTSGGTAAL | |
| [N33D], S-60-15.6 [N33H], | GCLVKDYFPEPVTVSWNSGALTSGVHTFPAVLQSSGL | |
| S-60-15.7 [N33K], S-60-15.8 | YSLSSVVTVPSSSLGTQTYICNVNHKPSNTKVDKKVE | |
| [N33Q], S-60-15.9 [N33Y], | PKSCDKTHTCPPCPAPEAAGGPSVFLFPPKPKDTLMIS | |
| S-60-15.10 [N33E], S-60- | RTPEVTCVVVDVSHEDPEVKFNWYVDGVEVHNAKT | |
| 15.11 [N33W], S-60-15.12 | KPREEQYNSTYRVVSVLTVLHQDWLNGKEYKCKVS | |
| [N33F], S-60-15.13 [N33I], | NKALPASIEKTISKAKGQPREPQVYTLPPSRDELTKNQ | |
| S-60-15.14 [N33V], S-60- | VSLTCLVKGFYPSDIAVEWESNGQPENNYKTTPPVLD | |
| 15.15 [N33A], S-60-15.16 | SDGSFFLYSKLTVDKSRWQQGNVFSCSVMHEALHNH | |
| [N33M], S-60-15.17 [N33L], | YTQKSLSLSPGK | |
| S-60-16, S-60-18, S-60-24 | | |
| with Fc LALAPS with C- | | |
| terminal Lysine | | |
| S-60, S-60-15 [N33(wt)], S- | QVQLQESGPGLVKPSETLSLTCAVSGYSISSGYYWGW | 91 |
| 60-15.1 [N33T], S-60-15.2 | IRQPPGKGLEWIGTIYHSGSTYYNPSLKSRVTISVDTS | |
| [N33S], S-60-15.3 [N33G], | KNQFSLKLSSVTAADTAVYYCARQGSIKQGYYGMD | |
| S-60-15.4 [N33R], S-60-15.5 | VWGQGTTVTVSSASTKGPSVFPLAPSSKSTSGGTAAL | |
| [N33D], S-60-15.6 [N33H], | GCLVKDYFPEPVTVSWNSGALTSGVHTFPAVLQSSGL | |
| S-60-15.7 [N33K], S-60-15.8 | YSLSSVVTVPSSSLGTQTYICNVNHKPSNTKVDKKVE | |
| [N33Q], S-60-15.9 [N33Y], | PKSCDKTHTCPPCPAPEAAGGPSVFLFPPKPKDTLMIS | |
| S-60-15.10 [N33E], S-60- | RTPEVTCVVVDVSHEDPEVKFNWYVDGVEVHNAKT | |
| 15.11 [N33W], S-60-15.12 | KPREEQYNSTYRVVSVLTVLHQDWLNGKEYKCKVS | |
| [N33F], S-60-15.13 [N33I], | NKALPASIEKTISKAKGQPREPQVYTLPPSRDELTKNQ | |
| S-60-15.14 [N33V], S-60- | VSLTCLVKGFYPSDIAVEWESNGQPENNYKTTPPVLD | |
| 15.15 [N33A], S-60-15.16 | SDGSFFLYSKLTVDKSRWQQGNVFSCSVMHEALHNH | |
| [N33M], S-60-15.17 [N33L], | YTQKSLSLSPG | |
| S-60-16, S-60-18, S-60-24 | | |
| with Fc LALAPS without | | |
| C-terminal Lysine | | |

TABLE 20
Full-length light chain amino acid sequences

| | | SEQ |
|---|---|---|
| | | ID |
| Description | Sequence | NO |
| S-60-10; S-60-13; | DIVMTQSPLSLPVTPGEPASIS | 92 |
| S-60-15 [N33(wt)] | CRSSQSLLRSNGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-11; S-60-14 | DIVMTQSPLSLPVTPGEPASIS | 93 |
| | CRSSQSLLRSNGYNYLDWYL | |
| | QKPGQSPQLLIYLGSNRASGV | |
| | PDRFSGSGSGTDFTLKISRVEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-12 | DIVMTQSPLSLPVTPGEPASIS | 94 |
| | CRSSQSLLRSNGYNYLDWYL | |
| | QKPGQPPQLLIYLGSNRVSGV | |
| | PDRFSGSGSGTDFTLKISRVEA | |
| | EDVGVYYCMQQQETPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.1 [N33T] | DIVMTQSPLSLPVTPGEPASIS | 95 |
| | CRSSQSLLRSTGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.2 [N33S] | DIVMTQSPLSLPVTPGEPASIS | 96 |
| | CRSSQSLLRSSGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.3 [N33G] | DIVMTQSPLSLPVTPGEPASIS | 97 |
| | CRSSQSLLRSGGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.4 [N33R] | DIVMTQSPLSLPVTPGEPASIS | 98 |
| | CRSSQSLLRSRGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.5 [N33D] | DIVMTQSPLSLPVTPGEPASIS | 99 |
| | CRSSQSLLRSDGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.6 [N33H] | DIVMTQSPLSLPVTPGEPASIS | 100 |
| | CRSSQSLLRSHGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.7 [N33K] | DIVMTQSPLSLPVTPGEPASIS | 101 |
| | CRSSQSLLRSKGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.8 [N33Q] | DIVMTQSPLSLPVTPGEPASIS | 102 |
| | CRSSQSLLRSQGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.9 [N33Y] | DIVMTQSPLSLPVTPGEPASIS | 103 |
| | CRSSQSLLRSYGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.10 [N33E] | DIVMTQSPLSLPVTPGEPASIS | 104 |
| | CRSSQSLLRSEGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.11 [N33W] | DIVMTQSPLSLPVTPGEPASIS | 105 |
| | CRSSQSLLRSWGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.12 [N33F] | DIVMTQSPLSLPVTPGEPASIS | 106 |
| | CRSSQSLLRSFGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.13 [N33I] | DIVMTQSPLSLPVTPGEPASIS | 107 |
| | CRSSQSLLRSIGYNYLDWYLQ | |
| | KPGQSPQLLIYLGSNRASGVP | |
| | DRFSGSGSGTDFTLKISRAEAE | |
| | DVGVYYCMQQQEAPLTFGGG | |
| | TKVEIKRTVAAPSVFIFPPSDE | |
| | QLKSGTASVVCLLNNFYPREA | |
| | KVQWKVDNALQSGNSQESVT | |
| | EQDSKDSTYSLSSTLTLSKAD | |
| | YEKHKVYACEVTHQGLSSPV | |
| | TKSFNRGEC | |
| S-60-15.14 [N33V] | DIVMTQSPLSLPVTPGEPASIS | 108 |
| | CRSSQSLLRSVGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.15 [N33A] | DIVMTQSPLSLPVTPGEPASIS | 109 |
| | CRSSQSLLRSAGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.16 [N33M] | DIVMTQSPLSLPVTPGEPASIS | 110 |
| | CRSSQSLLRSMGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-15.17 [N33L] | DIVMTQSPLSLPVTPGEPASIS | 111 |
| | CRSSQSLLRSLGYNYLDWYL | |
| | QKPGQSPQLLIYLGSNRASGV | |
| | PDRFSGSGSGTDFTLKISRAEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-16 | DIVMTQSPLSLPVTPGEPASIS | 112 |
| | CRSSQSLLRSNGYNYLDWYL | |
| | QKPGQSPQLLIYLGSNRASGV | |
| | PDRFSGSGSGTDFTLKISRVEA | |
| | EDVGAYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-18 | DIVMTQSPLSLPVTPGGPASIS | 113 |
| | CRSSQSLLRSNGYNYLDWYL | |
| | QKPGQSPQLLIYLGSNRASGV | |
| | PDRLSGSGSGTDFTLKISRVEA | |
| | EDVGVYYCMQQQETPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60, S-60-19 | DIVMTQSPLSLPVTPGEPASIS | 114 |
| | CRSSQSLLHSNGYNYLDWYL | |
| | QKPGQSPQLLIYLGSNRASGV | |
| | PDRFSGSGSGTDFTLKISRVEA | |
| | EDVGVYYCMQQQETPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |
| S-60-24 | DIVMTQSPLSLPVTPGESASIS | 115 |
| | CRSSQGLLRSNGYNYLDWYL | |
| | QKPGQSPQLLIYLGSNRASGV | |
| | PDRFSGSGSGTDFTLKISRVEA | |
| | EDVGVYYCMQQQEAPLTFGG | |
| | GTKVEIKRTVAAPSVFIFPPSD | |
| | EQLKSGTASVVCLLNNFYPRE | |
| | AKVQWKVDNALQSGNSQES | |
| | VTEQDSKDSTYSLSSTLTLSK | |
| | ADYEKHKVYACEVTHQGLSS | |
| | PVTKSFNRGEC | |

The present disclosure will be more fully understood by reference to the following Examples. They should not, however, be construed as limiting the scope of the present disclosure. All citations throughout the disclosure are hereby expressly incorporated by reference.

### EXAMPLES

### Example 1: A Phase 3 Study of an Anti-Sortilin Antibody in Individuals at Risk for or with Frontotemporal Dementia Due to Heterozygous Mutations in the Progranulin Gene

This Example describes a Phase 3, multicenter, randomized, double blind, placebo controlled study to evaluate the efficacy and safety of anti-Sortilin antibody S-60-15.1 [N33T] LALAPS in individuals at risk for or with frontotemporal dementia (FTD) due to heterozygous mutations in the Progranulin gene (GRN).

### I. Study Objectives and Endpoints

This study includes two parts: Part 1 of this study is a double-blind, placebo-controlled treatment period, and Part 2 is an open-label extension phase. The objectives and endpoints of Parts 1 and 2 of the study are provided below.

A. Part 1

The primary efficacy objective of Part 1 of this study is to evaluate the efficacy of anti-Sortilin antibody S-60-15.1 [N33T] LALAPS compared to placebo, as measured by the Clinical Dementia Rating Dementia Staging Instrument PLUS National Alzheimer's Disease Coordinating Center Frontotemporal Lobar Degeneration Behavior and Language Domains Sum of Boxes (CDR® plus NACC FTLD SB) in carriers of GRN mutations causative of FTD. This objective is assessed based on change in the CDR® plus NACC FTLD-SB from baseline to Weeks 48, 72, and 96.

The secondary efficacy objectives of Part 1 of this study include:
Evaluation of the efficacy of anti-Sortilin antibody S-60-15.1 [N33T] LALAPS compared with placebo as measured by Clinical Global Impression-Severity (CGI-S) in carriers of GRN mutations causative of FTD. This objective is assessed based on change from baseline to Weeks 48, 72, and 96 in the CGI-S.
Evaluation of the efficacy of anti-Sortilin antibody S-60-15.1 [N33T] LALAPS compared with placebo as measured by Clinical Global Impression-Improvement (CGI-I) in carriers of GRN mutations causative of FTD. This objective is assessed based on CGI-I at Weeks 48, 72, and 96.
Evaluation of the efficacy of anti-Sortilin antibody S-60-15.1 [N33T] LALAPS compared with placebo as measured by Repeatable Battery for the Assessment of Neuropsychological Status (RBANS) in carriers of GRN mutations causative of FTD. This objective is assessed based on change from baseline to Weeks 48, 72, and 96 in the RBANS.

The pharmacodynamic (PD) objectives of Part 1 of this study include evaluation of the PD of anti-Sortilin antibody S-60-15.1 [N33T] LALAPS compared with placebo as measured by disease pathology biomarkers. Assessments include: structural volumetric magnetic resonance imaging (MRI) whole and regional brain volume; Progranulin protein (PGRN) concentrations in plasma and cerebrospinal fluid (CSF); and neurofilament-light chain (NfL) concentrations in serum and CSF.

The safety objectives of Part 1 of this study include evaluation of the safety and tolerability of anti-Sortilin antibody S-60-15.1 [N33T] LALAPS compared with placebo as measured by safety assessments and anti-drug antibodies (ADAs). Safety assessments include: incidence, nature, and severity of adverse events (AEs) and serious adverse events (SAEs); abnormalities in physical examination; abnormalities in neurological examination; changes in vital signs from baseline over time; changes in electrocardiograms from baseline over time; MRI abnormalities; changes in clinical laboratory tests from baseline over time; Sheehan-Suicidality Tracking Scale (Sheehan STS); and incidence of ADAs to anti-Sortilin antibody S-60-15.1 [N33T] LALAPS.

The tertiary efficacy objectives of Part 1 of this study include evaluation of the effects of anti-Sortilin antibody S-60-15.1 [N33T] LALAPS compared with placebo as measured by:
Quality of Life clinical outcome assessments (COAs) based on European Quality of Life-5 Dimensions (EQ 5D) score and Zarit Burden Interview (ZBI) score.
Pharmacoeconomic COAs based on Resource Utilization in Dementia-Lite Version (RUD-Lite) score.
Exposure-response relationships based on the exposure response relationships for relevant PD (e.g., PGRN, NfL), efficacy, or safety responses in relation to anti-Sortilin antibody S-60-15.1 [N33T] LALAPS exposure.
Exploratory PD imaging based on arterial spin labeling, diffusion-tensor imaging, and other MRI measures.
Frontotemporal Dementia Rating Scale (FRS) score.
Winterlight Labs Speech Assessments (WLA).

B. Part 2

The primary objective of Part 2 of this study is the assessment of the long-term safety and tolerability of anti-Sortilin antibody S-60-15.1 [N33T] LALAPS in participants who complete 96 weeks of treatment in Part 1 of the study. Endpoints include incidence, nature, and severity of AEs and SAEs; abnormalities in physical examination; abnormalities in neurological examination; changes in vital signs from baseline over time; changes in electrocardiograms from baseline over time; MRI abnormalities; changes in clinical laboratory tests from baseline over time; Sheehan-STS; and incidence of ADAs to anti-Sortilin antibody S-60-15.1 [N33T] LALAPS.

The tertiary objectives of Part 2 of this study include assessment of the long-term effect of anti-Sortilin antibody S-60-15.1 [N33T] LALAPS in participants who complete 96 weeks of treatment in Part 1 of the study. Assessments include:
PD of anti-Sortilin antibody S-60-15.1 [N33T] LALAPS as measured by disease pathology biomarkers based on structural volumetric MRI whole and regional brain volume; arterial spin labeling, diffusion-tensor imaging, and other MRI measures; PGRN concentrations in plasma; and NfL concentrations in serum.
Clinical progression as measured by COAs based on the overall change from baseline on the scores of: CDR® plus NACC FTLD; CGI-I; CGI-S; RBANS; FRS; and Winterlight Labs Speech Assessments.
Quality of life COAs, including EQ-5D score and ZBI score.
Pharmacoeconomic COAs, including RUD-Lite score.
Exposure-response relationships, including exposure-response relationships for relevant PD (e.g., PGRN, NfL), efficacy, or safety responses in relation to anti-Sortilin antibody S-60-15.1 [N33T] LALAPS exposure.

### II. Study Design

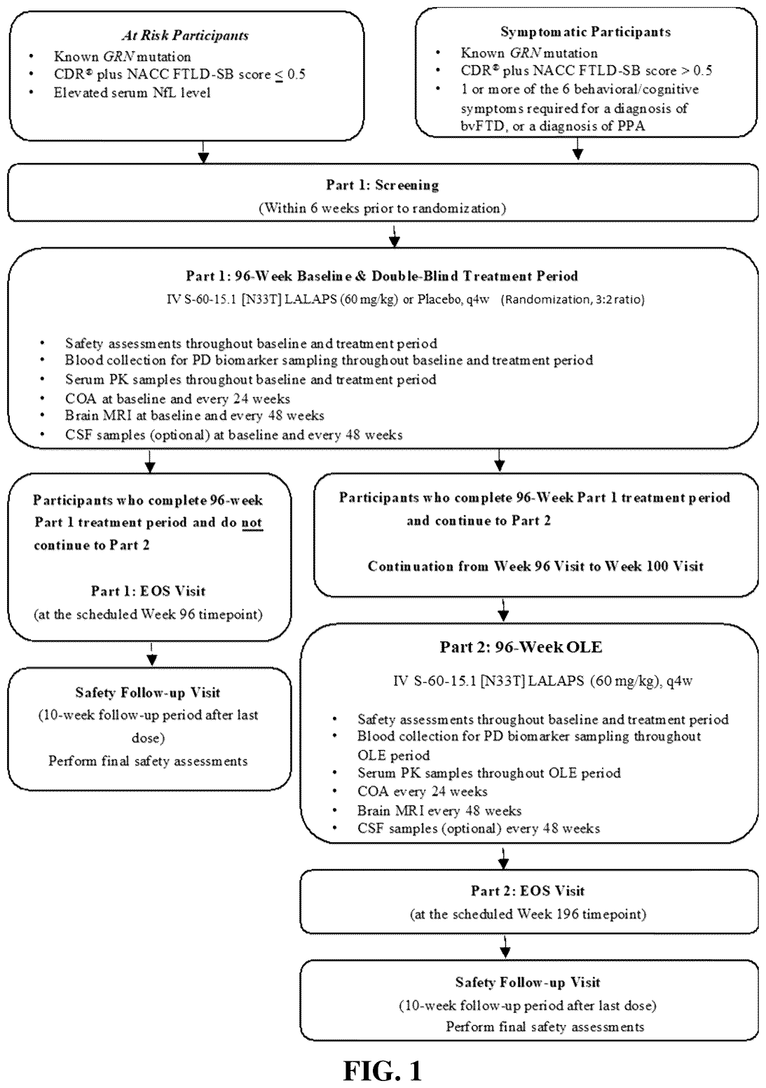

This study has two parts: a double-blind, placebo-controlled treatment period (Part 1), followed by an open-label extension (OLE) period (Part 2). An overview of the study design is provided in FIG. 1.

This study includes participants who are at risk for FTD or are symptomatic for FTD. Each study participant is a known carrier of a heterozygous loss-of-function GRN mutation causative of FTD, has a global CDR® plus NACC FTLD score of 0 to 2, and:
At-risk participants: Participants with a CDR® plus NACC FTLD-SB score≤0.5 and an elevated serum NfL. Elevated serum NfL may be predictive of clinical progression (see, e.g., Rojas-Martinez et al., Plasma neurofilament light chain predicts disease progression in asymptomatic familial frontotemporal lobar degeneration. Poster session presented at: American Academy of Neurology 2019 Annual Meeting; May 4-10; Philadelphia, PA).
Symptomatic participants: Participants with a CDR® plus NACC FTLD-SB score>0.5, and one or more of the six behavioral/cognitive symptoms required for a diagnosis of possible behavioral variant frontotemporal dementia (bvFTD; Rascovsky et al., Brain (2011) 134(Pt 9):2456-77), or a diagnosis of primary progressive aphasia (PPA; Gorno-Tempini et al., Neurology (2011) 76(11):1006-14).

A. Study Design: Part 1

Part 1 is a 96-week randomized, double-blind treatment period to evaluate the efficacy and safety of anti-Sortilin antibody S-60-15.1 [N33T] LALAPS at a dose of 60 mg/kg compared to placebo in participants who are carriers of heterozygous loss-of-function GRN mutations causative of FTD. Treatment is administered every 4 weeks (q4w; for a total of 25 doses).

Screening

Participants undergo a screening evaluation with MRI, blood sampling for PD biomarker measurement, and safety assessments. COAs are conducted at Screening. CSF is also collected at baseline prior to study treatment administration. Participants may be screened to determine the presence of GRN mutations. At-risk study participants (CDR® plus NACC FTLD-SB score<0.5) may be pre-screened to determine if their serum NfL level is elevated. Elevated serum NfL levels obtained ≤90 days prior to study treatment administration can be used to satisfy eligibility. Screening procedures may be performed within 6 weeks prior to randomization (Day −42 to Day 0).

Randomization

Eligible participants are randomly assigned, in a double-blind fashion, to receive anti-Sortilin antibody S-60-15.1 [N33T] LALAPS (60 mg/kg) or placebo in a 3:2 ratio (S-60-15.1 [N33T] LALAPS:placebo), and stratified by:
CDR® plus NACC FTLD-SB score≤0.5
CDR® plus NACC FTLD global score 0.5 (with a CDR® plus NACC FTLD-SB>0.5)
CDR® plus NACC FTLD global score 1
CDR® plus NACC FTLD global score 2

Treatment

Randomly assigned participants are administered study treatment (S-60-15.1 [N33T] LALAPS 60 mg/kg or placebo) q4w intravenously (IV) for 96 weeks starting on Day 0 and then q4w for a total of 25 doses. Treatment is administered IV over approximately 60 minutes.

Clinical outcome assessments are conducted every 24 weeks. Imaging is performed every 48 weeks. Lumbar puncture for CSF collection is performed every 48 weeks.

B. Study Design: Part 2

Part 2 is a 96-week OLE period for eligible participants who complete Part 1. Part 2 evaluates the long-term safety and tolerability of open-label anti-Sortilin antibody S-60-15.1 [N33T] LALAPS at a dose of 60 mg/kg administered q4w. Treatment is administered IV over approximately 60 minutes for up to 25 doses over a 96-week period.

All participants in the OLE, whether they received anti-Sortilin antibody S-60-15.1 [N33T] LALAPS or placebo in Part 1, receive open-label anti-Sortilin antibody S-60-15.1 [N33T] LALAPS at a dose of 60/mg/kg, q4w.

Blood sampling for safety assessments, COAs, and imaging are performed.

### III. Study Participants

This study includes adult male and female participants aged 25 to 85 years who are at risk for or have symptomatic FTD due to heterozygous loss-of-function mutations in the GRN gene.

A. Inclusion Criteria

Participants that meet all of the following criteria are enrolled in this study:
Is a known carrier of a heterozygous loss-of-function GRN mutation causative of FTD with a global CDR® plus NACC FTLD score of 0 to 2, and:
   At-risk participants: A CDR® plus NACC FTLD-SB score≤0.5 with an elevated level of serum NfL, or
   Symptomatic participants: A CDR® plus NACC FTLD-SB score of >0.5 with 1 or more of the 6 behavioral/cognitive symptoms required for a diagnosis of possible bvFTD (Rascovsky et al., Brain (2011) 134(Pt 9):2456-77), or a diagnosis of PPA (Gorno-Tempini et al., Neurology (2011) 76(11):1006-14).
Participant has the availability of a person (“study partner”) who has frequent and sufficient contact with the participant (at least 5 hours per week of in-person contact) and who can provide accurate information regarding the participant's behavior, cognitive, and functional abilities, as well as their health, throughout the study.

B. Exclusion Criteria

Participants that meet any of the following criteria are excluded from the study:
Dementia due to a condition other than FTD including, but not limited to, Alzheimer's disease, Parkinson's disease, dementia with Lewy bodies, Huntington disease, or vascular dementia.
Known mutation causative of neurodegenerative disorder(s) other than heterozygous loss-of-function GRN mutations causative of FTD.
Known history of severe allergic, anaphylactic, or other hypersensitivity reactions to chimeric, human, or humanized antibodies or fusion proteins.
Signs or symptoms of progressive supranuclear palsy or bulbar dysfunction, such as postural instability, eye problems, and swallowing difficulties.
History of moderate or severe substance use disorder within the past 2 years, with the exception of nicotine, as defined by the Diagnostic and Statistical Manual of Mental Disorders, fifth edition criteria (American Psychiatric Association 2013).
Currently has or has had an acute illness that requires or required systemic antibiotics within 30 days prior to first study treatment administration.
Clinically significant vitamin B12 or folate deficiency (if treated, must be on a stable regimen for at least 3 months prior to first study treatment administration).
Untreated hypothyroidism (if treated, thyroid supplementation dose must be stable for at least 3 months with a normal thyroid-stimulating hormone level prior to study treatment administration).
Insufficiently controlled diabetes mellitus (e.g., hemoglobin A1C≥8%).
Any surgery (major or emergent) or hospitalization within 30 days prior to first study treatment administration.
History of cancer within the last 5 years with the exception of basal cell or squamous cell carcinoma.
Positive for hepatitis B surface antigen, human immunodeficiency virus-1 or -2 antibodies or antigen, or history of spirochetal infection of the CNS (e.g., syphilis, borreliosis, or Lyme disease). Participants with a positive hepatitis C virus antibody are allowed if hepatitis C RNA is negative.
Significant kidney disease as indicated by either of the following:
   a. Estimated glomerular filtration rate (eGFR)<30 mL/min/1.73 m², according to the re-expressed abbreviated (four-variable) Modification of Diet in Renal Disease (MDRD) Study equation,
      Note: MDRD equation is as follows:

eGFR(mL/min/1.73 m²)=175×(standardized serum creatinine)^{-1.154}×(Age)^{-0.203}×(0.742 if female)×(1.212 if black), or
b. Creatinine≥2 mg/dL
Impaired hepatic function as indicated by screening aspartate aminotransferase (AST) or alanine aminotransferase (ALT)≥2.5×the upper limit of normal (ULN), or total bilirubin≥1.5×ULN. Note: Participants with Gilbert's syndrome may be eligible to participate.
Hematologic abnormalities as indicated by hemoglobin≤10 g/dL; white blood cells (WBC)≤3 000/mm³; absolute neutrophil count≤1 1,000/mm³; or platelet count≤150,000/mm³.
Has or has had unstable or clinically significant cardiovascular disease (e.g., myocardial infarction, angina pectoris, New York Heart Association Class III or IV cardiac failure) within the past 2 years.
Uncontrolled hypertension (e.g., repeated supine diastolic blood pressure [BP]>95 mm Hg or systolic BP>150 mm Hg) at screening.
History or presence of an abnormal ECG that is clinically significant, including complete left bundle branch block, second- or third-degree atrioventricular block, or evidence of acute or subacute myocardial infarction or ischemia.
History of ventricular dysrhythmias or risk factors for ventricular dysrhythmias such as structural heart disease (e.g., severe left ventricular systolic dysfunction, left ventricular hypertrophy) or clinically significant electrolyte abnormalities (e.g., hypokalemia, hypomagnesemia, hypocalcemia). Note: Participants with premature ventricular contractions are eligible to participate.
For participants who consent to lumbar puncture, contraindication to lumbar dural puncture, including coagulopathy, concomitant anticoagulation medication (except for a platelet inhibitor such as aspirin), thrombocytopenia, or other factor(s) that precludes safe lumbar puncture.
History or presence of clinically evident vascular disease potentially affecting the brain (e.g., clinically significant carotid or vertebral artery stenosis or plaque; cerebral hemorrhage or infarct greater than 1 cm³; 3 or more lacunar infarcts in any location; cerebral contusion; encephalomalacia; intracranial aneurysm; arteriovenous malformation; subdural hematoma); hydrocephalus; space-occupying lesions (e.g., abscess or brain tumor such as meningioma) that have the potential to affect cognitive function; or intracranial tumor that is clinically relevant (e.g., glioma, cerebral metastasis).
History of a clinically significant, persistent neurologic deficit, structural brain damage, or CNS trauma.
Unable to tolerate MRI procedures (e.g., due to anxiety or claustrophobia) or has a contraindication to MRI, including, but not limited to, the presence of pacemakers, aneurysm clips, artificial heart valves, ear implants, or foreign metal objects in the eyes, skin, or body that are not compatible with an MRI scan; or any other clinical history or examination finding that would pose a potential hazard in combination with MRI.

The following medications are prohibited for a pre-specified duration prior to Part 1, as indicated herein, and during the entire period of study participation (Part 1 and Part 2). Participants who start these medications during the study at any time may be withdrawn from study treatment.
Any cannabinoids at least 90 days prior to first study treatment administration on Part 1, unless approved by a Medical Monitor. Use during the study is not permitted within 8 hours before any COA.
Any benzodiazepines and tricyclic antidepressants at least 90 days prior to first study treatment administration on Part 1. Short-term use of benzodiazepines (not more than 3 times per month during the study) or use as premedication prior to lumbar puncture or MRI procedures during the study is allowed; however, use is not permitted within 8 hours before any COA.
Any stimulant medication (e.g., amphetamine, dextroamphetamine, dexmethylphenidate, lisdexamfetamine, methylphenidate) unless prescribed as a stable regimen for at least 90 days prior to the first study treatment administration on Part 1.
Any passive immunotherapy (immunoglobulin) or other long-acting biologic agent that is under evaluation to prevent or postpone cognitive decline within 1 year of Screening for Part 1.
Any exposure to an anti-Sortilin antibody prior to enrollment in Part 1; use of any experimental oral therapy, biologic therapy, or any other investigational treatment within 90 days or 5 half-lives prior to the first study treatment administration on Part 1, whichever is longer.
Any experimental vaccine or gene therapy; routinely recommended vaccinations are allowed, as well as any vaccine against SARS-CoV-2 administered under an Emergency Use Authorization.
Typical (first-generation) antipsychotic or neuroleptic medication within 6 months prior to the first study treatment administration on Part 1, except as needed for brief treatment of a nonpsychiatric indication (e.g., emesis).
Use of atypical (second-generation) antipsychotic medications or use of pimavanserin is allowed during the study if treated with a stable regimen for at least 90 days prior to the first study treatment administration on Part 1.
Anticoagulation (e.g., coumadin, heparinoids, apixaban) medications within 90 days prior to the first study treatment administration on Part 1. Use of aspirin or antiplatelet medication is allowed pre-study and during the study.
Systemic immunosuppressive therapy or use or anticipated systemic immunosuppressive therapy use during the study. Use of prednisone≤10 mg/day or an equivalent corticosteroid is allowed during the study if treated with a stable regimen for at least 90 days prior to the first study treatment administration on Part 1, and participant has hemoglobin>9 g/dL, WBC count>3 000/mm³, absolute neutrophil count>1 500/mm³, and platelet count>100 000/mm³.
Chronic use of opioids (including long-acting opioid medication) within 90 days prior to the first study treatment administration on Part 1. Intermittent short-term use (<1 week) of opioid medications for pain is permitted pre-study and during the study, except within 2 days or 5 half-lives (whichever is longer) prior to any COA.
Chronic use of barbiturates or hypnotics starting from 3 months prior to study treatment administration. Intermittent short-term (<1 week) use of a short-acting hypnotic medication for sleep or anxiety is allowed pre-study and during the study, except within 2 days or 5 half-lives (whichever is longer) prior to any COA.

### IV. Study Assessments

Efficacy COAs include the following:
Clinical Dementia Rating Dementia Staging Instrument PLUS National Alzheimer's Disease Coordinating Center frontotemporal lobar degeneration Behavior and Language Domains (CDR® plus NACC FTLD)
Clinical Dementia Rating Dementia Staging Instrument PLUS National Alzheimer's Disease Coordinating Center Frontotemporal Lobar Degeneration Behavior and Language Domains Sum of Boxes (CDR® plus NACC FTLD-SB)
Clinical Global Impression-Severity (CGI-S)
Clinical Global Impression-Improvement (CGI-I)
Repeatable Battery for the Assessment of Neuropsychological Status (RBANS).

Quality of life COAs include the following:
European Quality of Life-5 Dimensions (EQ-5D)
Zarit Burden Interview (ZBI)

Pharmacoeconomic COAs include the Resource Utilization in Dementia-Lite Version (RUD-Lite).

Exploratory COAs include the following:
Frontotemporal Dementia Rating Scale (FRS)
Winterlight Labs Speech Assessments

Safety assessments include monitoring AEs; physical examinations; neurological examinations; vital signs and weight; ECGs; MRIs; clinical laboratory analyses in blood and urine; suicidality assessments; and ADAs throughout the study.

PD assessments include the following:
Blood-based biomarkers (blood plasma samples for Progranulin protein (PGRN) and blood serum samples for NfL levels).
Exploratory blood-based biomarkers.
Imaging biomarkers: global and regional brain volumes, volume of white matter hyperintensities (measured by volumetric MRI); brain perfusion (measured by arterial spin labeling MRI); and fractional anisotropy, mean diffusivity, axial diffusivity, and radial diffusivity (measured by diffusion-tensor imaging).
CSF-based biomarkers (for PGRN, NfL, and exploratory biomarkers).

Additional whole blood, plasma, and CSF PD biomarker samples are collected for evaluation of neurodegeneration (e.g., tau, phosphorylated tau), lysosomal function (e.g., cathepsins), glial activity (e.g., YKL40, IL-6), and messenger RNA expression in peripheral cells, and for evaluation of levels of other analytes relevant to disease biology and response to study treatment.

Blood and CSF samples are collected throughout Part 1 to measure anti-Sortilin antibody S-60-15.1 [N33T] LALAPS concentrations. Blood samples are collected throughout Part 2 to measure anti-Sortilin antibody S-60-15.1 [N33T] LALAPS concentrations.

### Example 2: Results of a Phase 2 Study to Evaluate an Anti-Sortilin Antibody in Heterozygous Carriers of Granulin or C9orf72 Mutations Causative of Frontotemporal Dementia

This Example provides results of a Phase 2, multicenter, open-label study that evaluated the safety, tolerability, pharmacokinetics, and pharmacodynamics of anti-Sortilin antibody S-60-15.1 [N33T] LALAPS in heterozygous carriers of Granulin or C9orf72 mutations causative of frontotemporal dementia (FTD).

A. Study Design

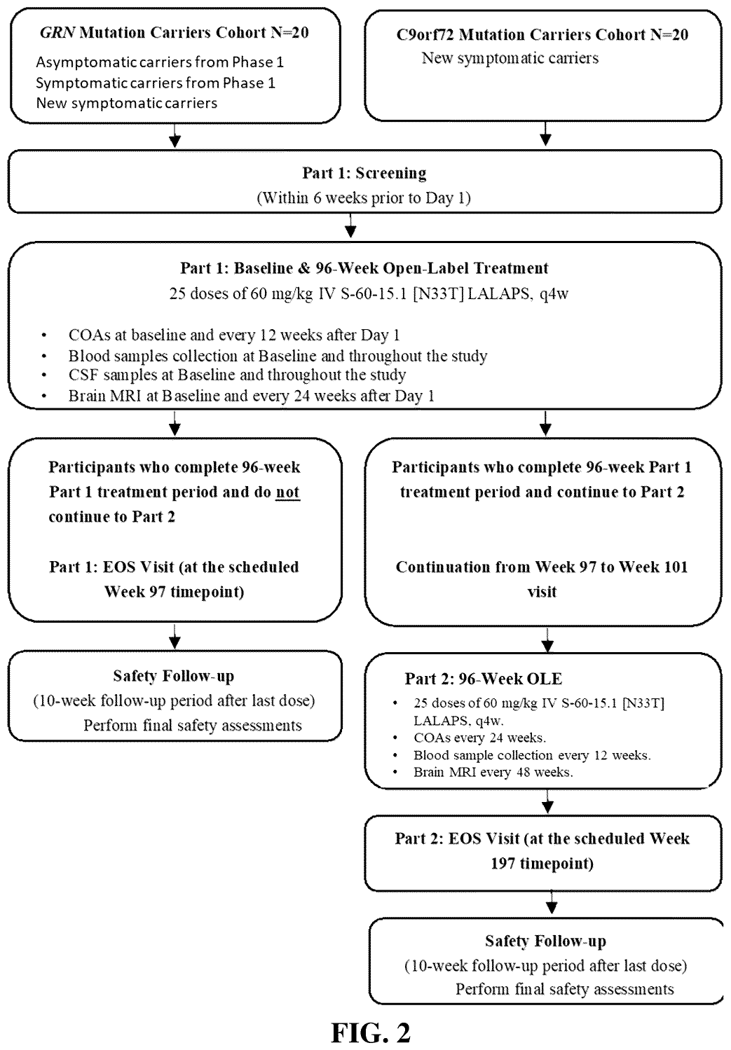

This Phase 2 study was conducted as described in Example 3 of WO2020252066, which is incorporated herein by reference in its entirety, and as further described below. An overview of the design of this study is provided in FIG. 2.

Part 1—Open-Label Treatment Period

As described in Example 3 of WO2020252066, during the open-label treatment period, termed “Part 1” herein, asymptomatic carriers (aFTD-GRN) and symptomatic carriers (FTD-GRN) of loss-of-function Granulin mutations causative of FTD, and symptomatic carriers of C9orf72 hexanucleotide repeat expansion mutations causative of FTD, were administered anti-Sortilin antibody S-60-15.1 [N33T] LALAPS intravenously (IV) at a dose of 60 mg/kg every 4 weeks (q4w).

The cohorts included in this part of the study were as described in Example 3 of WO2020252066, and as further described below:
Participant Category 1—Symptomatic Granulin mutation carriers from the previous Phase 1 study (as described in Example 3 of WO2020252066).
   Completed the previous Phase I study through the Day 57 visit and did not experience adverse events (AEs) that would prevent safe participation in this Phase 2 study.
   All previous participants in the Phase I study were rescreened and met all inclusion/exclusion criteria applicable to this study.
   Had one or more of the six behavioral/cognitive symptoms required for a diagnosis of possible behavioral variant frontotemporal dementia (bvFTD; Rascovsky et al., Brain. (2011) 134(9):2456-2477), or had a diagnosis of primary progressive aphasia (PPA; Gorno-Tempini et al., Neurology (2011) 76(11):1006-1014).
Participant Category 2—Asymptomatic Granulin mutation carriers from the previous Phase 1 study.
   Completed the previous Phase I study through the Day 43 visit and did not experience AEs that would prevent safe participation in this Phase 2 study.
   All previous participants in the Phase I study were rescreened and met all inclusion/exclusion criteria applicable to this study.
   If a participant became symptomatic during or after the previous Phase I study, they were screened under Participant Category 3 (symptomatic).
Participant Category 3—New symptomatic Granulin mutation carriers.
   Carriers of a loss-of-function Granulin mutation causative of FTD that knew their mutation status.
   Had a Clinical Dementia Rating Dementia Staging Instrument PLUS National Alzheimer's Disease Coordinating Center Frontotemporal Lobar Degeneration Behavior and Language Domains (CDR® plus NACC FTLD) global score of 0.5, 1, or 2; and one or more of the six behavioral/cognitive symptoms required for a diagnosis of possible bvFTD (Rascovsky et al., Brain (2011) 134(9):2456-2477), or a diagnosis of PPA (Gorno-Tempini et al., Neurology (2011) 76(11):1006-1014).
Participant Category 4—New symptomatic C9orf72 mutation carriers.
   Carriers of a hexanucleotide repeat expansion C9orf72 mutation causative of FTD that knew their mutation status.
   Had a CDR® plus NACC FTLD global score of 0.5, 1, or 2; and one or more of the six behavioral/cognitive symptoms required for a diagnosis of possible bvFTD (Rascovsky et al., Brain (2011) 134(9):2456-2477), or a diagnosis of PPA (Gorno-Tempini et al., Neurology (2011) 76(11):1006-1014).

The 48-week treatment period as described in Example 3 of WO2020252066 was extended to a 96-week treatment period in this part of the study, including a total of 25 doses of anti-Sortilin antibody S-60-15.1 [N33T] LALAPS.

Part 2—Open-Label Extension

An open-label extension (OLE) “Part 2” was added to this Phase 2 study. Part 2 of the study included study participants who completed the first 96-week open-label treatment period in Part 1, as described above. Participants with a CDR® plus NACC FTLD global score>2 were not eligible for participation in Part 2.

In Part 2 of the study, participants were administered up to an additional 25 doses of anti-Sortilin antibody S-60-15.1 [N33T] LALAPS (60 mg/kg IV q4w, for a total OLE treatment period of 96 weeks).

The primary objective of Part 2 of the study was to assess the long-term safety and tolerability of IV administration of anti-Sortilin antibody S-60-15.1 [N33T] LALAPS in participants who completed 96 weeks of treatment in Part 1 of the study.

The exploratory objectives of Part 2 of the study were to assess the long-term effect of IV administration of anti-Sortilin antibody S-60-15.1 [N33T] LALAPS in participants who completed 96 weeks of treatment in Part 1 of the study based on the following:
Pharmacokinetics (PK).
Longitudinal plasma Progranulin (PGRN) concentration levels.
Longitudinal blood and plasma levels of exploratory pharmacodynamic (PD) biomarkers of neurodegeneration, lysosomal function, and glial activity.
Magnetic resonance imaging (MRI) measures to evaluate changes in the brain.
Correlations among exploratory fluid PD biomarkers, imaging PD measures, and clinical outcome assessments (COAs).
Clinical progression as measured by COAs.

### B. Study Assessments

The endpoints and assessments of this Phase 2 study were as described in Example 3 of WO2020252066, and also included clinical outcome assessments (COAs) based on the Clinical Dementia Rating Dementia Staging Instrument PLUS National Alzheimer's Disease Coordinating Center Frontotemporal Lobar Degeneration Behavior and Language Domains (CDR® plus NACC FTLD), and the Clinical Global Impression of Severity (CGI-S).

### C. Results

Safety

Anti-Sortilin antibody S-60-15.1 [N33T] LALAPS (administered at a dose of 60 mg/kg IV q4w over approximately 60 minutes) was generally safe and well-tolerated across all cohorts in this study. All adverse events (AEs) were mild. Three symptomatic carriers of loss-of-function Granulin mutations causative of FTD (FTD-GRN participants) experienced a serious AE, but the AEs were considered unrelated to anti-Sortilin antibody S-60-15.1 [N33T] LALAPS. The safety profile was comparable between asymptomatic and symptomatic participants. An overview of safety results of the study is provided in Table 21.

TABLE 21
Safety profile of anti-Sortilin antibody
S-60-15.1 [N33T] LALAPS.

| | aFTD-GRN | FTD-GRN | FTD-C9orf72 | Total |
|---|---|---|---|---|
| | (N = 5) | (N = 12) | (N = 10) | (N = 27) |
| | n (%) | n (%) | n (%) | n (%) |
| Any TEAE | 5 | 9 | 8 | 22 |
| | (100.0) | (75.0) | (80.0) | (81.5) |
| Any treatment- | 1 | 1 | 4 | 6 |
| related TEAE | (20.0) | (8.3) | (40.0) | (22.2) |
| Any SAE | 0 | 3 | 0 | 3 |
| | | (25.0) | | (11.1) |
| Any treatment- | 0 | 0 | 0 | 0 |
| related SAE | | | | |
| Any TEAE | 0 | 1* | 0 | 1* |
| leading to study | | (8.3) | | (3.7) |
| drug | | | | |
| discontinuation | | | | |

aFTD-GRN, asymptomatic carriers of loss-of-function Granulin mutations causative of FTD; FTD-GRN, symptomatic carriers of loss-of-function Granulin mutations causative of FTD; FTD-C9orf72, symptomatic carriers of C9orf72 hexanucleotide repeat expansion mutations causative of FTD; TEAE, treatment emergent adverse event; SAE, serious adverse event.
*Discontinuation from study was not related to treatment of anti-Sortilin antibody S-60-15.1 [N33T] LALAPS.

Similar safety results to those provided in Table 21 were observed when safety data were obtained from additional patients (asymptomatic and symptomatic FTD-GRN participants and FTD-C9orf72 participants) in this study. Anti-Sortilin antibody S-60-15.1 [N33T] LALAPS was found to be generally safe and well-tolerated for a median treatment duration of 12 months for all study participants.

Biomarkers

PGRN

To assess levels of PGRN in plasma and cerebrospinal fluid (CSF) in symptomatic carriers of loss-of-function Granulin mutations causative of FTD (FTD-GRN participants), plasma samples were collected at each monthly visit of the study, and CSF samples were collected at baseline (week 0), 6 months (week 25), and 12 months (week 49) after the start of study treatment. The plasma and CSF samples were collected prior to dosing of anti-Sortilin antibody S-60-15.1 [N33T] LALAPS at each visit. Plasma and CSF samples were also obtained from age-matched procured controls to establish the range of PGRN in the plasma and CSF of individuals who were not FTD-Granulin mutation carriers. PGRN concentrations in plasma and CSF were determined via an enzyme-linked immunosorbent assay (ELISA).

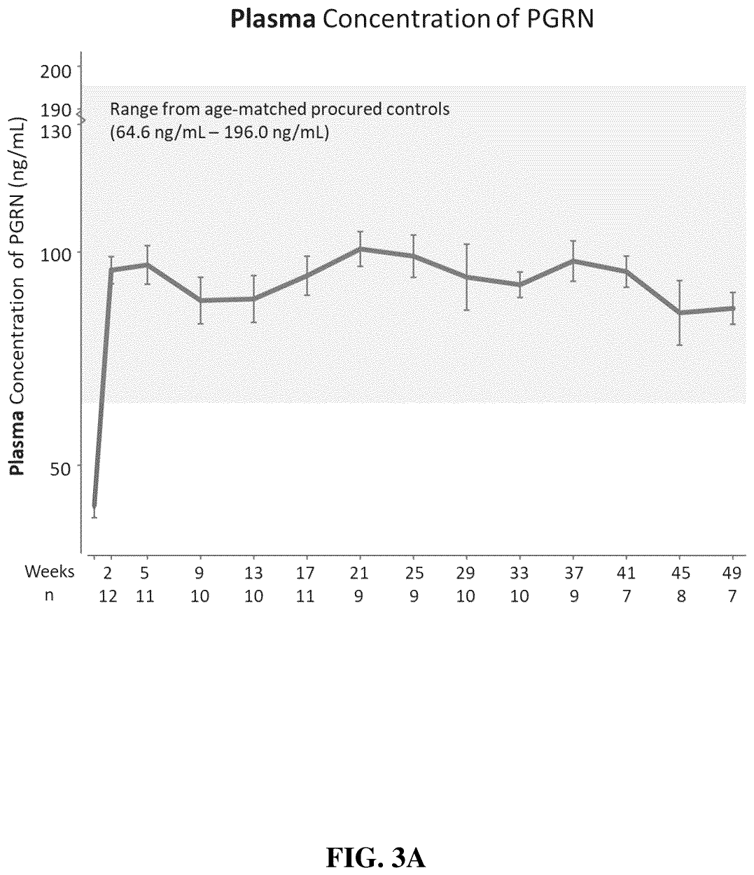

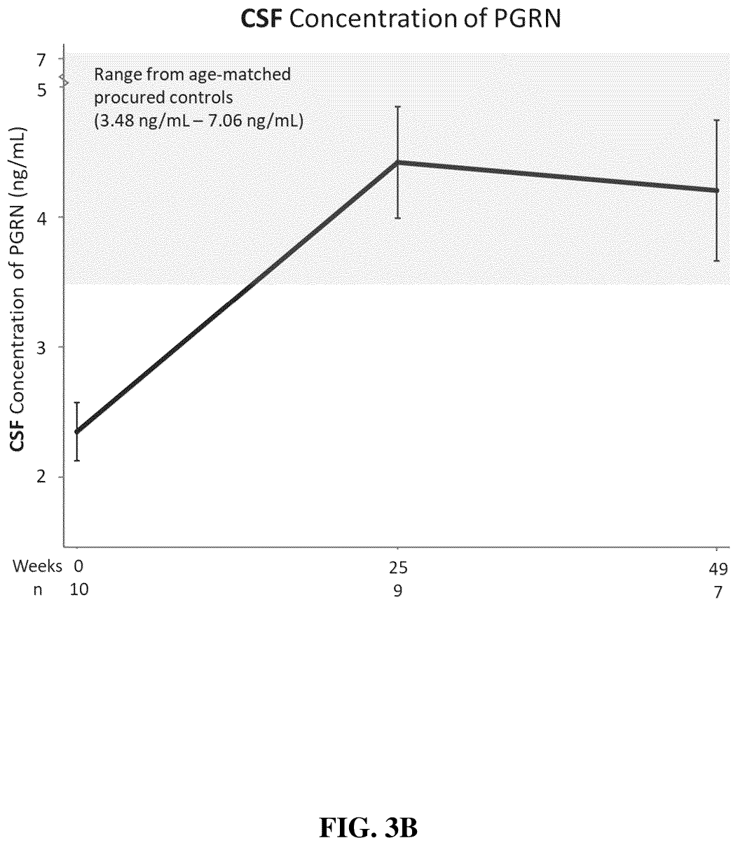

As shown in FIGS. 3A-3B, all FTD-GRN participants (N=12) had baseline plasma and CSF PGRN concentrations substantially below the normal levels observed in age-matched procured controls (64.6 ng/mL-196.0 ng/mL in plasma; 3.48 ng/mL-7.06 ng/mL in CSF). Administration of anti-Sortilin antibody S-60-15.1 [N33T] LALAPS elevated plasma (FIG. 3A) and CSF (FIG. 3B) PGRN to levels within the range observed in the age-matched controls. The elevated levels of PGRN in the plasma and CSF of FTD-GRN participants were sustained with chronic dosing of anti-Sortilin antibody S-60-15.1 [N33T] LALAPS through one year (week 49) of treatment. These results demonstrated that chronic treatment with anti-Sortilin antibody S-60-15.1 [N33T] LALAPS restored PGRN levels to normal levels in plasma and CSF in FTD-GRN patients.

Additional data on the levels of PGRN in plasma and CSF of FTD-GRN participants in the study were obtained, and the results were similar to those shown in FIGS. 3A and 3B.

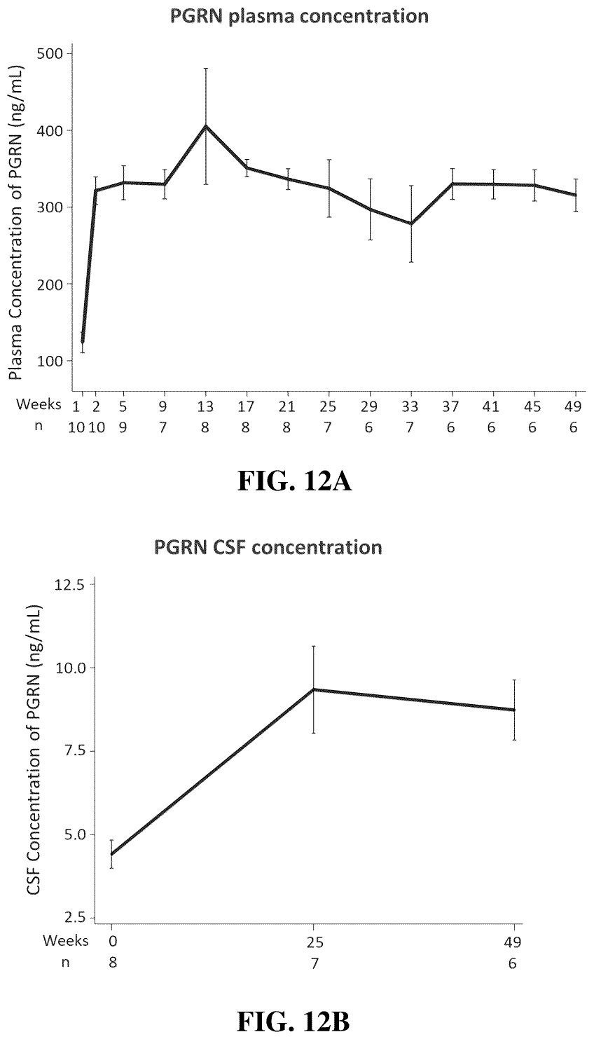

The levels of PGRN in plasma and CSF were also assessed in FTD-C9orf72 participants in the study using an ELISA as described above. As shown in FIGS. 12A-12B, administration of anti-Sortilin antibody S-60-15.1 [N33T] LALAPS elevated plasma (FIG. 12A) and CSF (FIG. 12B) PGRN in FTD-C9orf72 participants to levels above baseline. The elevated levels of PGRN in the plasma and CSF of FTD-C9orf72 participants were sustained with chronic dosing of anti-Sortilin antibody S-60-15.1 [N33T] LALAPS through one year (week 49) of treatment. These results demonstrated that chronic treatment with anti-Sortilin antibody S-60-15.1 [N33T] LALAPS elevated PGRN levels in plasma and CSF in FTD-C9orf72 participants, which is indicative of target engagement and the potential for clinical benefit.

Lysosomal and Complement Biomarkers

The levels of lysosomal and complement biomarkers, which represent key pathophysiological pathways impacted by PGRN deficiency, were assessed in the CSF of FTD-GRN participants and in procured age-matched controls by targeted mass spectrometry (Multiple Reaction Monitoring Liquid Chromatography-Mass Spectrometry).

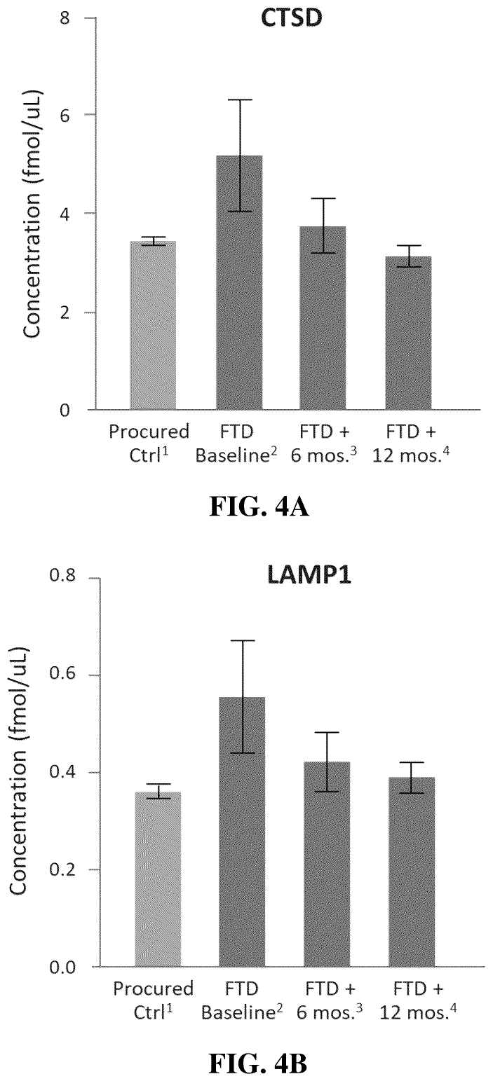

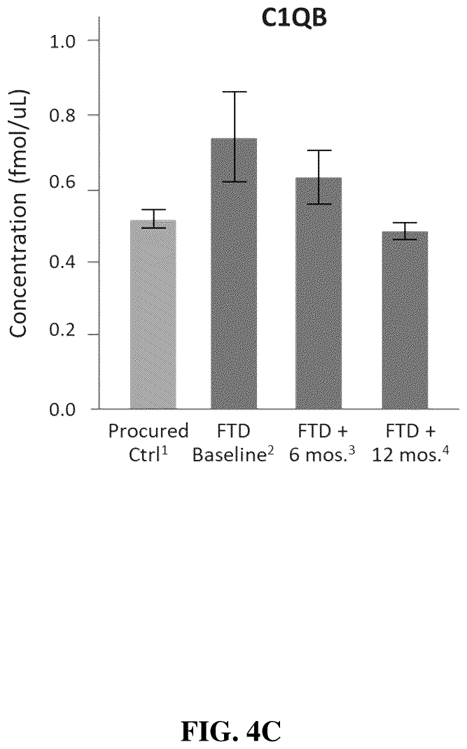

As shown in FIGS. 4A-4C, Cathepsin D and LAMP1, which are biomarkers of lysosomal function, and C1QB, a component of the complement cascade that indicates inflammation, were elevated in the CSF of FTD-GRN participants at baseline when compared to age-matched controls. The elevated levels of these biomarkers suggested the presence of dysfunctional lysosomes and aberrant complement activation in FTD-GRN participants. A reduction of these biomarkers to near control levels was observed at 6 months and 12 months after the start of treatment with anti-Sortilin antibody S-60-15.1 [N33T] LALAPS (FIGS. 4A-4C), suggesting that anti-Sortilin antibody S-60-15.1 [N33T] LALAPS restored normal lysosome and complement function in FTD-GRN participants.

Additional data on the levels of Cathepsin D, LAMP1, and C1QB in CSF of FTD-GRN participants in the study were obtained, and the results were similar to the results shown in FIGS. 4A-4C at 6 months and 12 months after the start of treatment with anti-Sortilin antibody S-60-15.1 [N33T] LALAPS.

Neurofilament Light Chain (NfL)

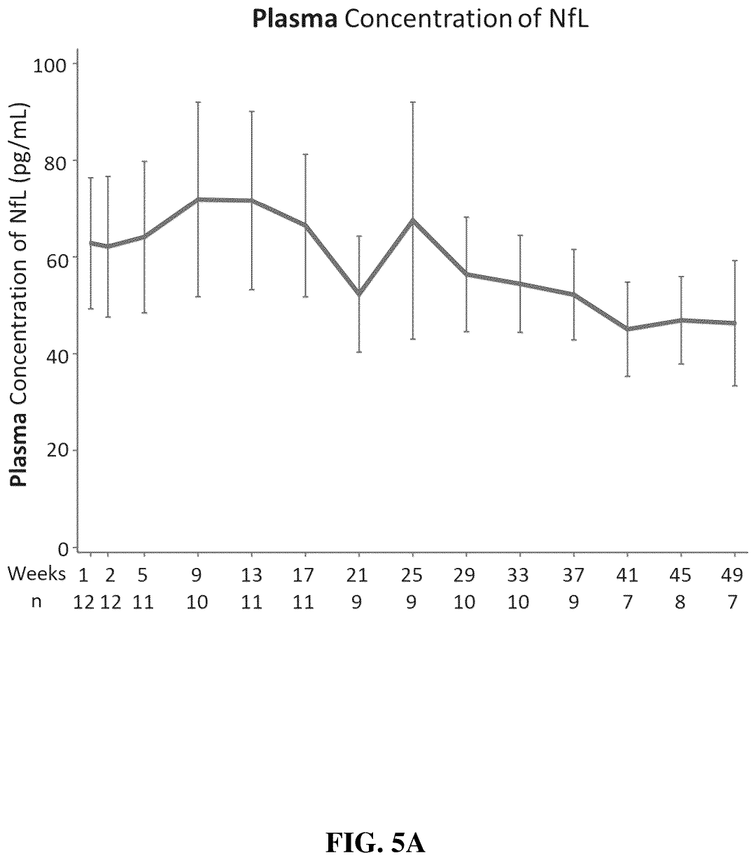

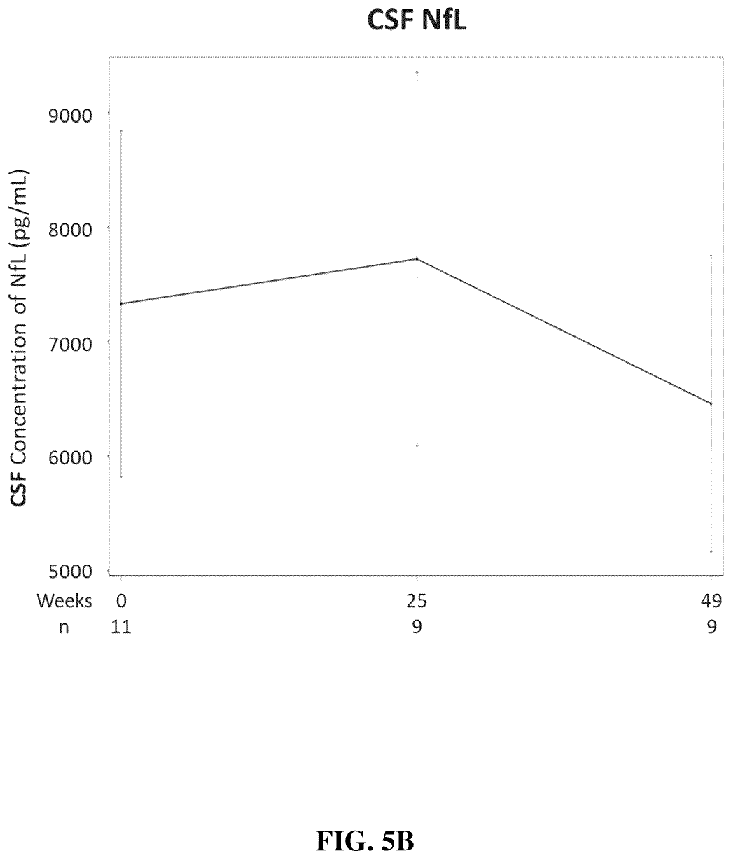

The levels of neurofilament light chain (NfL), a biomarker of neuronal health, were assessed in both plasma and CSF of FTD-GRN participants by a Simoa assay. As shown in FIGS. 5A-5B, longitudinal NfL measurements exhibited variability, and the data suggested that plasma (FIG. 5A) and CSF (FIG. 5B) NfL levels were stable relative to baseline over one year (49 weeks).

Additional data on the levels of NfL in plasma and CSF of FTD-GRN participants in the study were obtained, and the results were similar to the results shown in FIGS. 5A-5B.

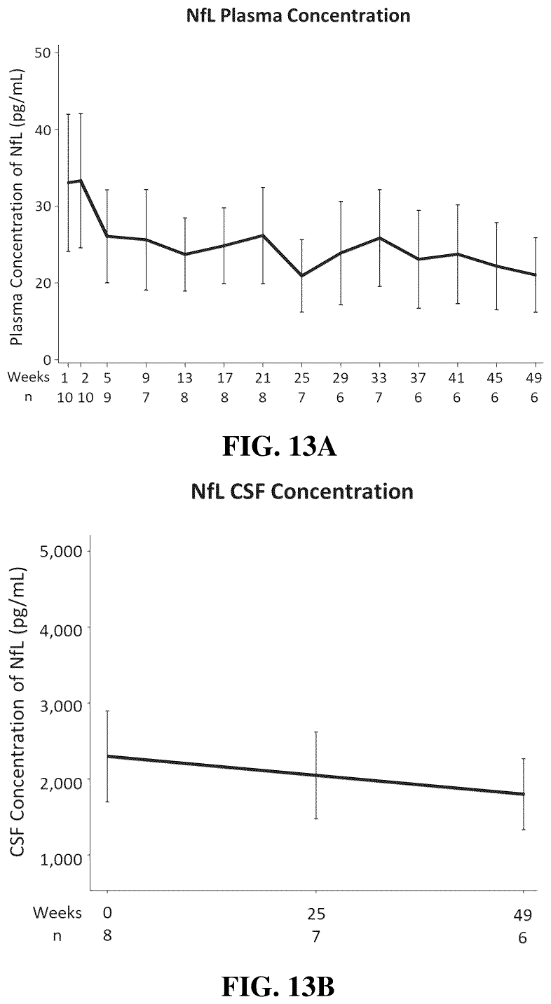

The levels of NfL were also assessed in both plasma and CSF of FTD-C9orf72 participants in the study using a Simoa assay as described above. As shown in FIGS. 13A-13B, NfL levels in plasma and CSF were stable over 12 months in FTD-C9orf72 participants treated with anti-Sortilin antibody S-60-15.1 [N33T] LALAPS.

Biomarkers of Astrogliosis

Astrocytes are the most abundant cell type in the adult central nervous system (CNS), and execute vital functions in the maintenance and homeostasis of the CNS. Glial fibrillary acidic protein (GFAP) is a major component of the astrocytic cytoskeleton and serves as a marker of astrogliosis, which is an abnormal proliferation of astrocytes due to neuronal damage. GFAP has been found to be elevated in symptomatic FTD-GRN patients, as compared to age-matched controls. See, Heller et al. J. Neurol Neurosurg Psychiatry 2020; 91:263-270. In addition, higher levels of GFAP at baseline are correlated with faster rates of atrophy in the temporal lobe of symptomatic FTD-GRN patients. See, Heller et al. Plasma GFAP levels in pre-symptomatic FTD-GRN patients were also found to be similar to plasma GFAP levels in controls. See id.

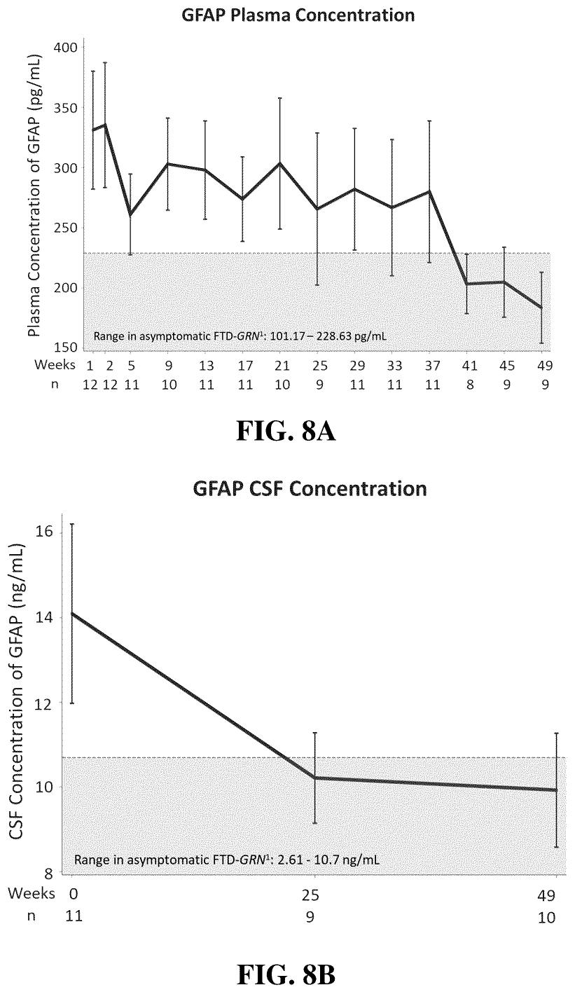

The levels of GFAP were assessed in both plasma and CSF of FTD-GRN participants in the study. Plasma and CSF GFAP levels were measured using the Simoa (single molecule array technology) assay by Quanterix. As shown in FIGS. 8A-8B, administration of anti-Sortilin antibody S-60-15.1 [N33T] LALAPS resulted in decreases of GFAP levels that fall within the range of GFAP levels in plasma (FIG. 8A) and CSF (FIG. 8B) of asymptomatic FTD-GRN patients, suggesting a reduction in astrogliosis.

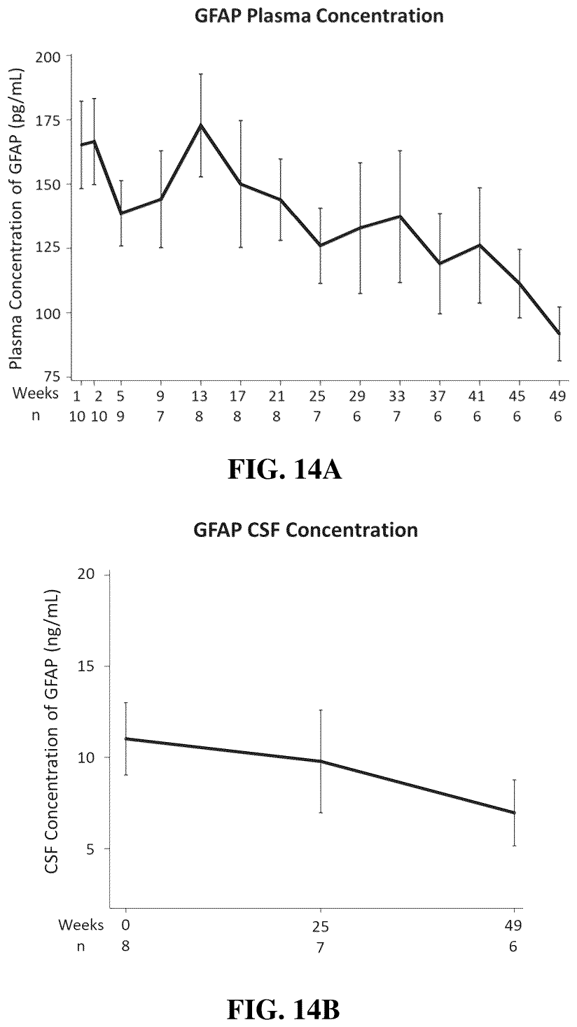

The levels of GFAP were also assessed in both plasma and CSF of FTD-C9orf72 participants in the study using a Simoa assay as described above. As shown in FIGS. 14A-14B, administration of anti-Sortilin antibody S-60-15.1 [N33T] LALAPS resulted in decreases of GFAP levels over 12 months, suggesting a reduction in astrogliosis.

Biomarkers of Neuroinflammation

Macrophage migration inhibitory factor (MIF) is a pleiotropic pro-inflammatory cytokine that is highly and widely expressed in human neural tissues, including neurons, microglia, astrocytes, and ependymal cells. MIF can promote the secretion of other inflammatory response mediators, including IL6 and TNF-α, and can activate the inflammasome. Thus, MIF may serve as a biomarker of neuroinflammation.

While elevated levels of MIF have been observed in the CSF of Alzheimer's disease patients compared to age-matched controls (see, e.g., Zhang et al., Alzheimers Res Ther. 2019; 11(1):54), the levels of MIF in FTD have not been previously studied.

To evaluate MIF in FTD, the levels of MIF protein were assessed in the CSF of FTD-GRN and FTD-C9orf72 participants in the Phase 2 study of anti-Sortilin antibody S-60-15.1 [N33T] LALAPS described herein, as well as in age-matched procured controls. In addition, the levels of MIF protein were also assessed in the CSF of FTD-GRN participants and healthy volunteers from a Phase 1 study of anti-Sortilin antibody S-60-15.1 [N33T] LALAPS described in Examples 2 and 5 of WO2020252066, which is incorporated herein by reference in its entirety.

Methods

MIF protein levels in CSF were assessed using a quantitative colorimetric sandwich enzyme-linked immunosorbent assay (ELISA).

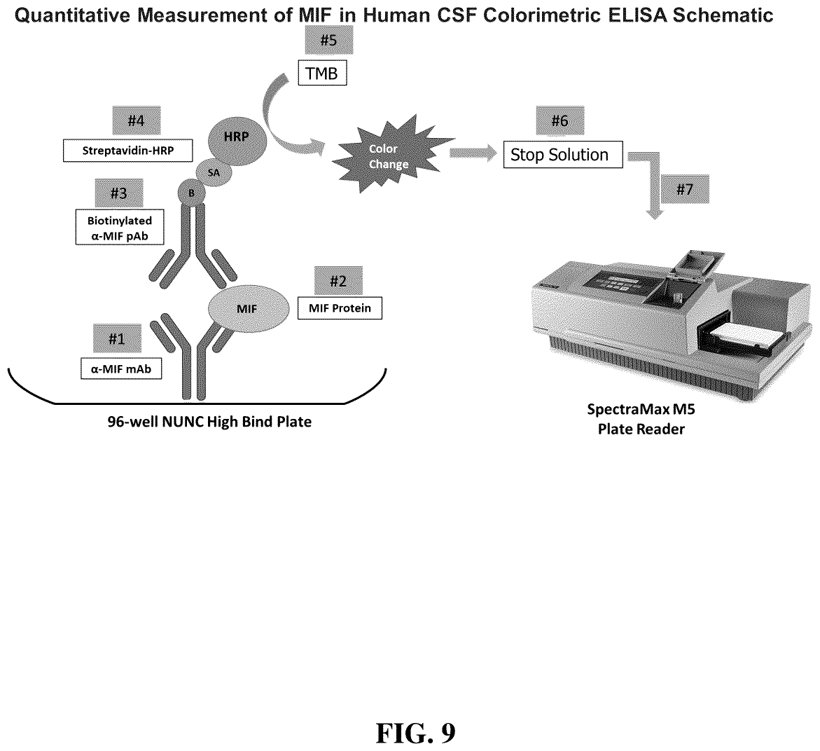

The design of the colorimetric ELISA method for the measurement of MIF levels in CSF is depicted in FIG. 9. In the ELISA method, a monoclonal antibody against MIF was coated onto 96-well microtiter plates (e.g., 96-well NUNC High Binding ELISA Plates). Standards and samples were pipetted into the wells for binding to the coated antibody. After washing to remove unbound compounds, MIF was detected by the addition of a biotinylated polyclonal antibody against MIF. Following a wash to remove excess biotinylated antibody, horseradish peroxidase (HRP)-labeled streptavidin (STREP-HRP) was added. A final wash was performed, and 3,3′,5,5′-tetramethylbenzidine (TMB) substrate solution was added to the wells to allow color development, which occurs in proportion to the concentration of MIF in the samples. The color development was stopped with stop solution, and the intensity of the color development was measured using a plate reader (e.g., a SpectraMax M5 Plate Reader). The ELISA method requirements and characteristics are provided in Table 22.

TABLE 22
Detailed description of a colorimetric ELISA
used to measure MIF levels in human CSF.

| Analyte Name | Macrophage Migration Inhibitory |
|---|---|
| | Factor (MIF) |
| Assay Type | Colorimetric ELISA |
| Biological Matrix | Human Cerebrospinal Fluid (CSF) |
| Minimum Required Dilution (MRD) | 1:5 |
| Calibration Standards Range (12 | 20,000 pg/mL to 350 pg/mL |
| Points) | |
| Quantifiable Range (10 Points) | 15,000 pg/mL to 500 pg/mL |
| Anchor Point(s) | 20,000 pg/mL and 350 pg/mL |
| Regression, Weighting | 4-Parameter Logistic curve fit, |
| | with 1/y² weighing |
| # of Samples per Assay Plate | 26 |

Results

The levels of MIF protein in CSF were assessed using the ELISA method described above.

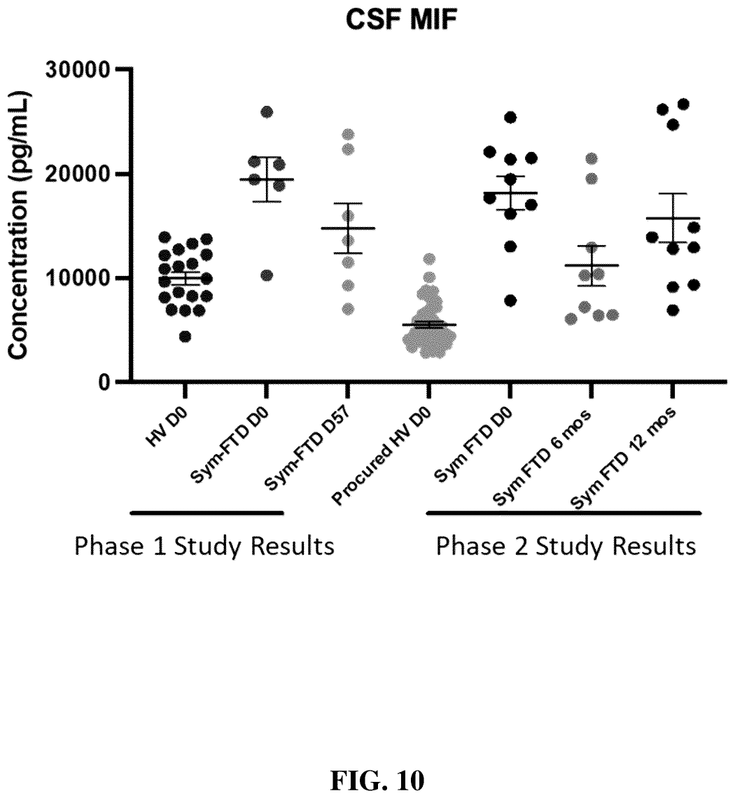

MIF protein levels were found to be elevated at baseline (Day 0) in the CSF of FTD-GRN participants in the Phase 2 study described herein, as compared to age-matched procured controls (FIG. 10). Administration of anti-Sortilin antibody S-60-15.1 [N33T] LALAPS resulted in a decrease in the levels of MIF in the CSF of FTD-GRN participants at 6 months and 12 months after the start of treatment with the anti-Sortilin antibody, as compared to baseline levels of MIF (FIG. 10). The mean and median percent decreases in CSF levels of MIF in FTD-GRN participants at 12 months after the start of treatment with the anti-Sortilin antibody as compared to baseline levels of MIF were −19.2% and −21.6%, respectively.

Similarly, as shown in FIG. 10, MIF protein levels were found to be elevated at baseline (Day 0) in the CSF of FTD-GRN participants in the Phase 1 study described above, as compared to healthy volunteers (FIG. 10). Administration of anti-Sortilin antibody S-60-15.1 [N33T] LALAPS resulted in a decrease in the levels of MIF in the CSF of FTD-GRN participants at Day 57 of the Phase 1 study, as compared to baseline levels of MIF (FIG. 10).

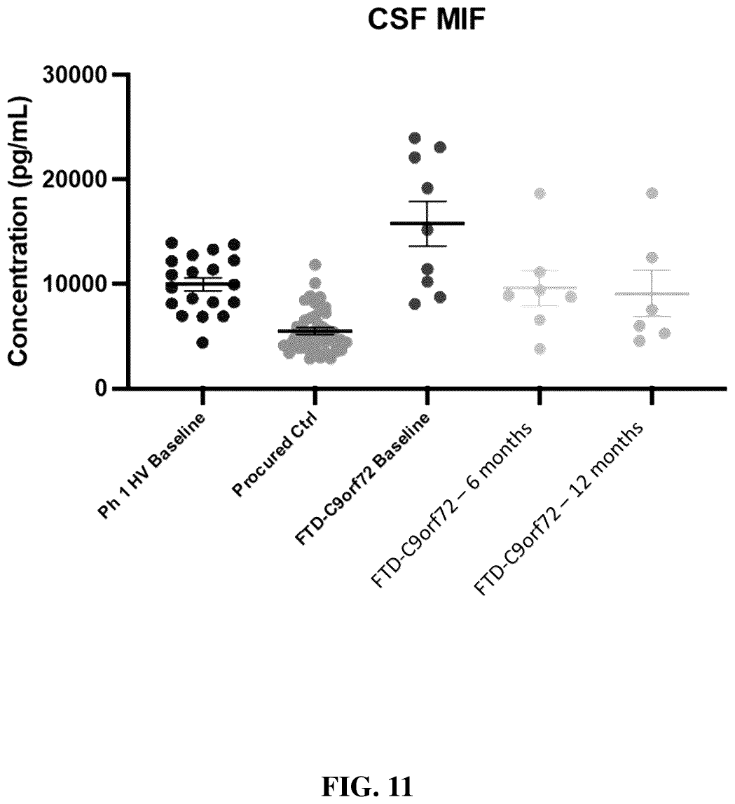

The levels of MIF protein in CSF were also found to be elevated at baseline in symptomatic carriers of C9orf72 hexanucleotide repeat expansion mutations causative of FTD (FTD-C9orf72 participants) in the Phase 2 study described herein, as compared to procured age-matched controls and to healthy volunteers from the Phase 1 study described above (FIG. 11). Specifically, FTD-C9orf72 participants had a median percent increase in CSF MIF levels of +211% and +53% as compared to procured age-matched controls and to healthy volunteers from the Phase 1 study, respectively. Administration of anti-Sortilin antibody S-60-15.1 [N33T] LALAPS resulted in decreases in CSF MIF levels in FTD-C9orf72 participants at 6 months and 12 months after the start of treatment with the anti-Sortilin antibody, with a mean and median percent change at 12 months of −25.0% and −51.5%, respectively.

Brain Assessments

The effect of anti-Sortilin antibody S-60-15.1 [N33T] LALAPS on brain atrophy, a hallmark of all neurodegenerative diseases, was assessed using volumetric magnetic resonance imaging (vMRI) in FTD-GRN participants. Volume changes of the whole brain, the frontotemporal lobe, and the ventricles were measured over one year. Tensor-based Morphometry (TBM) was used for evaluation of frontotemporal cortex and ventricles, and volume subtraction was used for evaluation of whole-brain (gray+white matter). The vMRI results were compared to a historical, synthetic negative control, which was generated for this Phase 2 study using the Propensity score method, which is intended to mimic randomization. Propensity score matching is an established statistical technique to construct a synthetic control group by matching treated subjects with non-treated subjects of similar characteristics so as to more accurately estimate the effect of a treatment. The synthetic control included 10 patients with Granulin mutations that matched the FTD-GRN participants in the Phase 2 study with respect to disease severity as measured by CDR®-NACC-FTLD-SB, age, NfL plasma levels and FTD subtype at baseline. The 10 patients were selected in a blinded fashion, and longitudinal data was obtained for the 10 patients (www.genfi.org). Through the matching process, the matched historical controls have comparable baseline characteristics as the study participants, allowing for a meaningful comparison and estimation of the treatment effect.

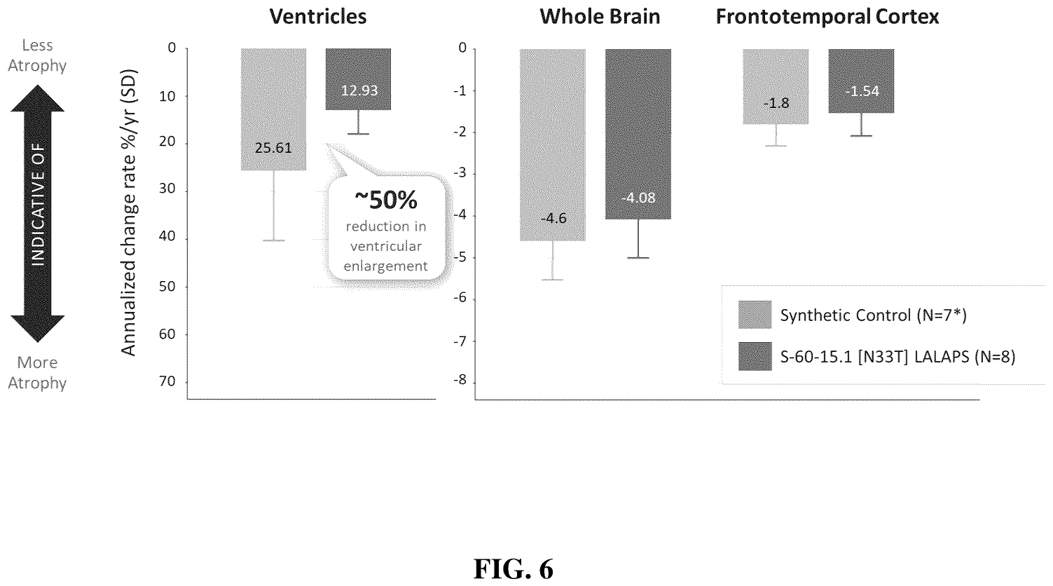

As shown in FIG. 6, volumetric changes in brain tissue were observed in the synthetic control group over one year, as indicated by the annualized change rate percentage per year (y-axis), with the largest change observed being enlargement of the ventricles. Treatment with anti-Sortilin antibody S-60-15.1 [N33T] LALAPS demonstrated a trend towards a reduction in ventricle enlargement (approximately 50% reduction) observed in patients treated with anti-Sortilin antibody S-60-15.1 [N33T] LALAPS, as compared to the synthetic controls.

Clinical Assessments

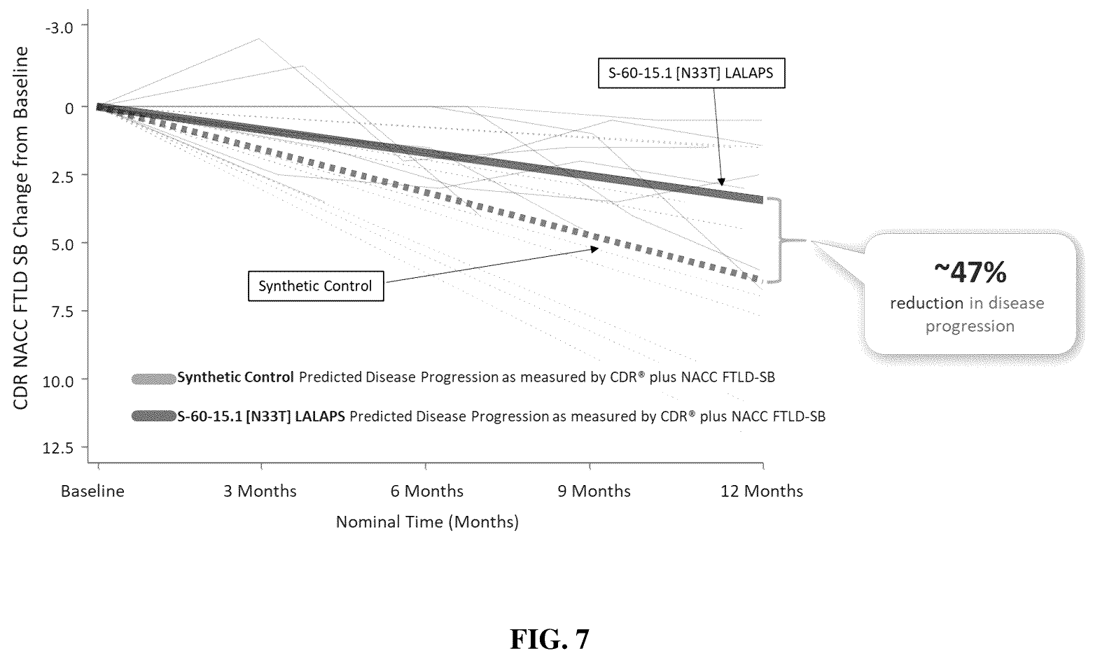

To assess the effect of anti-Sortilin antibody S-60-15.1 [N33T] LALAPS on clinical disease progression, CDR® plus NACC FTLD-SB scores were measured in FTD-GRN participants, and were compared to the synthetic control group described above. A Random Coefficient Model with Repeated Measurements (including baseline and all available post-baseline measurements up to 12 months) was used to estimate clinical disease progression from baseline to 12 months for the FTD-GRN participants (n=12) and for the synthetic control participants (n=10). In the control group, CDR® plus NACC FTLD-SB scores increased by 6.4 points from baseline over one year, indicating rapid disease progression (data not shown). In contrast, the estimated annual change of CDR® plus NACC FTLD-SB scores in FTD-GRN participants in this study was 3.4 points, which is a 3.0 point difference from the synthetic control group (data not shown), representing an approximately 47% reduction (or delay) in disease progression compared to the synthetic control group, as shown in FIG. 7.

Additional data on CDR® plus NACC FTLD-SB scores of FTD-GRN participants in the study were obtained, and the results were similar to the results shown in FIG. 7.

To assess the effect of anti-Sortilin antibody S-60-15.1 [N33T] LALAPS on clinical disease progression in FTD-C9orf72 study participants, CDR® plus NACC FTLD-SB scores were measured and compared to a matched historical control cohort. The matched historical control cohort was generated from ALLFTD (www.allftd.org) participants with a confirmed C9orf72 genetic mutation (herein, “historical control FTD-C9orf72 participants”) in a 2-step blinded matching process. In the first step, a propensity score matching (as described above in the “Brain Assessments” section) based on the baseline CDR® plus NACC FTLD-SB scores was performed between the FTD-C9orf72 study participants (n=10) and the historical control FTD-C9orf72 participants from the ALLFTD database who had a baseline and at least one post-baseline CDR® plus NACC FTLD-SB score (n=84). Propensity score matching identified 29 historical control FTD-C9orf72 participants with comparable baseline CDR® plus NACC FTLD-SB scores to each of the FTD-C9orf72 study participants. In the second step, a blinded clinical adjudication matching was performed between the 29 historical control FTD-C9orf72 participants and each of the 10 FTD-C9orf72 study participants based on baseline NfL levels, age, diagnosis, and sex. Following these 2 steps, a cohort of 10 historical control FTD-C9orf72 participants formed the matched historical control cohort for the FTD-C9orf72 study participants. Since this was an open-label single-arm study, the matching was intended to mimic randomization, thus allowing for a meaningful comparison and estimation of treatment effect. The matching was considered successful, as evidenced by comparable baseline characteristics between the FTD-C9orf72 study participants and the matched historical control cohort.

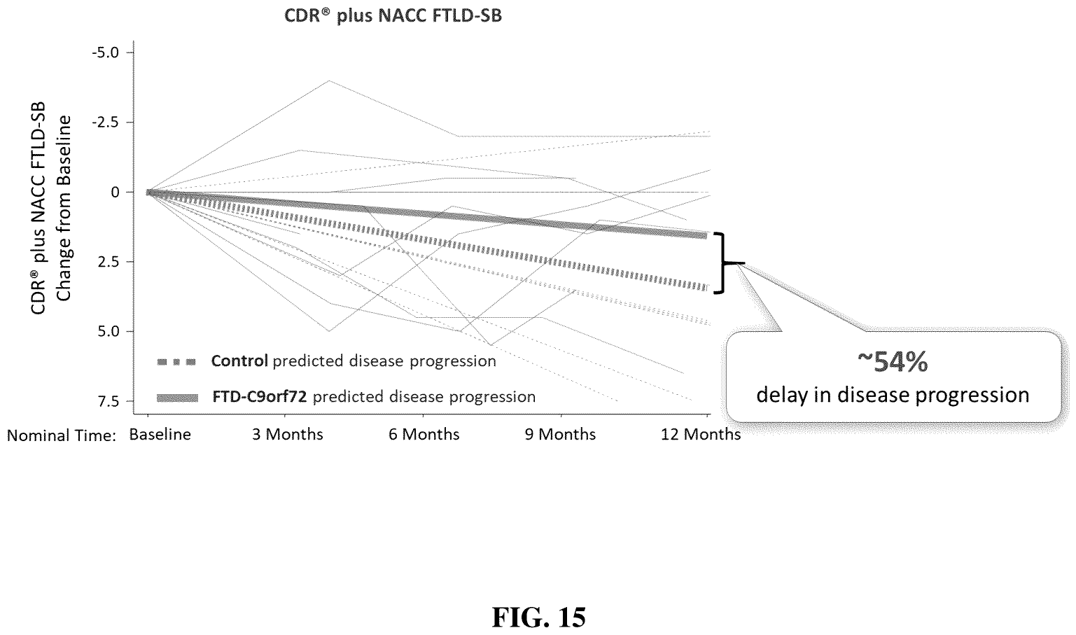

A Random Coefficient Model with Repeated Measurements was used to estimate clinical disease progression from baseline to 12 months for FTD-C9orf72 study participants (n=10; including baseline and all available post-baseline assessments ranging from 3 to 12 months) and for the matched historical control cohort (n=10; including baseline and one post-baseline time point at about 12 months). In the matched historical control cohort, CDR® plus NACC FTLD-SB scores increased by 3.4 points from baseline over one year (95% Confidence Interval: 1.30, 5.60; data not shown). In contrast, the estimated annual change of CDR® plus NACC FTLD-SB scores in FTD-C9orf72 participants in this study was 1.6 points (95% Confidence Interval: −0.63, 3.78), which represents a 1.9 point (95% Confidence Interval: −1.21, 4.95) difference from the matched historical control cohort (data not shown). As shown in FIG. 15, the difference in the annual change of CDR® plus NACC FTLD-SB scores between FTD-C9orf72 study participants treated with anti-Sortilin antibody S-60-15.1 [N33T] LALAPS and the matched historical control cohort represents an approximately 54% delay in disease progression compared to the matched historical control cohort.

Another sensitivity analysis using a Random Coefficient with Repeated Measurement Model was performed for FTD-C9orf72 study participants as compared to matched historical controls. This model expanded the matched historical control cohort described above to include all historical control FTD-C9orf72 participants (from the ALLFTD database) having propensity score-matched baseline CDR® plus NACC FTLD-SB scores (n=29), and did not require clinical adjudication based on baseline NfL levels, age, diagnosis, and sex. Using this statistical method, the estimated difference in the annual change of CDR® plus NACC FTLD-SB scores between FTD-C9orf72 study participants treated with anti-Sortilin antibody S-60-15.1 [N33T] LALAPS and the matched historical control cohort was approximately 0.9 points, representing a 36% delay in disease progression compared to the matched historical control cohort.

Results of the clinical assessments described above indicate a potential for a clinically meaningful benefit for FTD-C9orf72 patients.

### D. Conclusions

Anti-Sortilin antibody S-60-15.1 [N33T] LALAPS was found to be generally safe and well-tolerated. Administration of anti-Sortilin antibody S-60-15.1 [N33T] LALAPS at a dose of 60 mg/kg IV q4w to symptomatic FTD-GRN and FTD-C9orf72 patients over 12 months resulted in consistent, positive changes across multiple parameters along the disease cascade of FTD-GRN and FTD-C9orf72 patients, suggesting a slowing of disease progression.

In particular, such treatment restored PGRN to normal physiological levels in plasma and CSF of FTD-GRN patients. Treatment also resulted in a time-dependent and durable reduction of lysosomal, inflammatory, neuroinflammation, and astrogliosis biomarkers (e.g., Cathepsin D, LAMP1, C1QB, GFAP, and MIF), stable plasma and CSF NfL levels over 12 months, a trend toward reduced ventricular enlargement measured by volumetric MRI as compared to a synthetic matched control group, and a predicted 47% delay in disease progression as measured by CDR® plus NACC FTLD-SB compared to a synthetic matched control group.

Similarly, treatment of FTD-C9orf72 patients with anti-Sortilin antibody S-60-15.1 [N33T] LALAPS resulted in a time-dependent and durable reduction of an astrogliosis biomarker (i.e., GFAP) in plasma and CSF; stable plasma and CSF NfL levels over 12 months; increases of PGRN levels in plasma and CSF beyond physiological levels, which is indicative of the potential for clinical benefit; and a predicted 54% delay in disease progression as measured by CDR® plus NACC FTLD-SB compared to a historical control cohort.

What is claimed is:

1. A method of treating and/or delaying the progression of frontotemporal dementia in an individual at risk for developing symptomatic frontotemporal dementia, comprising administering to the individual an anti-Sortilin antibody intravenously at a dose of about 60 mg/kg about once every four weeks, wherein the individual has an elevated serum neurofilament light chain level, and wherein the antibody comprises:
(i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33);
(iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32);
(vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33);
(vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or
(viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32).

2. A method of treating and/or delaying the progression of frontotemporal dementia in an individual at risk for developing symptomatic frontotemporal dementia, wherein the individual has a serum neurofilament light chain level of at least about 13.6 pg/mL or at least about 19.8 pg/mL, and further wherein the method comprises administering to the individual an anti-Sortilin antibody intravenously at a dose of about 60 mg/kg about once every four weeks, wherein the antibody comprises:
(i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33);
(iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32);
(vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33);
(vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or
(viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32).

3. The method of claim 2, further comprising assessing the serum neurofilament light chain level in the individual prior to administration of the anti-Sortilin antibody.

4. A method of treating and/or delaying the progression of frontotemporal dementia in an individual, comprising administering to the individual an anti-Sortilin antibody intravenously at a dose of about 60 mg/kg about once every four weeks, wherein the antibody comprises:
(i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33);
(iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32);
(vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33);
(vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or
(viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32); and
wherein administration of the anti-Sortilin antibody to the individual results in a reduction or delay of frontotemporal dementia disease progression of at least about 40% as compared to disease progression in a corresponding individual not treated with the anti-Sortilin antibody.

5. The method of claim 4, wherein frontotemporal dementia disease progression is assessed using the Clinical Dementia Rating Dementia Staging Instrument PLUS National Alzheimer's Disease Coordinating Center frontotemporal lobar degeneration Behavior and Language Domains Sum of Boxes (CDR® plus NACC FTLD-SB) assessment.

6. The method of any one of claims 1-5, wherein the heavy chain variable region comprises an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and the light chain variable region comprises an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32).

7. The method of any one of claims 1-5, wherein the heavy chain variable region comprises an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and the light chain variable region comprises an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32).

8. The method of any one of claims 1-5, wherein the antibody comprises:
a heavy chain variable region comprising the amino acid sequence of SEQ ID NO: 54, and a light chain variable region comprising the amino acid sequence of SEQ ID NO: 57;
a heavy chain variable region comprising the amino acid sequence of SEQ ID NO: 54, and a light chain variable region comprising the amino acid sequence of SEQ ID NO: 58;
a heavy chain variable region comprising the amino acid sequence of SEQ ID NO: 54, and a light chain variable region comprising the amino acid sequence of SEQ ID NO: 59;
a heavy chain variable region comprising the amino acid sequence of SEQ ID NO: 55, and a light chain variable region comprising the amino acid sequence of SEQ ID NO: 57;
a heavy chain variable region comprising the amino acid sequence of SEQ ID NO: 55, and a light chain variable region comprising the amino acid sequence of SEQ ID NO: 58;
a heavy chain variable region comprising the amino acid sequence of SEQ ID NO: 56, and a light chain variable region comprising the amino acid sequence of SEQ ID NO: 57;
a heavy chain variable region comprising the amino acid sequence of SEQ ID NO: 56, and a light chain variable region comprising the amino acid sequence of SEQ ID NO: 60;
a heavy chain variable region comprising the amino acid sequence of SEQ ID NO: 56, and a light chain variable region comprising the amino acid sequence of SEQ ID NO: 77;
a heavy chain variable region comprising the amino acid sequence of SEQ ID NO: 56, and a light chain variable region comprising the amino acid sequence of SEQ ID NO: 78;
a heavy chain variable region comprising the amino acid sequence of SEQ ID NO: 54, and a light chain variable region comprising the amino acid sequence of SEQ ID NO: 79; or
a heavy chain variable region comprising the amino acid sequence of SEQ ID NO: 56, and a light chain variable region comprising the amino acid sequence of SEQ ID NO: 80.

9. The method of any one of claims 1-5, wherein the antibody comprises:
(i) a heavy chain variable region comprising the amino acid sequence of SEQ ID NO: 56, and a light chain variable region comprising the amino acid sequence of SEQ ID NO: 57; or
(ii) a heavy chain variable region comprising the amino acid sequence of SEQ ID NO: 56, and a light chain variable region comprising the amino acid sequence of SEQ ID NO: 60.

10. The method of any one of claims 1-5, wherein the antibody comprises a heavy chain comprising the amino acid sequence of SEQ ID NO: 90 or SEQ ID NO: 91, and a light chain comprising the amino acid sequence of SEQ ID NO: 95.

11. The method of any one of claims 1-10, wherein the antibody has an IgG1 isotype and the Fc region comprises amino acid substitutions at positions L234A, L235A, and P331S, wherein the numbering of the residue position is according to EU numbering.

12. The method of any one of claims 1-11, wherein the individual is heterozygous for a mutation in the Progranulin gene (GRN).

13. The method of claim 12, wherein the GRN mutation is a loss-of-function mutation.

14. The method of claim 12 or claim 13, wherein the GRN mutation is causative of frontotemporal dementia.

15. The method of any one of claims 1-14, wherein the individual does not show symptoms of frontotemporal dementia prior to administration of the anti-Sortilin antibody.

16. The method of any one of claims 4-15, wherein the individual is at risk for developing symptomatic frontotemporal dementia prior to administration of the anti-Sortilin antibody.

17. The method of claim 16, wherein the individual has an elevated serum neurofilament light chain level prior to administration of the anti-Sortilin antibody.

18. The method of any one of claims 1, 6-15 and 17, wherein the elevated serum neurofilament light chain level comprises a serum neurofilament light chain level of at least about 13.6 pg/mL.

19. The method of any one of claims 1, 6-15 and 17, wherein the elevated serum neurofilament light chain level comprises a serum neurofilament light chain level of at least about 19.8 pg/mL.

20. The method of any one of claims 1-19, wherein the individual has a Clinical Dementia Rating Dementia Staging Instrument PLUS National Alzheimer's Disease Coordinating Center Frontotemporal Lobar Degeneration Behavior and Language Domains Sum of Boxes (CDR plus NACC FTLD-SB) score of 0.5 or less prior to administration of the anti-Sortilin antibody.

21. The method of any one of claims 4-14, wherein the individual has symptomatic frontotemporal dementia prior to administration of the anti-Sortilin antibody.

22. The method of any one of claims 4-14 and 21, wherein the individual has a CDR plus NACC FTLD-SB score greater than 0.5 prior to administration of the anti-Sortilin antibody.

23. The method of any one of claims 1-11, wherein the individual is heterozygous for a hexanucleotide repeat expansion C9orf72 mutation.

24. The method of claim 23, wherein the hexanucleotide repeat expansion C9orf72 mutation is causative of FTD.

25. The method of claim 23 or claim 24, wherein the individual has symptomatic frontotemporal dementia prior to administration of the anti-Sortilin antibody.

26. The method of any one of claims 4-14 and 21-25, wherein the individual has one or more symptoms required for a diagnosis of possible behavioral variant frontotemporal dementia (bvFTD) prior to administration of the anti-Sortilin antibody.

27. The method of claim 26, wherein the one or more symptoms are selected from the group consisting of: disinhibition, apathy or inertia, loss of sympathy or empathy, perseverative or compulsive behaviors, hyperorality, and dysexecutive neuropsychological profile.

28. The method of any one of claims 4-14 and 21-25, wherein the individual has a diagnosis of primary progressive aphasia (PPA) prior to administration of the anti-Sortilin antibody.

29. The method of any one of claims 1-28, wherein the individual has a Clinical Dementia Rating Dementia Staging Instrument PLUS National Alzheimer's Disease Coordinating Center frontotemporal lobar degeneration Behavior and Language Domains (CDR plus NACC FTLD) score of between 0 and 2 prior to administration of the anti-Sortilin antibody.

30. The method of claim 29, wherein the individual has a CDR plus NACC FTLD score of 0.5, 1, or 2.

31. The method of any one of claims 1-30, wherein the individual is treated for a treatment period of 96 weeks.

32. The method of claim 31, wherein administration of the anti-Sortilin antibody occurs on the first day of the treatment period and every four weeks thereafter.

33. The method of claim 31 or claim 32, wherein a total of 25 doses of the anti-Sortilin antibody are administered to the individual during the treatment period.

34. The method of any one of claims 31-33, further comprising continuing administration of the anti-Sortilin antibody to the individual once every four weeks after the end of the 96-week treatment period.

35. The method of claim 34, wherein administration of the anti-Sortilin antibody to the individual continues once every four weeks for up to 96 weeks.

36. The method of claim 34 or claim 35, wherein administration of the anti-Sortilin antibody to the individual continues once every four weeks for up to 25 doses.

37. The method of any one of claims 1-36, wherein the individual is a human adult.

38. The method of any one of claims 1-37, further comprising assessing the individual for the presence of one or more GRN mutations prior to administration of the anti-Sortilin antibody.

39. The method of any one of claims 1-38, further comprising assessing the individual for the presence of a hexanucleotide repeat expansion C9orf72 mutation prior to administration of the anti-Sortilin antibody.

40. The method of any one of claims 1-39, further comprising assessing the individual for the presence of an elevated level of neurofilament light chain prior to administration of the anti-Sortilin antibody to the individual, wherein the level of neurofilament light chain is assessed in a sample of serum obtained from the individual.

41. The method of any one of claims 1-40, further comprising performing one or more clinical outcome assessments on the individual before and after the individual has received one or more doses of the anti-Sortilin antibody, wherein the one or more clinical outcome assessments are selected from the group consisting of: CDR plus NACC FTLD, CDR plus NACC FTLD-SB, Clinical Global Impression-Severity (CGI-S), Clinical Global Impression-Improvement (CGI-I), Repeatable Battery for the Assessment of Neuropsychological Status (RBANS), European Quality of Life-5 Dimensions (EQ-5D), Zarit Burden Interview (ZBI), Resource Utilization in Dementia-Lite Version (RUD-Lite), Frontotemporal Dementia Rating Scale (FRS), and Winterlight Labs Speech Assessment (WLA).

42. The method of any one of claims 1-41, further comprising measuring the level of Progranulin protein (PGRN) in a sample of blood plasma obtained from the individual before and after the individual has received one or more doses of the anti-Sortilin antibody.

43. The method of any one of claims 1-42, further comprising measuring the level of neurofilament light chain in a sample of serum or plasma obtained from the individual before and after the individual has received one or more doses of the anti-Sortilin antibody.

44. The method of any one of claims 1-43, further comprising measuring the level of Progranulin protein (PGRN) in a sample of cerebrospinal fluid obtained from the individual before and after the individual has received one or more doses of the anti-Sortilin antibody.

45. The method of any one of claims 1-44, further comprising measuring the level of neurofilament light chain in a sample of cerebrospinal fluid obtained from the individual before and after the individual has received one or more doses of the anti-Sortilin antibody.

46. The method of any one of claims 1-45, further comprising measuring the level of one or more biomarkers of neurodegeneration in a sample of whole blood, plasma, or cerebrospinal fluid obtained from the individual before and after the individual has received one or more doses of the anti-Sortilin antibody.

47. The method of claim 46, wherein the one or more biomarkers of neurodegeneration comprise tau and phosphorylated tau.

48. The method of any one of claims 1-47, further comprising measuring the level of one or more biomarkers of lysosomal function in a sample of whole blood, plasma, or cerebrospinal fluid obtained from the individual before and after the individual has received one or more doses of the anti-Sortilin antibody.

49. The method of claim 48, wherein the one or more biomarkers of lysosomal function comprise one or more cathepsins.

50. The method of any one of claims 1-49, further comprising measuring the level of one or more biomarkers of glial activity in a sample of whole blood, plasma, or cerebrospinal fluid obtained from the individual before and after the individual has received one or more doses of the anti-Sortilin antibody.

51. The method of claim 50, wherein the one or more biomarkers of glial activity comprise YKL40 and IL-6.

52. The method of any one of claims 1-51, further comprising assessing global and regional brain volumes in the individual before and after the individual has received one or more doses of the anti-Sortilin antibody.

53. The method of any one of claims 1-52, further comprising assessing volume of white matter hyperintensities in the individual before and after the individual has received one or more doses of the anti-Sortilin antibody.

54. The method of any one of claims 1-53, further comprising assessing brain perfusion in the individual before and after the individual has received one or more doses of the anti-Sortilin antibody.

55. The method of any one of claims 1-54, further comprising assessing fractional anisotropy, mean diffusivity, axial diffusivity, and/or radial diffusivity in the individual before and after the individual has received one or more doses of the anti-Sortilin antibody.

56. The method of any one of claims 1-55, further comprising measuring the level of one or more biomarkers of astrogliosis in a sample of whole blood, plasma, or cerebrospinal fluid obtained from the individual before and after the individual has received one or more doses of the anti-Sortilin antibody.

57. The method of claim 56, wherein the one or more biomarkers of astrogliosis comprise glial fibrillary acidic protein (GFAP).

58. The method of any one of claims 1-57, further comprising measuring the level of one or more biomarkers of neuroinflammation in a sample of cerebrospinal fluid obtained from the individual before and after the individual has received one or more doses of the anti-Sortilin antibody.

59. The method of claim 58, wherein the one or more biomarkers of neuroinflammation comprise macrophage migration inhibitory factor (MIF).

60. The method of any one of claims 1-59, further comprising measuring the level of the anti-Sortilin antibody in a sample of blood or cerebrospinal fluid obtained from the individual before and after the individual has received one or more doses of the anti-Sortilin antibody.

61. A method of monitoring treatment of an individual being administered an anti-Sortilin antibody, comprising performing one or more clinical outcome assessments on the individual before and after the individual has received one or more doses of an anti-Sortilin antibody, wherein the one or more clinical outcome assessments are selected from the group consisting of: CDR plus NACC FTLD, CDR plus NACC FTLD-SB, CGI-S, CGI-I, RBANS, EQ-5D, ZBI, RUD-Lite, FRS, and WLA.

62. The method of claim 61, further comprising assessing the activity of the anti-Sortilin antibody in the individual based on a result of the one or more clinical outcome assessments.

63. The method of claim 62, wherein the anti-Sortilin antibody is determined to be active in the individual if a result of the one or more clinical outcome assessments improves after the individual has received one or more doses of the anti-Sortilin antibody compared to a corresponding result before the individual received one or more doses of the anti-Sortilin antibody.

64. A method of monitoring treatment of an individual being administered an anti-Sortilin antibody, comprising measuring the level of Progranulin protein (PGRN) in a sample obtained from the individual before and after the individual has received one or more doses of an anti-Sortilin antibody.

65. The method of claim 64, further comprising assessing the activity of the anti-Sortilin antibody in the individual based on the level of PGRN in a sample obtained from the individual.

66. The method of claim 65, wherein the anti-Sortilin antibody is determined to be active in the individual if the level of PGRN in a sample obtained after the individual has received one or more doses of the anti-Sortilin antibody is increased compared to the level of PGRN in a sample obtained before the individual received one or more doses of the anti-Sortilin antibody.

67. The method of any one of claims 64-66, wherein the sample is a blood plasma sample or a cerebrospinal fluid sample.

68. A method of monitoring treatment of an individual being administered an anti-Sortilin antibody, comprising measuring the level of neurofilament light chain in a sample obtained from the individual before and after the individual has received one or more doses of an anti-Sortilin antibody.

69. The method of claim 68, further comprising assessing the activity of the anti-Sortilin antibody in the individual based on the level of neurofilament light chain in a sample obtained from the individual.

70. The method of claim 69, wherein the anti-Sortilin antibody is determined to be active in the individual if the level of neurofilament light chain in a sample obtained after the individual has received one or more doses of the anti-Sortilin antibody is decreased compared to the level of neurofilament light chain in a sample obtained before the individual received one or more doses of the anti-Sortilin antibody.

71. The method of any one of claims 68-70, wherein the sample is a serum sample or a cerebrospinal fluid sample.

72. A method of monitoring treatment of an individual being administered an anti-Sortilin antibody, comprising measuring the level of one or more biomarkers of neurodegeneration, lysosomal function, astrogliosis, neuroinflammation, or glial activity in a sample obtained from the individual before and after the individual has received one or more doses of an anti-Sortilin antibody.

73. The method of claim 72, further comprising assessing the activity of the anti-Sortilin antibody in the individual based on the level of the one or more biomarkers of neurodegeneration, lysosomal function, astrogliosis, neuroinflammation, or glial activity in a sample obtained from the individual.

74. The method of claim 72 or claim 73, wherein the sample is a whole blood, plasma, or cerebrospinal fluid sample.

75. The method of any one of claims 72-74, wherein the one or more biomarkers of neurodegeneration comprise tau and phosphorylated tau.

76. The method of any one of claims 72-74, wherein the one or more biomarkers of lysosomal function comprise one or more cathepsins.

77. The method of any one of claims 72-74, wherein the one or more biomarkers of glial activity comprise YKL40 and IL-6.

78. The method of any one of claims 72-74, wherein the one or more biomarkers of astrogliosis comprise GFAP.

79. The method of any one of claims 72-74, wherein the one or more biomarkers of neuroinflammation comprise macrophage migration inhibitory factor (MIF).

80. A method of monitoring treatment of an individual being administered an anti-Sortilin antibody, comprising assessing global and regional brain volumes, volume of white matter hyperintensities, brain perfusion, fractional anisotropy, mean diffusivity, axial diffusivity, and/or radial diffusivity in the individual before and after the individual has received one or more doses of an anti-Sortilin antibody.

81. The method of claim 80, further comprising assessing the activity of the anti-Sortilin antibody in the individual based on global and regional brain volumes, volume of white matter hyperintensities, brain perfusion, fractional anisotropy, mean diffusivity, axial diffusivity, and/or radial diffusivity.

82. An anti-sortilin antibody at a dose of about 60 mg/kg intravenously about once every four weeks for use in a method of treating and/or delaying the progression of a disease or injury in an individual, wherein the antibody comprises:
(i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33);
(iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32);
(vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33);
(vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or
(viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32).

83. An anti-sortilin antibody at a dose of about 60 mg/kg intravenously about once every four weeks for use in a method of treating and/or delaying the progression of frontotemporal dementia in an individual at risk for developing symptomatic frontotemporal dementia, wherein the individual has an elevated serum neurofilament light chain level, and wherein the antibody comprises:
(i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33);
(iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32);
(vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33);
(vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or
(viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32).

84. A use of an anti-sortilin antibody at a dose of about 60 mg/kg intravenously about once every four weeks in the manufacture of a medicament for treating and/or delaying the progression of a disease or injury in an individual, wherein the antibody comprises:
(i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33);
(iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32);
(vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33);
(vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or
(viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32).

85. A use of an anti-sortilin antibody at a dose of about 60 mg/kg intravenously about once every four weeks in the manufacture of a medicament for treating and/or delaying the progression of frontotemporal dementia in an individual at risk for developing symptomatic frontotemporal dementia, wherein the individual has an elevated serum neurofilament light chain level, and wherein the antibody comprises:
(i) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(ii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRVS (SEQ ID NO: 30), and an HVR-L3 comprising the amino acid sequence MQQQETPLT (SEQ ID NO: 33);
(iii) a heavy chain variable region comprising an HVR-H1 comprising the amino acid sequence YSISSGYYWG (SEQ ID NO: 1), an HVR-H2 comprising the amino acid sequence TIYHSGSTYYNPSLES (SEQ ID NO: 3), and an HVR-H3 comprising the amino acid sequence ARQGSIQQGYYGMDV (SEQ ID NO: 5); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(iv) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQEAPLT (SEQ ID NO: 32);
(v) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSTGYNYLD (SEQ ID NO: 9), an HVR-L2 comprising the amino acid sequence LGSNRAS (SEQ ID NO: 29), and an HVR-L3 comprising the amino acid sequence MQQQEAPLT (SEQ ID NO: 32);
(vi) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQSLLRSNGYNYLD (SEQ ID NO: 8), an HVR-L2 comprising the amino acid sequence LGSNRAS (SEQ ID NO: 29), and an HVR-L3 comprising the amino acid sequence MQQQETPLT (SEQ ID NO: 33);
(vii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIQQGYYGMDV (SEQ ID NO: 5); and a light chain variable region comprising an HVR-L1 comprising the amino acid sequence RSSQSLLHSNGYNYLD (SEQ ID NO: 26), an HVR-L2 comprising the amino acid sequence LGSNRAS (SEQ ID NO: 29), and an HVR-L3 comprising the amino acid sequence MQQQETPLT (SEQ ID NO: 33); or
(viii) a heavy chain variable region comprising an HVR-H1 comprising the amino acid sequence YSISSGYYWG (SEQ ID NO: 1), an HVR-H2 comprising the amino acid sequence TIYHSGSTYYNPSLKS (SEQ ID NO: 2), and an HVR-H3 comprising the amino acid sequence ARQGSIKQGYYGMDV (SEQ ID NO: 6); and a light chain variable region comprising an HVR-L1 comprising the amino acid sequence RSSQGLLRSNGYNYLD (SEQ ID NO: 27), an HVR-L2 comprising the amino acid sequence LGSNRAS (SEQ ID NO: 29), and an HVR-L3 comprising the amino acid sequence MQQQEAPLT (SEQ ID NO: 32).
